(19) United States
(12) Patent Application Publication
(10) Pub. No.: US 2008/0200448 A1
(43) Pub. Date: Aug. 21, 2008

(54) New Pyridine Analogues VIII 518

(75) Inventors: Thomas Antonsson, Molndal (SE); Peter Bach, Molndal (SE); Kay Brickmann, Molndal (SE); Ruth Bylund, Molndal (SE); Fabrizio Giordanetto, Molndal (SE); Johan Johansson, Molndal (SE); Fredrik Zetterberg, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 11/972,876

(22) Filed: Jan. 11, 2008

Related U.S. Application Data
(60) Provisional application No. 60/949,687, filed on Jul. 13, 2007.
(60) Provisional application No. 60/949,680, filed on Jul. 13, 2007.
(60) Provisional application No. 60/884,660, filed on Jan. 12, 2007.

(51) Int. Cl. A61K 31/4427; C07D 401/04; A61K 31/4439; A61K 31/4545; A61P 7/02

(52) U.S. Cl. 514/210.18; 546/208; 546/276.4; 546/268.1; 514/343; 514/318

(57) ABSTRACT

The present invention relates to certain new pyridin analogues of Formula (I)

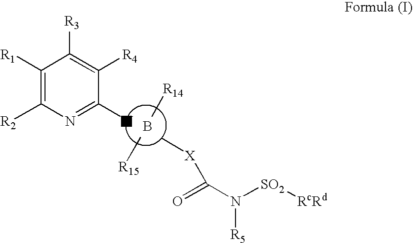

to processes for preparing such compounds, to their utility as P2Y₁₂ inhibitors and as anti-trombotic agents etc, their use as medicaments in cardiovascular diseases as well as pharmaceutical compositions containing them.

### FIELD OF THE INVENTION

The present invention provides novel pyridine compounds, their use as medicaments, compositions containing them and processes for their preparation.

### BACKGROUND OF THE INVENTION

Platelet adhesion and aggregation are initiating events in arterial thrombosis. Although the process of platelet adhesion to the sub-endothelial surface may have an important role to play in the repair of damaged vessel walls, the platelet aggregation that this initiates can precipitate acute thrombotic occlusion of vital vascular beds, leading to events with high morbidity such as myocardial infarction and unstable angina. The success of interventions used to prevent or alleviate these conditions, such as thrombolysis and angioplasty is also compromised by platelet mediated occlusion or re-occlusion.

Haemostasis is controlled via a tight balance between platelet aggregation, coagulation and fibrinolysis. Thrombus formation under pathological conditions, like e.g. arteriosclerotic plaque rupture, is firstly initiated by platelet adhesion, activation and aggregation. This results not only in the formation of a platelet plug but also in the exposure of negatively charged phospholipids on the outer platelet membrane promoting blood coagulation. Inhibition of the build-up of the initial platelet plug would be expected to reduce thrombus formation and reduce the number of cardiovascular events as was demonstrated by the anti-thrombotic effect of e.g. Aspirin (BMJ 1994; 308: 81-106 Antiplatelet Trialists' Collaboration. Collaborative overview of randomised trials of antiplatelet therapy, I: Prevention of death, myocardial infarction, and stroke by prolonged antiplatelet therapy in various categories of patients).

Platelet activation/aggregation can be induced by a variety of different agonists. However, distinct intracellular signalling pathways have to be activated to obtain full platelet aggregation, mediated via G-proteins G_{q}, G_{12/13} and Gᵢ (Platelets, A D Michelson ed., Elsevier Science 2002, ISBN 0-12-493951-1; 197-213: D Woulfe, et al. Signal transduction during the initiation, extension, and perpetuation of platelet plug formation) In platelets, the G-protein coupled receptor P2Y₁₂ (previously also known as the platelet P_{2T}, P2T_{ac}, or P2Y_{cyc} receptor) signals via Gi, resulting in a lowering of intra-cellular cAMP and full aggregation (Nature 2001; 409: 202-207 G Hollopeter, et al. Identification of the platelet ADP receptor targeted by antithrombotic drugs). Released ADP from dense-granules will positively feedback on the P2Y12 receptor to allow full aggregation. WO 2002/098856 and WO 2004/052366 describe piperazino-carbonylmethylaminocarbonyl-naphtyl or -quinolyl derivatives as ADP receptor antagonist.

Clinical evidence for the key-role of the ADP-P2Y₁₂ feedback mechanism is provided by the clinical use of clopidogrel, an thienopyridine prodrug which active metabolite selectively and irreversibly binds to the P2Y₁₂ receptor, that has shown in several clinical trials to be effective in reducing the risk for cardiovascular events in patients at risk (Lancet 1996; 348: 1329-39: CAPRIE Steering committee, A randomised, blinded, trial of clopidogrel versus aspirin in patients at risk of ischaemic events (CAPRIE); N Engl J Med 2001; 345 (7): 494-502): The Clopidogrel in Unstable Angina to prevent Recurrent Events Trial Investigators. Effects of clopidogrel in addition to aspirin in patients with acute coronary syndromes without ST-segment elevation). In these studies, the clinical benefit with a reduced bleeding risk as compared to thienopyridines (Sem Thromb Haemostas 2005; 31 (2): 195-204 J J J van Giezen & R G Humphries. Preclinical and clinical studies with selective reversible direct P2Y₁₂ antagonists. WO 2005/000281 describes a series of pyrazolidine-3,5-dione derivatives and WO 2006/1147742 describes a series of phenyl-pyrimidine derivatives which both series have been described as P2Y12 antagonists for the potential treatment of thrombosis. WO 2006/073361 discloses some P2Y12 antagonists for the potential treatment of thrombosis.

It is an object of the present invention to provide improved potent, reversible and selective P2Y₁₂-antagonists as anti-trombotic agents.

### SUMMARY OF THE INVENTION

We have now surprisingly found that certain pyridine compounds of Formula (I) or a pharmaceutically acceptable salt thereof are reversible and selective P2Y₁₂ antagonists, hereinafter referred to as the compounds of the invention. The compounds of the invention unexpectedly exhibit beneficial properties that render them particularly suitable for use in the treatment of diseases/conditions as described below (See p. 94-95). Examples of such beneficial properties are high potency, high selectivity, and an advantageous therapeutic window.

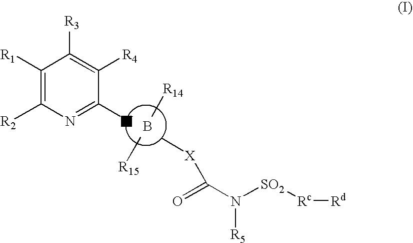

### DETAILED DESCRIPTION OF THE INVENTION

According to the present invention there is provided a novel compound of formula (I) or a pharmaceutically acceptable salt thereof:

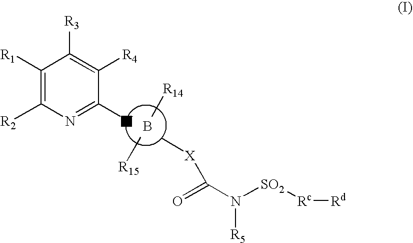

wherein

R₁ represents R₆OC(O) or R₁₆SC(O);

preferably R₁ represents R₆OC(O);

R₂ represents substituted (C₁-C₁₂)alkyl optionally interrupted by sulphur, substituted (C₁-C₁₂)alkoxy or substituted (C₁-C₁₂)alkylthio, wherein any one of these groups is substituted by one or more of azido, carboxy, cyano, (C₁-C₁₂)alkylcarbonyloxy, hydroxy(C₁-C₁₂)alkylcarbonyloxy, arylcarbonyloxy, heterocyclylcarbonyloxy, (C₁-C₁₂)alkyloxycarbonyl, (C₁-C₁₂)alkyl(C(S)), (C₁-C₁₂)alkyl(S(CO)), (C₁-C₁₂)alkylthio, hydroxy(C₁-C₁₂)alkylthio, (C₁-C₁₂)alkylsulfinyl, (C₁-C₁₂)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₁₂)alkylthio, aryl(C₁-C₁₂)alkylsulfinyl, aryl(C₁-C₁₂)alkylsulfonyl, heterocyclyl(C₁-C₁₂)alkyl thio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, (C₁-C₁₂)alkylcarbonyl, arylcarbonyl, heterocyclylcarbonyl, aryl(C₁-C₁₂)alkylcarbonyl, heterocyclyl(C₁-C₁₂)alkylcarbonyl or of a group of formula NR^{a(2)}R^{b(2)} or —(CO)NR^{a(2)}R^{b(2)}, in which R^{a(2)} and R^{b(2)} each and independently represent H, (C₁-C₁₂)alkyl, (C₁-C₁₂)alkylcarbonyl or R^{a(2)} and R^{b(2)} together with the nitrogen atom represent piperidine, pyrrolidine, azetidine or aziridine or any one of the groups

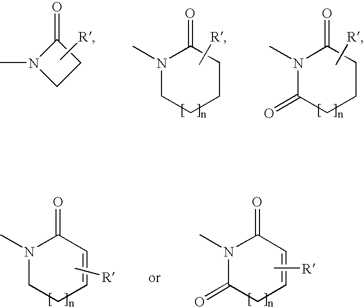

wherein n is an integer chosen from 0, 1 and 2, and R′ is H, CN, OH, a halogen (F, Cl, Br, I) atom, or one of the groups (C₁-C₈)alkyl, aryl, (C₁-C₈)alkoxy, (C₁-C₈)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen (F, Cl, Br, I) atoms;
Further R₂ represents substituted (C₁-C₁₂)alkoxy or substituted (C₁-C₁₂)alkylthio, wherein any one of these groups is substituted by one or more of any one of OH, aryl, (C₃-C₆)cycloalkyl or heterocyclyl; Further R₂ represents (C₁-C₁₂)alkylthio, substituted by one or more halogen (F, Cl, I, Br) atom(s); Further R₂ represents (C₁-C₁₂)alkylcarbonyloxy, aryl carbonyloxy, heterocyclylcarbonyloxy of which any one optionally is substituted by one or more of any one of the following groups or atoms; azido, cyano, halogen (F, Cl, Br, I) atom(s), OH, (C₁-C₁₂)alkoxy, (C₁-C₁₂)alkylthio, (C₁-C₁₂)alkylsulfinyl, (C₁-C₁₂)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₁₂)alkylthio, aryl(C₁-C₁₂)alkylsulfinyl, aryl(C₁-C₁₂)alkylsulfonyl, heterocyclyl(C₁-C₁₂)alkyl thio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, (C₁-C₁₂)alkylcarbonyl, arylcarbonyl, heterocyclylcarbonyl, aryl(C₁-C₁₂)alkylcarbonyl and heterocyclyl(C₁-C₁₂)alkylcarbonyl; Further R₂ represents unsubstituted (C₁-C₁₂)alkyl with the proviso that at the same time R₅ represents carboxy(C₁-C₁₂)alkyl; Further R₂ represents a group of formula ((R^{a(2)})N(R^{b(2)}))(CO)—, in which R^{a(2)} and R^{b(2)} each and independently represent H, (C₁-C₁₂)alkyl, aryl, aryl(C₁-C₁₂)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine;

R₃ represents H, CN, NO₂, halogen (F, Cl, Br, I), (C₁-C₁₂)alkyl optionally interrupted by oxygen and/or optionally substituted by OH, aryl, cycloalkyl, heterocyclyl or one or more halogen (F, Cl, Br, I) atoms; further R₃ represents (C₁-C₁₂)alkoxy optionally substituted by one or more halogen (F, Cl, Br, I) atoms; further R₃ represents (C₃-C₆)cycloalkyl, hydroxy(C₁-C₁₂)alkyl, (C₁-C₁₂)alkylC(O), (C₁-C₁₂)alkylthioC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(3)}R^{b(3)} in which R^{a(3)} and R^{b(3)} independently represent H, (C₁-C₁₂)alkyl, (C₁-C₁₂)alkylC(O) or R^{a(3)} and R^{b(3)} together with the nitrogen atom represent piperidine, pyrrolidine, azetidine or aziridine;

R₄ represents H, CN, NO₂, halogen (F, Cl, Br, I), (C₁-C₁₂)alkyl optionally interrupted by oxygen and/or optionally substituted by OH, COOH, (C₁-C₆)alkoxycarbonyl, aryl, cycloalkyl, heterocyclyl or one or more halogen (F, Cl, Br, I) atoms; further R₄ represents (C₃-C₆)cycloalkyl, hydroxy(C₁-C₁₂)alkyl, (C₁-C₁₂)alkylC(O), (C₁-C₁₂)alkylcycloalkyl, (C₁-C₁₂)alkoxy wherein the alkoxy group may optionally be substituted by one or more halogen (F, Cl, Br, I) atoms, OH and/or COOH and/or (C₁-C₆)alkoxycarbonyl; further R₄ represents (C₁-C₁₂)alkylthioC(O), (C₁-C₁₂)alkylC(S), (C₁-C₁₂)alkoxyC(O), (C₃-C₆)cycloalkoxy, aryl, aryl(C₁-C₁₂)alkoxy, aryl(C₁-C₁₂)alkyl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkoxy, (C₃-C₆)cycloalkyl(C₁-C₁₂)alkylsulfinyl, (C₃-C₆)cycloalkyl(C₁-C₁₂)alkylsulfonyl or a group of formula NR^{a(4)}R^{b(4)} in which R^{a(4)} and R^{b(4)} independently represent H, (C₁-C₁₂)alkyl, (C₁-C₁₂)alkylC(O) or R^{a(4)} and R^{b(4)} together with the nitrogen atom represent piperidine, pyrrolidine, azetidine or aziridine;

R₅ represents H or (C₁-C₁₂)alkyl or carboxy(C₁-C₆)alkyl; with the proviso that when R₂ is unsubstituted (C₁-C₁₂)alkyl, R₅ represents carboxy(C₁-C₁₂)alkyl;

R₆ represents (C₁-C₁₂)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms; further R₆ represents (C₂-C₁₂)alkenyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms; further R₆ represents (C₂-C₁₂)alkynyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl, hydroxy(C₂-C₁₂)alkyl, aryl or heterocyclyl;

R₁₄ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{e}; wherein R^{e} represents aryl, cycloalkyl, heterocyclyl or (C₁-C₁₂)alkyl optionally substituted by one or more of halogen (F, Cl, Br, I) atoms, OH, aryl, cycloalkyl and heterocyclyl; further R₁₄ represents aryl, aryl(C₁-C₁₂)alkoxy, aryl(C₁-C₁₂)alkyl, (C₃-C₆)cycloalkyl(C₁-C₁₂)alkoxy, heterocyclyl, a halogen (F, Cl, Br, I) atom, (C₃-C₆)cycloalkyl, hydroxy(C₁-C₁₂)alkyl, (C₁-C₁₂)alkoxy, (C₃-C₆)cycloalkoxy, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkoxy, (C₃-C₆)cycloalkyl(C₁-C₁₂)alkylsulfinyl or (C₃-C₆)cycloalkyl(C₁-C₁₂)alkylsulfonyl, a group of formula NR^{a(14)}R^{b(14)} in which R^{a(14)} and R^{b(14)} independently represent H, (C₁-C₁₂)alkyl, (C₁-C₁₂)alkylC(O), (C₁-C₁₂)alkoxyC(O) or R^{a(14)} and R^{b(14)} together with the nitrogen atom represent piperidine, pyrrolidine, azetidine or aziridine;

R₁₅ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{e}; wherein R^{e} represents aryl, cycloalkyl, heterocyclyl or (C₁-C₁₂)alkyl optionally substituted by one or more of halogen (F, Cl, Br, I) atoms, OH, aryl, cycloalkyl and heterocyclyl; further R₁₅ represents aryl, aryl(C₁-C₁₂)alkoxy, aryl(C₁-C₁₂)alkyl, (C₃-C₆)cycloalkyl(C₁-C₁₂)alkoxy, heterocyclyl, a halogen (F, Cl, Br, I) atom, (C₃-C₆)cycloalkyl, hydroxy(C₁-C₁₂)alkyl, (C₁-C₁₂)alkoxy, (C₃-C₆)cycloalkoxy, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkoxy, (C₃-C₆)cycloalkyl(C₁-C₁₂)alkylsulfinyl, (C₃-C₆)cycloalkyl(C₁-C₁₂)alkylsulfonyl or a group of formula NR^{a(15)}R^{b(15)} in which R^{a(15)} and R^{b(15)} independently represent H, (C₁-C₁₂)alkyl, (C₁-C₁₂)alkylC(O)), (C₁-C₁₂)alkoxyC(O) or R^{a(15)} and R^{b(15)} together with the nitrogen atom represent piperidine, pyrrolidine, azetidine or aziridine;

R₁₆ represents (C₁-C₁₂)alkyl optionally interrupted by oxygen (with the proviso that any such oxygen must be at least 2 carbon atoms away from the thioester-sulfur connecting the R₁₆ group) and/or optionally substituted by OH, aryl, cycloalkyl, heterocyclyl or one or more halogen (F, Cl, Br, I) atoms; further R₁₆ represents (C₃-C₆)cycloalkyl, hydroxy(C₂-C₁₂)alkyl, (C₁-C₁₂)alkoxy, (C₃-C₆)cycloalkoxy, aryl or heterocyclyl;

R^{c} is a single bond or represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; Further R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)alkylene group or (C₁-C₄)oxoalkylene group with any substituents according to above;

R₁₉ represents H or (C₁-C₄)alkyl;

R^{d} represents (C₁-C₁₂)alkyl, (C₃-C₈)cycloalkyl, aryl or heterocyclyl, and any one of these groups optionally substituted with one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, halogen substituted (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, tri(C₁-C₄)alkylsilyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine;

X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; further X may represent a group (—CH₂-)n wherein n=2-6, which optionally is unsaturated and/or substituted by one or more substituent chosen among halogen, hydroxyl or (C₁-C₆)alkyl;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections).

Preferred values of each variable group or specific embodiments of variable groups or terms are as follows. Such values or embodiments may be used where appropriate with any of the values, definitions, claims, aspects, embodiments or embodiments of the invention defined hereinbefore or hereinafter. In particular, each may be used as an individual limitation on the broadest definition of formula (I).
For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the particular definitions for that group.

It will be understood that when formula I compounds contain a chiral centre, the compounds of the invention may exist in, and be isolated in, optically active or racemic form. The invention includes any optically active or racemic form of a compound of formula I which act as P2Y₁₂ receptor antagonists. The synthesis of optically active forms may be carried out by standard techniques of organic chemistry well known in the art, for example by, resolution of a racemic mixture, by chiral chromatography, synthesis from optically active starting materials or by asymmetric synthesis.

It will also be understood that the compounds of the formula I may exhibit the phenomenon of tautomerism, the present invention includes any tautomeric form of a compound of formula I which is a P2Y₁₂ receptor antagonist.

It will also be understood that in so far as compounds of the present invention exist as solvates, and in particular hydrates, these are included as part of the present invention.

It is also to be understood that generic terms such as “alkyl” include both the straight chain and branched chain groups such as butyl and tert-butyl. However, when a specific term such as “butyl” is used, it is specific for the straight chain or “normal” butyl group, branched chain isomers such as “t-butyl” being referred to specifically when intended.
In one embodiment alkyl is, unless otherwise specified, unsubstituted, with the proviso that when R₂ is the unsubstituted alkyl, then R₅ represents carboxy(C₁-C₁₂)alkyl.
In another embodiment alkyl is unsubstituted or substituted by one or more of the following groups, CN, (C₁-C₁₂)alkoxyC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine, with the proviso that when R₂ is the unsubstituted alkyl, then R₅ represents carboxy(C₁-C₁₂)alkyl.

In a further embodiment alkyl is unsubstituted or substituted by one or more of the following groups, CN, NO₂, (C₁-C₁₂)alkoxyC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine, or any one of the groups

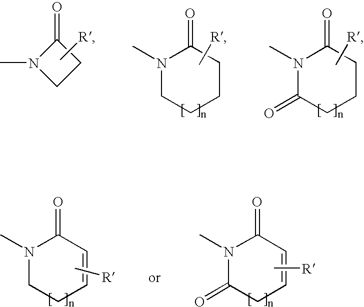

wherein n is an integer chosen from 0, 1 and 2, and R′ is H, CN, OH, a halogen (F, Cl, Br, I) atom, or one of the groups (C₁-C₈)alkyl, aryl, (C₁-C₈)alkoxy, (C₁-C₈)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen (F, Cl, Br, I) atoms, with the proviso that when R₂ is the unsubstituted alkyl, then R₅ represents carboxy(C₁-C₁₂)alkyl.

The term “alkyl” includes both linear or branched chain groups.

In R^{d} any “alkyl” generally is optionally substituted with one or more halogens (F, Cl, Br or I). E.g. (C₁-C₁₂)alkylthio may be embodiefied by —SCF₃.

The term “cycloalkyl” generally denotes a substituted or unsubstituted (C₃-C₆), unless other chain length specified, cyclic hydrocarbon.

In one embodiment cycloalkyl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

The term “alkoxy” includes both linear or branched chain groups.

The term aryl denotes a substituted or unsubstituted (C₆-C₁₄) aromatic hydrocarbon and includes, but is not limited to, phenyl, naphthyl, tetrahydronaphtyl, indenyl, indanyl, antracenyl, fenantrenyl, and fluorenyl.

In one embodiment aryl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

The term “heterocyclyl” denotes a substituted or unsubstituted, 4- to 10-membered monocyclic or multicyclic ring system in which one or more of the atoms in the ring or rings is an element other than carbon, for example nitrogen, oxygen or sulfur, especially 4-, 5- or 6-membered aromatic or aliphatic hetorocyclic groups, and includes, but is not limited to azetidine, oxetan, furan, thiophene, pyrrole, pyrroline, pyrrolidine, 2-oxopyrrolidine, 2,5-dioxopyrrolidine, dioxolane, oxathiolane, oxazolane, oxazole, thiazole, imidazole, imidazoline, imidazolidine, pyrazole, pyrazoline, pyrazolidine, isothiazole, oxadiazole, furazan, triazole, thiadiazole, pyran, tetrahydro-2H-pyran, pyridine as well as pyridine-N-oxide, piperidine, 2-oxopiperidine, dioxane, morpholine, dithiane, oxathiane, thiomorpholine, pyridazine, pyrimidine, pyrazine, piperazine, triazine, thiadiazine, dithiazine, azaindole, azaindoline, indole, indoline, naphthyridine, benzoxadiazole, dihydrobenzodioxin, benzothiophene, benzothiadiazole, imidazothiazole, 2,3-dihydrobenzofuran, isoxazole, 3-benzisoxazole, 1,2-benzisoxazole, dihydropyrazole groups, and shall be understood to include all isomers of the above identified groups. For the above groups, e.g. azetidinyl, the term “azetidinyl” as well as “azetidinylene”, etc., shall be understood to include all possible regio isomers. It is further to be understood that the term heterocyclyl may be embodified by one selection among the given possible embodiments for a variable and embodified by another (or the same) selection for another variable, e.g. R₄ when selected as heterocyclyl may be a furan, when R^{d} (also when selected as heterocyclyl) may be a pyrrole.

In one embodiment of the invention the term “heterocyclyl” denotes a substituted or unsubstituted, 4- to 10-membered monocyclic or multicyclic ring system in which one or more of the atoms in the ring or rings is an element other than carbon, for example nitrogen, oxygen or sulfur, especially 4-, 5- or 6-membered aromatic or aliphatic hetorocyclic groups, and includes, but is not limited to azetidine, furan, thiophene, pyrrole, pyrroline, pyrrolidine, dioxolane, oxathiolane, oxazolane, oxazole, thiazole, imidazole, imidazoline, imidazolidine, pyrazole, pyrazoline, pyrazolidine, isothiazole, oxadiazole, furazan, triazole, thiadiazole, pyran, pyridine as well as pyridine-N-oxide, piperidine, dioxane, morpholine, dithiane, oxathiane, thiomorpholine, pyridazine, pyrimidine, pyrazine, piperazine, triazine, thiadiazine, dithiazine, azaindole, azaindoline, indole, indoline, naphthyridine, benzoxadiazole, dihydrobenzodioxin, benzothiophene, benzothiadiazole, imidazothiazole, 2,3-dihydrobenzofuran, isoxazole, 3-benzisoxazole, 1,2-benzisoxazole, dihydropyrazole groups, and shall be understood to include all isomers of the above identified groups. For the above groups, e.g. azetidinyl, the term “azetidinyl” as well as “azetidinylene”, etc., shall be understood to include all possible regio isomers. It is further to be understood that the term heterocyclyl may be embodified by one selection among the given possible embodiments for a variable and embodified by another (or the same) selection for another variable, e.g. R₄ when selected as heterocyclyl may be a furan, when R^{d} (also when selected as heterocyclyl) may be a pyrrole.

In one embodiment heterocyclyl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

In another embodiment of the invention the heterocyclyl group comprises an aromatic 5-membered or 6-membered heterocyclic ring containing one, two or three heteroatoms selected from nitrogen, oxygen and sulphur, and an aromatic 5-membered or 6-membered heterocyclic ring containing one, two or three heteroatoms selected from nitrogen, oxygen and sulphur which is fused to a benzene ring;

In an alternative embodiment of the invention the heterocyclyl group is a non-aromatic 5-membered or 6-membered heterocyclic ring containing one, two or three heteroatoms selected from nitrogen, oxygen and sulphur, fused to a benzene ring.

In a further embodiment of the invention the heterocyclyl group is a group chosen among furyl, pyrrolyl, thienyl, pyridyl, N-oxido-pyridyl, pyrazinyl, pyrimidinyl, pyridazinyl, imidazolyl, oxazolyl, isooxazolyl, thiazolyl, isothiazolyl, oxadiazolyl, 1,2,3-triazolyl, 1,2,4-triazolyl, benzfuranyl, quinolyl, isoquinolyl, benzimidazolyl, indolyl, benzdihydrofuranyl, benzodioxolyl (such as 1,3-benzodioxolyl), benzoxadiazole, dihydrobenzodioxin, benzothiophene, benzothiadiazole, imidazothiazole, 2,3-dihydrobenzofuran, isoxazole, dihydropyrazole and benzdioxanyl (such as 1,4-benzdioxanyl). More particular values include, for example, furyl, pyrrolyl, thienyl, pyridyl, pyrazinyl, pyrimidinyl, pyridazinyl, benzoxadiazole, dihydrobenzodioxin, benzothiophene, benzothiadiazole, imidazothiazole, 2,3-dihydrobenzofuran, isoxazole, 1,2-benzisoxazole, dihydropyrazole and benzdioxanyl (such as 1,4-benzdioxanyl).

In an even further embodiment of the invention the heterocyclyl group is a group chosen among furyl, pyrrolyl, thienyl, pyridyl, N-oxido-pyridyl, pyrazinyl, pyrimidinyl, pyridazinyl, benzoxadiazole, dihydrobenzodioxin, benzothiophene, benzothiadiazole, imidazothiazole, 2,3-dihydrobenzofuran, isoxazole, 1,2-benzisoxazole or dihydropyrazole.

In one embodiment of the invention R₁ represents R₆OC(O).
In another embodiment of the invention R₁ represents R₁₆SC(O).

In a further embodiment of the invention R₁ is selected among R₆OC(O) and R₁₆SC(O) wherein R₆ can be methyl, ethyl, 2-hydroxyethyl, 2,2,2-trifluoroethyl, isopropyl, cyclo-propyl, iso-butyl, n-butyl, cyclo-butyl, n-propyl, tertbutyl, cyclo-pentyl, 2,2-dimethylpropyl, benzyl and 4-fluorobenzyl and wherein R₁₆ is ethyl.

In one embodiment R₂ represents substituted (C₁-C₁₂)alkyl optionally interrupted by sulphur, substituted (C₁-C₁₂)alkoxy or substituted (C₁-C₁₂)alkylthio, wherein any one of these groups is substituted by one or more of azido, carboxy, cyano, (C₁-C₁₂)alkylcarbonyloxy, hydroxy(C₁-C₁₂)alkylcarbonyloxy, arylcarbonyloxy, heterocyclylcarbonyloxy, (C₁-C₁₂)alkyloxycarbonyl, (C₁-C₁₂)alkyl(C(S)), (C₁-C₁₂)alkyl(S(CO)), (C₁-C₁₂)alkylthio, hydroxy(C₁-C₁₂)alkylthio, (C₁-C₁₂)alkylsulfinyl, (C₁-C₁₂)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₁₂)alkylthio, aryl(C₁-C₁₂)alkylsulfinyl, aryl(C₁-C₁₂)alkylsulfonyl, heterocyclyl(C₁-C₁₂)alkyl thio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, (C₁-C₁₂)alkylcarbonyl, arylcarbonyl, heterocyclylcarbonyl, aryl(C₁-C₁₂)alkylcarbonyl, heterocyclyl(C₁-C₁₂)alkylcarbonyl or of a group of formula NR^{a(2)}R^{b(2)} or —(CO)NR^{a(2)}R^{b(2)}, in which R^{a(2)} and R^{b(2)} each and independently represent H, (C₁-C₁₂)alkyl, (C₁-C₁₂)alkylcarbonyl or R^{a(2)} and R^{b(2)} together with the nitrogen atom represent piperidine, pyrrolidine, azetidine or aziridine or any one of the groups

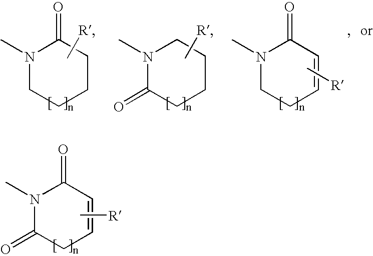

wherein n is an integer chosen from 0, 1 and 2, and R′ is H, CN, OH, a halogen (F, Cl, Br, I) atom, or one of the groups (C₁-C₈)alkyl, aryl, (C₁-C₈)alkoxy, (C₁-C₈)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen (F, Cl, Br, I) atoms;
Further R₂ represents substituted (C₁-C₁₂)alkoxy or substituted (C₁-C₁₂)alkylthio, wherein any one of these groups is substituted by one or more of any one of OH, aryl, (C₃-C₆)cycloalkyl or heterocyclyl; Further R₂ represents (C₁-C₁₂)alkylthio, substituted by one or more halogen (F, Cl, I, Br) atom(s); Further R₂ represents (C₁-C₁₂)alkylcarbonyloxy, aryl carbonyloxy, heterocyclylcarbonyloxy of which any one optionally is substituted by one or more of any one of the following groups or atoms; azido, cyano, halogen (F, Cl, Br, I) atom(s), OH, (C₁-C₁₂)alkoxy, (C₁-C₁₂)alkylthio, (C₁-C₁₂)alkylsulfinyl, (C₁-C₁₂)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₁₂)alkylthio, aryl(C₁-C₁₂)alkylsulfinyl, aryl(C₁-C₁₂)alkylsulfonyl, heterocyclyl(C₁-C₁₂)alkyl thio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, (C₁-C₁₂)alkylcarbonyl, arylcarbonyl, heterocyclylcarbonyl, aryl(C₁-C₁₂)alkylcarbonyl and heterocyclyl(C₁-C₁₂)alkylcarbonyl; Further R₂ represents unsubstituted (C₁-C₁₂)alkyl with the proviso that at the same time R₅ represents carboxy(C₁-C₁₂)alkyl; Further R₂ represents a group of formula ((R^{a(2)})N(R^{b(2)}))(CO)—, in which R^{a(2)} and R^{b(2)} each and independently represent H, (C₁-C₁₂)alkyl, aryl, aryl(C₁-C₁₂)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine;

In another embodiment of the invention R₂ represents substituted (C₁-C₁₂)alkyl optionally interrupted by sulphur, substituted (C₁-C₁₂)alkoxy or substituted (C₁-C₁₂)alkylthio, wherein any one of these groups is substituted by one or more of azido, carboxy, cyano, (C₁-C₁₂)alkylcarbonyloxy, hydroxy(C₁-C₁₂)alkylcarbonyloxy, arylcarbonyloxy, heterocyclylcarbonyloxy, (C₁-C₁₂)alkyloxycarbonyl, (C₁-C₁₂)alkyl(C(S)), (C₁-C₁₂)alkyl(S(CO)), (C₁-C₁₂)alkylthio, hydroxy(C₁-C₁₂)alkylthio, (C₁-C₁₂)alkylsulfinyl, (C₁-C₁₂)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₁₂)alkylthio, aryl(C₁-C₁₂)alkylsulfinyl, aryl(C₁-C₁₂)alkylsulfonyl, heterocyclyl(C₁-C₁₂)alkyl thio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, (C₁-C₁₂)alkylcarbonyl, arylcarbonyl, heterocyclylcarbonyl, aryl(C₁-C₁₂)alkylcarbonyl, heterocyclyl(C₁-C₁₂)alkylcarbonyl or of a group of formula NR^{a(2)}R^{b(2)} or —(CO)NR^{a(2)}R^{b(2)} in which R^{a(2)} and R^{b(2)} each and independently represent H, (C₁-C₁₂)alkyl, (C₁-C₁₂)alkylcarbonyl or R^{a(2)} and R^{b(2)} together with the nitrogen atom represent piperidine, pyrrolidine, azetidine or aziridine or any one of the groups

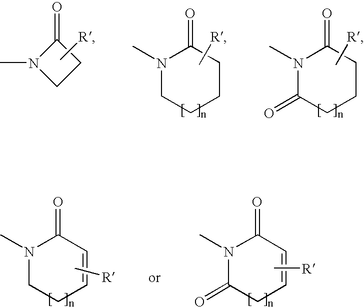

wherein n is an integer chosen from 0, 1 and 2, and R′ is H, CN, OH, a halogen (F, Cl, Br, I) atom, or one of the groups (C₁-C₈)alkyl, aryl, (C₁-C₈)alkoxy, (C₁-C₈)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen (F, Cl, Br, I) atoms;
Further R₂ represents substituted (C₁-C₁₂)alkoxy or substituted (C₁-C₁₂)alkylthio, wherein any one of these groups is substituted by one or more of any one of OH, aryl, (C₃-C₆)cycloalkyl or heterocyclyl; Further R₂ represents (C₁-C₁₂)alkylthio, substituted by one or more halogen (F, Cl, I, Br) atom(s); Further R₂ represents (C₁-C₁₂)alkylcarbonyloxy, aryl carbonyloxy, heterocyclylcarbonyloxy of which any one optionally is substituted by one or more of any one of the following groups or atoms; azido, cyano, halogen (F, Cl, Br, I) atom(s), OH, (C₁-C₁₂)alkoxy, (C₁-C₁₂)alkylthio, (C₁-C₁₂)alkylsulfinyl, (C₁-C₁₂)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₁₂)alkylthio, aryl(C₁-C₁₂)alkylsulfinyl, aryl(C₁-C₁₂)alkylsulfonyl, heterocyclyl(C₁-C₁₂)alkyl thio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, (C₁-C₁₂)alkylcarbonyl, arylcarbonyl, heterocyclylcarbonyl, aryl(C₁-C₁₂)alkylcarbonyl and heterocyclyl(C₁-C₁₂)alkylcarbonyl; Further R₂ represents a group of formula ((R^{a(2)})N(R^{b(2)}))(CO)—, in which R^{a(2)} and R^{b(2)} each and independently represent H, (C₁-C₁₂)alkyl, aryl, aryl(C₁-C₁₂)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine;
In a further embodiment R₂ represents unsubstituted (C₁-C₁₂)alkyl with the proviso that at the same time R₅ represents carboxy(C₁-C₁₂)alkyl;

In an even further embodiment, R₂ represents methyl substituted by any one of the groups

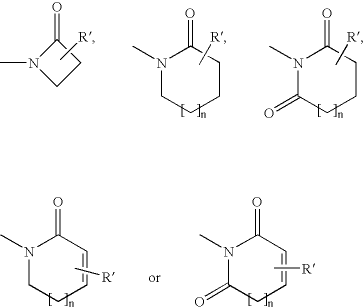

wherein n is an integer chosen from 0, 1 and 2, and R′ is H, CN, OH, a halogen (F, Cl, Br, I) atom, or one of the groups (C₁-C₈)alkyl, aryl, (C₁-C₈)alkoxy, (C₁-C₈)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen (F, Cl, Br, I) atoms; or methyl substituted by (C₁-C₆)alkylcarbonyloxy, a group NR^{a(2)}R^{b(2)} wherein R^{a(2)} and R^{b(2)} each and independently represent H, (C₁-C₁₂)alkyl, aryl, aryl(C₁-C₁₂)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine; (C₁-C₆)alkoxycarbonyl(C₁-C₆)alkyl, (C₁-C₆)alkylcarbonyl(C₁-C₆)alkyl, azido, (C₁-C₆)alkylsulfonyl, heterocyclylthio, heterocyclyl(C₁-C₆)alkylthio, (C₁-C₆)alkoxycarbonyl(C₁-C₆)alkylthio, a group NR^{a(2)}R^{b(2)}carbonyl(C₁-C₆)alkylthio wherein R^{a(2)} and R^{b(2)} each and independently represent H, (C₁-C₁₂)alkyl, aryl, aryl(C₁-C₁₂)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine; hydroxy(C₁-C₆)alkylcarbonyloxy, (C₁-C₁₂)alkylthio, hydroxy(C₁-C₁₂)alkylthio or (C₁-C₆)alkylcarbonylamino(C₁-C₆)alkylthio.
In an even further embodiment, R₂ represents —S—R″ wherein R″ represents hydroxy(C₁-C₁₂)alkyl, (C₁-C₆)alkylcarbonylamino(C₁-C₆)alkyl, carboxy(C₁-C₆)alkyl, (C₁-C₆)alkyloxycarbonyl(C₁-C₆)alkyl, a group NR^{a(2)}R^{b(2)}carbonyl(C₁-C₆)alkyl wherein R^{a(2)} and R^{b(2)} each and independently represent H, (C₁-C₁₂)alkyl, aryl, aryl(C₁-C₁₂)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine.
In another further embodiment, R₂ represents —O—R′″, wherein R′″ represents (C₁-C₆)alkylcarbonyl, (C₁-C₆)alkyloxycarbonyl(C₁-C₆)alkyl, cyano(C₁-C₆)alkyl, hydroxy(C₁-C₁₂)alkyl, (C₁-C₆)alkylcarbonylamino(C₁-C₆)alkyl, heterocyclyl(C₁-C₆)alkyl, carboxy(C₁-C₆)alkyl, (C₁-C₆)alkylaminocarbonyl(C₁-C₆)alkyl, (C₁-C₆)alkylcarbonyl(C₁-C₆)alkyl, a group NR^{a(2)}R^{b(2)}carbonyl(C₁-C₆)alkyl wherein R^{a(2)} and R^{b(2)} each and independently represent H, (C₁-C₁₂)alkyl, aryl, aryl(C₁-C₁₂)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine.
In a specific further embodiment, R₂ is selected from the group consisting of pyrrolidin-1-yl-methyl, (2-oxopyrrolidin-1-yl-)methyl, (2,5-dioxopyrrolidin-1-yl-)methyl and (2-oxopiperidin-1-yl)methyl.
In a another specific further embodiment, R₂ is (2-oxopyrrolidin-1-yl-)methyl.
In an even further specific embodiment, R₂ is (2-oxopiperidin-1-yl)methyl.

Embodiments for R₃ include, for example, H, methyl, methylsulfinyl, hydroxymethyl, methoxy or amino unsubstituted or optionally substituted with one or two methyl groups.

Other embodiments for R₃ include H or amino unsubstituted or optionally substituted with one or two methyl groups.

In a further embodiment of the invention R₃ is H.

Embodiments for R₄ include H, halogen such as chloro, methyl, cyano, nitro, amino unsubstituted or optionally substituted with one or two methyl groups and further includes 4-methoxy-4-oxobutoxy, 3-carboxy-propoxy and methylcarbonyl.

In a specific embodiment of the invention R₄ is selected from the group consisting of hydrogen, cyano, fluoro, chloro, bromo and iodo.

In a further specific embodiment of the invention R₄ is selected from the group consisting of cyano, fluoro, chloro, bromo and iodo.

In an alternative further specific embodiment of the invention R₄ is cyano.

In another further alternative specific embodiment of the invention R₄ is selected from the group consisting of fluoro and chloro.

In one embodiment R₅ represents hydrogen or methyl, with the proviso that when R₂ is unsubstituted alkyl, then R₅ represents carboxy(C₁-C₁₂)alkyl.

In an alternative embodiment R₅ is hydrogen, with the proviso that when R₂ is unsubstituted alkyl, then R₅ represents carboxy(C₁-C₁₂)alkyl.

In another embodiment R₅ represents carboxy(C₁-C₁₂)alkyl. In a further embodiment R₅ represents carboxy(C₁-C₆)alkyl. In an even further embodiment R₅ represents carboxymethyl.

In an alternative further embodiment R₅ represents hydrogen or carboxy(C₁-C₆)alkyl, with the proviso that when R₂ is unsubstituted alkyl, then R₅ represents carboxy(C₁-C₆)alkyl.

In an even further alternative embodiment R₅ represents hydrogen.

In one embodiment of the invention R₆ represents (C₁-C₆)alkyl, optionally substituted by OH, aryl, cycloalkyl, heterocyclyl or one or more halogen (F, Cl, Br, I) atoms; further R₆ represents (C₃-C₆)cycloalkyl, hydroxy(C₂-C₁₂)alkyl, aryl or heterocyclyl;

In another embodiment of the invention R₆ represents (C₁-C₆)alkyl, optionally substituted by OH, aryl, cycloalkyl, heterocyclyl or one or more halogen (F, Cl, Br, I) atoms.
In a further, other embodiment of the invention R₆ represents (C₁-C₆)alkyl, optionally substituted by one or more halogen (F, Cl, Br, I) atoms.
In a further embodiment of the invention R₆ represents (C₁-C₆)alkyl.

Embodiments for R₁₄ include, for example, hydrogen, methyl, amino, tert-butyloxycarbonyl, tert-butyloxycarbonyl-imino, 2-carboxyethyl and 3-tert-butoxy-3-oxo-propyl.

Other further embodiments for R₁₄ include, for example, hydrogen, methyl, tert-butyloxycarbonyl-imino, and amino.

In one embodiment of the invention R₁₅ represents H.

In a further embodiment of the invention both R₁₄ and R₁₅ represents H.

In one embodiment of the invention R₁₆ represents (C₁-C₆)alkyl, optionally substituted by OH, aryl, cycloalkyl, heterocyclyl or one or more halogen (F, Cl, Br, I) atoms; further R₆ represents (C₃-C₆)cycloalkyl, hydroxy(C₂-C₁₂)alkyl, aryl or heterocyclyl.

In another embodiment of the invention R₁₆ represents (C₁-C₆)alkyl, optionally substituted by OH, aryl, cycloalkyl, heterocyclyl or one or more halogen (F, Cl, Br, I) atoms.
In a further embodiment of the invention R₁₆ represents (C₁-C₆)alkyl.

In one embodiment R^{d} represents (C₁-C₁₂)alkyl, (C₃-C₈)cycloalkyl, aryl or heterocyclyl, and any one of these groups optionally substituted with one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine;

In one embodiment R^{d} represents (C₃-C₈)cycloalkyl, aryl or heterocyclyl, and any one of these groups optionally substituted with one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, halogen substituted (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, tri(C₁-C₄)alkylsilyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine;

Further embodiments for R^{d} includes aryl or heterocyclyl, more particularly, aryl or aromatic heterocyclyl.

Another embodiment for R^{d} include, aryl such as phenyl and aromatic heterocyclyl such as thienyl.

Other embodiments of R^{d} include phenyl which optionally may be substituted.

In a special embodiment R^{d} represents aryl, heterocyclyl or (C₃-C₆)cycloalkyl, and any one of these groups are optionally substituted with one or more halogen (F, Cl, Br, I) atoms or mixed halogen atoms, and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine.

In a further special embodiment R^{d} represents aryl or (C₃-C₆)cycloalkyl, and any one of these groups are optionally substituted with one or more halogen (F, Cl, Br, I) atoms or mixed halogen atoms, and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, tri(C₁-C₄)alkylsilyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine;

Another further embodiment for R^{d} include phenyl optionally substituted at the 2, 3, 4, 5 or 6-positions as well as any combination thereof. Example of substituents are cyano, tetrazol-5-yl, methoxy, trifluoromethoxy, methyl, trifluoromethyl, fluoro, chloro, bromo, methylsulfonyl, nitro, 3-methyl-5-oxo-4,5-dihydro-1H-pyrazol-1-yl. Two adjacent positions (e.g. 2, 3) may also be connected to form a ring. Example of such a substituent is 2-naphtyl. Further more specific values for heteroaryls are 2-chloro-5-thienyl, 3-bromo-5-chloro-2-thienyl, 2,1,3-benzoxadiazol-4-yl, 2,4-dimethyl-1,3-thiazol-5-yl, 2,3-dihydro-1,4-benzodioxin-6-yl, 5-chloro-3-methyl-1-benzothien-2-yl, 2,1,3-benzothiadiazol-4-yl, 2,5-dimethyl-3-furyl, 6-chloroimidazo[2,1-b][1,3]thiazol-5-yl, 2,3-dihydro-1-benzofuran-5-yl, 5-chloro-3-thienyl, 5-isoxazol-5-yl-2-thienyl, 5-isoxazol-3-yl-2-thienyl, 4-bromo-5-chloro-2-thienyl, 5-bromo-6-chloropyridin-3-yl, 5-bromo-2-thienyl, 5-pyridin-2-yl-2-thienyl, 2,5-dichloro-3-thienyl, 4,5-dichloro-2-thienyl,benzothien-3-yl, 2,5-dimethyl-3-thienyl, 3-thienyl,2-thienyl, 5-methylisoxazol-4-yl, pyridin-3-yl, [1-methyl-5-(trifluoromethyl)-1H-pyrazol-3-yl]-2-thienyl, 5-chloro-1,3-dimethyl-1H-pyrazol-4-yl, 4-[(4-chlorophenyl)sulfonyl]-3-methyl-2-thienyl, 5-(methoxycarbonyl)-2-furyl and 4-(methoxycarbonyl)-5-methyl-2-furyl.

Even further embodiments for R^{d} include phenyl optionally substituted at the 2, 3, 4 or 5-positions as well as any combination thereof. Example of substituents are cyano, tetrazol-5-yl, methoxy, trifluoromethoxy, methyl, trifluoromethyl, fluoro, chloro, bromo, methylsulfonyl, nitro, 3-methyl-5-oxo-4,5-dihydro-1H-pyrazol-1-yl. Two adjacent positions (e.g. 2, 3) may also be connected to form a ring. Example of such a substituent is 2-naphtyl. Further more specific values for heteroaryls are 2-chloro-5-thienyl, 3-bromo-5-chloro-2-thienyl, 2,1,3-benzoxadiazol-4-yl, 2,4-dimethyl-1,3-thiazol-5-yl, 2,3-dihydro-1,4-benzodioxin-6-yl, 5-chloro-3-methyl-1-benzothien-2-yl, 2,1,3-benzothiadiazol-4-yl, 2,5-dimethyl-3-furyl, 6-chloroimidazo[2,1-b][1,3]thiazol-5-yl, 2,3-dihydro-1-benzofuran-5-yl, 5-chloro-3-thienyl, 5-isoxazol-5-yl-2-thienyl, 5-isoxazol-3-yl-2-thienyl, 4-bromo-5-chloro-2-thienyl, 5-bromo-6-chloropyridin-3-yl, 5-bromo-2-thienyl, 5-pyridin-2-yl-2-thienyl, 2,5-dichloro-3-thienyl, 4,5-dichloro-2-thienyl,benzothien-3-yl, 2,5-dimethyl-3-thienyl, 3-thienyl,2-thienyl, 5-methylisoxazol-4-yl, pyridin-3-yl, [1-methyl-5-(trifluoromethyl)-1H-pyrazol-3-yl]-2-thienyl, 5-chloro-1,3-dimethyl-1H-pyrazol-4-yl, 4-[(4-chlorophenyl)sulfonyl]-3-methyl-2-thienyl, 5-(methoxycarbonyl)-2-furyl and 4-(methoxycarbonyl)-5-methyl-2-furyl.

In one embodiment of the invention R^{c} represents an unsubstituted or monosubstituted or disubstituted (C₁-C₄)alkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents aryl, i.e. R^{c}R^{d} represents an aryl-(C₁-C₄)alkylene group with any substituents according to above.

In another embodiment of the invention R^{c} represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; Further R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)alkylene group or (C₁-C₄)oxoalkylene group with any substituents according to above; In an alternative embodiment of the invention R^{c} is a single bond.

In a further alternative embodiment of the invention R^{c} represents imino (—NH—) or substituted imino (—NR₁₉—), wherein R₁₉ represents H or (C₁-C₄)alkyl;

In a preferred embodiment of the invention R^{c} represents an unsubstituted or monosubstituted or disubstituted (C₁-C₃)alkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)}and R^{b(Rc)} individually and independently from each other represents hydrogen, (C₁-C₄)alkyl or R^{a(Rc)}and R^{b(Rc)} together with the nitrogen atom represent piperidine, pyrrolidine, azetidine or aziridine, and R^{d} represents aryl, i.e. R^{c}R^{d} represents an aryl-(C₁-C₃)alkylene group with any substituents according to above.

In a further embodiment of the invention R^{c} represents an unsubstituted or monosubstituted or disubstituted (C₁-C₄)alkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents heterocyclyl, i.e. R^{c}R^{d} represents a heterocyclyl-(C₁-C₄)alkylene group with any substituents according to above.

In a further preferred embodiment of the invention R^{c} represents an unsubstituted or monosubstituted or disubstituted (C₁-C₃)alkylene group wherein any substituents each individually and independently are selected from (C₁-C₄)alkyl, (C₁-C₄)alkoxy, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents heterocyclyl, i.e. R^{c}R^{d} represents a heterocyclyl-(C₁-C₃)alkylene group with any substituents according to above.

In a particular embodiment of the invention R^{c} represents a C₁-alkylene group wherein any substituents each individually and independently are selected from (C₁-C₄)alkyl, (C₁-C₄)alkoxy, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents aryl, i.e. R^{c}R^{d} represents an aryl-C₁-alkylene group with any substituents according to above.

In a further particular embodiment of the invention R^{c} represents an unsubstituted or monosubstituted or disubstituted methylene group, imino (—NH—) or methylimino (—N(CH₃)—), wherein any substituents each and individually are selected from (C₁-C₄)alkyl, (C₁-C₄)alkoxy, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine.

In one embodiment of the invention R₁₉ represents hydrogen.

In another embodiment of the invention R₁₉ represents methyl.

In a most particular embodiment of the invention R^{c}R^{d} represents a benzyl group, or a benzyl group which is substituted according to what is described in connection to substitution of the aryl group.

In one embodiment of the invention X represents a single bond.

In another embodiment of the invention X represents imino (—NH—) or methylene (—CH₂—).

In yet another embodiment X represents imino (—NH—).

In a further embodiment X represents methylene (—CH₂—).

Suitable values for the B ring/ring system include, for example, diazepanylene, piperazinylene, piperidinylene, pyrrolidinylene and azetidinylene, wherein any one of them may be presents in any of their isomeric forms (e.g. piperazin-tetrahydropyridazin-tetrahydropyrimidin).

Embodiments for the B ring/ring system include, for example, diazepanylene, piperazinylene, piperidinylene, pyrrolidinylene and azetidinylene.

Further embodiments include these groups which are substituted with R₁₄ having a (C₁-C₆)alkyl group, wherein the (C₁-C₆)alkyl group optionally is substituted with OH, COOH or COOR^{e} group(s), e.g. a 2-carboxyethyl group, and wherein R^{e} represents H, aryl, cycloalkyl, heterocyclyl or (C₁-C₁₂)alkyl optionally substituted by one or more of halogen (F, Cl, Br, I) or mixed halogen atoms, OH, aryl, cycloalkyl and heterocyclyl.

In an alternative to the embodiment for the B ring/ring system above, the embodiment include, for example, diazepanylene, piperazinylene, piperidinylene, pyrrolidinylene or azetidinylene groups which are substituted with R₁₄ having a (C₁-C₆)alkyl group, wherein the (C₁-C₆)alkyl group optionally is substituted with OH, COOH or COOR^{e} group(s), e.g. a 2-carboxyethyl group, and wherein R^{e} represents H, aryl, cycloalkyl, heterocyclyl or (C₁-C₆)alkyl optionally substituted by one or more of halogen (F, Cl, Br, I) or mixed halogen atoms, OH, aryl, cycloalkyl and heterocyclyl.

In a further alternative to the embodiment for the B ring/ring system above, B is chosen from an azetidinylene group or a piperidinylene group, any of which optionally is substituted with R₁₄ having a (C₁-C₆)alkyl group, wherein the (C₁-C₆)alkyl group optionally is substituted with OH, COOH or COOR^{e} group(s), e.g. a 2-carboxyethyl group, and wherein R^{e} represents H, aryl, cycloalkyl, heterocyclyl or (C₁-C₆)alkyl optionally substituted by one or more of halogen (F, Cl, Br, I) or mixed halogen atoms, OH, aryl, cycloalkyl and heterocyclyl.

In an even further alternative to the embodiment for the B ring/ring system above, B is chosen from an unsubstituted azetidinylene group or an unsubstituted piperidinylene group.

In a special alternative to the embodiment for the B ring/ring system above, B is an unsubstituted piperidinylene group.

A 2nd embodiment of formula I is defined by;
R₁ represents R₆OC(O) or R₁₆SC(O);
preferably R₁ represents R₆OC(O);

R₂ represents substituted (C₁-C₆)alkyl optionally interrupted by sulphur, substituted (C₁-C₆)alkoxy or substituted (C₁-C₆)alkylthio, wherein any one of these groups is substituted by one or more of azido, carboxy, cyano, (C₁-C₆)alkylcarbonyloxy, hydroxy(C₁-C₆)alkylcarbonyloxy, arylcarbonyloxy, heterocyclylcarbonyloxy, (C₁-C₆)alkyloxycarbonyl, (C₁-C₆)alkyl(C(S)), (C₁-C₆)alkyl(S(CO)), (C₁-C₆)alkylthio, hydroxy(C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl, heterocyclyl(C₁-C₆)alkylcarbonyl or of a group of formula NR^{a(2)}R^{b(2)} or —(CO)NR^{a(2)}R^{b(2)}, in which R^{a(2)} and R^{b(2)} each and independently represent H, (C₁-C₆)alkyl, (C₁-C₆)alkylcarbonyl or R^{a(2)} and R^{b(2)} together with the nitrogen atom represent piperidine, pyrrolidine, azetidine or aziridine or any one of the groups

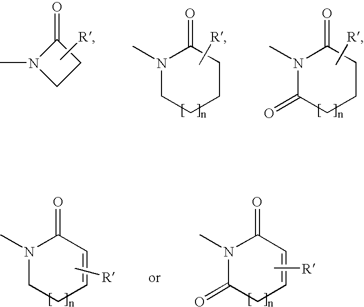

wherein n is an integer chosen from 0, 1 and 2, and R′ is H, CN, OH, a halogen (F, Cl, Br, I) atom, or one of the groups (C₁-C₈)alkyl, aryl, (C₁-C₈)alkoxy, (C₁-C₈)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen (F, Cl, Br, I) atoms;
Further R₂ represents substituted (C₁-C₆)alkoxy or substituted (C₁-C₆)alkylthio, wherein any one of these groups is substituted by one or more of any one of OH, aryl, (C₃-C₆)cycloalkyl or heterocyclyl; Further R₂ represents (C₁-C₆)alkylthio, substituted by one or more halogen (F, Cl, I, Br) atom(s); Further R₂ represents (C₁-C₆)alkylcarbonyloxy, aryl carbonyloxy, heterocyclylcarbonyloxy of which any one optionally is substituted by one or more of any one of the following groups or atoms; azido, cyano, halogen (F, Cl, Br, I) atom(s), OH, (C₁-C₆)alkoxy, (C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl and heterocyclyl(C₁-C₆)alkylcarbonyl; Further R₂ represents unsubstituted (C₁-C₆)alkyl with the proviso that at the same time R₅ represents carboxy(C₁-C₆)alkyl; Further R₂ represents a group of formula ((R^{a(2)})N(R^{b(2)}))(CO)—, in which R^{a(2)} and R^{b(2)} each and independently represent H, (C₁-C₆)alkyl, aryl, aryl(C₁-C₆)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine;

R₃ represents H, CN, NO₂, halogen (F, Cl, Br, I), (C₁-C₆)alkyl optionally interrupted by oxygen and/or optionally substituted by OH, aryl, cycloalkyl, heterocyclyl or one or more halogen (F, Cl, Br, I) atoms; further R₃ represents (C₁-C₆)alkoxy optionally substituted by one or more halogen (F, Cl, Br, I) atoms; further R₃ represents (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;

R₄ represents H, CN, NO₂, halogen (F, Cl, Br, I), (C₁-C₆)alkyl optionally interrupted by oxygen and/or optionally substituted by OH, COOH, (C₁-C₆)alkoxycarbonyl, aryl, cycloalkyl, heterocyclyl or one or more halogen (F, Cl, Br, I) atoms; further R₄ represents (C₃-C₆)cycloalkyl, hydroxy(C₁-C₆)alkyl, (C₁-C₆)alkylC(O), (C₁-C₆)alkylcycloalkyl, (C₁-C₆)alkoxy wherein the alkoxy group may optionally be substituted by one or more halogen (F, Cl, Br, I) atoms, OH and/or COOH and/or (C₁-C₆)alkoxycarbonyl; further R₄ represents (C₁-C₆)alkylthioC(O), (C₁-C₆)alkylC(S), (C₁-C₆)alkoxyC(O), (C₃-C₆)cycloalkoxy, aryl, aryl(C₁-C₆)alkoxy, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkoxy, (C₃-C₆)cycloalkyl(C₁-C₆)alkylsulfinyl, (C₃-C₆)cycloalkyl(C₁-C₆)alkylsulfonyl or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;

R₅ represents H or (C₁-C₆)alkyl or carboxy(C₁-C₆)alkyl; with the proviso that when R₂ is unsubstituted (C₁-C₆)alkyl, R₅ represents carboxy(C₁-C₆)alkyl;

R₆ represents (C₁-C₆)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms; further R₆ represents (C₂-C₆)alkenyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms; further R₆ represents (C₂-C₆)alkynyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl, hydroxy(C₂-C₆)alkyl, aryl or heterocyclyl;

R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₄ represents aryl, aryl(C₁-C₆)alkoxy, aryl(C₁-C₆)alkyl, (C₃-C₆)cycloalkyl(C₁-C₆)alkoxy, heterocyclyl, a halogen (F, Cl, Br, I) atom, (C₃-C₆)cycloalkyl, hydroxy(C₁-C₆)alkyl, (C₁-C₆)alkoxy, (C₃-C₆)cycloalkoxy, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkoxy, (C₃-C₆)cycloalkyl(C₁-C₆)alkylsulfinyl or (C₃-C₆)cycloalkyl(C₁-C₆)alkylsulfonyl, a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;

R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₅ represents aryl, aryl(C₁-C₆)alkoxy, aryl(C₁-C₆)alkyl, (C₃-C₆)cycloalkyl(C₁-C₆)alkoxy, heterocyclyl, a halogen (F, Cl, Br, I) atom, (C₃-C₆)cycloalkyl, hydroxy(C₁-C₆)alkyl, (C₁-C₆)alkoxy, (C₃-C₆)cycloalkoxy, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkoxy, (C₃-C₆)cycloalkyl(C₁-C₆)alkylsulfinyl, (C₃-C₆)cycloalkyl(C₁-C₆)alkylsulfonyl or a group of formula NR^{a(15)}R^{b(15)} in which R^{a(15)} and R^{b(15)} independently represent H, (C₁-C₆)alkyl, (C₁-C₆)alkylC(O)), (C₁-C₆)alkoxyC(O) or R^{a(15)} and R^{b(15)} together with the nitrogen atom represent piperidine, pyrrolidine, azetidine or aziridine;

R₁₆ represents (C₁-C₆)alkyl optionally interrupted by oxygen and/or optionally substituted by OH, aryl, cycloalkyl, heterocyclyl or one or more halogen (F, Cl, Br, I) atoms; further R₁₆ represents (C₃-C₆)cycloalkyl, hydroxy(C₂-C₆)alkyl, (C₁-C₆)alkoxy, (C₃-C₆)cycloalkoxy, aryl or heterocyclyl;

R^{c} is a single bond or represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; Further R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)alkylene group or (C₁-C₄)oxoalkylene group with any substituents according to above;

R₁₉ represents H or (C₁-C₄)alkyl;

R^{d} represents (C₁-C₆)alkyl, (C₃-C₈)cycloalkyl, aryl or heterocyclyl, and any one of these groups optionally substituted with one or more halogen (F, Cl, Br, I) atoms and/or one or more of the following groups, OH, CN, NO₂, (C₁-C₆)alkyl, (C₁-C₆)alkoxyC(O), (C₁-C₆)alkoxy, halogen substituted (C₁-C₆)alkyl, (C₃-C₆)cycloalkyl, aryl, heterocyclyl, (C₁-C₆)alkylsulfinyl, (C₁-C₆)alkylsulfonyl, (C₁-C₆)alkylthio, halogen substituted (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, tri(C₁-C₄)alkylsilyl or a group of formula NR^{a(Rd)}R^{b(Rd)} in which R^{a(Rd)} and R^{b(Rd)} independently represent H, (C₁-C₆)alkyl, (C₁-C₆)alkylC(O) or R^{a(Rd)} and R^{b(Rd)} together with the nitrogen atom represent piperidine, pyrrolidine, azetidine or aziridine;

X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; further X may represent a group (—CH₂-)n wherein n=2-6, which optionally is unsaturated and/or substituted by one or more substituent chosen among halogen, hydroxyl or (C₁-C₆)alkyl;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections).

A 3rd embodiment of formula I is defined by;
R₁ represents R₆OC(O), or R₁₆SC(O);

R₂ represents substituted (C₁-C₆)alkyl optionally interrupted by sulphur, substituted (C₁-C₆)alkoxy or substituted (C₁-C₆)alkylthio, wherein any one of these groups is substituted by one or more of azido, carboxy, cyano, (C₁-C₆)alkylcarbonyloxy, hydroxy(C₁-C₆)alkylcarbonyloxy, arylcarbonyloxy, heterocyclylcarbonyloxy, (C₁-C₆)alkyloxycarbonyl, (C₁-C₆)alkyl(C(S)), (C₁-C₆)alkyl(S(CO)), (C₁-C₆)alkylthio, hydroxy(C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl, heterocyclyl(C₁-C₆)alkylcarbonyl or of a group of formula NR^{a(2)}R^{b(2)} or —(CO)NR^{a(2)}R^{b(2)}, in which R^{a(2)} and R^{b(2)} each and independently represent H, (C₁-C₆)alkyl, (C₁-C₆)alkylcarbonyl or R^{a(2)} and R^{b(2)} together with the nitrogen atom represent piperidine, pyrrolidine, azetidine or aziridine or any one of the groups

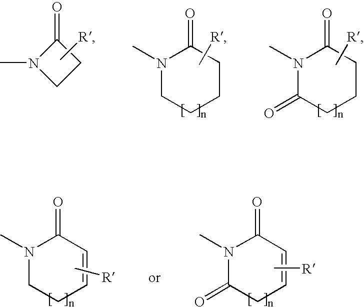

wherein n is an integer chosen from 0, 1 and 2, and R′ is H, CN, OH, a halogen (F, Cl, Br, I) atom, or one of the groups (C₁-C₆)alkyl, aryl, (C₁-C₆)alkoxy, (C₁-C₆)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen (F, Cl, Br, I) atoms;
Further R₂ represents substituted (C₁-C₆)alkoxy or substituted (C₁-C₆)alkylthio, wherein any one of these groups is substituted by one or more of any one of OH, aryl, (C₃-C₆)cycloalkyl or heterocyclyl; Further R₂ represents (C₁-C₆)alkylthio, substituted by one or more halogen (F, Cl, I, Br) atom(s); Further R₂ represents (C₁-C₆)alkylcarbonyloxy, aryl carbonyloxy, heterocyclylcarbonyloxy of which any one optionally is substituted by one or more of any one of the following groups or atoms; azido, cyano, halogen (F, Cl, Br, I) atom(s), OH, (C₁-C₆)alkoxy, (C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl and heterocyclyl(C₁-C₆)alkylcarbonyl; Further R₂ represents unsubstituted (C₁-C₆)alkyl with the proviso that at the same time R₅ represents carboxy(C₁-C₆)alkyl; Further R₂ represents a group of formula ((R^{a(2)})N(R^{b(2)}))(CO)—, in which R^{a(2)} and R^{b(2)} each and independently represent H, (C₁-C₆)alkyl, aryl, aryl(C₁-C₆)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine;

R₃ represents H, CN, NO₂, halogen (F, Cl, Br, I), (C₁-C₆)alkyl optionally interrupted by oxygen and/or optionally substituted by OH, aryl, cycloalkyl, heterocyclyl or one or more halogen atoms; further R₃ represents (C₁-C₆)alkoxy optionally substituted by one or more halogen (F, Cl, Br, I) atoms; further R₃ represents (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;

R₄ represents H, CN, NO₂, halogen (F, Cl, Br, I), (C₁-C₆)alkyl optionally interrupted by oxygen and/or optionally substituted by OH, COOH, aryl, cycloalkyl, heterocyclyl or one or more halogen atoms; further R₄ represents (C₃-C₆)cycloalkyl, hydroxy(C₁-C₆)alkyl, (C₁-C₆)alkylC(O), (C₁-C₆)alkoxy wherein the alkoxy group may optionally be substituted by one or more halogen (F, Cl, Br, I) atoms, OH and/or COOH and/or methoxycarbonyl; further R₄ represents (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O) or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;

R₅ represents H or (C₁-C₆)alkyl or carboxy(C₁-C₆)alkyl; with the proviso that when R₂ is unsubstituted (C₁-C₆)alkyl, R₅ represents carboxy(C₁-C₆)alkyl;

R₆ represents (C₁-C₆)alkyl optionally interrupted by oxygen, (with the proviso that any such oxygen must be at least 1 carbon atom away from the ester-oxygen connecting the R₆ group) and/or optionally substituted by OH, aryl, cycloalkyl, heterocyclyl or one or more halogen (F, Cl, Br, I) atoms; further R₆ represents (C₂-C₆)alkenyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms; further R₆ represents (C₂-C₆)alkynyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl, hydroxy(C₂-C₆)alkyl, aryl or heterocyclyl;

R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₄ represents aryl, aryl(C₁-C₆)alkoxy, aryl(C₁-C₆)alkyl, (C₃-C₆)cycloalkyl(C₁-C₆)alkoxy, heterocyclyl, a halogen (F, Cl, Br, I) atom, (C₃-C₆)cycloalkyl, hydroxy(C₁-C₆)alkyl, (C₁-C₆)alkoxy, (C₃-C₆)cycloalkoxy, or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;

R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₅ represents aryl, aryl(C₁-C₆)alkoxy, aryl(C₁-C₆)alkyl, (C₃-C₆)cycloalkyl(C₁-C₆)alkoxy, heterocyclyl, a halogen (F, Cl, Br, I) atom, (C₃-C₆)cycloalkyl, hydroxy(C₁-C₆)alkyl, (C₁-C₆)alkoxy, (C₃-C₆)cycloalkoxy, or a group of formula NR^{a(15)}R^{b(15)} in which R^{a(15)} and R^{b(15)} independently represent H, (C₁-C₆)alkyl, (C₁-C₆)alkylC(O), (C₁-C₆)alkoxyC(O) or R^{a(15)} and R^{b(15)} together with the nitrogen atom represent piperidine, pyrrolidine, azetidine or aziridine;

R₁₆ represents (C₁-C₆)alkyl optionally interrupted by oxygen and/or optionally substituted by OH, aryl, cycloalkyl, heterocyclyl or one or more halogen (F, Cl, Br, I) atoms; further R₁₆ represents (C₃-C₆)cycloalkyl, hydroxy(C₂-C₆)alkyl, (C₁-C₆)alkoxy, (C₃-C₆)cycloalkoxy, aryl or heterocyclyl;

R^{c} is a single bond or represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; Further R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)alkylene group or (C₁-C₄)oxoalkylene group with any substituents according to above;

R₁₉ represents H or (C₁-C₄)alkyl;

R^{d} represents (C₁-C₆)alkyl, (C₃-C₈)cycloalkyl, aryl or heterocyclyl, and any one of these groups optionally substituted with one or more halogen (F, Cl, Br, I) atoms and/or one or more of the following groups, OH, CN, NO₂, (C₁-C₆)alkyl, (C₁-C₆)alkoxyC(O), (C₁-C₆)alkoxy, halogen substituted (C₁-C₆)alkyl, (C₃-C₆)cycloalkyl, aryl, heterocyclyl, (C₁-C₆)alkylsulfinyl, (C₁-C₆)alkylsulfonyl, (C₁-C₆)alkylthio, halogen substituted (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, tri(C₁-C₄)alkylsilyl or a group of formula NR^{a(Rd)}R^{b(Rd)} in which R^{a(Rd)} and R^{b(Rd)} independently represent H, (C₁-C₆)alkyl, (C₁-C₆)alkylC(O) or R^{a(Rd)} and R^{b(Rd)} together with the nitrogen atom represent piperidine, pyrrolidine, azetidine or aziridine;

X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; further X may represent a group (—CH₂-)n wherein n=2-6, which optionally is unsaturated and/or substituted by one or more substituent chosen among halogen, hydroxyl or (C₁-C₆)alkyl;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections).

A 4rth embodiment of formula I is defined by;
R₁ represents R₆OC(O) or R₁₆SC(O);

R₂ represents substituted (C₁-C₆)alkyl optionally interrupted by sulphur, substituted (C₁-C₆)alkoxy or substituted (C₁-C₆)alkylthio, wherein any one of these groups is substituted by one or more of azido, carboxy, cyano, (C₁-C₆)alkylcarbonyloxy, hydroxy(C₁-C₆)alkylcarbonyloxy, arylcarbonyloxy, heterocyclylcarbonyloxy, (C₁-C₆)alkyloxycarbonyl, (C₁-C₆)alkyl(C(S)), (C₁-C₆)alkyl(S(CO)), (C₁-C₆)alkylthio, hydroxy(C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl, heterocyclyl(C₁-C₆)alkylcarbonyl or of a group of formula NR^{a(2)}R^{b(2)} or —(CO)NR^{a(2)}R^{b(2)}, in which R^{a(2)} and R^{b(2)} each and independently represent H, (C₁-C₆)alkyl, (C₁-C₆)alkylcarbonyl or R^{a(2)} and R^{b(2)} together with the nitrogen atom represent piperidine, pyrrolidine, azetidine or aziridine or any one of the groups

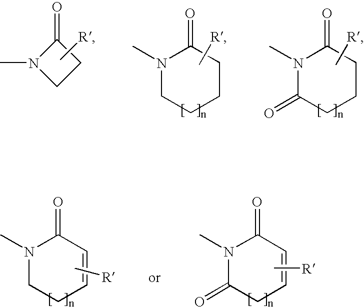

wherein n is an integer chosen from 0, 1 and 2, and R′ is H, CN, OH, a halogen (F, Cl, Br, I) atom, or one of the groups (C₁-C₄)alkyl, aryl, (C₁-C₄)alkoxy, (C₁-C₄)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen (F, Cl, Br, I) atoms;
Further R₂ represents substituted (C₁-C₆)alkoxy or substituted (C₁-C₆)alkylthio, wherein any one of these groups is substituted by one or more of any one of OH, aryl, (C₃-C₆)cycloalkyl or heterocyclyl; Further R₂ represents (C₁-C₆)alkylthio, substituted by one or more halogen (F, Cl, I, Br) atom(s); Further R₂ represents (C₁-C₆)alkylcarbonyloxy, aryl carbonyloxy, heterocyclylcarbonyloxy of which any one optionally is substituted by one or more of any one of the following groups or atoms; azido, cyano, halogen (F, Cl, Br, I) atom(s), OH, (C₁-C₆)alkoxy, (C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl and heterocyclyl(C₁-C₆)alkylcarbonyl; Further R₂ represents unsubstituted (C₁-C₆)alkyl with the proviso that at the same time R₅ represents carboxy(C₁-C₆)alkyl;

R₃ represents H or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;

R₄ represents CN, halogen (F, Cl, Br, I), further R₄ represents (C₁-C₆)alkylC(O), (C₁-C₆)alkoxy wherein the alkoxy group may optionally be substituted by one or more halogen (F, Cl, Br, I) atoms, OH and/or COOH and/or methoxycarbonyl;

R₅ represents H or carboxy(C₁-C₆)alkyl; with the proviso that when R₂ is unsubstituted (C₁-C₆)alkyl, R₅ represents carboxy(C₁-C₆)alkyl;

R₆ represents (C₁-C₆)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl or hydroxy(C₂-C₆)alkyl;

R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₄ represents or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;

R₁₅ represents H;

R₁₆ represents (C₁-C₆)alkyl optionally interrupted by oxygen and/or optionally substituted by OH, aryl, cycloalkyl, heterocyclyl or one or more halogen (F, Cl, Br, I) atoms; further R₁₆ represents (C₃-C₆)cycloalkyl, hydroxy(C₂-C₆)alkyl, (C₁-C₆)alkoxy, (C₃-C₆)cycloalkoxy, aryl or heterocyclyl;

R^{c} represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; Further R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)alkylene group or (C₁-C₄)oxoalkylene group with any substituents according to above;

R₁₉ represents H or (C₁-C₄)alkyl;

R^{d} represents (C₁-C₆)alkyl, (C₃-C₈)cycloalkyl, aryl or heterocyclyl, and any one of these groups optionally substituted with one or more halogen (F, Cl, Br, I) atoms and/or one or more of the following groups, OH, CN, NO₂, (C₁-C₆)alkyl, (C₁-C₆)alkoxyC(O), (C₁-C₆)alkoxy, halogen substituted (C₁-C₆)alkyl, (C₃-C₆)cycloalkyl, aryl, heterocyclyl, (C₁-C₆)alkylsulfinyl, (C₁-C₆)alkylsulfonyl, (C₁-C₆)alkylthio, halogen substituted (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, tri(C₁-C₄)alkylsilyl or a group of formula NR^{a(Rd)}R^{b(Rd)} in which R^{a(Rd)} and R^{b(Rd)} independently represent H, (C₁-C₆)alkyl, (C₁-C₆)alkylC(O) or R^{a(Rd)} and R^{b(Rd)} together with the nitrogen atom represent piperidine, pyrrolidine, azetidine or aziridine;

X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; further X may represent a group (—CH₂-)n wherein n=2-6, which optionally is unsaturated and/or substituted by one or more substituent chosen among halogen, hydroxyl or (C₁-C₆)alkyl;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections).

A 5th embodiment of formula I is defined by that;
R₁ is chosen from a group consisting of methoxycarbonyl, ethoxycarbonyl, n-propyloxycarbonyl, isopropyloxycarbonyl and ethylthiocarbonyl;
R₂ is chosen from a group consisting of methyl, acetoxy, pyrrolidin-1-yl-methyl, (2-oxopyrrolidin-1-yl)methyl, 2-(2-oxopyrrolidin-1-yl)ethoxy, (2-oxopiperidin-1-yl)methyl, (dimethylamino)methyl, acetoxymethyl, 3-oxobutyl, 2-ethoxy-2-oxoethoxy, 3-ethoxy-3-oxopropyl, 4-ethoxy-4-oxobutyl, azidomethyl, (methylsulfonyl)methyl, (2-ethoxy-2-oxoethyl)thio, ((2-ethoxy-2-oxoethyl)thio)methyl, (2-isopropoxy-2oxoethyl)thio, (2,5-dioxopyrrolidin-1-yl)methyl, (glycoloyloxy)methyl, 2-cyanoethoxy, 2-hydroxyethoxy, ethylthiomethyl, (2-hydroxyethyl)thio, (2-hydroxyethyl)thiomethyl, 2-acetamidoethoxy, (2-acetamidoethyl)thio, (2-acetamidoethyl)thiomethyl, 2-amino-2-oxoethoxy, oxetan-2-yl-methoxy, carboxymethyloxy, 2-(methylamino)-2-oxoethoxy and (carboxy)methylthio, ((carboxy)methylthio)methyl, ethylthiomethyl, 2-(1H-pyrrol-1-yl)ethoxy, (((1-methyl-1H-imidazol-2-yl)methyl)thio)methyl, (1,3-thiazol-2-ylthio)methyl, ((3-methoxy-3-oxopropyl)thio)methyl, (1S)-2-ethoxy-1-methyl-2-oxoethoxy, 4-oxopentyl, ((2-(dimethylamino)-2-oxoethyl)thio)methyl, (2-azetidin-1-yl-2-oxoethyl)thio, 2-(dimethylamino)-2-oxoethoxy with the proviso that when R₂ is methyl R₅ is carboxymethyl;
R₃ is H;
R₄ is chosen from a group consisting of hydrogen, fluoro, chloro, bromo and cyano;
R₅ is chosen from H, methyl or carboxymethyl;
R₆ is chosen from a group consisting of methyl, ethyl, isopropyl and n-propyl;
R₁₄ is H;
R₁₅ is H;
R₁₆ is ethyl;
R^{c} is chosen from a group consisting of methylene (—CH₂—), imino (—NH—) and methylimino (—N(CH₃)—);
R₁₉ is chosen from H or methyl;
   R^{d} is chosen from a group consisting of cyclobutyl, cyclopentyl, cyclohexyl, 4-methylcyclohexyl, tetrahydro-2H-pyran-4-yl, phenyl, 4-methylphenyl, 4-isopropylphenyl, 4-tert-butylphenyl, 4-methoxyphenyl, 4-fluorophenyl, 4-chlorophenyl, 2,4-difluorophenyl, 2,4-dichlorophenyl, 2-chloro-4-fluoro-phenyl, 4-chloro-2-fluoro-phenyl, 4-trifluoromethylphenyl, 4-((trifluoromethyl)thio)-phenyl and 4-(trimethylsilyl)-phenyl;
X represents a single bond;

B is chosen from the group consisting of 4-piperidin-1-ylene and 3-azetidin-1-ylene, and the substituents R₁₄ and R₁₅ are connected to the B ring/ring system, in such a way that no quarternary ammonium compounds are formed (by these connections).

An alternative 5th embodiment of formula I is defined by that;
R₁ is ethoxycarbonyl;
R₂ is chosen from a group consisting of methyl, acetoxy, pyrrolidin-1-yl-methyl, (dimethylamino)methyl, acetoxymethyl, (ethoxycarbonyl)methoxyl, azidomethyl, (methylsulfonyl)methyl, (2,5-dioxopyrrolidin-1-yl)methyl, (glycoloyloxy)methyl, 2-cyanoethoxy, 2-hydroxyethoxy, ethylthiomethyl, (2-hydroxyethyl)thio, (2-hydroxyethyl)thiomethyl, 2-acetamidoethoxy, (2-acetamidoethyl)thio, (2-acetamidoethyl)thiomethyl, 2-amino-2-oxoethoxy, oxetan-2-yl-methoxy, carboxymethoxy, 2-methylamino-2-oxoethoxy and (carboxy)methylthio, with the proviso that when R₂ is methyl R₅ is carboxymethyl;
R₃ is H;
R₄ is cyano;
R₅ is chosen from H or carboxymethyl;
R₆ is ethyl;
R₁₄ is H;
R₁₅ is H;
R^{c} is methylene (—CH₂—);
   R^{d} is phenyl;

X represents a single bond;

B is chosen from the group consisting of 4-piperidin-1-ylene and 3-azetidin-1-ylene, and the substituents R₁₄ and R₁₅ are connected to the B ring/ring system, in such a way that no quarternary ammonium compounds are formed (by these connections).

In a 6th embodiment of formula (I), formula (I) is defined as being any compound(s) of formula (Ia)-(Ii):

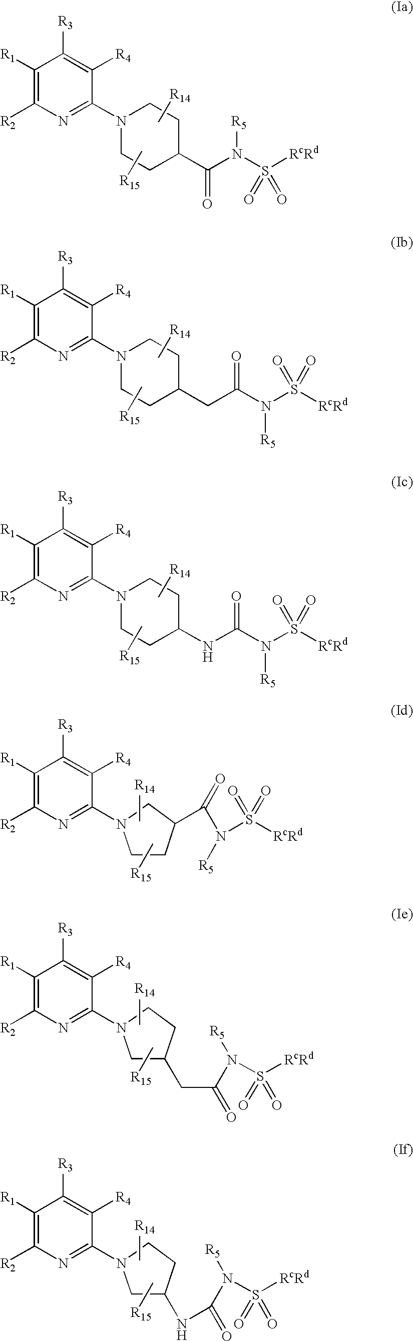

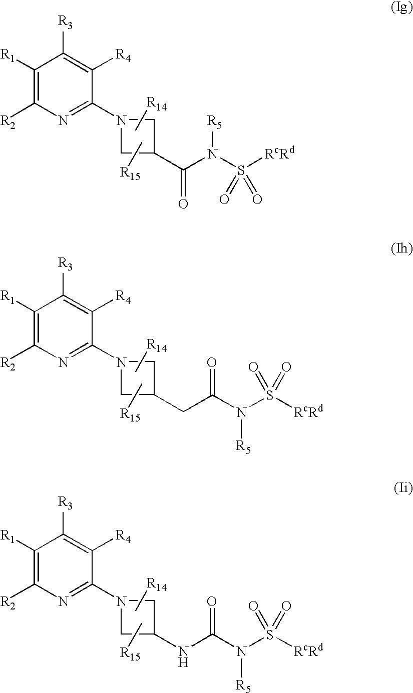

In the above Ia to Ii the various values of R are as defined above and include any of the previously mentioned embodiments.

In a 7th embodiment formula (I) is defined as being any compound(s) of formula (Iaa)-(Igg);

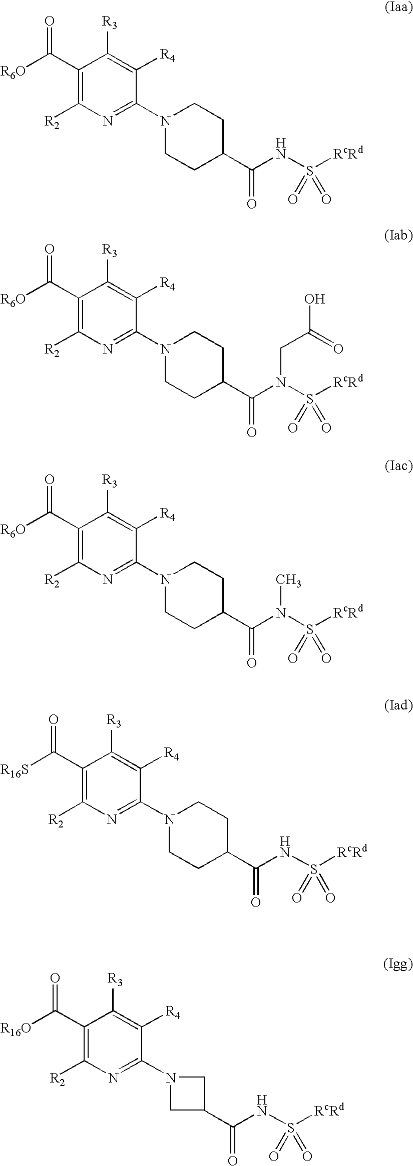

In the above Iaa to Igg the various values of R (except R₁₄ and R₁₅ being H) are as defined above and include any of the previously mentioned embodiments.

Examples of specific compounds according to the invention can be selected from;
N-(benzylsulfonyl)-N-({1-[3-cyano-5-(ethoxycarbonyl)-6-methylpyridin-2-yl]piperidin-4-yl}carbonyl)glycine
ethyl 2-acetoxy-6-{4-[(benzylsulfonyl)carbamoyl]piperidin-1-yl}-5-cyanonicotinate
ethyl 6-{4-[(benzylsulfonyl)carbamoyl]piperidin-1-yl}-5-cyano-2-(pyrrolidin-1-ylmethyl)nicotinate
ethyl 6-{4-[(benzylsulfonyl)carbamoyl]piperidin-1-yl}-5-cyano-2-[(dimethylamino)methyl]nicotinate
ethyl 2-(acetoxymethyl)-6-{4-[(benzylsulfonyl)carbamoyl]piperidin-1-yl}-5-cyanonicotinate
ethyl 6-{3-[(benzylsulfonyl)carbamoyl]azetidin-1-yl}-5-cyano-2-[(dimethylamino)methyl]nicotinate
ethyl 2-(acetoxymethyl)-6-{3-[(benzylsulfonyl)carbamoyl]azetidin-1-yl}-5-cyanonicotinate
ethyl 6-{4-[(benzylsulfonyl)carbamoyl]piperidin-1-yl}-5-cyano-2-(2-ethoxy-2-oxoethoxy)nicotinate
ethyl 2-(azidomethyl)-6-{3-[(benzylsulfonyl)carbamoyl]azetidin-1-yl}-5-cyanonicotinate
ethyl 6-{3-[(benzylsulfonyl)carbamoyl]azetidin-1-yl}-5-cyano-2-[(methylsulfonyl)methyl]nicotinate
ethyl 2-(azidomethyl)-6-{4-[(benzylsulfonyl)carbamoyl]piperidin-1-yl}-5-cyanonicotinate
ethyl 6-{4-[(benzylsulfonyl)carbamoyl]piperidin-1-yl}-5-cyano-2-[(methylsulfonyl)methyl]nicotinate
ethyl 6-{3-[(benzylsulfonyl)carbamoyl]azetidin-1-yl}-5-cyano-2-[(2,5-dioxopyrrolidin-1-yl)methyl]nicotinate
ethyl 6-{3-[(benzylsulfonyl)carbamoyl]azetidin-1-yl}-5-cyano-2-[(glycoloyloxy)methyl]nicotinate
ethyl 6-{4-[(benzylsulfonyl)carbamoyl]piperidin-1-yl}-5-cyano-2-[(2,5-dioxopyrrolidin-1-yl)methyl]nicotinate
ethyl 6-{4-[(benzylsulfonyl)carbamoyl]piperidin-1-yl}-5-cyano-2-[(glycoloyloxy)methyl]nicotinate
ethyl 6-{4-[(benzylsulfonyl)carbamoyl]piperidin-1-yl}-5-cyano-2-(2-cyanoethoxy)nicotinate
ethyl 6-{4-[(benzylsulfonyl)carbamoyl]piperidin-1-yl}-5-cyano-2-(2-hydroxyethoxy)nicotinate
ethyl 6-{4-[(benzylsulfonyl)carbamoyl]piperidin-1-yl}-5-cyano-2-[(2-hydroxyethyl)thio]nicotinate
ethyl 2-(2-acetamidoethoxy)-6-{4-[(benzylsulfonyl)carbamoyl]piperidin-1-yl}-5-cyanonicotinate
ethyl 2-[(2-acetamidoethyl)thio]-6-{4-[(benzylsulfonyl)carbamoyl]piperidin-1-yl}-5-cyanonicotinate
ethyl 2-(2-amino-2-oxoethoxy)-6-{4-[(benzylsulfonyl)carbamoyl]piperidin-1-yl}-5-cyanonicotinate
ethyl 6-{4-[(benzylsulfonyl)carbamoyl]piperidin-1-yl}-5-cyano-2-(oxetan-2-ylmethoxy)nicotinate
{[6-{4-[(benzylsulfonyl)carbamoyl]piperidin-1-yl}-5-cyano-3-(ethoxycarbonyl)pyridin-2-yl]oxy}acetic acid
Ethyl 6-{4-[(benzylsulfonyl)carbamoyl]piperidin-1-yl}-5-cyano-2-[2-(methylamino)-2-oxoethoxy]nicotinate
{[6-{4-[(benzylsulfonyl)carbamoyl]piperidin-1-yl}-5-cyano-3-(ethoxycarbonyl)pyridin-2-yl]thio}acetic acid
Ethyl 6-{4-[(benzylsulfonyl)carbamoyl]piperidin-1-yl}-5-cyano-2-[(ethylthio)methyl]nicotinate
Ethyl 6-{4-[(benzylsulfonyl)carbamoyl]piperidin-1-yl}-5-cyano-2-{[(2-hydroxyethyl)thio]methyl}nicotinate
Ethyl 2-{[(2-acetamidoethyl)thio]methyl}-6-{4-[(benzylsulfonyl)carbamoyl]piperidin-1-yl}-5-cyanonicotinate
Ethyl 6-{3-[(benzylsulfonyl)carbamoyl]azetidin-1-yl}-5-cyano-2-[(ethylthio)methyl]nicotinate
Ethyl 6-{3-[(benzylsulfonyl)carbamoyl]azetidin-1-yl}-5-cyano-2-{[(2-hydroxyethyl)thio]methyl}nicotinate
Ethyl 2-{[(2-acetamidoethyl)thio]methyl}-6-{3-[(benzylsulfonyl)carbamoyl]azetidin-1-yl}-5-cyanonicotinate
ethyl 6-{4-[(benzylsulfonyl)carbamoyl]piperidin-1-yl}-5-cyano-2-[(2-oxopyrrolidin-1-yl)methyl]nicotinate
isopropyl 6-{4-[(benzylsulfonyl)carbamoyl]piperidin-1-yl}-5-cyano-2-[(2-oxopyrrolidin-1-yl)methyl]nicotinate
ethyl 5-cyano-2-[(2-oxopyrrolidin-1-yl)methyl]-6-[4-({[4-(trifluoromethyl)benzyl]sulfonyl}carbamoyl)piperidin-1-yl]nicotinate
ethyl 5-cyano-6-(4-{[(cyclopentylmethyl)sulfonyl]carbamoyl}piperidin-1-yl)-2-[(2-oxopyrrolidin-1-yl)methyl]nicotinate
ethyl 5-cyano-6-[4-({[(4-methylcyclohexyl)methyl]sulfonyl}carbamoyl)piperidin-1-yl]-2-[(2-oxopyrrolidin-1-yl)methyl]nicotinate
ethyl 5-cyano-6-(4-{[(2,4-difluorobenzyl)sulfonyl]carbamoyl}piperidin-1-yl)-2-[(2-oxopyrrolidin-1-yl)methyl]nicotinate
ethyl 6-(4-{[(4-chlorobenzyl)sulfonyl]carbamoyl}piperidin-1-yl)-5-cyano-2-[(2-oxopyrrolidin-1-yl)methyl]nicotinate
ethyl 5-cyano-6-(4-{[(4-fluorobenzyl)sulfonyl]carbamoyl}piperidin-1-yl)-2-[(2-oxopyrrolidin-1-yl)methyl]nicotinate
ethyl 5-cyano-6-(4-{[(2,4-dichlorobenzyl)sulfonyl]carbamoyl}piperidin-1-yl)-2-[(2-oxopyrrolidin-1-yl)methyl]nicotinate
ethyl 5-cyano-6-(4-{[(4-methylbenzyl)sulfonyl]carbamoyl}piperidin-1-yl)-2-[(2-oxopyrrolidin-1-yl)methyl]nicotinate
ethyl 6-(4-{[(2-chloro-4-fluorobenzyl)sulfonyl]carbamoyl}piperidin-1-yl)-5-cyano-2-[(2-oxopyrrolidin-1-yl)methyl]nicotinate
ethyl 6-(4-{[(4-chloro-2-fluorobenzyl)sulfonyl]carbamoyl}piperidin-1-yl)-5-cyano-2-[(2-oxopyrrolidin-1-yl)methyl]nicotinate
isopropyl 5-cyano-6-(4-{[(4-methylbenzyl)sulfonyl]carbamoyl}piperidin-1-yl)-2-[(2-oxopyrrolidin-1-yl)methyl]nicotinate
isopropyl 5-cyano-6-(4-{[(2,4-difluorobenzyl)sulfonyl]carbamoyl}piperidin-1-yl)-2-[(2-oxopyrrolidin-1-yl)methyl]nicotinate
isopropyl 5-cyano-6-(4-{[(cyclopentylmethyl)sulfonyl]carbamoyl}piperidin-1-yl)-2-[(2-oxopyrrolidin-1-yl)methyl]nicotinate
isopropyl 6-(4-{[(4-chlorobenzyl)sulfonyl]carbamoyl}piperidin-1-yl)-5-cyano-2-[(2-oxopyrrolidin-1-yl)methyl]nicotinate
isopropyl 6-(4-{[(2-chloro-4-fluorobenzyl)sulfonyl]carbamoyl}piperidin-1-yl)-5-cyano-2-[(2-oxopyrrolidin-1-yl)methyl]nicotinate
isopropyl 5-cyano-2-[(2-oxopyrrolidin-1-yl)methyl]-6-[4-({[4-(trifluoromethyl)benzyl]sulfonyl}carbamoyl)piperidin-1-yl]nicotinate
isopropyl 5-cyano-6-(4-{[(4-fluorobenzyl)sulfonyl]carbamoyl}piperidin-1-yl)-2-[(2-oxopyrrolidin-1-yl)methyl]nicotinate
isopropyl 6-(4-{[(4-chloro-2-fluorobenzyl)sulfonyl]carbamoyl}piperidin-1-yl)-5-cyano-2-[(2-oxopyrrolidin-1-yl)methyl]nicotinate
isopropyl 5-cyano-6-(4-{[(2,4-dichlorobenzyl)sulfonyl]carbamoyl}piperidin-1-yl)-2-[(2-oxopyrrolidin-1-yl)methyl]nicotinate
isopropyl 5-cyano-6-[4-({[(4-methylcyclohexyl)methyl]sulfonyl}carbamoyl)piperidin-1-yl]-2-[(2-oxopyrrolidin-1-yl)methyl]nicotinate
ethyl 6-{4-[(anilinosulfonyl)carbamoyl]piperidin-1-yl}-5-cyano-2-[(2-oxopyrrolidin-1-yl)methyl]nicotinate
ethyl 6-{4-[(benzylsulfonyl)carbamoyl]piperidin-1-yl}-5-cyano-2-[(2-oxopiperidin-1-yl)methyl]nicotinate
ethyl 5-cyano-6-[4-({[methyl(phenyl)amino]sulfonyl}carbamoyl)piperidin-1-yl]-2-[(2-oxopyrrolidin-1-yl)methyl]nicotinate
ethyl 5-cyano-6-[4-({[(4-methylcyclohexyl)methyl]sulfonyl}carbamoyl)piperidin-1-yl]-2-[(2-oxopiperidin-1-yl)methyl]nicotinate
ethyl 5-cyano-2-[(2-oxopiperidin-1-yl)methyl]-6-[4-({[4-(trifluoromethyl)benzyl]sulfonyl}carbamoyl)piperidin-1-yl]nicotinate
ethyl 5-cyano-6-(4-{[(cyclopentylmethyl)sulfonyl]carbamoyl}piperidin-1-yl)-2-[(2-oxopiperidin-1-yl)methyl]nicotinate
ethyl 5-cyano-6-(4-{[(cyclohexylmethyl)sulfonyl]carbamoyl}piperidin-1-yl)-2-[(2-oxopiperidin-1-yl)methyl]nicotinate
ethyl 5-cyano-6-(4-{[(4-fluorobenzyl)sulfonyl]carbamoyl}piperidin-1-yl)-2-[(2-oxopiperidin-1-yl)methyl]nicotinate
ethyl 5-cyano-6-(4-{[(4-methylbenzyl)sulfonyl]carbamoyl}piperidin-1-yl)-2-[(2-oxopiperidin-1-yl)methyl]nicotinate
ethyl 6-{4-[(anilinosulfonyl)carbamoyl]piperidin-1-yl}-5-cyano-2-[(2-oxopiperidin-1-yl)methyl]nicotinate
ethyl 5-cyano-6-(4-{[(cyclohexylmethyl)sulfonyl]carbamoyl}piperidin-1-yl)-2-[(2-oxopyrrolidin-1-yl)methyl]nicotinate
ethyl 5-cyano-2-[(2-oxopyrrolidin-1-yl)methyl]-6-{4-[({4-[(trifluoromethyl)thio]benzyl}sulfonyl)carbamoyl]piperidin-1-yl}nicotinate
ethyl 5-cyano-6-(4-{[(2,4-difluorobenzyl)sulfonyl]carbamoyl}piperidin-1-yl)-2-[(2-oxopiperidin-1-yl)methyl]nicotinate
ethyl 5-cyano-6-(4-{[(4-methoxybenzyl)sulfonyl]carbamoyl}piperidin-1-yl)-2-[(2-oxopiperidin-1-yl)methyl]nicotinate
ethyl 5-cyano-6-(4-{[(4-isopropylbenzyl)sulfonyl]carbamoyl}piperidin-1-yl)-2-[(2-oxopiperidin-1-yl)methyl]nicotinate
isopropyl 6-{4-[(benzylsulfonyl)carbamoyl]piperidin-1-yl}-5-cyano-2-[(2-oxopiperidin-1-yl)methyl]nicotinate
ethyl 5-cyano-6-[4-({[methyl(phenyl)amino]sulfonyl}carbamoyl)piperidin-1-yl]-2-[(2-oxopiperidin-1-yl)methyl]nicotinate
ethyl 5-cyano-2-[(2-oxopyrrolidin-1-yl)methyl]-6-(4-{[(tetrahydro-2H-pyran-4-ylmethyl)sulfonyl]carbamoyl}piperidin-1-yl)nicotinate
isopropyl 5-cyano-2-[(2-oxopyrrolidin-1-yl)methyl]-6-(4-{[(tetrahydro-2H-pyran-4-ylmethyl)sulfonyl]carbamoyl}piperidin-1-yl)nicotinate
ethyl 5-cyano-6-(4-{[(cyclobutylmethyl)sulfonyl]carbamoyl}piperidin-1-yl)-2-[(2-oxopyrrolidin-1-yl)methyl]nicotinate
ethyl 5-cyano-6-(4-{[(4-methoxybenzyl)sulfonyl]carbamoyl}piperidin-1-yl)-2-[(2-oxopyrrolidin-1-yl)methyl]nicotinate
ethyl 5-cyano-6-[4-({[(4-fluorophenyl)(methyl)amino]sulfonyl}carbamoyl)piperidin-1-yl]-2-[(2-oxopyrrolidin-1-yl)methyl]nicotinate
ethyl 5-cyano-6-[4-({[(4-fluorophenyl)amino]sulfonyl}carbamoyl)piperidin-1-yl]-2-[(2-oxopyrrolidin-1-yl)methyl]nicotinate
isopropyl 5-cyano-6-[4-({[methyl(phenyl)amino]sulfonyl}carbamoyl)piperidin-1-yl]-2-[(2-oxopyrrolidin-1-yl)methyl]nicotinate
isopropyl 5-cyano-6-[4-({[(4-fluorophenyl)(methyl)amino]sulfonyl}carbamoyl)piperidin-1-yl]-2-[(2-oxopyrrolidin-1-yl)methyl]nicotinate
isopropyl 5-cyano-6-[4-({[(4-fluorophenyl)amino]sulfonyl}carbamoyl)piperidin-1-yl]-2-[(2-oxopyrrolidin-1-yl)methyl]nicotinate
isopropyl 5-cyano-6-(4-{[(cyclobutylmethyl)sulfonyl]carbamoyl}piperidin-1-yl)-2-[(2-oxopyrrolidin-1-yl)methyl]nicotinate
isopropyl 5-cyano-6-(4-{[(cyclohexylmethyl)sulfonyl]carbamoyl}piperidin-1-yl)-2-[(2-oxopyrrolidin-1-yl)methyl]nicotinate
isopropyl 5-cyano-6-(4-{[(4-methoxybenzyl)sulfonyl]carbamoyl}piperidin-1-yl)-2-[(2-oxopyrrolidin-1-yl)methyl]nicotinate
ethyl 6-{4-[(benzylsulfonyl)carbamoyl]piperidin-1-yl}-5-bromo-2-[(2-oxopyrrolidin-1-yl)methyl]nicotinate
ethyl 6-{4-[(benzylsulfonyl)carbamoyl]piperidin-1-yl}-5-chloro-2-[(2-oxopyrrolidin-1-yl)methyl]nicotinate
ethyl 6-{4-[(benzylsulfonyl)carbamoyl]piperidin-1-yl}-2-[(2-oxopyrrolidin-1-yl)methyl]nicotinate
ethyl 5-chloro-6-(4-{[(4-methylbenzyl)sulfonyl]carbamoyl}piperidin-1-yl)-2-[(2-oxopyrrolidin-1-yl)methyl]nicotinate
ethyl 5-chloro-6-(4-{[(4-methoxybenzyl)sulfonyl]carbamoyl}piperidin-1-yl)-2-[(2-oxopyrrolidin-1-yl)methyl]nicotinate
ethyl 5-chloro-6-(4-{[(2,4-difluorobenzyl)sulfonyl]carbamoyl}piperidin-1-yl)-2-[(2-oxopyrrolidin-1-yl)methyl]nicotinate
ethyl 6-{3-[(benzylsulfonyl)carbamoyl]azetidin-1-yl}-5-cyano-2-[(2-oxopyrrolidin-1-yl)methyl]nicotinate
ethyl 5-chloro-6-[4-({[(4-fluorophenyl)amino]sulfonyl}carbamoyl)piperidin-1-yl]-2-[(2-oxopyrrolidin-1-yl)methyl]nicotinate
ethyl 5-chloro-6-[4-({[(4-fluorophenyl)(methyl)amino]sulfonyl}carbamoyl)piperidin-1-yl]-2-[(2-oxopyrrolidin-1-yl)methyl]nicotinate
ethyl 5-chloro-6-[4-({[methyl(phenyl)amino]sulfonyl}carbamoyl)piperidin-1-yl]-2-[(2-oxopyrrolidin-1-yl)methyl]nicotinate
ethyl 5-chloro-6-(4-{[(cyclohexylmethyl)sulfonyl]carbamoyl}piperidin-1-yl)-2-[(2-oxopyrrolidin-1-yl)methyl]nicotinate
propyl 5-chloro-6-[4-({[(4-fluorophenyl)(methyl)amino]sulfonyl}carbamoyl)piperidin-1-yl]-2-[(2-oxopyrrolidin-1-yl)methyl]nicotinate
isopropyl 6-{4-[(benzylsulfonyl)carbamoyl]piperidin-1-yl}-5-chloro-2-[(2-oxopyrrolidin-1-yl)methyl]nicotinate
S-ethyl 5-cyano-6-(4-{[(cyclopentylmethyl)sulfonyl]carbamoyl}piperidin-1-yl)-2-[(2-oxopyrrolidin-1-yl)methyl]pyridine-3-carbothioate
S-ethyl 5-cyano-6-(4-{[(4-methylbenzyl)sulfonyl]carbamoyl}piperidin-1-yl)-2-[(2-oxopyrrolidin-1-yl)methyl]pyridine-3-carbothioate
isopropyl 5-cyano-2-[(2-oxopyrrolidin-1-yl)methyl]-6-[4-({[4-(trimethylsilyl)benzyl]sulfonyl}carbamoyl)piperidin-1-yl]nicotinate
ethyl 5-cyano-2-[(2-oxopyrrolidin-1-yl)methyl]-6-[4-({[4-(trimethylsilyl)benzyl]sulfonyl}carbamoyl)piperidin-1-yl]nicotinate
ethyl 5-cyano-2-[(2-oxopiperidin-1-yl)methyl]-6-{4-[({4-[(trifluoromethyl)thio]benzyl}sulfonyl)carbamoyl]piperidin-1-yl}nicotinate
ethyl 5-cyano-2-[(2-oxopiperidin-1-yl)methyl]-6-[4-({[4-(trimethylsilyl)benzyl]sulfonyl}carbamoyl)piperidin-1-yl]nicotinate
ethyl 6-(4-{[(4-tert-butylbenzyl)sulfonyl]carbamoyl}piperidin-1-yl)-5-cyano-2-[(2-oxopiperidin-1-yl)methyl]nicotinate
ethyl 2-[(2-acetamidoethyl)thio]-5-chloro-6-(4-{[(4-chlorobenzyl)sulfonyl]carbamoyl}piperidin-1-yl)nicotinate
ethyl 2-[(2-acetamidoethyl)thio]-6-{4-[(benzylsulfonyl)carbamoyl]piperidin-1-yl}-5-chloronicotinate
ethyl 6-{4-[(benzylsulfonyl)carbamoyl]piperidin-1-yl}-5-cyano-2-(4-ethoxy-4-oxobutyl)nicotinate
ethyl 6-{4-[(benzylsulfonyl)carbamoyl]piperidin-1-yl}-5-cyano-2-(3-ethoxy-3-oxopropyl)nicotinate
ethyl 6-(4-{[(2-chloro-4-fluorobenzyl)sulfonyl]carbamoyl}piperidin-1-yl)-5-cyano-2-[(2-ethoxy-2-oxoethyl)thio]nicotinate
ethyl 5-cyano-6-(4-{[(2,4-dichlorobenzyl)sulfonyl]carbamoyl}piperidin-1-yl)-2-[(2-ethoxy-2-oxoethyl)thio]nicotinate
ethyl 5-cyano-6-(4-{[(cyclopentylmethyl)sulfonyl]carbamoyl}piperidin-1-yl)-2-[(2-ethoxy-2-oxoethyl)thio]nicotinate
ethyl 6-{4-[(benzylsulfonyl)carbamoyl]piperidin-1-yl}-5-cyano-2-[(2-isopropoxy-2-oxoethyl)thio]nicotinate
ethyl 6-{4-[(benzylsulfonyl)carbamoyl]piperidin-1-yl}-5-cyano-2-(3-oxobutyl)nicotinate
ethyl 6-{4-[(benzylsulfonyl)carbamoyl]piperidin-1-yl}-5-cyano-2-[2-(1H-pyrrol-1-yl)ethoxy]nicotinate
ethyl 6-{4-[(benzylsulfonyl)carbamoyl]piperidin-1-yl}-5-cyano-2-[2-(2-oxopyrrolidin-1-yl)ethoxy]nicotinate ({[6-{4-[(benzylsulfonyl)carbamoyl]piperidin-1-yl}-5-cyano-3-(ethoxycarbonyl)pyridin-2-yl]methyl}thio)acetic acid
ethyl 6-{4-[(benzylsulfonyl)carbamoyl]piperidin-1-yl}-5-cyano-2-({[(1-methyl-1H-imidazol-2-yl)methyl]thio}methyl)nicotinate
ethyl 6-{4-[(benzylsulfonyl)carbamoyl]piperidin-1-yl}-5-cyano-2-[(1,3-thiazol-2-ylthio)methyl]nicotinate
ethyl 6-{4-[(benzylsulfonyl)carbamoyl]piperidin-1-yl}-5-cyano-2-{[(1-methyl-1H-imidazol-2-yl)thio]methyl}nicotinate
ethyl 6-{4-[(benzylsulfonyl)carbamoyl]piperidin-1-yl}-5-cyano-2-{[(3-methoxy-3-oxopropyl)thio]methyl}nicotinate
ethyl 6-{4-[(benzylsulfonyl)carbamoyl]piperidin-1-yl}-5-cyano-2-[(1S)-2-ethoxy-1-methyl-2-oxoethoxy]nicotinate
ethyl 6-{4-[(benzylsulfonyl)carbamoyl]piperidin-1-yl}-5-cyano-2-[(4-oxopentyl)oxy]nicotinate
ethyl 6-{4-[(benzylsulfonyl)carbamoyl]piperidin-1-yl}-5-cyano-2-{[(2-ethoxy-2-oxoethyl)thio]methyl}nicotinate
ethyl 6-{4-[(benzylsulfonyl)carbamoyl]piperidin-1-yl}-5-cyano-2-({[2-(dimethylamino)-2-oxoethyl]thio}methyl)nicotinate
ethyl 2-[(2-azetidin-1-yl-2-oxoethyl)thio]-6-{4-[(benzylsulfonyl)carbamoyl]piperidin-1-yl}-5-cyanonicotinate
ethyl 6-{4-[(benzylsulfonyl)carbamoyl]piperidin-1-yl}-5-cyano-2-[2-(dimethylamino)-2-oxoethoxy]nicotinate
ethyl 6-{4-[(benzylsulfonyl)carbamoyl]piperidin-1-yl}-5-cyano-2-[(2-ethoxy-2-oxoethyl)thio]nicotinate
ethyl 6-{4-[(benzylsulfonyl)carbamoyl]piperidin-1-yl}-5-fluoro-2-[(2-oxopyrrolidin-1-yl)methyl]nicotinate
ethyl 6-{4-[(benzylsulfonyl)(methyl)carbamoyl]piperidin-1-yl}-5-cyano-2-[(2-oxopiperidin-1-yl)methyl]nicotinate
methyl 6-{4-[(benzylsulfonyl)carbamoyl]piperidin-1-yl}-5-chloro-2-[(2-oxopiperidin-1-yl)methyl]nicotinate
propyl 6-{4-[(benzylsulfonyl)carbamoyl]piperidin-1-yl}-5-fluoro-2-[(2-oxopyrrolidin-1-yl)methyl]nicotinate;

and pharmaceutically acceptable salts thereof.

Processes

The following processes together with the intermediates are provided as a further feature of the present invention.

Compounds of formula (I) may be prepared by the following processes a1-a12;

a1) Compounds of formula (I) in which R₁, R₂, R₃, R₄, B, R₅, R₁₄, R₁₅, R^{c} and R^{d} are defined as in formula (I) above, X is a single bond or a carbon, can be formed by reacting a compound of formula (II), in which R₁, R₂, R₃, R₄, B, R₁₄, and R₁₅ are defined

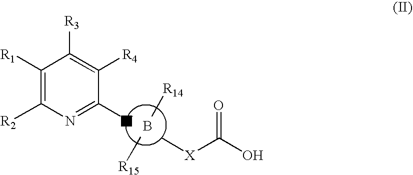

as in formula (I) above, X is a single bond or a carbon, with a compound of formula (III) in which R₅, R^{c} and R^{d} are defined as in formula (I) above.

R₅—NHSO₂—R^{c}—R^{d} (III)

The reaction is generally carried out in an inert organic solvent such as dichloromethane at ambient temperature. The reaction may be carried out using standard conditions or in the presence of TBTU, EDCI, PyBrop or the combination of EDCI and HOBt. Optionally, the reaction may be carried out in the presence of an organic base such as triethylamine or DIPEA.

a2) Compounds of formula (I) in which R₁, R₂, R₃, R₄, B, R₅, R₁₄, R₁₅, R^{c} and R^{d} are defined as in formula (I) above, X is a nitrogen, (—CH₂—NH—) or a single bond connected to a nitrogen which is a member of the B ring, can be formed by reacting a compound of formula (IV), in which R₁, R₂, R₃, R₄, B, R₁₄, and R₁₅ are defined as in formula (I) above and X is a nitrogen, (—CH₂—NH₂) or a hydrogen that is connected to a nitrogen which is a member of the B-ring, with a compound of the general

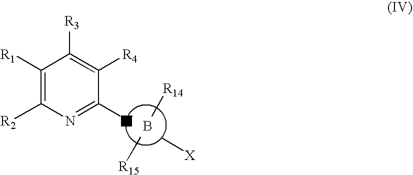

formula (III) which is defined as above.
The reaction is generally carried out in an inert solvent such as DCM. The reaction may be carried out in the presence of CDI. Optionally, the reaction may be carried out in the presence of an organic base such as triethylamine, DBU or DIPEA.

a3) Compounds of formula (I) in which R₁, R₂, R₃, R₄, B, R₁₄, R₁₅, R^{c} and R^{d} are defined as in formula (I) above, R₅ is a hydrogen, X is a nitrogen, (—CH₂—NH—) or a single bond connected to a nitrogen which is a member of the B ring, can be formed by reacting a compound of formula (IV) which is defined in a2) above, with a compound of formula (V)

O═C═N—SO₂—R^{c}R^{d} (V)

in which R^{c} and R^{d} are defined as in formula (I) above.

The reaction is generally carried out in an inert solvent such as THF. Optionally, the reaction may be carried out in the presence of an organic base such as triethylamine or DIPEA.

a4) Compounds of formula (I) in which R₁, R₂, R₃, R₄, B, R₅, R₁₄, R₁₅, R^{c} and R^{d} are defined as in formula (I) above, X is a nitrogen, (—CH₂—NH—) or a single bond connected to a nitrogen which is a member of the B ring, can be formed by reacting a compound of formula (IV) which is defined in above, with a compound of formula (VI),

R^{d}R^{c}—SO₂NR₅—COOCH₂CCl₃ (VI)

in which R₅, R^{c} and R^{d} are defined as in formula (I) above. The reaction is generally carried out in an inert solvent such as DMA. Optionally, the reaction may be carried out in the presence of an organic base such as triethylamine or DIPEA.

a5) Compounds of formula (I) may also be prepared by reacting a compound of formula (VII) in which R₁, R₂, R₃, and R₄ are defined as in formula (I) above and L is a suitable leaving group, such as chloro, bromo, iodo, fluoro, triflate (OTf) mesylate (OMs) or tosylate (OTs),

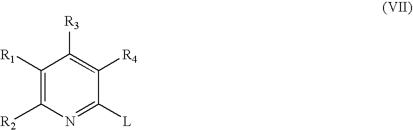

with a compound of the general formula (VIII) in which B, X, R₅, R₁₄, R₁₅, R^{c} and R^{d} are defined as in formula (I) above.

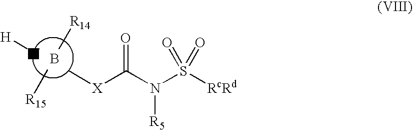

The reaction is generally carried out in an inert solvent such as DMA. Optionally, the reaction may be carried out in the presence of an organic base such as triethylamine or DIPEA.
The reaction is generally carried out at elevated temperatures using standard equipment or in a single-node microwave oven.
For some compounds, it is advantageous to carry out the reaction in ethanol in the presence of an organic base such as triethylamine.

a6) Compounds of formula (I) where R₁ represents R₆OC(O) and R₂, R₃, R₄, B, R₅, R₆, R₁₄, R₁₅, X, R^{c} and R^{d} are defined as in formula (I) above, can be transesterified using standard procedures or by reacting with R_{6′}—O⁻Li⁺ reagent, to become another compound of the general formula (I) wherein R₁ becomes R_{6′}OC(O).

a7) A compound of formula (I) in which R₁, R₂, R₃, R₄, B, R₅, R₁₄, R₁₅, and R^{d} are defined as in formula (I) above and R^{c} represents imino (—NH—) or (C₁-C₄)alkylimino in which the imino group could be substituted using standard conditions or using an alkylating agent like L-R₁₉, in which R₁₉ is defined as in formula (I) above and L is a leaving group exemplified by chloro, bromo, iodo, triflate (OTf) or tosylate (OTs), to give compounds of formula (I) in which R₁, R₂, R₃, R₄, B, R₅, R₁₄, R₁₅, and R^{d} are defined as in formula (I) above and R^{c} represents N-substituted imino (—NR₁₉—) or N-substituted (C₁-C₄)alkylimino (—N(R₁₉)—((C₁-C₄)alkyl), optionally in the presence of a strong base such as NaH.

a8) Compounds of formula (I) in which R₁ is R₆OC(O) and R₃, R₄, B, R₅, R₆, R₁₄, R₁₅, X, R^{c} and R^{d} are as defined in formula (I) above, R₂ is a substituted (C₁-C₁₂)alkoxy group defined as in formula (I) above may be prepared by reacting a compound of formula (IX)

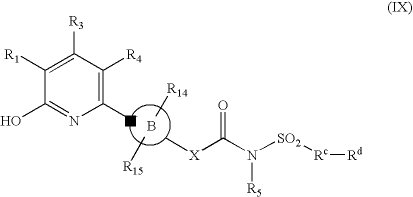

in which R₁ is R₆OC(O) and R₃, R₄, B, R₅, R₆, R₁₄, R₁₅, X, R^{c} and R^{d} are as defined in formula (I) above with a compound of formula (X)

L-R_{2′} (X)

in which R2′ is a substituted (C₁-C₁₂)alkyl defined as in formula (I) above and L is a leaving group such as chloro, bromo, iodo, triflate (OTf) or tosylate (OTs).

The reaction may be carried out in an inert organic solvent such as DMA, THF or CH₃CN. The reaction may be carried out using standard conditions or in the presence of a suitable base such as sodium hydride, DIPEA or silver carbonate or potassium carbonate. Preferentially silvercarbonate is used.

The reaction may be carried out at ambient temperature or at elevated temperatures using standard equipment or a single node microwave oven.

a9) Compounds of formula (I) in which R₁ is R₆OC(O) and R₃, R₄, B, R₅, R₆, R₁₄, R₁₅, X, R^{c} and R^{d} are as defined in formula (I) above, R₂ is a (C₁-C₁₂)alkylcarbonyloxy, arylcarbonyloxy or heterocyclylcarbonyloxy group defined as above can be prepared by reacting a compound of formula (IX) defined as above with the corresponding carboxylic acid chloride or a carboxylic acid anhydride.

The reaction may be carried out in an inert organic solvent such as DCM or THF. The reaction may be carried out using standard conditions or in the presence of a suitable base such as DIPEA, Pyridine or DMAP.

The reaction may be carried out at ambient temperature or at elevated temperatures using standard equipment or a single node microwave oven.

a10) Compounds of formula (I) in which R₁ is R₆OC(O) and R₃, R₄, B, R₅, R₆, R₁₄, R₁₅, X, R^{c} and R^{d} are as defined in formula (I) above, R₂ is a substituted (C₁-C₁₂)alkoxy group or a substituted (C₁-C₁₂)alkylthio group defined as in formula (I) above can be prepared by reacting a compound of formula (XI)

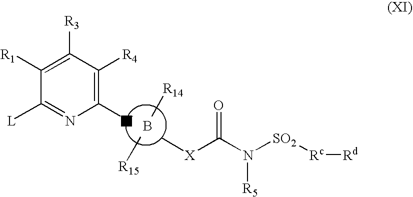

in which R₁ is R₆OC(O) and R₃, R₄, B, R₅, R₆, R₁₄, R₁₅, X, R^{c} and R^{d} are as defined in formula (I) above and L is a suitable leaving group such as Cl, Br, I or triflate (OTf) with the corresponding substituted (C₁-C₁₂)alcohol and substituted (C₁-C₁₂)alkylthiol respectively.

The reaction may be performed using standard conditions in the presence of a palladium catalyst such as or Pd(PPh₃)₄ or Pd₂(dba)₃ in combination with a suitable phosphine ligand such as PPh₃ or XANTPHOS. The reaction may be carried out in an inert solvent such as DCM, THF or dioxane optionally in the presence of a base such as DIPEA.

The reaction may be carried out at ambient temperature or at elevated temperatures using standard equipment or a single node microwave oven.

a11) Compounds of formula (I) in which R₁ is R₆OC(O) and R₃, R₄, B, R₅, R₆, R₁₄, R₁₅, X, R^{c} and R^{d} are as defined in formula (I) above, R₂ is a substituted C₁-alkyl group defined as in formula (I) above can be prepared by reacting a compound of formula (XII)

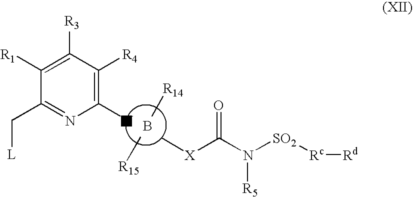

in which R₁ is R₆OC(O) and R₃, R₄, B, R₅, R₆, R₁₄, R₁₅, X, R^{c} and R^{d} are as defined in formula (I) above and L is a suitable leaving group such as Cl, Br, I, triflate (OTf) or tosylate (OTs) with the corresponding nucleophile to give the substituted C₁-alkyl group described for R₂ above.

The reaction is carried out using standard conditions in an inert solvent such as EtOH, DMF or acetone.

Preferentially the reaction is carried out in the presence of a base such as DIPEA, TEA or Cs₂CO₃.

Optionally the reaction is performed in the presence of sodium iodide.

The reaction may be carried out at ambient temperature or at elevated temperatures using standard equipment or a single node microwave oven.

a12) Compounds of formula (I) in which R₁, R₂, R₃, R₄, X, B, R₁₄, R₁₅, R^{c} and R^{d} are defined as in formula (I) above and R₅ is (C₁-C₁₂)alkyl or carboxy(C₁-C₆)alkyl may be prepared by reaction of a compound of formula (I) in which R₁, R₂, R₃, R₄, X, B, R₁₄, R₁₅, R^{c} and R^{d} are defined as in formula (I) and R₅ is H with a compound of formula (C₁-C₁₂)alkyl-L or carboxy(C₁-C₆)alkyl-L respectively, wherein L is a leaving group such as Cl, Br, I, triflate (OTf) or tosylate (OTs).

The reaction is carried out in an inert organic solvent such as DMF, THF or CH₃CN. The reaction may be carried out using standard conditions or in the presence of a suitable base such as sodium hydride, DIPEA or silver carbonate or potassium carbonate.

The reaction may be carried out at ambient temperature or at elevated temperatures using standard equipment or a single node microwave oven.

a13) Compounds of formula (I) may also be prepared by reacting a compound of formula (VII) in which R₁, R₂, R₃, and R₄ are defined as in formula (I) above except that L is a hydroxy group with a compound of the general formula (VIII) in which B, X, R₅, R₁₄, R₁₅, R^{c} and R^{d} are defined as in formula (I) above.

The reaction is generally carried out in an inert organic solvent such as DCM or THF at ambient temperature. The reaction is carried out in the presence of a suitable coupling reagent such as for example PyBrop preferentially in the presence of an organic base such as TEA or DIPEA.

The intermediates referred to above may be prepared by, for example, the methods/processes outlined below.

b1) The compounds of formula (II) in which R₁, R₂, R₃, R₄, B, R₁₄, and R₁₅ are defined as in formula (I) above, X is a single bond or a carbon, may be prepared by reacting a compound of formula (VII) defined above and L is a suitable leaving group (such as fluoro, chloro, bromo, iodo, triflate (OTf) mesylate (OMs) or tosylate (OTs)), with a compound of the general formula (XIII),

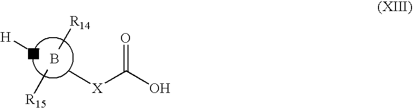

in which B, R₁₄, R₁₅ are defined as in formula (I) above and X is a single bond or a carbon.

The reaction is generally carried out at elevated temperatures using standard equipment or in a single-node microwave oven. The reaction can be carried out in an inert solvent such as ethanol, DMA or a mixture of solvents such as ethanol-water. Optionally the reaction may be carried out in the presence of an organic base such as TEA or DIPEA.

b2) The compounds of formula (II) in which R₁, R₂, R₃, R₄, B, R₁₄, and R₁₅ are defined as in formula (I) above, X is a single bond or a carbon, may be prepared by reacting a compound of formula (VII) defined above except that L is a hydroxy group with a compound of the general formula (XIII) in which B, X, R₅, R₁₄, and R₁₅ are defined as in formula (I) above.

The reaction is generally carried out in an inert organic solvent such as DCM or THF at ambient temperature. The reaction is carried out in the presence of a suitable coupling reagent such as for example PyBrop preferentially in the presence of an organic base such as TEA or DIPEA.

c1) Compounds of formula (IV) which are defined as above may be prepared by reacting the corresponding compound of formula (VII) which is defined above, with a compound of formula (XIV) in which B, R₁₄, R₁₅ are defined as in formula (I) above, X is a nitrogen, (—CH₂—NH—) or a single bond connected to a nitrogen which is a member of the B ring.

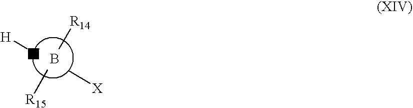

The reaction is generally carried out at elevated temperatures using standard equipment or in a single-node microwave oven. The reaction can be carried out in an inert solvent such as ethanol, DMA or a mixture of solvents such as ethanol-water. Optionally the reaction may be carried out in the presence of an organic base such as TEA or DIPEA.

c2) Compounds of formula (IV) which are defined as above may be prepared by reacting the corresponding compound of formula (VII) which is defined above except that L is a hydroxy group, with a compound of formula (XIV) in which B, R₁₄, R₁₅ are defined as in formula (I) above, X is a nitrogen, (—CH₂—NH—) or a single bond connected to a nitrogen which is a member of the B ring.

The reaction is generally carried out in an inert organic solvent such as DCM or THF at ambient temperature. The reaction is carried out in the presence of a suitable coupling reagent such as for example PyBrop preferentially in the presence of an organic base such as TEA or DIPEA.

d) Synthesis of compounds of the general formula (XV),

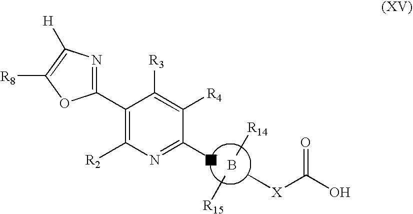

in which R₂, R₃, R₄, B, R₈, R₁₄ and R₁₅ are defined as in formula (I) above and X is a carbon or a single bond comprises the below steps. (d1-d5)

d1) Reacting the corresponding compounds of the general formula (XIII) which is defined as above with a compound of the general formula (XVI)

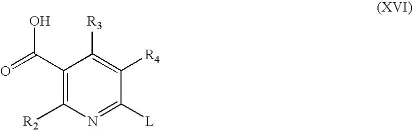

in which R₂, R₃ and R₄ are defined as in formula (I) above, and L is a suitable leaving group, such as chloro, bromo, iodo, triflate (OTf), mesylate (OMs) or tosylate (OTs), to give a compound of formula (XVII).
The reactions are carried out at elevated temperatures using standard equipment or a single-node microwave oven. Optionally the reaction may be carried out in the presence of an organic base such as TEA or DIPEA.

d2) The compounds of formula (XVII) can then be reacted

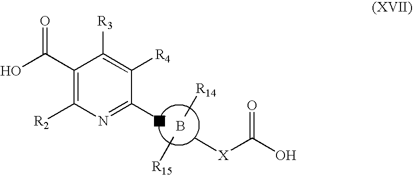

with a compound of the general formula (XVIII),

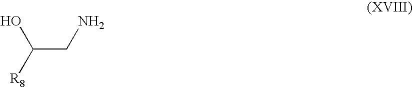

in which R₈ is defined as in formula (I) above, to give compounds of the general formula (XIX). The reactions may be carried out using standard conditions or in the presence of EDCI or the combination of EDCI and HOBt. Optionally the reaction may be carried out in the presence of an organic base such as TEA or DIPEA.

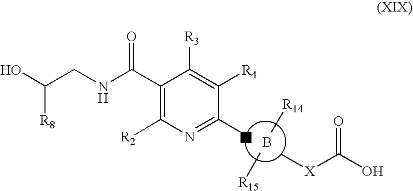

d3) This compound (XIX) can then be transformed to a compound of the general formula (XX)

d4) The preparation of compounds with the general formula (XX),

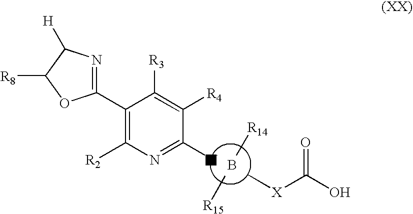

in which R₂, R₃, R₄, B, R₈, R₁₄ and R₁₅ are defined as in formula (I) above and X is a carbon or a single bond using known methods or a known reagent such as methanesulfonyl chloride. Optionally the reaction may be carried out in the presence of an organic base such as TEA.

d5) a compound of the general formula (XV) as defined above can be made by oxidizing the corresponding compound of the general formula (XX) using a known oxidation reagent such as DDQ.

e) The preparation of compounds of the general formula (XV) also comprises the steps (e1-e7) below;

e1) Reacting a compound the general formula (XXI),

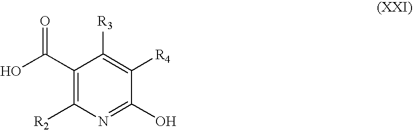

in which R₂, R₃ and R₄ are defined as in formula (I) above, with a compound of the general formula (XXII), in which R₈ is defined as in formula (I) above,

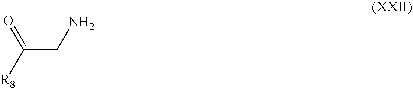

using standard conditions or in the presence of EDCI or the combination of EDCI and HOBt. Optionally the reaction may be carried out in the presence of an organic base such as TEA. This reaction gives a compound of the general formula (XXIII).

e2) The compound of the general formula (XXIII) obtained

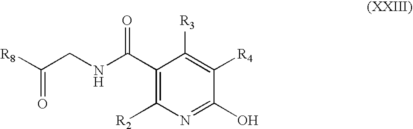

can then be transformed to a compound of the general formula (XXIV), in which R₂, R₃, R₄ and R₈ are defined as in formula (I) above, using known techniques or using a known reagent such as POCl₃ or in the presence of (Methoxycarbonylsulfamoyl)triethylammonium hydroxide (Burgess reagent).

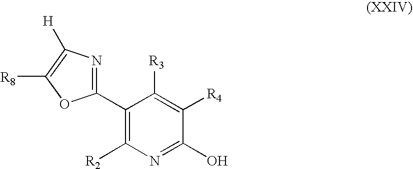

The preparation of compounds of the general formula (XXIV) which is defined as above can also comprise the steps (e3-e5) below;

e3) Reacting a compound of the general formula (XXI) above

with a compound of the general formula (XVIII), defined as above, to give a compound of the formula (XXV). The reaction is generally carried out in DCM at ambient temperature. The reaction may be carried out using standard conditions or in the presence of EDCI or the combination of EDCI and HOBt. Optionally the reaction may be carried out in the presence of an organic base such as TEA or DIPEA.

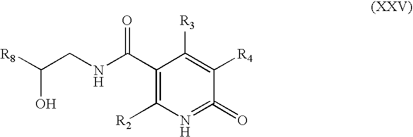

e4) The compound of formula (XXV) can be transformed to a compound (XXIII) using standard conditions or an oxidizing agent such as the mixture of oxalylchloride and DMSO.

e5) The compound of formula (XXIII) can then be transformed into a compound of the general formula (XXIV), using standard conditions or in the presence of (Methoxycarbonylsulfamoyl)triethylammonium hydroxide (Burgess reagent). The reaction is generally performed in an inert solvent such as THF. The reaction is carried out at elevated temperatures using standard equipment or a single-node microwave oven.

e6) A compound of the general formula (XXIV) can then be transformed to a compound of the general formula (XXVI),

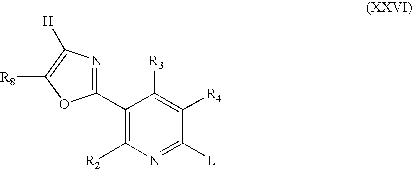

in which R₂, R₃, R₄, R₈ are defined as in formula (I) above and L is a sufficient leaving group, such as chloro, bromo, iodo, triflate (OTf), mesylate (OMs) or tosylate (OTs), using a known techniques or a reagent such as oxalyl chloride or thionyl chloride.

e7) The compound of formula (XXVI) can then be reacted with a compound of the general formula (XIII), which is defined as above, to give a compound of the general formula (XV), defined as above. The reactions are carried out at elevated temperatures using standard equipment or a single-node microwave oven. Optionally the reactions may be carried out in the presence of an organic base such as TEA or DIPEA.

f) Preparation of Compounds of the general formula (XXVII),

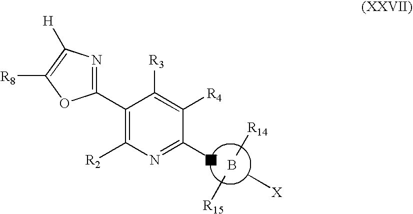

in which R₂, R₃, R₄, B, R₈, R₁₄ and R₁₅ are defined as in formula (I) above, X is a nitrogen, (—CH₂—NH—) or a single bond connected to a nitrogen which is a member of the B ring, comprises the below steps. (f1-f4)

f1) Reacting a compound of the general formula (XIV) which is defined as above with a compound of the general formula (XVI) which is defined as above, to give a compound of the general formula (XXVIII).

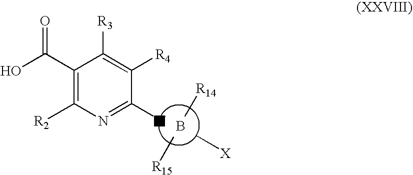

The reactions are carried out at elevated temperatures using standard equipment or a single-node microwave oven. Optionally the reaction may be carried out in the presence of an organic base such as TEA or DIPEA.

f2) The compound of formula (XXVIII) can be reacted with a compound of formula (XVIII), which is defined as above, to give compounds of the general formula (XXIX). The reactions are carried out using standard conditions or in the presence of EDCI or the combination of EDCI and HOBt. Optionally the reactions may be carried out in the presence of an organic base such as TEA or DIPEA.

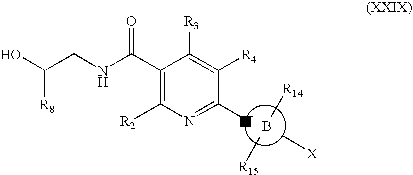

f3) This compound can then be transformed to a compound of the general formula (XXX) in which R₂, R₃, R₄, B, R₈, R₁₄ and R₁₅, are defined as in formula (I) above,

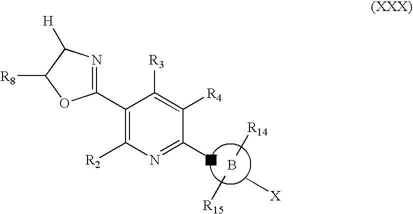

X is a nitrogen, (—CH₂—NH—) or a hydrogen connected to a nitrogen which is a member of the B ring, using known methods or a sufficient reagent such as methanesulfonyl chloride. Optionally the reaction may be carried out in the presence of an organic base such as TEA.

f4) (XXVII) can then prepared by oxidizing a compound of the general formula (XXX), which is defined as above. The reaction can be performed using standard conditions or a reagent like DDQ.

Compounds of the general formula (I), in which R₁ is R₇C(O) and R₂, R₃, R₄, R₇, B, R₁₄ and R₁₅ are defined as in formula (I) above, X is a single bond comprises the following steps (g1-g2):

g1) Reacting a compound of the general formula (XVII), described above, with N,O-dimethylhydroxylamine. The reaction can be performed using known reagents like CDI, EDCI or the combination of EDCI and HOBt to give a compound of the general formula (XXXI).

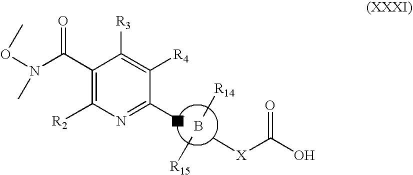

g2) Reacting compounds of the general formula (XXXI), defined as above, with a reagent of the general formula R₇—MgX, in which R₇ is defined as in formula (I) above and X is a halogen, or a reagent of the formula R₇-M, in which M is a metal exemplified by Zn and Li.

g3) Compounds of the general formula (II), in which R₁ is R₁₆SC(O) and R₂, R₃, R₄, B, R₁₄ and R₁₅ are defined as in formula (I) above, X is a single bond or a carbon atom can be made by reacting a compound of formula (XVII) with CDI and R₁₆SH or R₁₆SNa. The reaction is carried out in an inert solvent such as THF or DCM at ambient temperature or at elevated temperatures.

Compounds of the general formula (IV), in which R₁ is R₇C(O) and R₂, R₃, R₄, R₇, B, R₁₄ and R₁₅ are defined as in formula (I) above, X is a nitrogen, (—CH₂—NH—) or a single bond connected to a nitrogen which is a member of the B ring, comprises the following steps (h1-h2).

h1) Reacting a compound of the general formula (XXVIII), defined as above, with N,O-dimethylhydroxylamine. The reaction can be performed using known reagents like CDI, EDCI or the combination of EDCI and HOBt to give a compound of the general formula (XXXII).

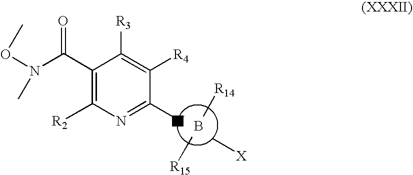

h2) A compound of the general formula (XXXII), which is defined as above can be reacted with a reagent of the general formula R₇—MgX, in which R₇ is defined as in formula (I) above and X is a halogen, or a reagent of the formula R₇-M, in which M is a metal exemplified by Zn and Li.

h3) Compounds of the general formula (IV), in which R₁ is R₁₆SC(O) and R₂, R₃, R₄, B, R₁₄ and R₁₅ are defined as in formula (I) above, X is a nitrogen, (—CH₂—NH—) or a single bond connected to a nitrogen which is a member of the B ring, can be made by reacting a compound of formula (XXVIII) with CDI and R₁₆SH or R₁₆SNa.

The reaction is carried out in an inert solvent such as THF or DCM at ambient temperature or at elevated temperatures.

Compounds of the general formula (VIII) can be formed in one of the processes (i1-i4). The compounds of formula (VIII) in which R₅ is a hydrogen are advantageously isolated as a zwitterion. A ring nitrogen of compounds of formula (XIII) and (XIV) used in the below steps may be protected by a protective group such as t-butyloxycarbonyl.

i1) Compounds of the general formula (VIII) in which B, R₅, R₁₄, R₁₅, R^{c} and R^{d} are defined as in formula (I) above, X is a single bond or a carbon, may be formed by reacting a compound of formula (XIII) with a compound of formula (III). The reaction is generally carried out in an inert organic solvent such as dichloromethane at ambient temperature. The reaction may be carried out using standard conditions or in the presence of EDCI or the combination of EDCI and HOBt. Optionally, the reaction may be carried out in the presence of an organic base such as triethylamine or DIPEA.

i2) Compounds of the general formula (VIII) in which R₅ is hydrogen, B, R₁₄, R₁₅, R^{c} and R^{d} are defined as in formula (I) above, X is a nitrogen, (—CH₂—NH—) or a single bond connected to a nitrogen which is a member of the B ring, can be formed by reacting a compound of formula (XIV) defined as above with a compound of formula (V), defined as above. The reaction is generally carried out in an inert solvent such as THF. The reaction may also be carried out in the presence of an organic base such as triethylamine or DIPEA.

i3) Compounds of the general formula (VIII) in which B, R₅, R₁₄, R₁₅, R^{c} and R^{d} defined as in formula (I) above, X is a nitrogen, (—CH₂—NH—) or a single bond connected to a nitrogen which is a member of the B ring, can also be formed by reacting a compound of formula (XIV) with a compound of formula (VI) which is defined as above. The reaction is generally carried out in a solvent such as DMA. This reaction may also be carried out in the presence of an organic base such as triethylamine or DIPEA

i4) A compound of formula (VIII) which is protected with t-butoxy carbonyl may be transformed into a compound without the protective group using standard procedures or a reagent such as HCl or TFA.

j1) Compounds of the general formula (VII) which are defined as above can be formed by reacting a compound of formula (XXXIII) using standard conditions or with a chlorinating reagent such as oxalyl chloride, thionyl chloride or POCl₃. Advantageously dimethylformamide may be used. The reaction may be performed in an inert solvent such as DCM. Advantageously the inert solvent is toluene.

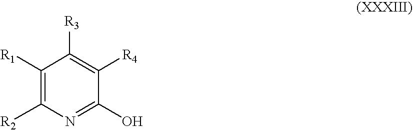

j1) Compounds of the general formula (VII) which are defined as above can be formed by reacting a compound of formula (XXXIII) using standard conditions or with a chlorinating reagent such as oxalyl chloride, thionyl chloride or POCl₃. Advantageously dimethylformamide may be used. The reaction may be performed in an inert solvent such as DCM. Advantageously the inert solvent is toluene.

The reaction may also be carried out with methyl sulfonyl chloride in the presence of a base, such as DIPEA, in an inert solvent such as DCM.

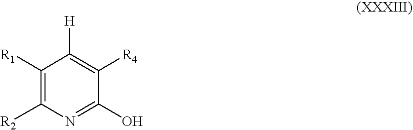

j2a) Compounds of the general formula (VII) in which R₁ is R₁₆S(CO), L is Cl, and R₂, R₃ and R₄ are as defined in Formula I may be formed by reacting a compound of formula L

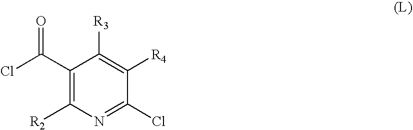

in which R₂, R₃ and R₄ are defined as in formula (I) with R₁₆SH or R₁₆SNa, wherein R₁₆ is defined as in formula (I), in an inert organic solvent such as DCM or THF, Optionally the reaction is carried out in the presence of an organic base such as DIPEA or TEA.

j2b) Compounds of the general formula (L) can be formed by reacting a compound of formula (XXI) defined as above using standard conditions or with a chlorinating reagent such as oxalyl chloride, thionyl chloride or POCl₃. Advantageously dimethylformamide may be used as catalyst. The reaction may be performed in an inert solvent such as DCM or toluene. The reaction is carried out at ambient temperature or at elevated temperatures.

l) Preparation of compounds of the general formula (XXI) which is defined as above except for R₃ which is hydrogen, comprises the following steps (l₁-l₃);

l1) Reacting a compound of the formula (XXXIV), in which R₂ and R₆ are defined as in formula (I) above with dimethoxy-N,N-dimethylmethaneamine to form a

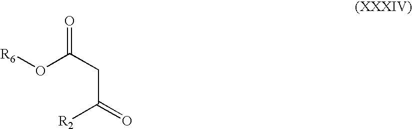

compound of formula (XXXV).

l2) This compound (XXXV) can then be reacted further with a compound of the

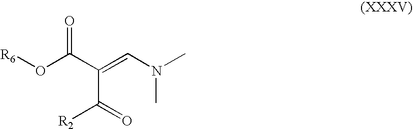

general formula R₄CH₂C(O)NH₂, in which R₄ is defined as in formula (I) above to give a compound of the general formula (XXXVI). The reaction is generally performed in an inert solvent such as ethanol, optionally in the presence of a strong base such as sodium ethoxide.

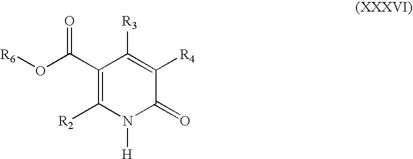

(l3) A compound of the general formula (XXXVI) can then be transformed to a compound of the general formula (XXI). The reaction is generally performed in a protic solvent such as water together with a co-solvent such as THF or methanol. The reaction can be performed using standard reagents or in the presence of LiOH, NaOH or KOH.

m) Compounds of the general formula (IX) wherein R₃, R₁₄, R₁₅, B, X, R₅, R^{c} and R^{d} are defined as in formula (I) R₁ is R₆OC(O) and R₄ is CN may be prepared by the following steps m1-m9 below

m1) Reacting a compound of the general formula (XXXVII)

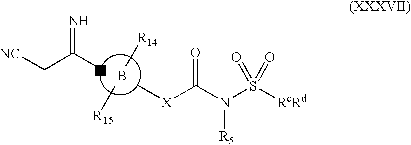

where R₅, B, R₁₄, R₁₅, X, R^{c} and R^{d} are as defined in formula (I) above with a compound of formula (XXXVIII)

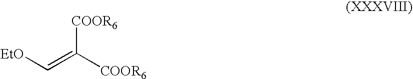

The reaction is generally carried out in an inert organic solvent such as EtOH or DMSO.

The reaction is carried out at ambient temperature or at elevated temperatures using standard equipment or a single node microwave oven.

m2) Compounds of the general formula (XXXVIII) defined above can be prepared by reacting a compound of the general formula (VIII) as defined above with a compound of formula (XXXIX)

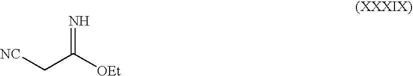

using essentially the same procedure as described in [Macconi, A et. Al., J. Heterocyclic chemistry, 26, p. 1859 (1989)].

m3) Compounds of general formula (IX) above wherein R₃, B, R₁₄, R₁₅, R₅, R^{c} and R^{d} are defined as in formula (I), R₁ is R₆OC(O), R₄ is CN and X is a single bond or a carbon atom may be prepared by reacting a compound of formula (XXXX)

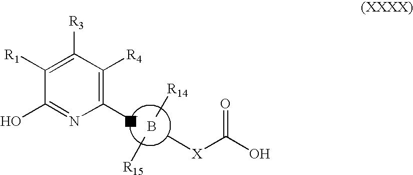

with a compound of formula (III) defined as above.

The reaction is generally carried out in an inert organic solvent such as dichloromethane at ambient temperature. The reaction may be carried out using standard conditions or in the presence of TBTU, EDCI, PyBrop or the combination of EDCI and HOBt. Optionally, the reaction may be carried out in the presence of an organic base such as triethylamine or DIPEA.

m4) Compounds of general formula (XXXX) may be prepared by reacting a compound of general formula (XXXXI)

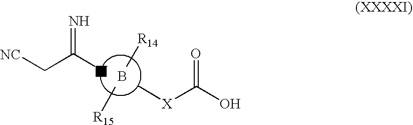

wherein R₁₄, R₁₅, and B is defined as in formula (I) and X is a single bond or a carbon atom with a compound of formula (XXXVIII) defined as above.

The reaction is generally carried out in an inert organic solvent such as EtOH or DMSO.

The reaction is carried out at ambient temperature or at elevated temperatures using standard equipment or a single node microwave oven.

m5) Compounds of the general formula (XXXXI) defined above can be prepared by reacting a compound of the general formula (XIII) as defined above with a compound

of formula (XXXIX) using essentially the same procedure as described in [Macconi, A et. Al., J. Heterocyclic chemistry, 26, p. 1859 (1989)].

m6) Compounds of general formula (IX) above wherein R₃, B, R₁₄, R₁₅, R₅, R^{c} and R^{d} are defined as in formula (I), R₁ is R₆OC(O), R₄ is CN and X is a nitrogen, (—CH₂—NH—) or a single bond connected to a nitrogen which is a member of the B ring may be prepared by reacting a compound of formula (XXXXII)

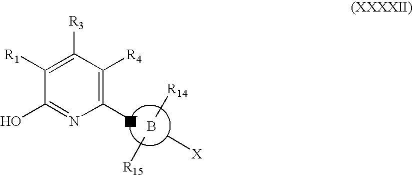

with a compound of formula (III) defined as above.

The reaction is generally carried out in an inert solvent such as DCM. The reaction may be carried out in the presence of CDI. Optionally, the reaction may be carried out in the presence of an organic base such as triethylamine, DBU or DIPEA.

m7) Compounds of general formula (IX) above wherein R₃, R₁₄, R₁₅, R^{c} and R^{d} are defined as in formula (I), R₁ is R₆OC(O), R₄ is CN, R₅ is hydrogen and X is a nitrogen, (—CH₂—NH—) or a single bond connected to a nitrogen which is a member of the B ring may be prepared by reacting a compound of formula (XXXXII) with a compound of general formula (V) as defined above.

The reaction is generally carried out in an inert solvent such as THF. Optionally, the reaction may be carried out in the presence of an organic base such as triethylamine or DIPEA.

m8) Compounds of general formula (IX) above wherein R₃, B, R₁₄, R₁₅, R₅, R^{c} and R^{d} are defined as in formula (I), R₁ is R₆OC(O), R₄ is CN and X is a nitrogen, (—CH₂—NH—) or a single bond connected to a nitrogen which is a member of the B ring may be prepared by reacting a compound of formula (XXXXII) with a compound of general formula (VI) as defined above.

The reaction is generally carried out in an inert solvent such as DMA. Optionally, the reaction may be carried out in the presence of an organic base such as triethylamine or DIPEA.

m9) Compounds of general formula (XXXXII) above may be prepared by essentially the same procedure described in steps m4)-m5) above from a compound of formula (XIV).

n1) Compounds of the general formula (XII) above in which R₁ is R₆OC(O)R₄, is CN and R₃, B, R₅, R₆, R₁₄, R₁₅, X, R^{c} and R^{d} are as defined in formula (I) above may be prepared by reacting a compound of formula (XXXXIII)

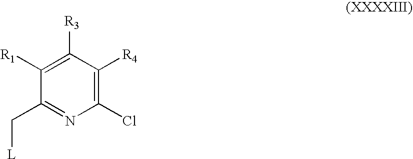

wherein R₁ is R₆OC(O)R₄ is CN, R₃ is as defined in formula (I) and L is a leaving group such as Cl, with a compound of formula (VIII) defined as above.

The reaction may be carried out in an inert solvent such as DMA or EtOH. Optionally, the reaction may be carried out in the presence of an organic base such as triethylamine or DIPEA.
The reaction is generally carried out at elevated temperatures using standard equipment or in a single-node microwave oven.
For some compounds, it is advantageous to carry out the reaction in ethanol in the presence of an organic base such as triethylamine.

n2) Compounds of general formula (XXXXIII) as defined above may be prepared by reacting a compound of formula (XXXXIV), wherein

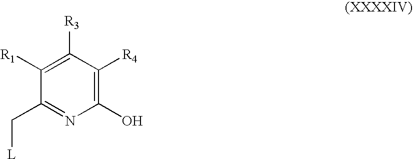

R₁ is R₆OC(O)R₄ is CN, R₃ is as defined in formula (I) and L is a leaving group such as for example Cl, with a chlorinating reagent such as oxalyl chloride, thionyl chloride or POCl₃. Advantageously dimethylformamide may be used. The reaction may be performed in an inert solvent such as DCM.

The reaction is generally carried out at elevated temperatures.

n3) Compounds of the general formula (XXXXIV) as defined above may be prepared by reacting a compound of general formula (XXXXV), wherein R₆ is as defined in formula (I),

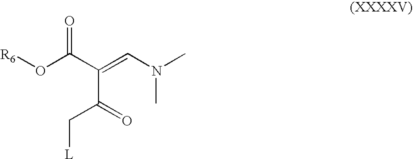

with NC—CH₂C(O)NH₂.
The reaction is generally performed in an inert solvent such as ethanol, optionally in the presence of a strong base such as sodium ethoxide.

o1) Compounds of general formula (II), wherein R₃, B, R₁₄, R₁₅, R₅, R^{c} and R^{d} are defined as in formula (I), R₁ is R₆OC(O), R₄ is CN, R₂ is a substituted (C₁-C₁₂)alkoxy group and X is a single bond or a carbon atom may be prepared by reacting a compound of formula (XXXX) as defined above, with a compound of formula (X)

L-R_{2′} (X)

in which R2′ is a substituted (C₁-C₁₂)alkyl defined as in formula (I) above and L is a leaving group such as chloro, bromo, iodo, triflate (OTf) or tosylate (OTs).

The reaction may be carried out in an inert organic solvent such as DMA, THF or CH₃CN. The reaction may be carried out using standard conditions or in the presence of a suitable base such as sodium hydride, DIPEA or silver carbonate or potassium carbonate. Preferentially silvercarbonate is used.

The reaction may be carried out at ambient temperature or at elevated temperatures using standard equipment or a single node microwave oven.

o2) Compounds of general formula (IV), wherein R₃, B, R₁₄, R₁₅, R₅, R^{c} and R^{d} are defined as in formula (I), R₁ is R₆OC(O), R₄ is CN, R₂ is a substituted (C₁-C₁₂)alkoxy group and X is a nitrogen atom, (—CH₂—NH—) or a single bond connected to a nitrogen atom which is a member of the B-ring may be prepared by reacting a compound of formula (XXXXII) as defined above, with a compound of formula (X)

L-R_{2′} (X)

in which R2′ is a substituted (C₁-C₁₂)alkyl defined as in formula (I) above and L is a leaving group such as chloro, bromo, iodo, triflate (OTf) or tosylate (OTs).

The reaction may be carried out in an inert organic solvent such as DMA, THF or CH₃CN. The reaction may be carried out using standard conditions or in the presence of a suitable base such as sodium hydride, DIPEA or silver carbonate or potassium carbonate. Preferentially silvercarbonate is used.

The reaction may be carried out at ambient temperature or at elevated temperatures using standard equipment or a single node microwave oven.

p) Compounds of general formula (XII) as defined above may be prepared by reacting a compound of formula (IX) with a halogenating reagent, such as thionylchloride, POCl₃ or oxalyl chloride. Optionally the reaction is performed in the presence of DMF.

The reaction may also be carried out in an inert solvent, such as DCM, using trifluoromethanesulfonic anhydride, optionally in the presence of an organic base such as TEA or DIPEA at or below r.t.

q) The preparation of compounds of the general formula (XXXXVI), in which B, R₁₄ and R₁₅ are defined as for formula (I) with the exception that R₁₄ is connected to the same atom as X, and X is defined as a single bond, comprises the below step;

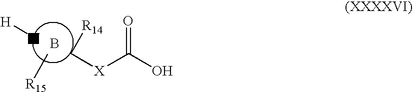

q1) Reacting the corresponding (XXXXVII) with R₁₄-L, wherein L is a suitable leaving group, such as chloro, bromo, iodo,

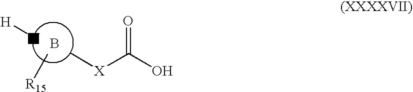

triflate (OTf), mesylate (OMs) or tosylate (OTs) to form compounds of the general formula (XXXXVI), using standard conditions or in the presence of a mixture of BuLi and diisopropylamine (to form LDA).
The preparation of compounds of the formula (III) comprises the below processes. (r1-r3)

r1) A compound of the formula LR^{c}R^{d} wherein L is a suitable leaving group, such as chloro, bromo, iodo could be transformed to the corresponding compound (III) using a sequence of reactions using first SMOPS* (*Baskin and Wang. Tetrahedron Letters, 2002, 43, 8479-83. See esp. page 8480, left hand column) followed by hydrolysis using a base like NaOMe in an inert solvent like DMSO at room temperature. Followed by treatment by NH₂OSO₃H and NaOAc to give a compound of formula (III).

r2) A compound of the formula LSO₂R^{c}R^{d} wherein L is a suitable leaving group, such as chloro, bromo, iodo could be reacted with ammonium hydroxide or H₂NR₅ in an inert solvent such as DCM to give a compound of formula (III).

r3) A compound of the formula LR^{c}R^{d} wherein L is a suitable leaving group, such as chloro, bromo, iodo could be transformed to the corresponding compound (III) using a sequence of reactions first Na₂SO₃, followed by a using a reagent such as PCl₅, POCl₃ or SOCl₂, followed by ammonium hydroxide or H₂NR₅ to give a compound of formula (III).

At any stage in the synthesis of amine substituted pyridines, a halogen substituent in the 2, 4 or 6 position of the pyridine can be substituted with azide using known techniques. The azide can be reduced to the corresponding amine. These amines can subsequently be alkylated or acylated using known methods or with an alkylhalide or acylhalide, respectively.

Persons skilled in the art will appreciate that an acid can be transformed to the corresponding activated ester such as an acid chloride, followed by reaction with a thiol, R₁₆SH to give thioesters, R₁₆SC(O).

Persons skilled in the art will appreciate that an acid can be transformed to the corresponding activated ester such as an acid chloride, followed by reaction with a alcohol, R₆OH to give esters, R₆OC(O).

Persons skilled in the art will appreciate that a compound of formula (III) could be alkylated at the carbon atom in the alpha position to the sulfonamide using an alkylhalide. Preferably under basic conditions using a strong base such as sodium hydride.

Persons skilled in the art will appreciate that a nitrogen substituent at the 3 position of a pyridine could be replaced by a thioether chain, R₁₇S—, using known techniques or R₁₇SSR₁₇ and tert-Butylnitrite.

Persons skilled in the art will appreciate that a thioketone could be made from the corresponding ketone using known techniques or using Lawessons reagent.

Persons skilled in the art will appreciate that a pyridine N-oxide could be formed by from a pyridine using an oxidizing agent such as Urea hydrogen peroxide or hydrogen peroxide, with or without the presence of trifluoroaceticanhydrid.

The compounds of the invention may be isolated from their reaction mixtures using conventional techniques.

It will be appreciated that by those skilled in the art that the processes described above and hereinafter the functional groups of intermediate compounds may need to be protected by protecting groups.

Functional groups that it is desirable to protect include hydroxy, amino and carboxylic acid. Suitable protecting groups for hydroxy include optionally substituted and/or unsaturated alkyl groups (e.g. methyl, allyl, benzyl or tert-butyl), trialkyl silyl or diarylalkylsilyl groups (e.g. t-butyldimethylsilyl, t-butyldiphenylsilyl or trimethylsilyl) and tetrahydropyranyl. Suitable protecting groups for carboxylic acids include (C₁-C₆)alkyl or benzyl esters. Suitable protecting groups for amino include allyl, t-butyloxycarbonyl, benzyloxycarbonyl, 2-(trimethylsilyl)ethoxymethyl or 2-trimethylsilylethoxycarbonyl (Teoc).

The protection and deprotection of functional groups may take place before or after any reaction in the above mentioned processes.

Persons skilled in the art will appreciate that, in order to obtain compounds of the invention in an alternative, and on some occasions, more convenient, manner, the individual process steps mentioned hereinbefore may be performed in different order, and/or the individual reactions may be performed at a different stage in the overall route (i.e. substituents may be added to and/or chemical transformations performed upon, different intermediates to those mentioned hereinbefore in conjunction with a particular reaction). This may negate, or render necessary, the need for protecting groups.

Persons skilled in the art will appreciate that starting materials for any of the above processes can in some cases be commercially available.

Persons skilled in the art will appreciate that processes could for some starting materials above be found in the general common knowledge.

The type of chemistry involved will dictate the need for protecting groups as well as sequence for accomplishing the synthesis.

The use of protecting groups is fully described in “Protective groups in Organic Chemistry”, edited by J W F McOmie, Plenum Press (1973), and “Protective Groups in Organic Synthesis”, 3^{rd} edition, T. W. Greene & P. G. M Wutz, Wiley-Interscience (1999).

Protected derivatives of the invention may be converted chemically to compounds of the invention using standard deprotection techniques (e.g. under alkaline or acidic conditions). The skilled person will also appreciate that certain compounds of Formula (II)-(XXXXVII) and (L) may also be referred to as being “protected derivatives”

Compounds of the invention may also contain one or more asymmetric carbon atoms and may therefore exhibit optical and/or diastereoisomerism. Diastereoisomers may be separated using conventional techniques, e.g. chromatography or crystallization. The various stereisomers may be isolated by separation of a racemic or other mixture of the compounds using conventional, e.g. HPLC techniques. Alternatively the desired optical isomers may be made by reaction of the appropriate optically active starting materials under conditions which will not cause racemisation or epimerization, or by derivatisation, for example with a homochiral acid followed by separation of the diasteromeric derivatives by conventional means (e.g. HPLC, chromatography over silica or crystallization). Stereo centers may also be introduced by asymmetric synthesis, (e.g. metalloorganic reactions using chiral ligands). All stereoisomers are included within the scope of the invention. It will also be understood that some of the compounds described in the processes above may exhibit the phenomenon of tautomerism and the processes described above includes any tautomeric form.

All novel intermediates form a further aspect of the invention.

Salts of the compounds of formula (I) may be formed by reacting the free acid, or a salt thereof, or the free base, or a salt or a derivative thereof, with one or more equivalents of the appropriate base (for example ammonium hydroxide optionally substituted by C₁-C₆-alkyl or an alkali metal or alkaline earth metal hydroxide) or acid (for example a hydrohalic (especially HCl), sulphuric, oxalic or phosphoric acid). The reaction may be carried out in a solvent or medium in which the salt is insoluble or in a solvent in which the salt is soluble, e.g. water, ethanol, tetrahydrofuran or diethyl ether, which may be removed in vacuo, or by freeze drying. The reaction may also carried out on an ion exchange resin. The non-toxic physiologically acceptable salts are preferred, although other salts may be useful, e.g. in isolating or purifying the product.

### Pharmacological Data

Functional inhibition of- the P2Y₁₂ receptor can be measured by in vitro assays using cell membranes from P2Y₁₂ transfected CHO-cells, the methodology is indicated below.

Functional inhibition of 2-Me-S-ADP induced P2Y₁₂ signalling: 5 μg of membranes were diluted in 200 μl of 200 mM NaCl, 1 mM MgCl₂, 50 mM HEPES (pH 7.4), 0.01% BSA, 30 μg/ml saponin and 10 μM GDP. To this was added an EC₈₀ concentration of agonist (2-methyl-thio-adenosine diphosphate), the required concentration of test compound and 0.1 μCi ³⁵S-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintilant and counted for the amount of ³⁵S-GTPγS retained by the filter. Maximum activity was that determined in the presence of the agonist and minimum activity in the absence of the agonist following subtraction of the value determined for non-specific activity. The effect of compounds at various concentrations was plotted according to the equation

y=A+((B−A)/(1+((C/x)̂D)))

and IC₅₀ estimated where
A is the bottom plateau of the curve i.e. the final minimum y value
B is the top of the plateau of the curve i.e. the final maximum y value
C is the x value at the middle of the curve. This represents the log EC₅₀ value when A+B=100
D is the slope factor.
x is the original known x values.
Y is the original known y values.
Most of the compounds of the invention have an activity, when tested in the functional inhibition of 2-Me-S-ADP induced P2Y₁₂ signalling assay described, at a concentration of around 2 μM or below.

For example the compounds described in Examples 4 and 12 gave the following test result in the functional inhibition of 2-Me-S-ADP induced P2Y₁₂ signalling assay described.

| | IC₅₀(μM) |
|---|---|
| Example 4 | 0.28 |
| Example 12 | 0.13 |

The compounds of the invention act as P2Y₁₂ receptor antagonists and are therefore useful in therapy. Thus, according to a further aspect of the invention there is provided a compound of formula (I), or a pharmaceutically acceptable salt thereof, for use in therapy.

Thus, according to another further aspect of the invention there is provided a compound of formula (I), or a pharmaceutically acceptable salt thereof, for use as a medicament.

In a further aspect there is provided the use of a compound of formula (I), or a pharmaceutically acceptable salt thereof, for the manufacture of a medicament for treatment of a platelet aggregation disorder. In another aspect of the invention there is provided the use of a compound of formula (I), or a pharmaceutically acceptable salt thereof, for the manufacture of a medicament for the inhibition of the P2Y₁₂ receptor.

In yet another aspect of the invention there is provided a compound of formula (I), or a pharmaceutically acceptable salt thereof, for use as an inhibitor of the P2Y₁₂ receptor.

In still another aspect of the invention there is provided a compound of formula (I), or a pharmaceutically acceptable salt thereof, for use in the treatment of platelet aggregation disorder.

The compounds are useful in therapy, especially adjunctive therapy, particularly they are indicated for use as: inhibitors of platelet activation, aggregation and degranulation, promoters of platelet disaggregation, anti-thrombotic agents or in the treatment or prophylaxis of unstable angina, coronary angioplasty (PTCA), myocardial infarction, perithrombolysis, primary arterial thrombotic complications of atherosclerosis such as thrombotic or embolic stroke, transient ischaemic attacks, peripheral vascular disease, myocardial infarction with or without thrombolysis, arterial complications due to interventions in atherosclerotic disease such as angioplasty, endarterectomy, stent placement, coronary and other vascular graft surgery, thrombotic complications of surgical or mechanical damage such as tissue salvage following accidental or surgical trauma, reconstructive surgery including skin and muscle flaps, conditions with a diffuse thrombotic/platelet consumption component such as disseminated intravascular coagulation, thrombotic thrombocytopaenic purpura, haemolytic uraemic syndrome, thrombotic complications of septicaemia, adult respiratory distress syndrome, anti-phospholipid syndrome, heparin-induced thrombocytopaenia and pre-eclampsia/eclampsia, or venous thrombosis such as deep vein thrombosis, venoocclusive disease, haematological conditions such as myeloproliferative disease, including thrombocythaemia, sickle cell disease; or in the prevention of mechanically-induced platelet activation in vivo, such as cardio-pulmonary bypass and extracorporeal membrane oxygenation (prevention of microthromboembolism), mechanically-induced platelet activation in vitro, such as use in the preservation of blood products, e.g. platelet concentrates, or shunt occlusion such as in renal dialysis and plasmapheresis, thrombosis secondary to vascular damage/inflammation such as vasculitis, arteritis, glomerulonephritis, inflammatory bowel disease and organ graft rejection, conditions such as migraine, Raynaud's phenomenon, conditions in which platelets can contribute to the underlying inflammatory disease process in the vascular wall such as atheromatous plaque formation/progression, stenosis/restenosis and in other inflammatory conditions such as asthma, in which platelets and platelet-derived factors are implicated in the immunological disease process.

According to the invention there is further provided the use of a compound according to the invention in the manufacture of a medicament for the treatment of the above disorders. In particular the compounds of the invention are useful for treating myocardial infarction, thrombotic stroke, transient ischaemic attacks, peripheral vascular disease and angina, especially unstable angina. The invention also provides a method of treatment of the above disorders which comprises administering to a patient suffering from such a disorder a therapeutically effective amount of a compound according to the invention.

In a further aspect the invention provides a pharmaceutical composition comprising a compound of formula (I), or a pharmaceutically acceptable salt thereof, together with a pharmaceutically acceptable diluent, adjuvant and/or carrier.

The compounds may be administered topically, e.g. to the lung and/or the airways, in the form of solutions, suspensions, HFA aerosols and dry powder formulations; or systemically, e.g. by oral administration in the form of tablets, pills, capsules, syrups, powders or granules, or by parenteral administration in the form of sterile parenteral solutions or suspensions, by subcutaneous administration, or by rectal administration in the form of suppositories or transdermally.

The compounds of the invention may be administered on their own or as a pharmaceutical composition comprising the compound of the invention in combination with a pharmaceutically acceptable diluent, adjuvant or carrier. Particularly preferred are compositions not containing material capable of causing an adverse, e.g. an allergic, reaction.

Dry powder formulations and pressurised HFA aerosols of the compounds of the invention may be administered by oral or nasal inhalation. For inhalation the compound is desirably finely divided. The compounds of the invention may also be administered by means of a dry powder inhaler. The inhaler may be a single or a multi dose inhaler, and may be a breath actuated dry powder inhaler.

One possibility is to mix the finely divided compound with a carrier substance, e.g. a mono-, di- or polysaccharide, a sugar alcohol or another polyol. Suitable carriers include sugars and starch. Alternatively the finely divided compound may be coated by another substance. The powder mixture may also be dispensed into hard gelatine capsules, each containing the desired dose of the active compound.

Another possibility is to process the finely divided powder into spheres, which break up during the inhalation procedure. This spheronized powder may be filled into the drug reservoir of a multidose inhaler, e.g. that known as the Turbuhaler® in which a dosing unit meters the desired dose which is then inhaled by the patient. With this system the active compound with or without a carrier substance is delivered to the patient.

The pharmaceutical composition comprising the compound of the invention may conveniently be tablets, pills, capsules, syrups, powders or granules for oral administration; sterile parenteral or subcutaneous solutions, suspensions for parenteral administration or suppositories for rectal administration.

For oral administration the active compound may be admixed with an adjuvant or a carrier, e.g. lactose, saccharose, sorbitol, mannitol, starches such as potato starch, corn starch or amylopectin, cellulose derivatives, a binder such as gelatine or polyvinylpyrrolidone, and a lubricant such as magnesium stearate, calcium stearate, polyethylene glycol, waxes, paraffin, and the like, and then compressed into tablets. If coated tablets are required, the cores, prepared as described above, may be coated with a concentrated sugar solution which may contain e.g. gum arabic, gelatine, talcum, titanium dioxide, and the like. Alternatively, the tablet may be coated with a suitable polymer dissolved either in a readily volatile organic solvent or an aqueous solvent.

For the preparation of soft gelatine capsules, the compound may be admixed with e.g. a vegetable oil or polyethylene glycol. Hard gelatine capsules may contain granules of the compound using either the above mentioned excipients for tablets, e.g. lactose, saccharose, sorbitol, mannitol, starches, cellulose derivatives or gelatine. Also liquid or semisolid formulations of the drug may be filled into hard gelatine capsules.

Liquid preparations for oral application may be in the form of syrups or suspensions, for example solutions containing the compound, the balance being sugar and a mixture of ethanol, water, glycerol and propylene glycol. Optionally such liquid preparations may contain colouring agents, flavouring agents, saccharine and carboxymethylcellulose as a thickening agent or other excipients known to those skilled in art.

The invention will be further illustrated with the following non-limiting examples:

### EXAMPLES

### Examples

### General Experimental Procedure

Mass spectra was recorded on a Finnigan LCQ Duo ion trap mass spectrometer equipped with an electrospray interface (LC-MS) or LC-MS system consisting of a Waters ZQ using a LC-Agilent 1100 LC system. ¹H NMR measurements were performed on a Varian Mercury VX 400 spectrometer, operating at a 1H frequency of 400 and Varian UNITY plus 400, 500 and 600 spectrometers, operating at 1H frequencies of 400, 500 and 600 respectively. Chemical shifts are given in ppm with the solvent as internal standard. Protones on heteroatoms such as NH and OH protons are only reported when detected in NMR and can therfore be missing.
HPLC separations were performed on a Waters YMC-ODS AQS-3 120 Angstrom 3×500 mm or on a Waters Delta Prep Systems using Kromasil C8, 10 μm columns.
Chromatography was performed using Biotage silica gel 40S, 40M, 12i or Merck silica gel 60 (0.063-0.200 mm). Flash-chromatography was performed using either standard glass- or plastic-columns column or on a Biotage Horizon system
Purification Method A: The purification system and LC-MS system used in purification Method A, referred to in some of the Examples below, was Waters Fraction Lynx I Purification System Column: Sunfire Prep C₁₈, 5 μm OBD, 19×150 mm column. Gradient 5-95% CH₃CN in 0.1 mM HCOOH (pH=3). MS triggered fraction collection was used. Mass spectra were recorded on either Micromass ZQ single quadropole or a Micromass quattro micro, both equipped with a pneumatically assisted electrospray interface.
Reactions performed in a microwave reactor were performed in a Personal Chemistry Smith Creator (Single node heating), Smith synthesizer or an Emrys Optimizer. IUPAC names were generated with ACDLabs Name: Release 9:00, Product version 9.04.

The GTPγS values (IC50 in μM) mentioned in the examples below were measured by the method described below:

Functional inhibition of 2-Me-S-ADP induced P2Y₁₂ signalling: 5 μg of membranes were diluted in 200 μl of 200 mM NaCl, 1 mM MgCl₂, 50 mM HEPES (pH 7.4), 0.01% BSA, 30 μg/ml saponin and 10 μM GDP. To this was added an EC₈₀ concentration of agonist (2-methyl-thio-adenosine diphosphate), the required concentration of test compound and 0.1 μCi ³⁵S-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintilant and counted for the amount of ³⁵S-GTPγS retained by the filter. Maximum activity was that determined in the presence of the agonist and minimum activity in the absence of the agonist following subtraction of the value determined for non-specific activity. The effect of compounds at various concentrations was plotted according to the equation

y=A+((B−A)/(1+((C/x)̂D)))

and IC₅₀ estimated where
A is the bottom plateau of the curve i.e. the final minimum y value
B is the top of the plateau of the curve i.e. the final maximum y value
C is the x value at the middle of the curve. This represents the log EC₅₀ value when A+B=100
D is the slope factor.
x is the original known x values.
Y is the original known y values.

List of used abbreviations:

| Abbreviation | Explanation |
|---|---|
| Ac | Acetyl |
| aq | Aqueous |
| Boc tert- | butyloxycarbonyl |
| br | Broad |
| BSA | Bovine Serum Albumine |
| CDI | Carbonyldiimidazole |
| d | Doublet |
| dba | 1,5-diphenylpenta-1,4-dien-3-one |
| DCM | Dichloromethane |
| DDQ | 2,3-Dichloro-5,6-dicyano-1,4-benzoquinone |
| DIPEA | N,N-Diisopropylethylamine |
| DMA | N,N-Dimethylacetamide |
| DMAP | 4-N,N-dimethylaminopyridine |
| DMF | N,N-dimethylformamide |
| DMSO | Dimethylsulphoxide |
| EDCI | N-[3-(dimethylamino)propyl]-N′- |
| | ethylcarbodiimide hydrochloride |
| EtOAc | Ethyl acetate |
| EtOH | Ethanol |
| FA | Formic acid |
| H | hours |
| HEPES | [4-(2-hydroxyethyl)-1-piperazineethanesulfonic |
| | acid |
| HFA | Hydrofluoroalkanes |
| HOAc | Acetic acid |
| HOBt | 1-Hydroxybenzotriazole |
| HPLC | High-performance liquid chromatography |
| Hz | Hertz |
| IPA | isopropyl alcohol |
| iPr | isopropyl |
| iPrOAc | isoPropylacetate |
| J | Coupling constant |
| LC | Liquid chromatography |
| m | Multiplet |
| MeCN | Acetonitrile |
| MeOH | Methanol |
| MHz | Megahertz |
| Min | Minutes |
| mL | Millilitre |
| MS | Mass spectra |
| Ms | methylsulfonyl |
| MTBE | methyl tert-butylether |
| NCS | N-chlorosuccinimide |
| NMR | Nuclear magnetic resonance |
| OAc | acetate |
| Ph | Phenyl |
| q | Quartet |
| r.t | Room temperature |
| s | Singlet |
| t | triplet |
| TB | Tyrodes Buffer |
| TBTU | N-[(1H-1,2,3-benzotriazol-1- |
| | yloxy)(dimethylamino)methylene]-N- |
| | methylmethanaminium tetrafluoroborate |
| TEA | Triethylamine |
| Tf | Trifluoromethylsulfonyl |
| TFA | Trifluoroacetic acid |
| THF | Tetrahydrofurane |
| TMEDA | N,N,N′,N′-tetramethylethylendiamine |
| Ts | p-toluenesulfonyl |

### Sulfone Amides

### Synthesis of Sulfone Amides

The synthesis of the sulfonamides used in the examples below was made with one of the three methods described below:
i) By reacting the corresponding sulfonyl chloride with ammonia in THF or MeOH or by treatment with ammonium hydroxide in methylene chloride. The sulfonamides obtained was used without further purification.
ii) By essentially following the procedure described by Seto, T. et. al. in J. Organic Chemistry, Vol 68, No 10 (2003), pp. 4123-4125.
or
iii) By essentially following the procedure described by Wang, Z et. al. in Tetrahedron Letters, Vol 43 (2002), pp 8479-8483.

### Synthesis of Sulfamides.

The different sulfamides in the examples below (like N-Methyl-N-phenylsulfamide) were prepared by essentially the same procedure as described in Example 57(a) by replacing N-methylaniline with the appropriate amine.

### N-Methyl-N-phenylsulfamide (typical procedure taken from Ex. 57(a))

Chlorosulfonyl isocyanate (3.7 mL, 42.4 mmol) was dissolved in dry DCM (40 mL), the solution was cooled to 0° C. and tert-butanol (3.98 mL, 42.4 mmol) was added drop wise. The reaction mixture was stirred at rt for 2 h, the solution was cooled to 0° C. and N-methylaniline (4.61 mL, 42.4 mmol) and TEA (8.85 mL, 63.6 mmol) dissolved in dry DCM (20 mL) were added drop wise through a dropping funnel. The reaction was stirred at rt for 3 h, water was added and the organic phase was separated and dried (phase separator, Isolute) and concentrated in vacuo. The residue was dissolved in DCM (40 mL) and trifluoroacetic acid (32.7 mL, 423 mmol) was added. The reaction was stirred at rt for 20 min, the solvent was concentrated in vacuo and co evaporated with DCM (3×). The crude product was purified with flash column chromatography, using a mixture of heptane:EtOAc 70:30 as eluent, to give N-methyl-N-phenylsulfamide. Yield: 5.96 g (76%).

¹H-NMR (500 MHz, CDCl₃) [symbol] 3.22 (3H, s), 4.77 (2H, s), 7.28-7.33 (1H, m), 7.36-7.42 (4H, m).

MS m/z: 187 (M+1).

### Example 1

### N-(benzylsulfonyl)-N-({1-[3-cyano-5-(ethoxycarbonyl)-6-methylpyridin-2-yl]piperidin-4-yl}carbonyl)glycine

### (a) Ethyl 2-((dimethylamino)methylene)-3-oxobutanoate

1,1-dimethoxy-N,N-dimethylmethanamine (500 g, 4195 mmol) was added to ethyl 3-oxobutanoate (461.6 g, 3547 mmol) under an atmosphere of nitrogen at r.t during 13 minutes (weak exotherm). The orange red solution was stirred for 22 hours and concentrated in vacuo. The residue was co-evaporated with toluene (3 times 200 ml each) and used without no need for further purification in the next step.

MS m/z: 186 (M+1).

### (b) Ethyl 5-cyano-2-methyl-6-oxo-1,6-dihydropyridine-3-carboxylate

Sodium ethoxide (1240.7 g of a 21 wt % solution in EtOH, 3829 mmol) was added to a stirred suspension of 2-cyanoacetamide (298 g, 3544 mmol) in EtOH (3000 mL) during 8 minutes under an atmosphere of nitrogen at r.t. The crude condensation product from step (a) above dissolved in 950 ml EtOH was added slowly (slightly exothermic reaction) and after about one third had been added further EtOH (1000 mL) was added to allow efficient stirring (suspension) followed by the addition of the rest of the condensation product (total addition time 30 min). After stirring over night at r.t. HOAc (526 g, 8759 mmol) was added to the reaction and the mixture was concentrated in vacuo leaving a thick orange slurry (volume about 3000 mL), 1 M HCl (4628 mL, 4628 mmol) was added during 10 min followed by water (500 mL). The stirring was stopped and the precipitate was filtered off and washed with water (200 mL). NMR showed the presence of about 5-10% of the corresponding acid and the solid was washed by stirring with further water (1500 mL+3×1000 mL), a solution of saturated NaHCO₃ (400 mL) in water (600 mL) and finally water (1000 mL). Filtration of the solid and drying in vacuo at 80° C. gave pure ethyl 5-cyano-2-methyl-6-oxo-1,6-dihydropyridine-3-carboxylate. Yield: 493 g (67%).

¹H NMR (400 MHz, DMSO-d₆): δ 1.36 (3H, t, J=7.1 Hz), 2.62 (3H, s), 4.25 (2H, q, J=7.1 Hz), 8.71 (1H, s), 12.79 (1H, br s).

### (c) Ethyl 6-chloro-5-cyano-2-methylnicotinate

Toluene (4000 mL) and thionylchloride (507 g, 4262 mmol) were added to ethyl 5-cyano-2-methyl-6-oxo-1,6-dihydropyridine-3-carboxylate (293 g, 1421 mmol) under an atmosphere of nitrogen and the mixture was heated to 50° C. (oil bath temperature) and DMF (100 g, 1368 mmol) was added during 2 minutes. The temperature was raised to 80° C. (oil bath temperature) and the stirring was continued for 2 hours. The mixture was concentrated in vacuo (about 3500 ml was evaporated off) leaving a red oil. EtOH (1000 mL, 99%) was added and then evaporated off. Dichloromethane (4000 mL) was added followed by 4% NaHCO₃ solution (4000 mL) and the mixture was stirred for 15 minutes. The organic phase was separated and evaporated to give ethyl 6-chloro-5-cyano-2-methylnicotinate as a dark red crude solid which was used without further purification. Yield: 301 g (75%).

¹H NMR (400 MHz, CDCl₃): δ 1.42 (3H, t, J=7.1 Hz), 2.91 (3H, s), 4.40 (2H, q, J=7.1 Hz), 8.49 (1H, s).

### (d) tert-Butyl 4-{[(benzylsulfonyl)amino]carbonyl}piperidine-1-carboxylat

Triethylamine (591 g, 5840 mmol) was added to a stirred suspension of 1-(tert-butoxycarbonyl)piperidine-4-carboxylic acid (448 g, 1954 mmol), LiCl (23.1 g, 545 mmol) and TBTU (657 g, 2046 mmol) in THF (3000 mL) under an atmosphere of nitrogen at r.t. A solution of 1-phenylmethanesulfonamide (352 g in 1300 mL THF, 2056 mmol) was added after 1.5 hours and the stirring was continued over night. The solvent was removed in vacuo to give a thick grey-beige slurry (volume about 2500 mL). EtOAc (3500 mL) was added followed by an aqueous solution of HCl (1960 mL 3.6 M HCl and 1960 mL water). The water phase was removed and the organic phase was washed with 2×1500 mL 1 M HCl. The organic phase was cooled to 0° C. which gave a precipitate of HOBt that was filtered off. Most of the solvent was removed in vacuo to give a thick grey-white slurry. EtOH (50%, 4000 mL) was added and the slurry was stirred for 1.5 hours. The precipitated product was filtered off, washed with 50% EtOH (500 mL+2×1500 mL) and dried in a vacuum oven at 25 oC to give tert-butyl 4-[(benzylsulfonyl)carbamoyl]piperidine-1-carboxylate as a white solid. Yield 584 g (78%).

¹H NMR (400 MHz, CDCl₃): δ 1.46 (9H, s), 1.54-1.61 (2H, m), 1.70-1.74 (2H, m), 2.19-2.27 (1H, m), 2.68-2.75 (2H, m), 4.07-4.12 (2H, m), 4.66 (2H, s), 7.32-7.41 (5H, m), 7.54 (1H, br s).

### (e) N-(benzylsulfonyl)piperidine-4-carboxamide

tert-Butyl 4-[(benzylsulfonyl)carbamoyl]piperidine-1-carboxylate (583 g, 1524 mmol) was suspended in formic acid (3000 mL) under a nitrogen atmosphere and the reaction was stirred for 20 minutes. The reaction was foaming due to the gas evolution and formic acid (500 mL) was used to wash down the foam from the reaction vessel walls. After 2 hours the foaming had stopped and the reaction was clear with a few solids left. The reaction was stirred over night and 2500 ml of formic acid was removed in vacuo. Water (1000 mL) was added and the reaction was filtered. The clear solution was evaporated and water (3000 mL) was added. A saturated ammonium hydroxide solution in water was used (totally 390 mL was added and the pH was going from 3.10 to 6.10) to neutralize the acidic solution and at the endpoint (pH=6.10) a heavy precipitate of the product was formed. The mixture was stirred over night and the precipitate was filtered off and washed with water (1000 mL). Drying in a vacuum oven at 25° C. gave N-(benzylsulfonyl)piperidine-4-carboxamide as a white powder. Yield 372.4 g (87%).

¹H NMR (400 MHz, DMSO-d₆): δ 1.60-1.72 (2H, m), 1.75-1.84 (2H, m), 2.10-2.19 (1H, m), 2.77-2.87 (2H, m), 3.10-3.18 (2H, m), 4.23 (2H, s), 7.18-7.28 (5H, m), 8.17 (1H, br s).

### (f) 6-(4-{[(Benzylsulfonyl)amino]carbonyl}piperidin-1-yl)-5-cyano-2-methylnicotinic acid ethyl ester

A solution of ethyl 6-chloro-5-cyano-2-methylnicotinate (47.5 g, 211 mmol) and triethyl amine (58.36 g, 577 mmol) in EtOH (314 ml) was added to a stirred mixture of N-(benzylsulfonyl)piperidine-4-carboxamide (53.55 g, 189.7 mmol, See Example 31(b)) and EtOH (100 ml) at r.t. and the mixture was heated to 100 oC (bath temperature, 20-100° C. during 40 minutes, 100° C. 15 minutes then cool to r.t.) for 15 minutes. A solution of KHSO₄ (142.93 g in 900 mL water) was added to make the product precipitate out. The precipitate was filtered off and washed with water (2×250 mL) to give 87 g of a crude product (84% pure). The crude product was slurried in 50% EtOH (1200 mL) and heated to 50° C. (bath temperature) for 2 hours and 45 minutes followed by stirring over night at r.t. Filtration gave a crude product which was further washed by stirring with 25% EtOH (1600 mL) at 50° C. for 2 hours followed by 20% EtOH (1000 mL) at 50° C. for 2 hours. (An attempt to purify the material by using a 50% EtOH/water solution was not successful because it dissolved to much of the product). The solid obtained after the washings above (89% pure) was dissolved in 700 mL EtOAc at 70° C. and the solution was left to crystallise at r.t. over night. The crystals was filtered off and washed with EtOAc (200 mL) to give pure 6-(4-{[(benzylsulfonyl)amino]carbonyl}piperidin-1-yl)-5-cyano-2-methylnicotinic acid ethyl ester as an orange solid (fine needles) after drying. Yield: 54.94 g of. Recrystallization of the solids from the mother liquor using EtOAc gave another 10.50 g. Yield 65.44 g (73%). The product can also be crystallized from CHCl₃.

¹H NMR (400 MHz, CDCl₃): δ 1.38 (3H, t, J=7.0 Hz), 1.77-1.91 (4H, m), 2.37-2.44 (1H, m), 2.73 (3H, s), 3.10-3.17 (2H, m), 4.33 (2H, q, J=7.0 Hz), 4.64-4.68 (4H, m), 7.36-7.41 (5H, m), 8.36 (1H, s).

MS m/z: 471 (M+1).

### (g) Ethyl 6-{4-[(benzylsulfonyl)(2-tert-butoxy-2-oxoethyl)carbamoyl]piperidin-1-yl}-5-cyano-2-methylnicotinate

Chloroacetic acid (24 mg, 0.255 mmol) and DIPEA (165 mg, 1.275 mmol) was added to 6-(4-{[(benzylsulfonyl)amino]carbonyl}piperidin-1-yl)-5-cyano-2-methylnicotinic acid ethyl ester (100 mg, 0.213 mg) in DMF (1 mL) in a Smith process vial and the mixture was stirred at r.t for 14 hours and heated in a microwave oven, single node heating, 120° C. for 20 minutes followed by 16 hours at 100° C. in an oil bath. LC-MS showed only starting material. tert-Butyl chloroacetate (160 mg, 1.063 mmol) and DIPEA (83 mg, 0.64) was added and the mixture was again heated in an oil bath at 100° C. for 16 hours. The solvent was evaporated and the crude product was purified by preparative HPLC (Kromasil C₈, 10 μm, using a gradient of CH₃CN/0.1 M ammonium formiate) to give ethyl 6-{4-[(benzylsulfonyl)(2-tert-butoxy-2-oxoethyl)carbamoyl]piperidin-1-yl}-5-cyano-2-methylnicotinate. Yield: 73 mg (59%).

### (h) N-(benzylsulfonyl)-N-({1-[3-cyano-5-(ethoxycarbonyl)-6-methylpyridin-2-yl]piperidin-4-yl}carbonyl)glycine

Ethyl 6-{4-[(benzylsulfonyl)(2-tert-butoxy-2-oxoethyl)carbamoyl]piperidin-1-yl}-5-cyano-2-methylnicotinate (73 mg, 0.125 mmol) was added to DCM/TFA 1/1 (2 mL) and the mixture was stirred at r.t for 1 hour. The solvent and excess TFA was evaporated to give N-(benzylsulfonyl)-N-({1-[3-cyano-5-(ethoxycarbonyl)-6-methylpyridin-2-yl]piperidin-4-yl}carbonyl)glycine. Yield: 43 mg (65%).

¹H NMR (500 MHz, CDCl₃): δ 1.38 (3H, t, J=7.1 Hz), 1.61-1.68 (4H, m), 2.53-2.62 (1H, m), 2.74 (3H, s), 2.85-2.95 (2H, m), 4.33 (2H, q, J=7.1 Hz), 4.44 (2H, s), 4.47-4.54 (2H, m), 4.64 (2H, s), 7.43-7.50 (5H, m), 8.38 (1H, s), 9.35 (2H, br s).

MS m/z: 529 (M+1)

GTPγS (IC₅₀ μM): 0.093

### Example 2

### Ethyl 2-acetoxy-6-{4-[(benzylsulfonyl)carbamoyl]piperidin-1-yl}-5-cyanonicotinate

### (a) tert-Butyl 4-{[(benzylsulfonyl)amino]carbonyl}piperidine-1-carboxylate

TEA (591 g, 5840 mmol) was added to a stirred suspension of 1-(tert-butoxycarbonyl)piperidine-4-carboxylic acid (448 g, 1954 mmol), LiCl (23.1 g, 545 mmol) and TBTU (657 g, 2046 mmol) in THF (3000 mL) under an atmosphere of nitrogen at r.t. A solution of 1-phenylmethanesulfonamide (352 g in 1300 mL THF, 2056 mmol) was added after 1.5 hours and the stirring was continued over night. The solvent was removed in vacuo to give a thick grey-beige slurry (volume about 2500 mL). EtOAc (3500 mL) was added followed by an aqueous solution of HCl (1960 mL 3.6 M HCl and 1960 mL water). The water phase was removed and the organic phase was washed with 2×1500 mL 1 M HCl. The organic phase was cooled to 0° C. which gave a precipitate of HOBt that was filtered off. Most of the solvent was removed in vacuo to give a thick grey-white slurry. EtOH (50%, 4000 mL) was added and the slurry was stirred for 1.5 hours. The precipitated product was filtered off, washed with 50% EtOH (500 mL+2×1500 mL) and dried in a vacuum oven at 25° C. to give tert-butyl 4-[(benzylsulfonyl)carbamoyl]piperidine-1-carboxylate as a white solid. Yield 584 g (78%).

¹H NMR (400 MHz, CDCl₃): δ 1.46 (9H, s), 1.54-1.61 (2H, m), 1.70-1.74 (2H, m), 2.19-2.27 (1H, m), 2.68-2.75 (2H, m), 4.07-4.12 (2H, m), 4.66 (2H, s), 7.32-7.41 (5H, m), 7.54 (1H, br s).

### (b) tert-Butyl 4-[allyl(benzylsulfonyl)carbamoyl]piperidine-1-carboxylate

A mixture of tert-butyl 4-[(benzylsulfonyl)carbamoyl]piperidine-1-carboxylate (11.47 g, 30 mmol), 3-bromoprop-1-ene (10.89 g, 90 mmol) and DIPEA (7.76 g, 60 mmol) in DMF (30 mL) was stirred at r.t. for 21 hours. Water (75 mL) was added and the aqueous phase was extracted with heptane/DCM 4/1 (3×75 mL). The combined organic phase was dried (MgSO₄), filtered and evaporated to give the product which was used without further purification.

### (c) N-allyl-N-(benzylsulfonyl)piperidine-4-carboxamide trifluoroacetate

TFA/DCM 2/1 (30 mL) was added to a stirred solution of tert-butyl 4-[allyl(benzylsulfonyl)carbamoyl]piperidine-1-carboxylate (12.676 g, 30 mmol) in DCM (10 mL) at 0° C. (ice/water bath) and the stirring was continued for 5 minutes followed by 4 hours at r.t. The solvent was evaporated and the mixture was co-evaporated with DCM twice to give the product as a TFA salt which was used in the next step without further purification.

### (d) N-allyl-N-(benzylsulfonyl)-1-(2-cyanoethanimidoyl)piperidine-4-carboxamide

N-allyl-N-(benzylsulfonyl)piperidine-4-carboxamide trifluoroacetate (30 mmol) was added to a cold (ice/water bath temperature) solution of ethyl 2-cyanoethanimidoate (See McElvain, S. M.; Schroeder, J. P.; J. Am. Chem. Soc. 71, p. 40 (1949)) (15.14 g, 101.25 mmol, 75% pure) and DIPEA (23.26 g, 180 mmol) in EtOH (200 mL) and the mixture was stirred for 10 minutes followed by 16 hours at r.t LC-MS showed complete conversion of the starting material. This solution was used in the next step as such.

### (e) Ethyl 6-{4-[allyl(benzylsulfonyl)carbamoyl]piperidin-1-yl}-5-cyano-2-oxo-1,2-dihydropyridine-3-carboxylate

Diethyl (ethoxymethylene)malonate (8.43 g, 39 mmol) was added to the solution from step (d) above and the reaction mixture was stirred for 18 hours at r.t. Evaporation of the solvent gave 32 g of a crude product. 8 g (¼) of this was taken out and purified by preparative HPLC (Kromasil C₈ 10 μm, Eluent: A: CH₃CN; B: 0.2% HOAc in water/CH₃CN 95/5; C: 0.1 M NH₄OAc/CH₃CN 95/5. Using A/B/C 5/0/95 during injection and then eluting with a gradient going from A/B/C 5/95/0 to 100/0/0) to give two fractions containing the product. Fraction 1: 308 mg (8% chemical yield, 100% purity according to LC-MS and Fraction 2: 853 mg (76% pure according to LC-MS).

¹H-NMR (400 MHz, CDCl₃): δ 1.40 (3H, t, J=7.2 Hz), 1.57-1.80 (4H, m), 2.60-2.70 (1H, m), 2.92-3.03 (2H, m), 4.11-4.16 (2H, m), 4.39/2H, q, J=7.2 Hz), 4.61 (2H, s), 4.64-4.72 (2H, m), 5.19-5.30 (2H, m), 6.62-5.75 (1H, m), 7.31-7.45 (5H, m), 8.24 (1H, s), 11.90 (1H, br. S, NH).

### (f) Ethyl 2-acetoxy-6-{4-[allyl(benzylsulfonyl)carbamoyl]piperidin-1-yl}-5-cyanonicotinate

A mixture of ethyl 6-{4-[allyl(benzylsulfonyl)carbamoyl]piperidin-1-yl}-5-cyano-2-oxo-1,2-dihydropyridine-3-carboxylate (103 mg, 0.2 mmol), acetic anhydride (1 mL) and pyridine (2 drops) was placed in a Smith process vial, sealed and heated to 100° C. in an oil bath for 90 minutes. The mixture was concentrated and co-evaporated with DCM (twice) to give ethyl 2-acetoxy-6-{4-[allyl(benzylsulfonyl)carbamoyl]piperidin-1-yl}-5-cyanonicotinate which was used without further purification in the next step.

### (g) Ethyl 2-acetoxy-6-{4-[(benzylsulfonyl)carbamoyl]piperidin-1-yl}-5-cyanonicotinate

Sodium 4-methylbenzenesulfinate (53 mg, 0.300 mmol) and Pd(PPh₃)₄ (16 mg, 0.014 mmol) were added to a solution of ethyl 2-acetoxy-6-{4-[allyl(benzylsulfonyl)carbamoyl]piperidin-1-yl}-5-cyanonicotinate (111 mg, 0.20 mmol) in DCM (2 mL) under an atmosphere of nitrogen and the mixture was stirred for 1.5 hours at r.t. Extra Pd(PPh₃)₄ was added and the stirring was continued for 1.5 hours. Evaporation of the solvent and purification of the crude product by preparative HPLC (Kromasil C₈, 10 μm using a gradient of CH₃CN/0.1 M ammoniumformiate in water) gave ethyl 2-acetoxy-6-{4-[(benzylsulfonyl)carbamoyl]piperidin-1-yl}-5-cyanonicotinate. Yield 31 mg (30%).

¹H NMR (400 MHz, CDCl₃): δ 1.36 (3H, t, J=7.2 Hz), 1.74-1.93 (4H, m), 2.37 (3H, s), 2.38-2.48 (1H, m), 3.14-3.24 (2H, m), 4.30 (2H, q, J=7.2 Hz), 4.52-4.63 (2H, m), 4.64 (2H, s), 7.31-7.35 (2H, m), 7.37-7.41 (2H, m), 8.48 (1H, s).

MS m/z: 515 (M+1)

GTPγS (IC₅₀ μM): 0.024

### Example 3

### Ethyl 6-{4-[(benzylsulfonyl)carbamoyl]piperidin-1-yl}-5-cyano-2-(pyrrolidin-1-ylmethyl)nicotinate

### (a) Ethyl 2-(chloromethyl)-5-cyano-6-oxo-1,6-dihydropyridine-3-carboxylate

A mixture of ethyl 4-chloro-3-oxobutanoate (10 g, 60.75 mmol), acetic anhydride (27.3 g, 267.3 mmol) and triethylorthoformate was heated at 120° C. (bath temperature) for 3 hours. The dark mixture was concentrated in vacuo and co-evaporated once with toluene (50 mL). Heptane (50 mL) was added to precipitate the product and then removed in vacuo. The crude material was dissolved in EtOH (50 mL).

In a separate flask, sodium ethoxide (50 mL, 60.75 mmol, prepared by reaction of sodium with EtOH (50 mL)) was added dropwise to a cold (<5° C.) solution of 2-cyanoacetamide (5.11 g, 60.75 mmol) in EtOH (50 mL) and the mixture was stirred for 30 minutes after which the solution of the crude material from above was added over 10 minutes and the stirring was continued at r.t over night. The solid formed was isolated by filtration and washed with MTBE (50 mL). Drying of the filtrate gave ethyl 2-(chloromethyl)-5-cyano-6-oxo-1,6-dihydropyridine-3-carboxylate as a beige solid. Yield: 8.15 g (56%).

¹H NMR (500 MHz, DMSO-d₆) δ 1.27 (3H, t, J=7.0 Hz), 4.16 (2H, q, J=7.0 Hz), 4.75 (2H, s), 8.02 (1H, s)

### (b) Ethyl 6-chloro-2-(chloromethyl)-5-cyanonicotinate

DMF (0.076 g, 1.04 mmol) was added to a stirred slurry of ethyl 2-(chloromethyl)-5-cyano-6-oxo-1,6-dihydropyridine-3-carboxylate (1.00 g, 4.16 mmol) and oxalyl chloride (10.55 g, 83.11 mmol) at r.t (immediate gas evolution was observed). The mixture was heated to 70° C. for 4 hours and then at 50° C. over night. The mixture was diluted with butyronitrile and evaporated (twice with 20 mL) to remove excess oxalylchloride. The residue was partioned between butyronitrile (50 mL) and water (50 ml) and the water phase was acidified with concentrated HCl (0.5 mL) followed by addition of MgCl₂ (aq) to aid phase separation. The organic phase was separated and washed with water (25 mL), 20% Na₂CO₃ (aq) (0.5 mL), MgCl₂ (aq) (10 mL) and dried (MgSO₄). The crude material was purified by chromatography on silica (Eluent: a gradient of 90:10 to 40:60 to give the desired product as a coulorless solid. Yield: 2.56 g (61%).

¹H NMR (500 MHz, DMSO-d₆) δ 1.36 (3H, t, J=7.1 Hz), 4.38 (2H, q, J=7.1 Hz), 5.09 (2H, s), 8.90 (1H, s)

MS m/z: 258 (M−1)

### (c) Ethyl 6-{4-[(benzylsulfonyl)carbamoyl]piperidin-1-yl}-2-(chloromethyl)-5-cyanonicotinate

A microwave vial was charged with ethyl 6-chloro-2-(chloromethyl)-5-cyanonicotinate (540 mg, 2.08 mmol), N-(benzylsulfonyl)piperidine-4-carboxamide (618 mg, 2.19 mmol) and TEA (527 mg, 5.21 mmol) and heated to 100° C. for 10 minutes using a microwave oven. The solvent was removed in vacuo and the residue was partioned between iPrOAc (20 mL) and aq HCl (435 μL 37% HCl in 15 mL water). The aqueous phase was separated and re-extracted with iPrOAc (10 mL). The combined organic phases was washed with aqueous MgCl₂ (10 mL), dried (MgSO₄) and evaporated to give the product which was used without further purification. Yield: 929 mg (88%).

¹H NMR (500 MHz, CDCl₃) δ 1.41 (3H, t, J=7.1 Hz), 1.75-1.94 (4H, m), 2.50 (1H, ddd, J=15.0, 10.8, 4.1 Hz), 3.19 (2H, dd, J=25.1, 2.3 Hz), 4.37 (2H, q, J=7.2 Hz), 4.63 (2H, s), 4.71 (2H, d, J=13.7 Hz), 4.98 (2H, s), 7.27-7.45 (5H, m), 8.41 (1H, s).

### (d) Ethyl 6-{4-[(benzylsulfonyl)carbamoyl]piperidin-1-yl}-5-cyano-2-(pyrrolidin-1-ylmethyl)nicotinate

A mixture of ethyl 6-{4-[(benzylsulfonyl)carbamoyl]piperidin-1-yl}-2-(chloromethyl)-5-cyanonicotinate (50 mg, 0.099 mmol), pyrrolidine (28.2 mg, 0.396 mmol) and sodium iodide (14.8 mg, 0.099 mmol) in EtOH (1 mL) was stirred over night at r.t (heterogenous mixture). The solvent was removed and the crude product was purified by preparative HPLC (Kromasil C₈, 10 μm, 21.2×250 mm column, using a gradient of CH₃CN/0.1 M NH₄OAc) to give the desired product as a colourless oil. Yield: 24 mg (45%).

¹H NMR (500 MHz, CD₃CN) δ 1.38 (3H, t, J=7.1 Hz), 1.68-1.78 (2H, m), 1.84-1.92 (2H, m), 1.97-2.04 (4H, m), 2.36 (1H, ddd, J=14.4, 10.1, 4.2 Hz), 3.32-3.42 (6H, m), 4.19 (2H, s), 4.33 (2H, q, J=7.1 Hz), 4.55 (2H, d, J=13.6 Hz), 4.74 (2H, s), 7.19-7.24 (2H, m), 7.25-7.30 (3H, m), 8.40 (1H, s)

MS m/z: 540 (M+1), 538 (M−1)

GTPγS (IC₅₀ μM): 0.545

### Example 4

### Ethyl 6-{4-[(benzylsulfonyl)carbamoyl]piperidin-1-yl}-5-cyano-2-[(dimethylamino)methyl]nicotinate

A mixture of ethyl 6-{4-[(benzylsulfonyl)carbamoyl]piperidin-1-yl}-2-(chloromethyl)-5-cyanonicotinate (50 mg, 0.099 mmol), dimethyl amine (44.6 mg, 0.396 mmol) and sodium iodide (14.8 mg, 0.099 mmol) in EtOH (1 mL) was stirred over night at r.t (heterogenous mixture). The solvent was removed and the crude product was purified by preparative HPLC (Kromasil C₈, 10 μm, 21.2×250 mm column, using a gradient of CH₃CN/0.1 M NH₄OAc) to give the desired product as a colourless oil. Yield: 21 mg (41%).

¹H NMR (500 MHz, CD₃CN) δ 1.38 (3H, t, J=7.1 Hz), 1.64-1.74 (2H, m), 1.86-1.92 (2H, m), 2.41 (1H, tt, J=10.9, 4.1 Hz), 2.77 (6H, s), 3.22-3.29 (2H, m), 4.32 (2H, q, J=6.9 Hz), 4.34 (2H, s), 4.50 (2H, s), 4.63 (2H, d, J=13.6 Hz), 7.26-7.35 (5H, m), 8.39 (1H, s).

MS m/z: 514 (M+1), 512 (M−1)

GTPγS (IC₅₀ μM): 0.282

### Example 5

### Ethyl 2-(acetoxymethyl)-6-{4-[(benzylsulfonyl)carbamoyl]piperidin-1-yl}-5-cyanonicotinate

A mixture of ethyl 6-{4-[(benzylsulfonyl)carbamoyl]piperidin-1-yl}-2-(chloromethyl)-5-cyanonicotinate (50 mg, 0.099 mmol), acetic acid (52.4 mg, 0.873 mmol) sodium carbonate (21 mg, 0.198 mmol) and sodium iodide (14.8 mg, 0.099 mmol) in acetone (1 mL) was heated to 50° C. in an oil bath for 40 minutes followed by 100° C. for 10 minutes in a microwave oven. LC-MS showed incomplete conversion of starting materials and acetic acid (524 mg, 8.73 mmol) was added and the mixture was again heated to 100° C. for 15 minutes in a microwave oven. The solvent was removed and the crude product was purified by preparative HPLC (Kromasil C₈, 10 μm, 21.2×250 mm column, using a gradient of CH₃CN/0.1 M NH₄OAc) to give the desired product as a colourless oil. Yield: 15 mg (29%).

¹H NMR (500 MHz, CD₃CN) δ 1.38 (3H, t, J=7.1 Hz), 1.70-1.79 (2H, m), 1.89-1.95 (2H, m), 2.18 (3H, s), 2.60 (1H, tt, J=11.2, 4.1 Hz), 3.21-3.28 (2H, m), 4.33 (2H, q, J=7.1 Hz), 4.63 (2H, d, J=13.6 Hz), 4.67 (2H, s), 5.50 (2H, s), 7.36-7.40 (2H, m), 7.44-7.47 (3H, m), 8.44 (1H, s)

MS m/z: 529 (M+1), 527 (M−1)

GTPγS (IC₅₀ μM): 0.044

### Example 6

### Ethyl 6-{3-[(benzylsulfonyl)carbamoyl]azetidin-1-yl}-5-cyano-2-[(dimethylamino)methyl]nicotinate

### (a) 1-(tert-butoxycarbonyl)azetidine-3-carboxylic acid

(Boc)₂O (25.535 g, 117 mmol) dissolved in MeOH (70 mL) was added dropwise during 20 minutes to a stirred slurry of azetidine-3-carboxylic acid (10.11 g, 100 mmol) and Et₃N (27.8 mL, 200 mmol) in MeOH (105 mL) at r.t (mildly exothermic reaction) and the mixture was stirred over night (18 hours). The reaction was evaporated to dryness and THF (120 mL) was added and evaporated to give crude 1-(tert-butoxycarbonyl)azetidine-3-carboxylic acid which was used without further purification in the next step. Yield: 25.89 g (128%)

¹H NMR (400 MHz, CDCl₃) δ 1.43 (9H, s), 3.21-3.34 (1H, m), 4.00-4.13 (4H, m).

### (b) tert-butyl 3-[(benzylsulfonyl)carbamoyl]azetidine-1-carboxylate

TBTU (33.71 g, 105 mmol) and TEA (30.3 g, 300 mmol) was added to a solution of 1-(tert-butoxycarbonyl)azetidine-3-carboxylic acid from above (25.89 g, assumed to contain 100 mmol) and the reaction was stirred at r.t for 30 minutes. 1-phenylmethanesulfonamide (17.97 g, 105 mmol) and LiCl (1.844 g, 43.5 mmol) was added and the stirring was continued at r.t over night (23 hours). The reaction was concentrated to about ⅓ was left and EtOAc (500 mL) was added and the organic phase was washed with 2 M HCl (1×150 mL, 2×50 mL), water (2×50 mL). Drying (MgSO₄), filtration and evaporation of the solvent gave a brown powder (48.6 g). The powder was slurried in 150 mL MTBE and stirred 3 hours. The solids was filtered off and washed with MTBE (40 mL). This procedure was repeated twice with 100 mL MTBE (washing with 25 mL) to give a brownish powder (33 g) still containing some HOBt. The powder was dissolved in about 100 mL warm EtOH and water (130 mL) was added to induce a crystallisation of the product. The crystals was filtered off and dried to give pure tert-butyl 3-[(benzylsulfonyl)carbamoyl]azetidine-1-carboxylate as an off white powder. Yield: 25.4 g (71%).

¹H NMR (400 MHz, DMSO-d₆) δ 1.39 (9H, s), 3.30 (1H, m, overlapping with the water signal in DMSO), 3.78-3.95 84H, m), 4.73 (2H, s), 7.28-7.34 (2H, m), 7.36-7.41 (3H, m), 11.71 (1H, br s).

MS m/z: 353 (M−1).

### (c) N-(benzylsulfonyl)azetidine-3-carboxamide

tert-butyl 3-[(benzylsulfonyl)carbamoyl]azetidine-1-carboxylate (25.4 g, 71.7 mmol) was added to HCOOH (300 mL) at r.t and the reaction was stirred over night (22 hours). The formic acid was removed in vacuo, water (40 mL) was added and removed in vacuo. Water (130 mL) was added to the residue followed by NH4OH (aq) until pH reached 7.4 when a crystallization started. The crystals was filtered off and dried to give pure N-(benzylsulfonyl)azetidine-3-carboxamide as a white solid. Yield 15.73 g (86%).

¹H NMR (400 MHz, DMSO-d₆) δ 3.22 (1H, m), 3.87-3.96 (4H, m), 4.28 (2H, s), 7.20-7.32 (5H, m).

MS m/z: 255 (M+1)

### (d) Ethyl 6-{3-[(benzylsulfonyl)carbamoyl]azetidin-1-yl}-2-(chloromethyl)-5-cyanonicotinate

A microwave vial was charged with 6-chloro-2-(chloromethyl)-5-cyanonicotinate (417 mg, 1.61 mmol), N-(benzylsulfonyl)azetidine-3-carboxamide (429 mg, 1.69 mmol), TEA (407 mg, 4.02 mmol) and EtOH (5 mL) and heated to 100° C. for 10 minutes. The mixture was diluted with DCM (25 mL), water (10 mL) and concentrated HCl (226 μL). The phases was separated and the organic phase dried (MgSO4) and evaporated to give the desired product as a pale yellow solid. Yield: 590 mg (77%).

¹H NMR (500 MHz, DMSO-d₆) δ 1.32 (3H, t, J=7.1 Hz), 3.55-3.63 (1H, m), 4.28 (2H, q, J=7.1 Hz), 4.31-4.53 (4H, m), 4.76 (2H, s), 4.95 (2H, s), 7.31-7.43 (5H, m), 8.42 (1H, s), 11.83 (1H, s)

### (e) Ethyl 6-{3-[(benzylsulfonyl)carbamoyl]azetidin-1-yl}-5-cyano-2-[(dimethylamino)methyl]nicotinate

A microwave vial was charged with ethyl 6-{3-[(benzylsulfonyl)carbamoyl]azetidin-1-yl}-2-(chloromethyl)-5-cyanonicotinate (38 mg, 0.08 mmol), sodium iodide (a catalytic amount), dimethyl amine (0.04 mL, 0.319 mmol) and EtOH (0.75 mL) and heated to 100° C. for 15 minutes in a microwave oven. The solvent was evaporated and the crude product purified according the purification Method A (See General Experimental Procedure) to give the desired product. Yield: 11.9 mg (31%).

¹H NMR (500 MHz, DMSO-d₆) δ 1.26 (3H, t, J=7.1 Hz), 2.92 (6H, s), 3.53-3.61 (1H, m), 4.22 (2H, q, J=7.1 Hz), 4.38-4.60 (4H, m), 4.69 (2H, s), 4.74 (2H, s), 7.28-7.38 (5H, m), 8.36 (1H, s)

MS m/z: 487 (M+1); ES—485 (M−1)

GTPγS (IC₅₀ μM): 1.07

### Example 7

### Ethyl 2-(acetoxymethyl)-6-{3-[(benzylsulfonyl)carbamoyl]azetidin-1-yl}-5-cyanonicotinate

A microwave vial was charged with ethyl 6-{3-[(benzylsulfonyl)carbamoyl]azetidin-1-yl}-2-(chloromethyl)-5-cyanonicotinate (150 mg, 0.315 mmol), sodium iodide (47.1 mg, 0.315 mmol), sodium acetate (171 mg 1.26 mmol) and EtOH (3 mL) and heated to 150° C. for 2 minutes in a microwave oven. The solvent was evaporated and the residue was partioned between EtOAc (20 mL) and water (10 mL). The organic phase was washed with water (10 mL), dried and evaporated to give the crude product as a pale crystalline solid. Yield: 155 mg (98%). An analytically pure sample was obtained by using purification Method A (See General Experimental Procedure)

¹H NMR (500 MHz, CD₃CN) δ 1.38 (3H, t, J=7.1 Hz), 2.17 (3H, s), 3.53 (1H, tt, J=8.9, 5.8 Hz), 4.32 (2H, q, J=7.1 Hz), 4.39-4.52 (4H, m), 4.69 (2H, s), 5.49 (2H, s), 7.40-7.46 (5H, m), 8.38 (1H, s)

MS m/z: 501 (M+1), 499.5 (M−1)

GTPγS (IC₅₀ μM): 0.076

### Example 8

### Ethyl 6-{4-[(benzylsulfonyl)carbamoyl]piperidin-1-yl}-5-cyano-2-(2-ethoxy-2-oxoethoxy)nicotinate

### (a) Ethyl 6-{4-[allyl(benzylsulfonyl)carbamoyl]piperidin-1-yl}-5-cyano-2-(2-ethoxy-2-oxoethoxy)nicotinate

Ethyl iodoacetate (186 mg, 0.871 mmol) was added to a solution of ethyl 6-{4-[allyl(benzylsulfonyl)carbamoyl]piperidin-1-yl}-5-cyano-2-oxo-1,2-dihydropyridine-3-carboxylate (see Example 2(e)) (105 mg, 0.174 mmol) and Ag₂CO₃ (240 mg, 0.871 mmol) in CH₃CN (10 mL) and the mixture was heated to reflux for 45 minutes. LC-MS showed some remaining starting material and therefore an additional amount of ethyl iodoacetate (74 mg, 0.348 mmol) was added and the heating continued for another 40 minutes. The solvent was removed and the crude product was used in the next step without further purification.

MS m/z: 599 (M+1)

### (b) Ethyl 6-{4-[(benzylsulfonyl)carbamoyl]piperidin-1-yl}-5-cyano-2-(2-ethoxy-2-oxoethoxy)nicotinate

Sodium 4-methylbenzenesulfinate (83 mg, 0.471 mmol) and Pd(PPh₃)₄ (202 mg, 0.175 mmol) were added to a solution of the crude ethyl 6-{4-[allyl(benzylsulfonyl)carbamoyl]piperidin-1-yl}-5-cyano-2-(2-ethoxy-2-oxoethoxy)nicotinate (104 mg, 0.174 mmol) from above in DCM (10 mL) and the mixture was stirred for 2 hours under an atmosphere of nitrogen. The solvent was removed in vacuo and the residue was purified with preparative HPLC (Kromasil C8, 10 mm, 21.2×250 mm column, using a gradient of 30% to 95% CH₃CN/0.1 M NH₄OAc) followed by purification on silica using a gradient of heptane/EtOAc (95/5 to 0:100) to give the product as a white solid. Yield: 11 mg (10%).

¹H NMR (400 MHz, DMSO-d₆) δ 1.17 (3H, t, J=7.0 Hz), 1.27 (3H, t, J=7.5 Hz), 1.50-1.64 (2H, m), 1.74-1.83 (2H, m), 3.10-3.20 (2H, m), 4.13 (2H, q, J=7.2 Hz), 4.21 (2H, q, J=7.2 Hz), 4.35-4.44 (2H, m), 4.58 (2H, s), 4.92 (2H, s), 7.22-7.40 (5H, m), 8.32 (1H, s) Note! One proton signal coincide with the DMSO signal.

MS m/z: 559 (M+1)

GTPγS (IC₅₀ μM): 0.039

### Example 9

### Ethyl 2-(azidomethyl)-6-{3-[(benzylsulfonyl)carbamoyl]azetidin-1-yl}-5-cyanonicotinate

A microwave vial was charged with ethyl 6-{3-[(benzylsulfonyl)carbamoyl]azetidin-1-yl}-2-(chloromethyl)-5-cyanonicotinate (25 mg, 0.052 mmol), sodium iodide (0.79 mg, 0.005 mmol), sodium azide (13.6 mg, 0.210 mmol) and EtOH (0.75 mL) and heated to 100° C. for 15 minutes in a microwave oven. The solvent was evaporated and the residue was partitioned between DCM (5 mL) and water (5 mL). The phases were separated and the organic phase was evaporated and the crude product purified according the purification Method A (See General Experimental Procedure) to give the desired product. Yield: 22.3 mg (86%).

¹H NMR (600 MHz, DMSO-d₆) δ 1.27 (3H, t, J=7.3 Hz), 3.52-3.58 (1H, m), 4.21 (2H, q, J=6.9 Hz), 4.28-4.50 (4H, m), 4.71 (4H, s), 7.29-7.37 (5H, m), 8.36 (1H, s)

MS m/z: 484 (M+1)

GTPγS (IC₅₀ μM): 0.069

### Example 10

### Ethyl 6-{3-[(benzylsulfonyl)carbamoyl]azetidin-1-yl}-5-cyano-2-[(methylsulfonyl)methyl]nicotinate

A microwave vial was charged with ethyl 6-{3-[(benzylsulfonyl)carbamoyl]azetidin-1-yl}-2-(chloromethyl)-5-cyanonicotinate (25 mg, 0.052 mmol), sodium iodide (0.79 mg, 0.005 mmol), sodium 4-methylbenzenesulfinate (21.4 mg, 0.210 mmol) and EtOH (0.75 mL) and heated to 100° C. for 15 minutes in a microwave oven. The solvent was evaporated and the residue was partitioned between DCM (5 mL) and water (5 mL). The phases were separated and the organic phase was evaporated and the crude product purified according the purification Method A (See General Experimental Procedure) to give the desired product. Yield: 23.3 mg (82%).

¹H NMR (600 MHz, DMSO-d₆) δ 1.28 (3H, t, J=7.0 Hz), 3.08 (3H, s), 3.52-3.58 (1H, m), 4.23 (2H, q, J=7.1 Hz), 4.26-4.50 (4H, m), 4.72 (2H, s), 5.03 (2H, s), 7.29-7.38 (5H, m), 8.41 (1H, s)

MS m/z: 521 (M+1)

GTPγS(IC₅₀ μM): 0.243

### Example 11

### Ethyl 2-(azidomethyl)-6-{4-[(benzylsulfonyl)carbamoyl]piperidin-1-yl}-5-cyanonicotinate

A microwave vial was charged with ethyl 6-{4-[(benzylsulfonyl)carbamoyl]piperidin-1-yl}-2-(chloromethyl)-5-cyanonicotinate (25 mg, 0.050 mmol), sodium iodide (7.4 mg, 0.05 mmol), sodium azide (12.9 mg, 0.198 mmol) and EtOH (0.5 mL) and heated to 100° C. for 15 minutes in a microwave oven. The solvent was evaporated and the residue was partitioned between DCM (5 mL) and water (5 mL). The phases were separated and the organic phase was evaporated and the crude product purified according the purification Method A (See General Experimental Procedure) to give the desired product. Yield: 23.2 mg (89%).

¹H NMR (600 MHz, DMSO-d₆) δ 1.28 (3H, t, J=7.3 Hz), 1.58-1.68 (2H, m), 1.78-1.85 (2H, m), 3.18-3.24 (2H, m), 4.23 (2H, q, J=7.3 Hz), 4.56-4.62 (2H, m), 4.66 (2H, s), 4.74 (2H, s), 7.22-7.29 (2H, m), 7.34-7.39 (3H, m), 8.39 (1H, s)

MS m/z: 512 (M+1)

GTPγS (IC₅₀ μM): 0.025

### Example 12

### Ethyl 6-{4-[(benzylsulfonyl)carbamoyl]piperidin-1-yl}-5-cyano-2-[(methylsulfonyl)methyl]nicotinate

A microwave vial was charged with ethyl 6-{4-[(benzylsulfonyl)carbamoyl]piperidin-1-yl}-2-(chloromethyl)-5-cyanonicotinate (25 mg, 0.050 mmol), sodium iodide (7.4 mg, 0.05 mmol), sodium 4-methylbenzenesulfinate (20.2 mg, 0.198 mmol) and EtOH (0.5 mL) and heated to 100° C. for 15 minutes in a microwave oven. The solvent was evaporated and the residue was partitioned between DCM (5 mL) and water (5 mL). The phases were separated and the organic phase was evaporated and the crude product purified according the purification Method A (See General Experimental Procedure) to give the desired product. Yield: 27.7 mg (99%).

¹H NMR (600 MHz, DMSO-d₆) δ 1.28 (3H, t, J=6.9 Hz), 1.59-1.68 (2H, br m), 1.79-1.85 (2H, br m), 3.03 (3H, s), 3.14-3.20 (2H, br m), 4.24 (2H, q, J=7.4 Hz), 4.51-4.57 (2H, m), 4.66 (2H, s), 5.02 (2H, s), 7.24-7.28 (2H, m), 7.34-7.40 (3H, m), 8.43 (1H, s)

MS m/z: 549 (M+1)

GTPγS (IC₅₀ μM): 0.129

### Example 13

### Ethyl 6-{3-[(benzylsulfonyl)carbamoyl]azetidin-1-yl}-5-cyano-2-[(2,5-dioxopyrrolidin-1-yl)methyl]nicotinate

A microwave vial was charged with ethyl 6-{3-[(benzylsulfonyl)carbamoyl]azetidin-1-yl}-2-(chloromethyl)-5-cyanonicotinate (25 mg, 0.052 mmol), sodium iodide (7.9 mg, 0.052 mmol), pyrrolidine-2,5-dione (20.8 mg, 0.210 mmol) Cs₂CO₃ (34.2 mg, 0.105 mmol) and EtOH (0.5 mL) and heated to 100° C. for 15 minutes in a microwave oven. The solvent was evaporated and the residue was partitioned between DCM (5 mL) and water (5 mL). The phases were separated and the organic phase was evaporated and the crude product purified according the purification Method A (See General Experimental Procedure) to give the desired product. Yield: 9.8 mg (33%).

¹H NMR (600 MHz, DMSO-d₆) δ 1.29 (3H, t, J=7.1 Hz), 2.73 (4H, s), 3.45-3.54 (1H, br m), 4.11-4.40 (4H, br m), 4.26 (2H, q, J=7.2 Hz), 4.69 (2H, s), 4.90 (2H, s), 7.26-7.37 (5H, m), 8.36 (1H, s)

MS m/z: 540 (M+1)

GTPγS (IC₅₀ μM): 0.064

### Example 14

### Ethyl 6-{3-[(benzylsulfonyl)carbamoyl]azetidin-1-yl}-5-cyano-2-[(glycoloyloxy)methyl]nicotinate

A microwave vial was charged with ethyl 6-{3-[(benzylsulfonyl)carbamoyl]azetidin-1-yl}-2-(chloromethyl)-5-cyanonicotinate (25 mg, 0.052 mmol), sodium iodide (7.8 mg, 0.052 mmol), ethyl glycolate (15.9 mg, 0.210 mmol) Cs₂CO₃ (34.1 mg, 0.105 mmol) and EtOH (0.5 mL) and heated to 100° C. for 15 minutes in a microwave oven. The solvent was evaporated and the residue was partitioned between DCM (5 mL) and water (5 mL). The phases were separated and the organic phase was evaporated and the crude product purified according the purification Method A (See General Experimental Procedure) to give the desired product. Yield: 8.2 mg (26%).

¹H NMR (600 MHz, DMSO-d₆) δ 1.28 (3H, t, J=8.9 Hz), 3.51 (1H, br s), 4.13 (2H, d, J=7.6 Hz), 4.23 (2H, q, J=7.2 Hz), 4.24-4.48 (4H, br m), 4.70 (2H, br s), 5.45 (2H, s), 7.28-7.39 (5H, m), 8.34 (1H, s)

MS m/z: 517 (M+1)

GTPγS (IC₅₀ μM): 0.044

### Example 15

### Ethyl 6-{4-[(benzylsulfonyl)carbamoyl]piperidin-1-yl}-5-cyano-2-[(2,5-dioxopyrrolidin-1-yl)methyl]nicotinate

A microwave vial was charged with ethyl 6-{4-[(benzylsulfonyl)carbamoyl]piperidin-1-yl}-2-(chloromethyl)-5-cyanonicotinate (25 mg, 0.050 mmol), sodium iodide (7.4 mg, 0.05 mmol), pyrrolidine-2,5-dione (19.6 mg, 0.198 mmol) Cs₂CO₃ (32.3 mg, 0.099 mmol) and EtOH (0.5 mL) and heated to 100° C. for 15 minutes in a microwave oven. The solvent was evaporated and the residue was partitioned between DCM (5 mL) and water (5 mL). The phases were separated and the organic phase was evaporated and the crude product purified according the purification Method A (See General Experimental Procedure) to give the desired product. Yield: 10.9 mg (37%).

¹H NMR (600 MHz, DMSO-d₆) δ 1.30 (3H, t, J=7.2 Hz), 1.47-1.55 (2H, m), 1.74-1.80 (2H, m), 2.75 (4H, s), 3.09-3.16 (2H, m), 4.27 (2H, q, J=7.2 Hz), 4.30-4.35 (2H, m), 4.65 (2H, s), 4.92 (2H, s), 7.23-7.28 (2H, m), 7.34-7.41 (3H, m), 8.39 (1H, s)

MS m/z: 569 (M+1)

GTPγS (IC₅₀ μM): 0.017

### Example 16

### Ethyl 6-{4-[(benzylsulfonyl)carbamoyl]piperidin-1-yl}-5-cyano-2-[(glycoloyloxy)methyl]nicotinate

A microwave vial was charged with ethyl 6-{4-[(benzylsulfonyl)carbamoyl]piperidin-1-yl}-2-(chloromethyl)-5-cyanonicotinate (25 mg, 0.050 mmol), sodium iodide (7.4 mg, 0.05 mmol), ethyl glycolate (15.1 mg, 0.198 mmol) Cs₂CO₃ (32.3 mg, 0.099 mmol) and EtOH (0.5 mL) and heated to 100° C. for 15 minutes in a microwave oven. The solvent was evaporated and the residue was partitioned between DCM (5 mL) and water (5 mL). The phases were separated and the organic phase was evaporated and the crude product purified according the purification Method A (See General Experimental Procedure) to give the desired product. Yield: 5.1 mg (17%).

¹H NMR (600 MHz, DMSO-d₆) δ 1.28 (3H, t, J=7.0 Hz), 1.53-1.63 (2H, m), 1.76-1.83 (2H, m), 3.12-3.19 (2H, m), 4.13 (2H, d, J=7.0 Hz), 4.24 (2H, q, J=7.4 Hz), 4.42-4.50 (2H, m), 4.64 (2H, s), 5.45 (1H, t, J=7.0 Hz), 5.46 (2H, s), 7.23-7.28 (2H, m), 7.33-7.41 (3H, m), 8.37 (1H, s)

MS m/z: 546 (M+1)

GTPγS (IC₅₀ μM): 0.015

### Example 17

### Ethyl 6-{4-[(benzylsulfonyl)carbamoyl]piperidin-1-yl}-5-cyano-2-(2-cyanoethoxy)nicotinate

### (a) Ethyl 6-{4-[allyl(benzylsulfonyl)carbamoyl]piperidin-1-yl}-5-cyano-2-{[(trifluoromethyl)sulfonyl]oxy}nicotinate

Trifluoromethanesulfonic anhydride (186 mg, 0.66 mmol) was added dropwise to a cold (ice/water bath temperature) solution of ethyl 6-{4-[allyl(benzylsulfonyl)carbamoyl]piperidin-1-yl}-5-cyano-2-oxo-1,2-dihydropyridine-3-carboxylate (See Example 2(e)) (308 mg, 0.6 mmol) and TEA (273 mg, 2.7 mmol) in DCM (7 mL). The reaction was stirred at 0° C. for 1 hour and NaHCO₃ (aq, sat) was added. The aqueous phase was extracted with DCM (2×10 mL). The combined organic phase was dried (Na₂SO₄), filtered and evaporated to give the product which was used without further purification.

### (b) Ethyl 6-{4-[(benzylsulfonyl)carbamoyl]piperidin-1-yl}-5-cyano-2-(2-cyanoethoxy)nicotinate

A microwave vial was charged with ethyl 6-{4-[allyl(benzylsulfonyl)carbamoyl]piperidin-1-yl}-5-cyano-2-{[(trifluoromethyl)sulfonyl]oxy}nicotinate (103 mg, 0.16 mmol), Pd₂(dba)₃ (27 mg, 0.029 mmol), Xantphos (15 mg, 0.026 mmol), 3-hydroxypropanenitrile (25 μL, 0.37 mmol), DIPEA (0.1 mL, 0.57 mmol) and dioxane (4 mL) and the reaction mixture was heated to 160° C. for 10 min using microwave single node heating. The mixture was filtered through a plug of Celite and washed with DCM. NH₄Cl (aq) was added and the mixture was extracted with DCM (3 times). The combined organic layer was run through a phase separator and evaporated. The crude was purified by preparative HPLC (Kromasil C₈, 10 μm, 21.5×250 mm column, flow rate 25 mL/Minute, using a gradient of 10-40% CH₃CN/0.1 M NH₄OAc) to give the desired compound. Yield: 26 mg (31%).

¹H NMR (500 MHz, DMSO-d₆): δ 1.30 (3H, t, J=7.1 Hz), 1.61-1.70 (2H, m), 1.81-1.87 (2H, m), 2.53-2.61 (1H, m), 3.03 (2H, t, J=5.7 Hz), 3.15-3.23 (2H, m), 4.22 (2H, q, J=7.1 Hz), 4.49-4.54 (2H, m), 4.54 (2H, t, J=5.7 Hz), 4.66 (2H, s), 7.28-7.31 (2H, m), 7.36-7.42 (3H, m), 8.31 (1H, s), 11.62 (1H, br s).

MS m/z: 526 (M+1), 524 (M−1).

GTPγS (IC₅₀ μM): 0.027

### Example 18

### Ethyl 6-{4-[(benzylsulfonyl)carbamoyl]piperidin-1-yl}-5-cyano-2-(2-hydroxyethoxy)nicotinate

A microwave vial was charged with ethyl 6-{4-[allyl(benzylsulfonyl)carbamoyl]piperidin-1-yl}-5-cyano-2-{[(trifluoromethyl)sulfonyl]oxy}nicotinate (150 mg, 0.233 mmol), Pd₂(dba)₃ (21.3 mg, 0.023 mmol), Xantphos (13.5 mg, 0.023 mmol), ethylene glycol (56 mg, 0.902 mmol), DIPEA (0.1 mL, 0.57 mmol) and dioxane (3 mL) and the reaction mixture was heated to 160° C. for 10 min using microwave single node heating. NaHCO₃ (aq) was added and the mixture was extracted with DCM (3 times). The combined organic layer was run through a phase separator and evaporated. The crude was purified by preparative HPLC (Kromasil C₈, 10 μm, 21.5×250 mm column, flow rate 25 mL/Minute, using a gradient of 10-40% CH₃CN/0.1 M NH₄OAc) to give the desired compound. Yield: 62 mg (51%).

¹H NMR (500 MHz, DMSO-d₆): δ 1.27 (3H, t, J=7.1 Hz), 1.54-1.63 (2H, m), 1.75-1.81 (2H, m), 2.21-2.27 (1H, m), 3.73 (2H, t, J=5.2 Hz), 4.19 (2H, q, J=7.1 Hz), 4.24 (2H, s), 4.36 (2H, t, J=5.4 Hz), 4.37-4.42 (2H, m), 7.18-7.26 (5H, m), 8.24 (1H, s). 4 protons concealed by the water signal at 3.32.

MS m/z: 517 (M+1), 515 (M−1).

GTPγS (IC₅₀ μM): 0.031

### Example 19

### Ethyl 6-{4-[(benzylsulfonyl)carbamoyl]piperidin-1-yl}-5-cyano-2-[(2-hydroxyethyl)thio]nicotinate

A microwave vial was charged with ethyl 6-{4-[allyl(benzylsulfonyl)carbamoyl]piperidin-1-yl}-5-cyano-2-{[(trifluoromethyl)sulfonyl]oxy}nicotinate (150 mg, 0.233 mmol), Pd₂(dba)₃ (21.3 mg, 0.023 mmol), Xantphos (13.5 mg, 0.023 mmol), 2-mercapto ethanol (70.3 mg, 0.900 mmol), DIPEA (0.1 mL, 0.57 mmol) and dioxane (3 mL) and the reaction mixture was heated to 160° C. for 10 min using microwave single node heating. NaHCO₃ (aq) was added and the mixture was extracted with DCM (3 times). The combined organic layer was run through a phase separator and evaporated. The crude was purified by preparative HPLC (Kromasil C₈, 10 μm, 21.5×250 mm column, flow rate 25 mL/Minute, using a gradient of 10-40% CH₃CN/0.1 M NH₄OAc) to give the desired compound. Yield: 57 mg (46%).

¹H NMR (500 MHz, DMSO-d₆): [symbol] 1.30 (3H, t, J=7.1 Hz), 1.59-1.69 (2H, m), 1.77-1.85 (2H, m), 2.42-2.49 (1H, m), 3.17 (2H, t, J=6.7 Hz), 3.20-3.27 (2H, m), 3.63 (2H, t, J=6.7 Hz), 4.24 (2H, q, J=7.1 Hz), 4.46-4.52 (2H, m), 4.50 (2H, s), 4.96 (1H, br s), 7.25-7.28 (2H, m), 7.31-7.37 (3H, m), 8.26 (1H, s). 1 proton (NH) concealed by the water signal at 3.33.

MS m/z: 533 (M+1), 531 (M−1).

GTPγS (IC₅₀ μM): 0.016

### Example 20

### Ethyl 2-(2-acetamidoethoxy)-6-{4-[(benzylsulfonyl)carbamoyl]piperidin-1-yl}-5-cyanonicotinate

A microwave vial was charged with ethyl 6-{4-[allyl(benzylsulfonyl)carbamoyl]piperidin-1-yl}-5-cyano-2-{[(trifluoromethyl)sulfonyl]oxy}nicotinate (150 mg, 0.233 mmol), Pd₂(dba)₃ (21.3 mg, 0.023 mmol), Xantphos (13.5 mg, 0.023 mmol), N-(2-hydroxyethyl)acetamide (51.6 mg, 0.500 mmol), DIPEA (0.1 mL, 0.57 mmol) and dioxane (3 mL) and the reaction mixture was heated to 160° C. for 10 min using microwave single node heating. NaHCO₃ (aq) was added and the mixture was extracted with DCM (3 times). The combined organic layer was run through a phase separator and evaporated. The crude was purified by preparative HPLC (Kromasil C₈, 10 μm, 21.5×250 mm column, flow rate 25 mL/Minute, using a gradient of 10-40% CH₃CN/0.1 M NH₄OAc) to give the desired compound. Yield: 60 mg (46%).

¹H NMR (500 MHz, DMSO-d₆): δ 1.27 (3H, t, J=7.1 Hz), 1.58-1.68 (2H, m), 1.77-1.85 (2H, m), 1.82 (3H, s), 2.42-2.50 (1H, m), 3.16-3.23 (2H, m), 3.42 (2H, q, J=5.8), 4.20 (2H, q, J=7.1 Hz), 4.35 (2H, t, J=5.8 Hz), 4.46-4.53 (2H, m), 4.53 (2H, s), 7.26-7.29 (2H, m), 7.32-7.37 (3H, m), 7.97 (1H, t, J=5.4 Hz), 8.27 (1H, s).

MS m/z: 558 (M+1), 556 (M−1).

GTPγS (IC₅₀ μM): 0.038

### Example 21

### Ethyl 2-[(2-acetamidoethyl)thio]-6-{4-[(benzylsulfonyl)carbamoyl]piperidin-1-yl}-5-cyanonicotinate

A microwave vial was charged with ethyl 6-{4-[allyl(benzylsulfonyl)carbamoyl]piperidin-1-yl}-5-cyano-2-{[(trifluoromethyl)sulfonyl]oxy}nicotinate (150 mg, 0.233 mmol), Pd₂(dba)₃ (21.3 mg, 0.023 mmol), Xantphos (13.5 mg, 0.023 mmol), N-(2-mercaptoethyl)acetamide (59.6 mg, 0.500 mmol), DIPEA (0.1 mL, 0.57 mmol) and dioxane (3 mL) and the reaction mixture was heated to 160° C. for 10 min using microwave single node heating. NaHCO₃ (aq) was added and the mixture was extracted with DCM (3 times). The combined organic layer was run through a phase separator and evaporated. The crude was purified by preparative HPLC (Kromasil C₈, 10 μm, 21.5×250 mm column, flow rate 25 mL/Minute, using a gradient of 10-40% CH₃CN/0.1 M NH₄OAc) to give the desired compound. Yield: 35 mg (26%).

¹H NMR (500 MHz, DMSO-d₆): 1.30 (3H, t, J=7.1 Hz), 1.61-1.70 (2H, m), 1.81 (3H, s), 1.83-1.88 (2H, m), 2.54-2.61 (1H, m), 3.13-3.17 (2H, t, J=6.5 Hz), 3.17-3.24 (2H, m), 3.32 (2H, concealed by water signal at 3.33), 4.24 (2H, q, J=7.1 Hz), 4.51-4.57 (2H, m), 4.65 (2H, s), 7.26-7.31 (2H, m), 7.35-7.42 (3H, m), 8.09 (1H, t, J=5.1 Hz), 8.29 (1H, s), 11.64 (1H, br s).

MS m/z: 574 (M+1), 572 (M−1).

GTPγS (IC₅₀ μM): 0.01

### Example 22

### Ethyl 2-(2-amino-2-oxoethoxy)-6-{4-[(benzylsulfonyl)carbamoyl]piperidin-1-yl}-5-cyanonicotinate

A microwave vial was charged with ethyl 6-{4-[allyl(benzylsulfonyl)carbamoyl]piperidin-1-yl}-5-cyano-2-{[(trifluoromethyl)sulfonyl]oxy}nicotinate (150 mg, 0.233 mmol), Pd₂(dba)₃ (21.3 mg, 0.023 mmol), Xantphos (13.5 mg, 0.023 mmol), 2-hydroxyacetamide (37.5 mg, 0.500 mmol), DIPEA (0.1 mL, 0.57 mmol) and dioxane (3 mL) and the reaction mixture was heated to 160° C. for 10 min using microwave single node heating. NaHCO₃ (aq) was added and the mixture was extracted with DCM (3 times). The combined organic layer was run through a phase separator and evaporated. The crude was purified by preparative HPLC (Kromasil C₈, 10 μm, 21.5×250 mm column, flow rate 25 mL/Minute, using a gradient of 10-40% CH₃CN/0.1 M NH₄OAc) to give the desired compound. Yield: 33 mg (27%).

¹H NMR (500 MHz, DMSO-d₆): δ 1.28 (3H, t, J=7.1 Hz), 1.55-1.63 (2H, m), 1.75-1.83 (2H, m), 2.23-2.29 (1H, m), 3.19-3.27 (2H, m), 4.22 (2H, q, J=7.1 Hz), 4.25 (2H, s), 4.37-4.42 (2H, m), 4.71 (2H, s), 7.20-7.28 (5H, m), 8.29 (1H, s). 3 protons concealed by the water signal at 3-5.

MS m/z: 530 (M+1), 528 (M−1).

GTPγS (IC₅₀ μM): 0.072

### Example 23

### Ethyl 6-{4-[(benzylsulfonyl)carbamoyl]piperidin-1-yl}-5-cyano-2-(oxetan-2-ylmethoxy)nicotinate

A microwave vial was charged with ethyl 6-{4-[allyl(benzylsulfonyl)carbamoyl]piperidin-1-yl}-5-cyano-2-{[(trifluoromethyl)sulfonyl]oxy}nicotinate (150 mg, 0.233 mmol), Pd₂(dba)₃ (21.3 mg, 0.023 mmol), Xantphos (13.5 mg, 0.023 mmol), oxetan-2-ylmethanol (44 mg, 0.500 mmol), DIPEA (0.1 mL, 0.57 mmol) and dioxane (3 mL) and the reaction mixture was heated to 160° C. for 10 min using microwave single node heating. NaHCO₃ (aq) was added and the mixture was extracted with DCM (3 times). The combined organic layer was run through a phase separator and evaporated. The crude was purified by preparative HPLC (Kromasil C₈, 10 μm, 21.5×250 mm column, flow rate 25 mL/Minute, using a gradient of 10-45% CH₃CN/0.1 M NH₄OAc) to give the desired compound. Yield: 24 mg (19%).

¹H NMR (500 MHz, DMSO-d₆): δ 1.28 (3H, t, J=7.1 Hz), 1.60-1.70 (2H, m), 1.80-1.87 (2H, m), 2.52-2.60 (1H, m), 2.71 (2H, q, J=7.6 Hz), 3.15-3.22 (2H, m), 4.23 (2H, q, J=7.1 Hz), 4.48-4.55 (4H, m), 4.65 (2H, s), 5.00-5.05 (1H, m), 7.27-7.31 (2H, m), 7.36-7.41 (3H, m), 8.31 (1H, s), 11.61 (1H, br s).

MS m/z: 543 (M+1), 541 (M−1).

GTPγS (IC₅₀ μM): 0.048

### Example 24

### {[6-{4-[(Benzylsulfonyl)carbamoyl]piperidin-1-yl}-5-cyano-3-(ethoxycarbonyl)pyridin-2-yl]oxy}acetic acid

A microwave vial was charged with ethyl 6-{4-[allyl(benzylsulfonyl)carbamoyl]piperidin-1-yl}-5-cyano-2-{[(trifluoromethyl)sulfonyl]oxy}nicotinate (150 mg, 0.233 mmol), Pd₂(dba)₃ (21.3 mg, 0.023 mmol), Xantphos (13.5 mg, 0.023 mmol), ethyl glycolate (38 mg, 0.500 mmol), DIPEA (0.1 mL, 0.57 mmol) and dioxane (3 mL) and the reaction mixture was heated to 160° C. for 10 min using microwave single node heating. The mixture was filtered through a plug of Celite and washed with DCM. NaHCO₃ (aq) was added and the mixture was extracted with DCM (2 times). The vasic aqueous phase was made acidic with 2 M HCl and extracted with DCM (3 times). The combined organic layer was passed through a phase separator and evaporated to give the desired product. Yield: 26 mg (21%).

¹H NMR (500 MHz, DMSO-d₆): δ 1.28 (3H, t, J=7.1 Hz), 1.55-1.63 (2H, m), 1.75-1.81 (2H, m), 2.35-2.41 (1H, m), 3.16-3.23 (2H, m), 4.22 (2H, q, J=7.1 Hz), 4.38-4.42 (2H, m), 4.42 (2H, s), 4.81 (2H, s), 7.24-7.34 (5H, m), 8.30 (1H, s). 2 protons concealed by water signal at 3.40.

MS m/z: 531 (M+1), 529 (M−1).

GTPγS (IC₅₀ μM): 0.025

### Example 25

### Ethyl 6-{4-[(benzylsulfonyl)carbamoyl]piperidin-1-yl}-5-cyano-2-[2-(methylamino)-2-oxoethoxy]nicotinate

DIPEA (0.1 mL, 0.574 mmol) was added to a solution of crude {[6-{4-[(benzylsulfonyl)carbamoyl]piperidin-1-yl}-5-cyano-3-(ethoxycarbonyl)pyridin-2-yl]oxy}acetic acid (122 mg, 0.23 mmol), TBTU (74 mg, 0.23 mmol) in dry THF (5 mL). The reaction mixture was stirred at r.t for 1 h and MeNH₂ (0.15 ml, 0.30 mmol) was added. The reaction mixture was stirred at r.t over night. The solvent was evaporated, NH4Cl (aq) was added and the mixture was extracted with DCM (×3). The combined organic layer was run through a phase separator and evaporated. The crude was purified by preparative HPLC (Kromasil C₈, 10 μm, 21.5×250 mm column, flow rate 25 mL/Minute, using a gradient of 10-45% CH₃CN/0.1 M NH₄OAc) to give the desired compound. Yield: 2 mg (2%).

MS m/z: 544 (M+1), 542 (M−1).

GTPγS (IC₅₀ μM): 0.075

### Example 26

### {[6-{4-[(Benzylsulfonyl)carbamoyl]piperidin-1-yl}-5-cyano-3-(ethoxycarbonyl)pyridin-2-yl]thio}acetic acid

### (a) 1-[3-cyano-5-(ethoxycarbonyl)-6-{[(trifluoromethyl)sulfonyl]oxy}pyridin-2-yl]piperidine-4-carboxylic acid

TFA (10 mL) was added to a solution of crude ethyl 6-[4-(tert-butoxycarbonyl)piperidin-1-yl]-5-cyano-2-{[(trifluoromethyl)sulfonyl]oxy}nicotinate (See Example 108 (c)) (3.99 g, 7.86 mmol) in DCM (20 mL) and the reaction mixture was stirred at rt for 30 min. The mixture was concentrated under reduced pressure and the crude product was used in the next step without further purification. Yield assumed quantitative.

### (b) Ethyl 2-(1H-benzotriazol-1-yloxy)-6-{4-[(benzylsulfonyl)carbamoyl]piperidin-1-yl}-5-cyanonicotinate

DIPEA (5 mL, 28.7 mmol) was added to a solution of crude 1-[3-cyano-5-(ethoxycarbonyl)-6-{[(trifluoromethyl)sulfonyl]oxy}pyridin-2-yl]piperidine-4-carboxylic acid (3.55 g, 7.86 mmol) and TBTU (3.66 g, 11.4 mmol) in dry DCM (25 mL). The mixture was stirred at rt for 100 min, phenylmethanesulfonamide (1.35 g, 7.88 mmol) was added and the reaction mixture was stirred at rt for an additional 20 h. NaHCO₃ (aq) was added and the mixture was extracted with DCM (×3). The combined organics was run through a phase separator and concentrated under reduced pressure. The crude product was purified by preparative HPLC (Kromasil C₈ 10 μm, 50.8×300 mm, using a gradient of 20-60% CH₃CN/0.1 M NH₄OAc) to give ethyl 2-(1H-benzotriazol-1-yloxy)-6-{4-[(benzylsulfonyl)carbamoyl]piperidin-1-yl}-5-cyanonicotinate as a white solid after freeze drying from water. Yield: 1.79 g (39%).

MS m/z: 590 (M+1), 588 (M−1).

### (c) {[6-{4-[(Benzylsulfonyl)carbamoyl]piperidin-1-yl}-5-cyano-3-(ethoxycarbonyl)pyridin-2-yl]thio}acetic acid

2-(1H-benzotriazol-1-yloxy)-6-{4-[(benzylsulfonyl)carbamoyl]piperidin-1-yl}-5-cyanonicotinate (270 mg, 0.46 mmol) dissolved in THF (10 mL), mercaptoacetic acid (0.038 mL, 0.55 mmol) and DIPEA (0.2 mL, 1.15 mmol) were placed in a microwave vial and the mixture was heated to 120° C. for 5 min in a single node microwave oven. LCMS showed mostly remaining starting material. Mercaptoacetic acid (0.1 mL, 1.44 mmol) and DIPEA (0.2 mL, 1.15 mmol) were added and the reaction mixture was heated to 120° C. for min in a microwave oven. LCMS showed most starting material and some product. More mercaptoacetic acid (0.3 mL, 4.3 mmol) and DIPEA (0.2 mL, 1.15 mmol) were added and the reaction mixture was again heated to 120° C. for 5+10 min. LCMS showed complete conversion of starting material. Water (0.9 mL, 50 mmol) was added and the reaction mixture was heated to 130° C. for 10 min in a microwave oven. NH₄Cl (aq) was added, the mixture was made acidic by addition of conc HCl until pH=4 and the mixture was extracted with DCM (×3). The combined organics was run through a phase separator and evaporated. The crude was purified by preparative HPLC (Kromasil C₈ 10 μm, 50.8×300 mm, using a gradient of 20-60% CH₃CN/0.1 M NH₄OAc) to give the product as a white solid after freeze drying from water. Yield: 196 mg (78%).

¹H NMR (500 MHz, DMSO-d₆): 1.30 (3H, t, J=7.1 Hz), 1.54-1.63 (2H, m), 1.74-1.80 (2H, m), 2.20-2.27 (1H, m), 3.18-3.26 (2H, m), 3.67 (2H, s), 4.24 (2H, s), 4.25 (2H, q, J=7.1 Hz), 4.41-4.47 (2H, m), 7.18-7.27 (5H, m), 8.22 (1H, s).

MS m/z: 547 (M+1), 545 (M−1).

GTPγS (IC₅₀ μM): 0.0081

### Example 27

### Ethyl 6-{4-[(benzylsulfonyl)carbamoyl]piperidin-1-yl}-5-cyano-2-[(ethylthio)methyl]nicotinate

A microwave vial was charged with ethyl 6-{4-[(benzylsulfonyl)carbamoyl]piperidin-1-yl}-2-(chloromethyl)-5-cyanonicotinate (53 mg, 0.105 mmol, See Example 3(c)), ethanethiol (26 mg, 0.419 mmol), DIPEA (34 mg, 0.262 mmol and EtOH (1 mL) and heated to 100° C. in a microwave oven (single node heating) for 15 minutes. The mixture was diluted with 1 M MgCl₂ (2 mL) and quenched by the addition of 1 M HCl (0.262 mL, 0.262 mmol). The water phase was extracted with DCM (10 mL). The organic solvent was removed in vacuo and the crude product was purified according to Purification Method A (See General Experimental Procedure) to give the desired product. Yield: 17 mg (31%).

¹H NMR (600 MHz, DMSO-d₆) δ 1.16 (3H, t, J=7.8 Hz), 1.27 (3H, t, J=7.1 Hz), 1.56-1.65 (2H, m), 1.77-1.84 (2H, m), 3.11-3.17 (2H, m), 4.02 (2H, s), 4.22 (2H, q, J=7.3 Hz), 4.49-4.56 (2H, m), 4.66 (2H, s), 7.24-7.29 (2H, m), 7.34-7.40 (3H, m), 8.35 (1H, s)

MS m/z: 531 (M+1).

GTPγS (IC₅₀ μM): 0.057

### Example 28

### Ethyl 6-{4-[(benzylsulfonyl)carbamoyl]piperidin-1-yl}-5-cyano-2-{[(2-hydroxyethyl)thio]methyl}nicotinate

Prepared according to Example 27 from ethyl 6-{4-[(benzylsulfonyl)carbamoyl]piperidin-1-yl}-2-(chloromethyl)-5-cyanonicotinate (53 mg, 0.105 mmol, See Example 3(c)), and 2-mercaptoethanol (33 mg, 0.419 mmol). Yield: 68 mg (39%).

¹H NMR (600 MHz, DMSO-d₆) δ 1.28 (3H, t, J=7.3 Hz), 1.57-1.66 (2H, m), 1.78-1.84 (2H, m), 2.57 (2H, t, J=6.4 Hz), 3.10-3.18 (2H, m), 3.49 (2H, q, J=6.7 Hz), 4.03 (2H, s), 4.22 (2H, q, J=7.3 Hz), 4.50-4.56 (2H, m), 4.66 (2H, s), 7.24-7.28 (2H, m), 7.34-7.40 (3H, m), 8.35 (1H, s)

MS m/z: 547 (M+1).

GTPγS (IC₅₀ μM): 0.037

### Example 29

### Ethyl 2-{[(2-acetamidoethyl)thio]methyl}-6-{4-[(benzylsulfonyl)carbamoyl]piperidin-1-yl}-5-cyanonicotinate

Prepared according to Example 27 from ethyl 6-{4-[(benzylsulfonyl)carbamoyl]piperidin-1-yl}-2-(chloromethyl)-5-cyanonicotinate (53 mg, 0.105 mmol, See Example 3(c)), and N-(2-mercaptoethyl)acetamide (50 mg, 0.419 mmol). Yield: 51 mg (32%).

¹H NMR (400 MHz, CDCl₃) δ 1.35 (3H, t, J=7.1 Hz), 1.75 (3H, s), 1.76-1.94 (4H, m), 2.54-2.64 (1H, m), 2.69 (2H, t, J=6.4 Hz), 3.09-3.19 (2H, m), 3.25 (2H, q, J=6.1 Hz), 4.03 (2H, s), 4.30 (2H, q, J=6.9 Hz), 4.60 (2H, s), 4.66-4.72 (2H, br m), 6.49 (1H, br m), 7.29-7.38 (5H, m), 8.35 (1H, s)

MS m/z: 588 (M+1).

GTPγS (IC₅₀ μM): 0.033

### Example 30

### Ethyl 6-{3-[(benzylsulfonyl)carbamoyl]azetidin-1-yl}-5-cyano-2-[(ethylthio)methyl]nicotinate

Prepared according to Example 27 from ethyl 6-{3-[(benzylsulfonyl)carbamoyl]azetidin-1-yl}-2-(chloromethyl)-5-cyanonicotinate (50 mg, 0.105 mmol, See Example 6(d)) and ethanethiol (26 mg, 0.419 mmol). Yield: 57 mg (30%).

¹H NMR (600 MHz, DMSO-d₆) δ 1.17 (3H, t, J=7.2 Hz), 1.27 (3H, t, J=7.2 Hz), 3.41 (2H, q, J=7.1 Hz), 3.50-3.57 (1H, m), 3.99 (2H, s), 4.21 (2H, q, J=7.5 Hz), 4.25-4.32 (2H, br), 4.35-4.44 (2H, br), 4.73 (2H, s), 7.29-7.38 (5H, m), 8.32 (1H, s)

MS m/z: 503 (M+1).

GTPγS (IC₅₀ μM): 0.106

### Example 31

### Ethyl 6-{3-[(benzylsulfonyl)carbamoyl]azetidin-1-yl}-5-cyano-2-{[(2-hydroxyethyl)thio]methyl}nicotinate

Prepared according to Example 27 from ethyl 6-{3-[(benzylsulfonyl)carbamoyl]azetidin-1-yl}-2-(chloromethyl)-5-cyanonicotinate (50 mg, 0.105 mmol, See Example 6(d)) and 2-mercaptoethanol (33 mg, 0.419 mmol). Yield: 64 mg (35%).

¹H NMR (600 MHz, DMSO-d₆) δ 1.27 (2H, t, J=6.9 Hz), 2.58 (2H, t, J=6.9 Hz), 3.47-3.57 (3H, m), 3.99 (2H, s), 4.21 (2H, q, J=7.3 Hz), 4.25-4.33 (2H, br), 4.35-4.45 (2H, br), 4.72 (2H, s), 7.29-7.39 (5H, m), 8.33 (1H, s)

MS m/z: 519 (M+1).

GTPγS (IC₅₀ μM): 0.066

### Example 32

### Ethyl 2-{[(2-acetamidoethyl)thio]methyl}-6-{3-[(benzylsulfonyl)carbamoyl]azetidin-1-yl}-5-cyanonicotinate

Prepared according to Example 27 from ethyl 6-{3-[(benzylsulfonyl)carbamoyl]azetidin-1-yl}-2-(chloromethyl)-5-cyanonicotinate (50 mg, 0.105 mmol, See Example 6(d)) and N-(2-mercaptoethyl)acetamide (50 mg, 0.419 mmol). Yield: 61 mg (36%).

¹H NMR (600 MHz, DMSO-d₆) d 1.27 (3H, t, J=7.1 Hz), 1.76 (3H, s), 3.19 (2H, q, J=6.9 Hz), 3.50-3.57 (1H, m), 4.00 (2H, s), 4.21 (2H, q, J=7.4 Hz), 4.26-4.33 (2H, br s), 4.35-4.45 (2H, br s), 4.72 (2H, s), 7.29-7.38 (5H, m), 7.91 (1H, t, J=5.9 Hz), 8.33 (1H, s)

MS m/z: 560 (M+1).

GTPγS (IC₅₀ μM): 0.081

### Example 33

### Ethyl 6-{4-[(benzylsulfonyl)carbamoyl]piperidin-1-yl}-5-cyano-2-[(2-oxopyrrolidin-1-yl)methyl]nicotinate

### (a) Ethyl 3-oxo-4-(2-oxopyrrolidin-1-yl)butanoate

NaH (15.31 g, 0.638 mol) was added to dry 2-methyl tetrahydrofurane (250 mL) at rt under N₂, the mixture was cooled to −5° C. Pyrrolidin-2-one (27.15 g, 0.319 mol) was dissolved in dry toluene (100 mL) and added drop wise at such a rate that the reaction temperature was kept below −2° C. The reaction mixture was stirred at −5° C. for 2 h. 4-Chloro-3-oxo-butyric acid ethyl ester (50.0 g, 0.304 mol) was dissolved in dry toluene (100 mL) and was added drop wise at such a rate that the reaction temperature was kept below −2° C. The reaction mixture was stirred at −5° C. to 20° C. over night. The reaction mixture was quenched with acetic acid (36.5 mL, 0.638 mol) and water (250 mL). The organic layer was separated and the aqueous layer was extracted with toluene (150 mL). The combined organics was dried (MgSO₄), filtered, concentrated in vacuo and co-evaporated with toluene to give the crude product as oil. The crude product was used in the next step without further purification. Yield: 57.48 g (89%).

¹H NMR (500 MHz, DMSO-d₆): δ 1.19 (3H, t), 1.96 (2H, pentet), 2.25 (2H, t), 3.31 (2H, d), 3.64 (2H, s), 4.10 (2H, q), 4.20 (2H, s).

### (b) Ethyl 5-cyano-6-hydroxy-2-[(2-oxopyrrolidin-1-yl)methyl]nicotinate

Ethyl 3-oxo-4-(2-oxopyrrolidin-1-yl)butanoate (Example 33 (a)) (4.11 g, 19.3 mmol) was dissolved in EtOH (80 mL, 99.5%), N,N.dimethylformamide dimethyl acetal (2.69 mL, 20.2 mmol) was added. The reaction mixture was stirred at 50° C. for 1.5 h. DIPEA (0.67 mL, 3.85 mmol) was added, stirred 30 min and malononitrile (1.4 mL, 22.24 mmol) in EtOH (20 mL) was added to the mixture. The reaction was stirred for 45 min, acetic acid (4 mL) diluted with water (40 mL) was added and the reaction mixture was cooled to rt. The organic solvent was concentrated in vacuo and the solid material was filtered, washed with water and dried to give ethyl 5-cyano-6-hydroxy-2-[(2-oxopyrrolidin-1-yl)methyl]nicotinate as beige solid. Yield: 4.3 g (77%).

¹H-NMR (500 MHz, DMSO-d₆) δ 1.28 (3H, t), 1.96 (2H, quintet), 2.25 (2H, t), 4.23 (2H, q), 4.72 (2H, s), 8.47 (1H, s), 12.61 (1H, s). Note! One proton signal (2H) overlaps with the H₂O signal (3.26-3.35).

MS m/z: 290 (M+1), 288 (M−1).

### (c) 1-(tert-Butoxycarbonyl)piperidine-4-carboxylic acid

Di-tert-butyl dicarbonate (7.2 g, 33 mmol) was added in portions to a solution of isonipecotic acid (3.9 g, 30 mmol) in THF/water/NaOH (60/30/30 1 M) at rt, the mixture was stirred for 19 h. The organic solvent was concentrated in vacuo and the alkaline water phase was washed with dimethylether (2×15 mL). The solution was then acidified with 1 M KHSO₄ (70 mL) and the water phase was extracted with dimethylether (1×100+1×50 mL). The combined organic layer was washed with brine, dried (MgSO₄), filtered and concentrated in vacuo to give 1-(tert-butoxycarbonyl)piperidine-4-carboxylic acid as a white powder. Yield: 6.60 g (96%).

¹H-NMR (400 MHz, CDCl₃) δ 1.46 (9H, s), 1.60-1.71 (2H, m), 1.87-1.96 (2H, m), 2.44-2.54 (1H, m), 2.81-2.92 (2H, m), 3.95-4.09 (2H, m).

### (d) tert-Butyl 4-[(benzylsulfonyl)carbamoyl]piperidine-1-carboxylate

TEA (66 g, 0.65 mol) was added drop wise to a solution of 1-(tert-butoxycarbonyl)piperidine-4-carboxylic acid (Example 33 (c)) (50 g, 0.22 mol) and TBTU (77 g, 0.24 mol) in THF (600 mL), the reaction mixture was stirred at rt for 1.5 h. N-Phenylsulfamide (41 g, 0.24 mol) was added and the reaction was stirred over night at rt. The organic solvent was concentrated in vacuo and the residue was dissolved in EtOAc (600 mL), washed with 2 M HCl, 1M HCl, water and concentrated in vacuo. The residue was dissolved in EtOH (250 mL) and water was added (600 mL), the precipitate was filtered, washed with EtOH and dried to give tert-butyl 4-[(benzylsulfonyl)carbamoyl]piperidine-1-carboxylate. Yield: 69 g (83%).

¹H-NMR (300 MHz, DMSO-d₆) δ 1.30-1.47 (2H, m), 1.39 (9H, s), 1.61-1.71 (2H, m), 2.32-2.42 (1H, m), 2.60-2.76 (2H, m), 3.86-3.98 (2H, m), 4.68 (2H, s), 7.25-7.30 (2H, m), 7.34-7.43 (3H, m), 11.54 (1H, s).

### (e) N-(Benzylsulfonyl)piperidine-4-carboxamide

tert-Butyl 4-[(benzylsulfonyl)carbamoyl]piperidine-1-carboxylate (Example 33 (d)) (14.7 g, 38 mmol) was dissolved in formic acid (160 g, 3.5 mol), the reaction mixture was stirred at rt for 25 h. The solvent was concentrated in vacuo and water was added followed by NH₄OH until pH 6.5. The precipitate was filtered and dried to give N-(benzylsulfonyl)piperidine-4-carboxamide. Yield: 8.4 g (77%).

¹H-NMR (400 MHz, DMSO-d₆) δ 1.58-1.71 (2H, m), 1.73-1.84 (2H, m), 2.09-2.18 (1H, m), 2.77-2.87 (2H, m), 3.08-3.18 (2H, m), 4.23 (2H, s), 7.16-7.28 (5H, m).

### (f) Ethyl 6-{4-[(benzylsulfonyl)carbamoyl]piperidin-1-yl}-5-cyano-2-[(2-oxopyrrolidin-1-yl)methyl]nicotinate

Ethyl 5-cyano-6-hydroxy-2-[(2-oxopyrrolidin-1-yl)methyl]nicotinate (Example 33 (b)) (500 mg, 1.73 mmol) was dissolved in DCM (20 mL), the reaction mixture was cooled to 0° C. N-(Benzylsulfonyl)piperidine-4-carboxamide (Example 33 (e)) (733, 2.59 mmol), bromo-tris-pyrrolidino-phosphonium hexafluorophosphate (886 mg, 1.9 mmol) and DIPEA (0.66 mL, 3.8 mmol) were added, the reaction mixture was allowed to warm to rt over night. 1M HCl (7 mL) was added, the organic phase was separated, concentrated in vacuo and purified by flash chromatography, EtOAc:heptane 80:20 to 100:0 and 1% acetic acid as eluent, to give ethyl 6-{4-[(benzylsulfonyl)carbamoyl]piperidin-1-yl}-5-cyano-2-[(2-oxopyrrolidin-1-yl)methyl]nicotinate. Yield: 327 mg (34%).

¹H-NMR (400 MHz, CDCl₃) δ 1.31 (3H, t), 1.63-1.83 (4H, m), 1.98 (2H, q), 2.24 (2H, t), 2.40-2.49 (1H, m), 3.08-3.17 (2H, m), 3.37 (2H, t), 4.26 (2H, q), 4.36-4.43 (2H, m), 4.56 (2H, s), 4.80 (2H, s), 7.23-7.33 (5H, m), 8.31 (1H, s).

MS m/z: 544 (M+1), 542 (M−1).

GTPγS (IC₅₀ μM): 0.0097

### Example 34

### Isopropyl 6-{4-[(benzylsulfonyl)carbamoyl]piperidin-1-yl}-5-cyano-2-[(2-oxopyrrolidin-1-yl)methyl]nicotinate

### (a) 6-{4-[(Benzylsulfonyl)carbamoyl]piperidin-1-yl}-5-cyano-2-[(2-oxopyrrolidin-1-yl)methyl]nicotinic acid

Ethyl 6-{4-[(benzylsulfonyl)carbamoyl]piperidin-1-yl}-5-cyano-2-[(2-oxopyrrolidin-1-yl)methyl]nicotinate (Example 33 (f)) (188 mg, 0.34 mmol) was added to 2-propanol (2 mL) and 1 M NaOH (1.36 mL), the reaction mixture was heated at 100° C. for 10 min. Acetic acid in water (3 mL) was added and the precipitate was filtered, washed with water and dried to give 6-{4-[(benzylsulfonyl)carbamoyl]piperidin-1-yl}-5-cyano-2-[(2-oxopyrrolidin-1-yl)methyl]nicotinic acid. Yield: 95 mg (53%).

¹H-NMR (500 MHz, CDCl₃) δ 1.55-1.74 (4H, m), 1.92 (2H, quintet), 2.26 (2H, t), 2.52-2.62 (1H, m). 2.97-3.07 (2H, m), 3.31 (2H, t), 4.38 (2H, d), 4.43 (2H, s), 4.75 (2H, s), 7.11-7.32 (5H, m), 8.21 (1H, s), 9.82 (bs, 1H).

MS m/z: 429 (M+1), 427 (M−1)

### (b) Isopropyl 6-{4-[(benzylsulfonyl)carbamoyl]piperidin-1-yl}-5-cyano-2-[(2-oxopyrrolidin-1-yl)methyl]nicotinate

6-{4-[(Benzylsulfonyl)carbamoyl]piperidin-1-yl}-5-cyano-2-[(2-oxopyrrolidin-1-yl)methyl]nicotinic acid (352 mg, 0.67 mmol) was dissolved in 2-propanol (5 mL), 1-ethyl-3-(3-dimethylaminopropyl)carbodiimide hydrochloride (193 mg, 1 mmol) and DMAP (8.2 mg, 0.07 mmol) were added. The reaction mixture was stirred at 40° C. over night. The solvent was concentrated in vacuo and purified first by preparative HPLC (Kromasil C₈, 250×50 mmID, using an increasing gradient of 20% to 80% MeCN over 30 min. with an acidic second eluent (H2O/MeCN/FA, 95/5/0.2)) and then by flash chromatography, EtOAc:heptane 80:20 to 100:0 and 1% acetic acid as eluent, to give isopropyl 6-{4-[(benzylsulfonyl)carbamoyl]piperidin-1-yl}-5-cyano-2-[(2-oxopyrrolidin-1-yl)methyl]nicotinate. Yield: 84 mg (22%)

¹H-NMR (500 MHz, CDCl₃) δ 1.36 (6H, d), 1.72-1.89 (4H, m), 2.03-2.11 (2H, m), 2.33 (2H, t), 2.48-2.56 (1H, m), 3.17-3.24 (2H, m), 3.45 (2H, t), 4.48 (2H, d), 4.64 (2H, s), 4.88 (2H, s), 5.20 (1H, septet), 7.32-7.41 (5H, m), 8.37 (1H, s), 9.50 (1H, s).

MS m/z: 568 (M+1), 566 (M−1).

GTPγS (IC₅₀ μM): 0.033

### Example 35

### Ethyl 5-cyano-2-[(2-oxopyrrolidin-1-yl)methyl]-6-[4-({[4-(trifluoromethyl)benzyl]sulfonyl}carbamoyl)piperidin-1-yl]nicotinate

### (a) Ethyl 6-[4-(tert-butoxycarbonyl)piperidin-1-yl]-5-cyano-2-[(2-oxopyrrolidin-1-yl)methyl]nicotinate

Ethyl 5-cyano-6-hydroxy-2-[(2-oxopyrrolidin-1-yl)methyl]nicotinate (Example 33 (b)) (5.46 g, 18.9 mmol was dissolved in DCM (50 mL), bromo-tris-pyrrolidino-phosphonium hexafluorophosphate (13.2 g, 28.3 mmol) and DIPEA (19.7 mL, 113 mmol) were added. The reaction mixture was stirred for 15 min at rt, 4-tert-butyl-carboxylic acid piperidine (4.14 g, 22.3 mmol) dissolved in DCM (10 mL) was added, the reaction mixture was stirred over night at rt. 1M HCl was added, the aqueous phase was extracted with DCM (×2), the combined organic phases were concentrated in vacuo and the residue was purified by preparative HPLC (Kromasil C8, 250×50 Idmm using an increasing gradient of MeCN with an acidic second eluent (H₂O/MeCN/FA: 95:5:0.2)) to give ethyl 6-[4-(tert-butoxycarbonyl)piperidin-1-yl]-5-cyano-2-[(2-oxopyrrolidin-1-yl)methyl]nicotinate as a white solid. Yield: 6.3 g (73).

¹H NMR (400 MHz, DMSO-d₆) δ 1.30 (3H, t), 1.39 (9H, s), 1.49-1.61 (2H, m), 1.86-1.94 (2H, m), 1.98 (2H, quintet), 2.27 (2H, t), 2.56-2.64 (1H, m), 3.24-3.30 (2H, m), 3.42 (2H, t), 4.26 (2H, q), 4.38 (2H, d), 4.74 (2H, s), 8.37 (1H, s).

MS m/z: 457 (M+1), 455 (M−1).

### (b) 1-{3-Cyano-5-(ethoxycarbonyl)-6-[(2-oxopyrrolidin-1-yl)methyl]pyridin-2-yl}piperidine-4-carboxylic acid

Ethyl 6-[4-(tert-butoxycarbonyl)piperidin-1-yl]-5-cyano-2-[(2-oxopyrrolidin-1-yl)methyl]nicotinate (Example 35 (a)) (480 mg, 1.1 mmol) was dissolved in DCM (2 mL), trifluoroacetic acid (2 mL) was added and the reaction mixture was stirred at rt for 1.5 h. The solvent was concentrated in vacuo and the crude product was used in the next step without further purification.

MS m/z: 401 (M+1), 399 (M−1).

### (c) Ethyl 5-cyano-2-[(2-oxopyrrolidin-1-yl)methyl]-6-[4-({[4-(trifluoromethyl)benzyl]sulfonyl}carbamoyl)piperidin-1-yl]nicotinate

1-[4-(Trifluoromethyl)phenyl]methanesulfonamide (72 mg, 0.30 mmol) was added to a solution of bromo-tris-pyrrolidinophosphonium hexafluorophosphate (168 mg, 0.36 mmol) in DCM (1 mL). 1-{3-cyano-5-(ethoxycarbonyl)-6-[(2-oxopyrrolidin-1-yl)methyl]pyridin-2-yl}piperidine-4-carboxylic acid (Example 35 (b)) (80 mg, 0.20 mmol), dissolved in DCM (2 mL) and DIPEA (348 μl, 2.0 mmol) were added and the reaction mixture was stirred at rt over night. 1% KHSO₄ was added, the aqueous phase was extracted with DCM and the combined organic phases was dried (phase separator) and concentrated in vacuo. The residue was purified by preparative HPLC (Kromasil C8, 250×50 Idmm using an increasing gradient of MeCN with an acidic second eluent (H₂O/MeCN/FA: 95:5:0.2)) to give ethyl 5-cyano-2-[(2-oxopyrrolidin-1-yl)methyl]-6-[4-({[4-(trifluoromethyl)benzyl]sulfonyl}carbamoyl)piperidin-1-yl]nicotinate as a white solid. Yield: 80 mg (64%).

¹H NMR (400 MHz, CDCl₃) δ 1.36 (3H, t), 1.70-1.90 (4H, m), 1.97-2.07 (2H, m), 2.19-2.27 (2H, m), 2.53-2.61 (1H, m), 3.09-3.20 (2H, m), 3.38-3.46 (2H, m), 4.31 (2H, q), 4.42-4.50 (2H, m), 4.66 (2H, s), 4.84 (2H, s), 7.47 (2H, d), 7.62 (2H, d), 8.37 (1H, s), 10.47 (1H, s).

MS m/z: 622 (M+1), 620 (M−1).

GTPγS (IC₅₀ μM): 0.0097

### Example 36

### Ethyl 5-cyano-6-(4-{[(cyclopentylmethyl)sulfonyl]carbamoyl}piperidin-1-yl)-2-[(2-oxopyrrolidin-1-yl)methyl]nicotinate

Prepared according to Example 35(c) from 1-{3-cyano-5-(ethoxycarbonyl)-6-[(2-oxopyrrolidin-1-yl)methyl]pyridin-2-yl}piperidine-4-carboxylic acid (Example 35 (b)) (1.1 g, 2.75 mmol) and 1-cyclopentylmethanesulfonamide (0.49 g, 3 mmol) to give ethyl 5-cyano-6-(4-{[(cyclopentylmethyl)sulfonyl]carbamoyl}piperidin-1-yl)-2-[(2-oxopyrrolidin-1-yl)methyl]nicotinate. Yield: 788 mg (53%).

¹H NMR (400 MHz, DMSO-d₆) δ 1.13-1.24 (2H, m), 1.27 (3H, t), 1.41-1.48 (2H, m), 1.48-1.60 (4H, m), 1.76-1.89 (4H, m), 1.95 (2H, pentet), 2.04-2.16 (1H, m), 2.24 (2H, t), 2.59-2.69 (1H, m), 3.13-3.23 (2H, m), 3.33-3.42 (4H, m), 4.22 (2H, q), 4.40-4.47 (2H, m), 4.71 (2H, s), 8.34 (1H, s).

MS m/z: 546 (M+1), 544 (M−1).

GTPγS (IC₅₀ μM): 0.017

### Example 37

### Ethyl 5-cyano-6-[4-({[(4-methylcyclohexyl)methyl]sulfonyl}carbamoyl)piperidin-1-yl]-2-[(2-oxopyrrolidin-1-yl)methyl]nicotinate

Prepared according to Example 35(c) from 1-{3-cyano-5-(ethoxycarbonyl)-6-[(2-oxopyrrolidin-1-yl)methyl]pyridin-2-yl}piperidine-4-carboxylic acid (Example 35 (b)) (80 mg, 0.2 mmol) and 1-(4-methylcyclohexyl)methanesulfonamide (57 mg, 0.3 mmol) to give ethyl 5-cyano-6-[4-({[(4-methylcyclohexyl)methyl]sulfonyl}carbamoyl)piperidin-1-yl]-2-[(2-oxopyrrolidin-1-yl)methyl]nicotinate. Yield: 72 mg (63%).

¹H NMR (400 MHz, CDCl₃) δ 0.86-0.92 (3H, m), 1.05-1.22 (2H, m), 1.38 (3H, t), 1.49-1.74 (6H, m), 1.77-1.89 (2H, m), 1.90-2.02 (3H, m), 2.05-2.16 (2H, m), 2.21-2.30 (1H, m), 2.43-2.51 (2H, m), 2.57-2.65 (1H, m), 3.24-3.34 (3H, m), 3.42-3.49 (3H, m), 4.33 (2H, q), 4.47-4.55 (2H, m), 4.92 (2H, s), 8.40 (1H, s), 9.34 (1H, s).

MS m/z: 574 (M+1), 572 (M−1).

GTPγS (IC₅₀ μM): 0.03

### Example 38

### Ethyl 5-cyano-6-(4-{[(2,4-difluorobenzyl)sulfonyl]carbamoyl}piperidin-1-yl)-2-[(2-oxopyrrolidin-1-yl)methyl]nicotinate

Prepared according to Example 35(c) from 1-{3-cyano-5-(ethoxycarbonyl)-6-[(2-oxopyrrolidin-1-yl)methyl]pyridin-2-yl}piperidine-4-carboxylic acid (Example 35 (b)) (60 mg, 0.15 mmol) and 1-(2,4-difluorophenyl)methanesulfonamide (64.2 mg, 0.17 mmol) to give ethyl 5-cyano-6-(4-{[(2,4-difluorobenzyl)sulfonyl]carbamoyl}piperidin-1-yl)-2-[(2-oxopyrrolidin-1-yl)methyl]nicotinate. Yield: 43 mg (49%).

¹H NMR (600 MHz, DMSO-d₆) δ 1.28 (3H, t), 1.53-1.62 (2H, m), 1.79-1.86 (2H, m), 1.94-2.01 (2H, m), 2.24-2.28 (2H, m), 2.58-2.63 (1H, m), 3.14-3.21 (2H, m), 3.39-3.42 (2H, m), 4.24 (2H, q), 4.43-4.49 (2H, m), 4.71 (2H, s), 4.73 (2H, s), 7.12-7.17 (1H, m), 7.28-7.33 (1H, m), 7.40-7.45 (1H, m), 8.36 (1H, s).

MS m/z: 590 (M+1), 588 (M−1).

GTPγS (IC₅₀ μM): 0.0033

### Example 39

### Ethyl 6-(4-{[(4-chlorobenzyl)sulfonyl]carbamoyl}piperidin-1-yl)-5-cyano-2-[(2-oxopyrrolidin-1-yl)methyl]nicotinate

Prepared according to Example 35(c) from 1-{3-cyano-5-(ethoxycarbonyl)-6-[(2-oxopyrrolidin-1-yl)methyl]pyridin-2-yl}piperidine-4-carboxylic acid (Example 35 (b)) (60 mg, 0.15 mmol) and 1-(4-chlorophenyl)methanesulfonamide (64 mg, 0.17 mmol) to give ethyl 6-(4-{[(4-chlorobenzyl)sulfonyl]carbamoyl}piperidin-1-yl)-5-cyano-2-[(2-oxopyrrolidin-1-yl)methyl]nicotinate. Yield: 46 mg (53%).

¹H NMR (600 MHz, DMSO-d₆) δ 1.29 (3H, t), 1.52-1.60 (2H, m), 1.77-1.82 (2H, m), 1.95-2.01 (2H, m), 2.25-2.29 (2H, m), 2.56-2.60 (1H, m), 3.14-3.20 (2H, m), 3.39-3.43 (2H, m), 4.24 (2H, q), 4.44-4.49 (2H, m), 4.68 (2H, s), 4.73 (2H, s), 7.25-7.29 (2H, m), 7.44-7.48 (2H, m), 8.37 (1H, s).

MS m/z: 588 (M+1), 586 (M−1).

GTPγS (IC₅₀ μM): 0.011

### Example 40

### Ethyl 5-cyano-6-(4-{[(4-fluorobenzyl)sulfonyl]carbamoyl}piperidin-1-yl)-2-[(2-oxopyrrolidin-1-yl)methyl]nicotinate

Prepared according to Example 35(c) from 1-{3-cyano-5-(ethoxycarbonyl)-6-[(2-oxopyrrolidin-1-yl)methyl]pyridin-2-yl}piperidine-4-carboxylic acid (Example 35(b)) (60 mg, 0.15 mmol) and 1-(4-fluorophenyl)methanesulfonamide (61 mg, 0.17 mmol) to give ethyl 5-cyano-6-(4-{[(4-fluorobenzyl)sulfonyl]carbamoyl}piperidin-1-yl)-2-[(2-oxopyrrolidin-1-yl)methyl]nicotinate. Yield: 42.6 mg (50%).

¹H NMR (600 MHz, DMSO-d₆) δ 1.28 (3H, t), 1.52-1.60 (2H, m), 1.76-1.81 (2H, m), 1.94-2.00 (2H, m), 2.24-2.28 (2H, m), 3.13-3.19 (2H, m), 3.39-3.43 (2H, m), 4.24 (2H, q), 4.44-4.48 (2H, m), 4.67 (2H, s), 4.73 (2H, s), 7.20-7.24 (2H, m), 7.28-7.31 (2H, m), 8.37 (1H, s). One signal (1H) is overlapping with the solvent signal (2.43-2.68 ppm)

MS m/z: 572 (M+1), 570 (M−1). GTPγS (IC₅₀ μM): 0.025

### Example 41

### Ethyl 5-cyano-6-(4-{[(2,4-dichlorobenzyl)sulfonyl]carbamoyl}piperidin-1-yl)-2-[(2-oxopyrrolidin-1-yl)methyl]nicotinate

Prepared according to Example 35(c) from 1-{3-cyano-5-(ethoxycarbonyl)-6-[(2-oxopyrrolidin-1-yl)methyl]pyridin-2-yl}piperidine-4-carboxylic acid (Example 35(b)) (60 mg, 0.15 mmol) and 1-(2,4-dichlorophenyl)methanesulfonamide (69.6 mg, 0.17 mmol) to give ethyl 5-cyano-6-(4-{[(2,4-dichlorobenzyl)sulfonyl]carbamoyl}piperidin-1-yl)-2-[(2-oxopyrrolidin-1-yl)methyl]nicotinate. Yield: 38.4 mg (41%).

¹H NMR (600 MHz, DMSO-d₆) δ 1.28 (3H, t), 1.54-1.62 (2H, m), 1.82-1.87 (2H, m), 1.94-2.00 (2H, m), 2.25-2.28 (2H, m), 2.58-2.65 (1H, m), 3.16-3.21 (2H, m), 3.39-3.42 (2H, m), 4.24 (2H, q), 4.44-4.48 (2H, m), 4.73 (2H, s), 4.82 (2H, s), 7.42-7.44 (1H, m), 7.49-7.52 (1H, m), 7.69 (1H, s), 8.37 (1H, s).

MS m/z: 622 (M+1), 620 (M−1).

GTPγS (IC₅₀ μM): 0.016

### Example 42

### Ethyl 5-cyano-6-(4-{[(4-methylbenzyl)sulfonyl]carbamoyl}piperidin-1-yl)-2-[(2-oxopyrrolidin-1-yl)methyl]nicotinate

Prepared according to Example 35(c) from 1-{3-cyano-5-(ethoxycarbonyl)-6-[(2-oxopyrrolidin-1-yl)methyl]pyridin-2-yl}piperidine-4-carboxylic acid (Example 35(b)) (60 mg, 0.15 mmol) and 1-(4-methylphenyl)methanesulfonamide (60.6 mg, 0.17 mmol) to give ethyl 5-cyano-6-(4-{[(4-methylbenzyl)sulfonyl]carbamoyl}piperidin-1-yl)-2-[(2-oxopyrrolidin-1-yl)methyl]nicotinate. Yield: 35 mg (41%).

¹H NMR (600 MHz, DMSO-d₆) δ 1.37 (3H, t), 1.71-1.91 (4H, m), 2.01-2.09 (2H, m), 2.28-2.33 (2H, m), 2.34 (3H, s), 2.48-2.58 (1H, m), 3.15-3.24 (2H, m), 3.41-3.47 (2H, m), 4.32 (2H, q), 4.44-4.52 (2H, m), 4.57 (2H, s), 4.87 (2H, s), 7.14-7.22 (4H, m), 8.38 (1H, s), 9.62 (1H, s).

MS m/z: 568 (M+1), 566 (M−1).

GTPγS (IC₅₀ μM): 0.012

### Example 43

### Ethyl 6-(4-{[(2-chloro-4-fluorobenzyl)sulfonyl]carbamoyl}piperidin-1-yl)-5-cyano-2-[(2-oxopyrrolidin-1-yl)methyl]nicotinate

Prepared according to Example 35(c) from 1-{3-cyano-5-(ethoxycarbonyl)-6-[(2-oxopyrrolidin-1-yl)methyl]pyridin-2-yl}piperidine-4-carboxylic acid (Example 35(b)) (60 mg, 0.15 mmol) and 1-(2-chloro-4-fluorophenyl)methanesulfonamide (67 mg, 0.17 mmol) to give ethyl 6-(4-{[(2-chloro-4-fluorobenzyl)sulfonyl]carbamoyl}piperidin-1-yl)-5-cyano-2-[(2-oxopyrrolidin-1-yl)methyl]nicotinate. Yield: 47.7 mg (53%).

¹H NMR (500 MHz, DMSO-d₆) δ 1.30 (3H, t), 1.56-1.66 (2H, m), 1.85-1.91 (2H, m), 1.96-2.03 (2H, m), 2.26-2.31 (2H, m), 2.60-2.68 (1H, m), 3.17-3.24 (2H, m), 3.41-3.45 (2H, m), 4.26 (2H, q), 4.45-4.52 (2H, m), 4.75 (2H, s), 4.83 (2H, s), 7.29-7.34 (1H, m), 7.46-7.55 (2H, m), 8.38 (1H, s), 11.80 (1H, s).

MS m/z: 606 (M+1), 604 (M−1).

GTPγS (IC₅₀ μM): 0.018

### Example 44

### Ethyl 6-(4-{[(4-chloro-2-fluorobenzyl)sulfonyl]carbamoyl}piperidin-1-yl)-5-cyano-2-[(2-oxopyrrolidin-1-yl)methyl]nicotinate

Prepared according to Example 35(c) from 1-{3-cyano-5-(ethoxycarbonyl)-6-[(2-oxopyrrolidin-1-yl)methyl]pyridin-2-yl}piperidine-4-carboxylic acid (Example 35(b)) (60 mg, 0.15 mmol) and 1-(4-chloro-2-fluorophenyl)methanesulfonamide (67 mg, 0.17 mmol) to give ethyl 6-(4-{[(4-chloro-2-fluorobenzyl)sulfonyl]carbamoyl}piperidin-1-yl)-5-cyano-2-[(2-oxopyrrolidin-1-yl)methyl]nicotinate. Yield: 60 mg (62%).

¹H NMR (500 MHz, DMSO-d₆) δ 1.31 (3H, t), 1.55-1.64 (2H, m), 1.83-1.89 (2H, m), 1.97-2.04 (2H, m), 2.27-2.31 (2H, m), 2.59-2.67 (1H, m), 3.17-3.24 (2H, m), 3.41-3.45 (2H, m), 4.26 (2H, q), 4.45-4.52 (2H, m), 4.74 (2H, s), 4.75 (2H, s), 7.35-7.44 (2H, m), 7.50-7.53 (1H, m), 8.38 (1H, s), 11.73 (1H, s).

MS m/z: 606 (M+1), 604 (M−1).

GTPγS (IC₅₀ μM): 0.01

### Example 45

### Isopropyl 5-cyano-6-(4-{[(4-methylbenzyl)sulfonyl]carbamoyl}piperidin-1-yl)-2-[(2-oxopyrrolidin-1-yl)methyl]nicotinate

### (a) 6-[4-(tert-Butoxycarbonyl)piperidin-1-yl]-5-cyano-2-[(2-oxopyrrolidin-1-yl)methyl]nicotinic acid

Ethyl 6-[4-(tert-butoxycarbonyl)piperidin-1-yl]-5-cyano-2-[(2-oxopyrrolidin-1-yl)methyl]nicotinate (Example 35 (a)) (6.3 g, 13.8 mmol) was dissolved in EtOH (40 mL) and MeCN (20 mL). NaOH in aqueous solution (4.97 M, 10 mL) was added, the reaction mixture was heated to 80° C. for 5 min in the microwave oven. Formic acid (pH<3) and water were added, the solution was cooled in fridge 1 h. The precipitate was filtered and washed with acidic water (pH<3) and 2-propanol. Filtrate contained a lot of product so the procedure was repeated twice. The solid material was dried under vacuum and co-concentrated from MeCN (×4) to give 6-[4-(tert-butoxycarbonyl)piperidin-1-yl]-5-cyano-2-[(2-oxopyrrolidin-1-yl)methyl]nicotinic acid as a white solid. Yield: 5.6 g (95%).

### (b) Isopropyl 6-[4-(tert-butoxycarbonyl)piperidin-1-yl]-5-cyano-2-[(2-oxopyrrolidin-1-yl)methyl]nicotinate

6-[4-(tert-Butoxycarbonyl)piperidin-1-yl]-5-cyano-2-[(2-oxopyrrolidin-1-yl)methyl]nicotinic acid (Example 45 (a)) (700 mg, 1.63 mmol) was dissolved in DCM (7 mL), the solution was cooled to −5° C. DMAP (199.6 mg, 1.64 mmol), DIPEA (0.6 mL, 3.6 mmol) and isopropyl chloroformate (3.6 mL, 3.6 mmol) were added, the reaction mixture was stirred over night at rt. HCl (0.27 mL) dissolved in water (10 mL) and DCM were added, the organic phase was separated, washed with 1M NaHCO₃, dried (MgSO₄) and concentrated in vacuo. The crude product was used in the next step without further purification. Yield: 690 mg (90%).

¹H NMR (400 MHz, CDCl₃) δ 1.32 (6H, d), 1.42 (9H, s), 1.69-1.80 (2H, m), 1.92-2.00 (2H, m), 2.02-2.11 (2H, m), 2.44 (2H, t), 2.46-2.55 (1H, m), 3.25-3.34 (2H, m), 3.46 (2H, t), 4.39-4.48 (2H, m), 4.88 (2H, s), 5.16 (1H, septet), 8.33 (1H, s).

MS m/z: 429 (M+1), 427 (M−1)

### (c) 1-{3-Cyano-5-(isopropoxycarbonyl)-6-[(2-oxopyrrolidin-1-yl)methyl]pyridin-2-yl}piperidine-4-carboxylic acid

Prepared according to Example 35(b) from isopropyl 6-[4-(tert-butoxycarbonyl)piperidin-1-yl]-5-cyano-2-[(2-oxopyrrolidin-1-yl)methyl]nicotinate (Example 45 (b)) (690 mg, 1.47 mmol) to give 1-{3-cyano-5-(isopropoxycarbonyl)-6-[(2-oxopyrrolidin-1-yl)methyl]pyridin-2-yl}piperidine-4-carboxylic acid. Yield: 577 mg (95%).

¹H NMR (400 MHz, DMSO-d₆) δ 1.20 (6H, d), 1.40-1.54 (2H, m), 1.78-1.94 (4H, m), 2.13-2.21 (2H, m), 2.47-2.58 (1H, m), 3.13-3.37 (4H, m), 4.29 (2H, d), 4.64 (2H, s), 4.99 (1H, septet), 8.25 (1H, s).

MS m/z: 415 (M+1), 413 (M−1)

### (d) Isopropyl 5-cyano-6-(4-{[(4-methylbenzyl)sulfonyl]carbamoyl}piperidin-1-yl)-2-[(2-oxopyrrolidin-1-yl)methyl]nicotinate

Prepared according to Example 35(c) from 1-{3-cyano-5-(isopropoxycarbonyl)-6-[(2-oxopyrrolidin-1-yl)methyl]pyridin-2-yl}piperidine-4-carboxylic acid (Example 45(c)) (55 mg, 0.12 mmol) and 1-(4-methylphenyl)methanesulfonamide (33 mg, 0.18 mmol) to give isopropyl 5-cyano-6-(4-{[(4-methylbenzyl)sulfonyl]carbamoyl}piperidin-1-yl)-2-[(2-oxopyrrolidin-1-yl)methyl]nicotinate. Yield: 12 mg (17%).

¹H NMR (600 MHz, DMSO-d₆) δ d 1.29 (6H, d), 1.52-1.62 (2H, m), 1.76-1.82 (2H, m), 1.98 (2H, quintet), 2.27 (2H, t), 2.27 (3H, s), 3.17 (2H, t), 3.42 (2H, t), 4.45 (2H, d), 4.59 (2H, s), 4.73 (2H, s), 5.07 (1H, septet), 7.13 (2H, d), 7.18 (2H, d), 8.35 (1H, s), 11.51 (1H, s).

MS m/z: 582 (M+1), 580 (M−1).

GTPγS (IC₅₀ μM): 0.019

### Example 46

### Isopropyl 5-cyano-6-(4-{[(2,4-difluorobenzyl)sulfonyl]carbamoyl}piperidin-1-yl)-2-[(2-oxopyrrolidin-1-yl)methyl]nicotinate

Prepared according to Example 35(c) from 1-{3-cyano-5-(isopropoxycarbonyl)-6-[(2-oxopyrrolidin-1-yl)methyl]pyridin-2-yl}piperidine-4-carboxylic acid (Example 45 (c)) (55 mg, 0.12 mmol) and 1-(2,4-difluorophenyl)methanesulfonamide (37.3 mg, 0.18 mmol) to give isopropyl 5-cyano-6-(4-{[(2,4-difluorobenzyl)sulfonyl]carbamoyl}piperidin-1-yl)-2-[(2-oxopyrrolidin-1-yl)methyl]nicotinate. Yield: 28.8 mg (40%).

¹H NMR (600 MHz, DMSO-d₆) δ 1.28 (6H, d), 1.53-1.61 (2H, m), 1.80-1.86 (2H, m), 1.98 (2H, quintet), 2.27 (2H, t), 3.15-3.21 (2H, m), 3.41 (2H, t), 4.43-4.49 (2H, m), 4.70 (2H, s), 4.73 (2H, s), 5.07 (1H, septet), 7.15 (1H, td), 7.31 (1H, td), 7.43 (1H, q), 8.35 (1H, s).

MS m/z: 604 (M+1), 602 (M−1).

GTPγS (IC₅₀ μM): 0.021

### Example 47

### Isopropyl 5-cyano-6-(4-{[(cyclopentylmethyl)sulfonyl]carbamoyl}piperidin-1-yl)-2-[(2-oxopyrrolidin-1-yl)methyl]nicotinate

Prepared according to Example 35(c) from 1-{3-cyano-5-(isopropoxycarbonyl)-6-[(2-oxopyrrolidin-1-yl)methyl]pyridin-2-yl}piperidine-4-carboxylic acid (Example 45 (c)) (55 mg, 0.12 mmol) and 1-cyclopentylmethanesulfonamide (29.4 mg, 0.18 mmol) to give isopropyl 5-cyano-6-(4-{[(cyclopentylmethyl)sulfonyl]carbamoyl}piperidin-1-yl)-2-[(2-oxopyrrolidin-1-yl)methyl]nicotinate. Yield: 10.7 mg (16%).

¹H NMR (600 MHz, DMSO-d₆) δ 1.16-1.24 (2H, m), 1.28 (6H, d), 1.42-1.59 (6H, m), 1.78-1.89 (4H, m), 1.96 (2H, quintet), 2.11 (1H, septet), 2.25 (2H, t), 3.19 (2H, t), 3.38 (1H, t), 3.40 (1H, t), 4.41-4.47 (2H, m), 4.72 (2H, s), 5.06 (1H, septet), 8.34 (1H, s).

MS m/z: 560 (M+1), 558 (M−1).

GTPγS (IC₅₀ μM): 0.065

### Example 48

### Isopropyl 6-(4-{[(4-chlorobenzyl)sulfonyl]carbamoyl}piperidin-1-yl)-5-cyano-2-[(2-oxopyrrolidin-1-yl)methyl]nicotinate

Prepared according to Example 35(c) from 1-{3-cyano-5-(isopropoxycarbonyl)-6-[(2-oxopyrrolidin-1-yl)methyl]pyridin-2-yl}piperidine-4-carboxylic acid (Example 45 (c)) (55 mg, 0.12 mmol) and 1-(4-chlorophenyl)methanesulfonamide (37 mg, 0.18 mmol) to give isopropyl 6-(4-{[(4-chlorobenzyl)sulfonyl]carbamoyl}piperidin-1-yl)-5-cyano-2-[(2-oxopyrrolidin-1-yl)methyl]nicotinate. Yield: 28.8 mg (40%).

¹H NMR (600 MHz, DMSO-d₆) δ 1.29 (6H, d), 1.52-1.60 (2H, m), 1.76-1.82 (2H, m), 1.98 (2H, quintet), 2.27 (2H, t), 3.13-3.20 (2H, m), 3.41 (2H, t), 4.43-4.49 (2H, m), 4.68 (2H, s), 4.73 (2H, s), 5.07 (1H, septet), 7.27 (2H, d), 7.46 (2H, d), 8.35 (1H, s).

MS m/z: 602 (M+1), 600 (M−1).

GTPγS (IC₅₀ μM): 0.012

### Example 49

### Isopropyl 6-(4-{[(2-chloro-4-fluorobenzyl)sulfonyl]carbamoyl}piperidin-1-yl)-5-cyano-2-[(2-oxopyrrolidin-1-yl)methyl]nicotinate

Prepared according to Example 35(c) from 1-{3-cyano-5-(isopropoxycarbonyl)-6-[(2-oxopyrrolidin-1-yl)methyl]pyridin-2-yl}piperidine-4-carboxylic acid (Example 45(c)) (55 mg, 0.12 mmol) and 1-(2-chloro-4-fluorophenyl)methanesulfonamide (40.3 mg, 0.18 mmol) to give isopropyl 6-(4-{[(2-chloro-4-fluorobenzyl)sulfonyl]carbamoyl}piperidin-1-yl)-5-cyano-2-[(2-oxopyrrolidin-1-yl)methyl]nicotinate. Yield: 32.8 mg (44%).

¹H NMR (600 MHz, DMSO-d₆) δ 1.28 (6H, d), 1.53-1.62 (2H, m), 1.82-1.87 (2H, m), 1.97 (2H, quintet), 2.26 (2H, t), 3.18 (2H, t), 3.41 (2H, t), 4.42-4.48 (2H, m), 4.72 (2H, s), 4.81 (2H, s), 5.06 (1H, septet), 7.30 (1H, td), 7.47 (1H, dd), 7.52 (1H, dd), 8.34 (1H, s).

MS m/z: 620 (M+1), 618 (M−1).

GTPγS (IC₅₀ μM): 0.066

### Example 50

### Isopropyl 5-cyano-2-[(2-oxopyrrolidin-1-yl)methyl]-6-[4-({[4-(trifluoromethyl)benzyl]sulfonyl}carbamoyl)piperidin-1-yl]nicotinate

Prepared according to Example 35(c) from 1-{3-cyano-5-(isopropoxycarbonyl)-6-[(2-oxopyrrolidin-1-yl)methyl]pyridin-2-yl}piperidine-4-carboxylic acid (Example 45(c)) (55 mg, 0.12 mmol) and 1-[4-(trifluoromethyl)phenyl]methanesulfonamide (43 mg, 0.18 mmol) to give Isopropyl 5-cyano-2-[(2-oxopyrrolidin-1-yl)methyl]-6-[4-({[4-(trifluoromethyl)benzyl]sulfonyl}carbamoyl)piperidin-1-yl]nicotinate. Yield: 26 mg (34%).

¹H NMR (600 MHz, DMSO-d₆) δ 1.29 (6H, d), 1.53-1.61 (2H, m), 1.77-1.82 (2H, m), 1.97 (2H, quintet), 2.26 (2H, t), 3.17 (2H, t), 3.41 (2H, t), 4.43-4.48 (2H, m), 4.73 (2H, s), 4.81 (2H, s), 5.07 (1H, septet), 7.49 (2H, d), 7.77 (2H, d), 8.35 (1H, s)

MS m/z: 636 (M+1), 634 (M−1).

GTPγS (IC₅₀ μM): 0.02

### Example 51

### Isopropyl 5-cyano-6-(4-{[(4-fluorobenzyl)sulfonyl]carbamoyl}piperidin-1-yl)-2-[(2-oxopyrrolidin-1-yl)methyl]nicotinate

Prepared according to Example 35(c) from 1-{3-cyano-5-(isopropoxycarbonyl)-6-[(2-oxopyrrolidin-1-yl)methyl]pyridin-2-yl}piperidine-4-carboxylic acid (Example 45(c)) (55 mg, 0.12 mmol) and 1-(4-fluorophenyl)methanesulfonamide (34 mg, 0.18 mmol) to give Isopropyl 5-cyano-6-(4-{[(4-fluorobenzyl)sulfonyl]carbamoyl}piperidin-1-yl)-2-[(2-oxopyrrolidin-1-yl)methyl]nicotinate. Yield: 31 mg (44%).

¹H NMR (600 MHz, DMSO-d₆) δ 1.28 (6H, d), 1.51-1.60 (2H, m), 1.76-1.81 (2H, m), 1.97 (2H, quintet), 2.26 (2H, t), 3.13-3.20 (2H, m), 3.41 (2H, t), 4.43-4.48 (2H, m), 4.67 (2H, s), 4.73 (2H, s), 5.07 (1H, septet), 7.22 (2H, t), 7.29 (2H, dd), 8.35 (1H, s).

MS m/z: 586 (M+1), 584 (M−1).

GTPγS (IC₅₀ μM): 0.023

### Example 52

### Isopropyl 6-(4-{[(4-chloro-2-fluorobenzyl)sulfonyl]carbamoyl}piperidin-1-yl)-5-cyano-2-[(2-oxopyrrolidin-1-yl)methyl]nicotinate

Prepared according to Example 35(c) from 1-{3-cyano-5-(isopropoxycarbonyl)-6-[(2-oxopyrrolidin-1-yl)methyl]pyridin-2-yl}piperidine-4-carboxylic acid (Example 45(c)) (55 mg, 0.12 mmol) and 1-(4-chloro-2-fluorophenyl)methanesulfonamide (40.3 mg, 0.18 mmol) to give isopropyl 6-(4-{[(4-chloro-2-fluorobenzyl)sulfonyl]carbamoyl}piperidin-1-yl)-5-cyano-2-[(2-oxopyrrolidin-1-yl)methyl]nicotinate. Yield: 23.8 mg (32%).

¹H NMR (600 MHz, DMSO-d₆) δ 1.31 (6H, d), 1.70-1.94 (4H, m), 2.05 (2H, quintet), 2.38 (2H, t), 3.21 (2H, t), 3.43 (2H, t), 4.45-4.55 (2H, m), 4.62 (2H, s), 4.86 (2H, s), 5.15 (1H, septet), 7.11-7.18 (2H, m), 7.30 (1H, t), 8.33 (1H, s).

MS m/z: 620 (M+1), 618 (M−1).

GTPγS (IC₅₀ μM): 0.014

### Example 53

### Isopropyl 5-cyano-6-(4-{[(2,4-dichlorobenzyl)sulfonyl]carbamoyl}piperidin-1-yl)-2-[(2-oxopyrrolidin-1-yl)methyl]nicotinate

Prepared according to Example 35(c) from 1-{3-cyano-5-(isopropoxycarbonyl)-6-[(2-oxopyrrolidin-1-yl)methyl]pyridin-2-yl}piperidine-4-carboxylic acid (Example 45(c)) (55 mg, 0.12 mmol) and 1-(2,4-dichlorophenyl)methanesulfonamide (43 mg, 0.18 mmol) to give isopropyl 5-cyano-6-(4-{[(2,4-dichlorobenzyl)sulfonyl]carbamoyl}piperidin-1-yl)-2-[(2-oxopyrrolidin-1-yl)methyl]nicotinate. Yield: 18.8 mg (25%).

¹H NMR (600 MHz, DMSO-d₆) δ 1.28 (6H, d), 1.54-1.62 (2H, m), 1.82-1.87 (2H, m), 1.98 (2H, quintet), 2.27 (2H, t), 3.15-3.21 (2H, m), 3.41 (2H, t), 4.43-4.48 (2H, m), 4.73 (2H, s), 4.82 (2H, s), 5.07 (1H, septet), 7.43 (1H, d), 7.50 (1H, dd), 7.69 (1H, d), 8.35 (1H, s).

MS m/z: 636 (M+1), 634 (M−1).

GTPγS (IC₅₀ μM): 0.032

### Example 54

### Isopropyl 5-cyano-6-[4-({[(4-methylcyclohexyl)methyl]sulfonyl}carbamoyl)piperidin-1-yl]-2-[(2-oxopyrrolidin-1-yl)methyl]nicotinate

Prepared according to Example 35(c) from 1-{3-cyano-5-(isopropoxycarbonyl)-6-[(2-oxopyrrolidin-1-yl)methyl]pyridin-2-yl}piperidine-4-carboxylic acid (Example 45(c)) (55 mg, 0.12 mmol) and 1-(4-methylcyclohexyl)methanesulfonamide (34.4 mg, 0.18 mmol) to give isopropyl 5-cyano-6-[4-({[(4-methylcyclohexyl)methyl]sulfonyl}carbamoyl)piperidin-1-yl]-2-[(2-oxopyrrolidin-1-yl)methyl]nicotinate. Yield: 13.8 mg (20%).

MS m/z: 588 (M+1), 586 (M−1).

GTPγS (IC₅₀ μM): 0.104

### Example 55

### Ethyl 6-{4-[(anilinosulfonyl)carbamoyl]piperidin-1-yl}-5-cyano-2-[(2-oxopyrrolidin-1-yl)methyl]nicotinate

Prepared according to Example 35(c) from 1-{3-cyano-5-(ethoxycarbonyl)-6-[(2-oxopyrrolidin-1-yl)methyl]pyridin-2-yl}piperidine-4-carboxylic acid (Example 35(b)) (251 mg, 0.63 mmol) and N-phenylsulfamide (152 mg, 0.88 mmol) to give Ethyl 6-{4-[(anilinosulfonyl)carbamoyl]piperidin-1-yl}-5-cyano-2-[(2-oxopyrrolidin-1-yl)methyl]nicotinate. Yield: 205 mg (59%).

¹H NMR (600 MHz, DMSO-d₆) δ 1.26 (3H, t), 1.28-1.39 (2H, m), 1.53-1.61 (2H, m), 1.85-1.95 (2H, m), 2.16-2.22 (2H, m), 3.08-3.17 (2H, m), 3.32-3.37 (2H, m), 4.17-4.29 (4H, m), 4.68 (2H, s), 6.99-7.05 (1H, m), 7.06-7.10 (2H, m), 7.21-7.27 (2H, m), 8.31 (1H, s). One signal (1H) is overlapping with the solvent signal (2.39-2.48 ppm).

MS m/z: 555 (M+1), 553 (M−1).

GTPγS (IC₅₀ μM): 0.03

### Example 56

### Ethyl 6-{4-[(benzylsulfonyl)carbamoyl]piperidin-1-yl}-5-cyano-2-[(2-oxopiperidin-1-yl)methyl]nicotinate

### (a) Ethyl 3-oxo-4-(2-oxopiperidin-1-yl)butanoate

Prepared according to Example 33(a) from ethyl 4-chloracetoacetate (5 g, 30 mmol) and delta-valerolactam (3.2 g, 32 mmol) to give ethyl 3-oxo-4-(2-oxopiperidin-1-yl)butanoate. Yield: 6.3 g (91%).

¹H NMR (500 MHz, CDCl₃) δ 1.26 (3H, t), 1.84 (4H, m), 2.42 (2H, m), 3.30 (2H, m), 3.64 (2H, s), 4.18 (2H, q), 4.26 (2H, s).

MS m/z: 228 (M+1).

### (b) Ethyl 5-cyano-6-hydroxy-2-[(2-oxopiperidin-1-yl)methyl]nicotinate

Prepared according to Example 33(b) from ethyl 3-oxo-4-(2-oxopiperidin-1-yl)butanoate (Example 56 (a)) (2.0 g, 6.6 mmol) to give ethyl 5-cyano-6-hydroxy-2-[(2-oxopiperidin-1-yl)methyl]nicotinate. Yield: 2.3 g (74%).

¹H NMR (500 MHz, DMSO-d₆) δ 1.29 (3H, t), 1.76 (4H, m), 2.28 (2H, m), 3.31 (2H, m), 4.24 (2H, q), 4.81 (2H, s), 8.47 (1H, s).

MS m/z: 304 (M+1)

### (c) Ethyl 6-[4-(tert-butoxycarbonyl)piperidin-1-yl]-5-cyano-2-[(2-oxopiperidin-1-yl)methyl]nicotinate

Prepared according to Example 35(a) from ethyl 5-cyano-6-hydroxy-2-[(2-oxopiperidin-1-yl)methyl]nicotinate (Example 56 (b)) (2.0 g, 6.6 mmol) to give ethyl 6-[4-(tert-butoxycarbonyl)piperidin-1-yl]-5-cyano-2-[(2-oxopiperidin-1-yl)methyl]nicotinate. Yield: 2.3 g (74%).

¹H NMR (500 MHz, DMSO-d₆) δ 1.30 (3H, t), 1.39 (9H, s), 1.50-1.60 (2H, m), 1.72-1.80 (4H, m), 1.87-1.93 (2H, m), 2.22-2.28 (2H, m), 2.57-2.64 (1H, m), 3.24-3.35 (4H, m), 4.26 (2H, q), 4.39-4.45 (2H, m), 4.79 (1H, s), 8.37 (1H, s).

MS m/z: 471 (M+1)

### (d) 1-{3-Cyano-5-(ethoxycarbonyl)-6-[(2-oxopiperidin-1-yl)methyl]pyridin-2-yl}piperidine-4-carboxylic acid

Prepared according to Example 35(b) from ethyl 6-[4-(tert-butoxycarbonyl)piperidin-1-yl]-5-cyano-2-[(2-oxopiperidin-1-yl)methyl]nicotinate (Example 56 (c)) (2.3 g, 4.89 mmol) to give 1-{3-cyano-5-(ethoxycarbonyl)-6-[(2-oxopiperidin-1-yl)methyl]pyridin-2-yl}piperidine-4-carboxylic acid. Yield: 2.4 g (99%).

¹H NMR (500 MHz, CD₃OD) δ 1.35 (3H, t), 1.69-1.78 (2H, m), 1.85-1.93 (4H, m), 1.99-2.05 (2H, m), 2.39-2.44 (2H, m), 2.63-2.71 (1H, m), 3.32-3.38 (2H, m), 3.42-3.46 (2H, m), 4.31 (2H, q), 4.50-4.55 (2H, m), 4.94 (2H, s), 8.38 (1H, s).

### (e) Ethyl 6-{4-[(benzylsulfonyl)carbamoyl]piperidin-1-yl}-5-cyano-2-[(2-oxopiperidin-1-yl)methyl]nicotinate

Prepared according to Example 35(c) from 1-{3-cyano-5-(ethoxycarbonyl)-6-[(2-oxopiperidin-1-yl)methyl]pyridin-2-yl}piperidine-4-carboxylic acid (Example 56 (d)) (500 mg, 1.65 mmol) and N-phenylsulfamide (698 mg, 2.47 mmol) to give ethyl 6-{4-[(benzylsulfonyl)carbamoyl]piperidin-1-yl}-5-cyano-2-[(2-oxopiperidin-1-yl)methyl]nicotinate. Yield: 686 mg (73%).

¹H-NMR (500 MHz, CDCl₃) δ 1.38 (3H, t), 1.70-1.87 (8H, m), 2.27 (2H, t), 2.44-2.50 (1H, m), 3.16-3.22 (2H, m), 3.32 (2H, t), 4.33 (2H, q), 4.44-4.49 (2H, m), 4.63 (2H, s), 4.93 (2H, s), 7.32-7.39 (4H, m), 8.39 (1H, s), 9.50 (1H, s).

GTPγS (IC₅₀ μM): 0.03

### Example 57

### Ethyl 5-cyano-6-[4-({[methyl(phenyl)amino]sulfonyl}carbamoyl)piperidin-1-yl]-2-[(2-oxopyrrolidin-1-yl)methyl]nicotinate

### (a) N-Methyl-N-phenylsulfamide

Chlorosulfonyl isocyanate (3.7 mL, 42.4 mmol) was dissolved in dry DCM (40 mL), the solution was cooled to 0° C. and tert-butanol (3.98 mL, 42.4 mmol) was added drop wise. The reaction mixture was stirred at rt for 2 h, the solution was cooled to 0° C. and N-methylaniline (4.61 mL, 42.4 mmol) and TEA (8.85 mL, 63.6 mmol) dissolved in dry DCM (20 mL) were added drop wise through a dropping funnel. The reaction was stirred at rt for 3 h, water was added and the organic phase was separated and dried (phase separator, Isolute) and concentrated in vacuo. The residue was dissolved in DCM (40 mL) and trifluoroacetic acid (32.7 mL, 423 mmol) was added. The reaction was stirred at rt for 20 min, the solvent was concentrated in vacuo and co evaporated with DCM (3×). The crude product was purified with flash column chromatography, using a mixture of heptane:EtOAc 70:30 as eluent, to give N-methyl-N-phenylsulfamide. Yield: 5.96 g (76%).

¹H-NMR (500 MHz, CDCl₃) [symbol] 3.22 (3H, s), 4.77 (2H, s), 7.28-7.33 (1H, m), 7.36-7.42 (4H, m).

MS m/z: 187 (M+1).

### (b) Ethyl 5-cyano-6-[4-({[methyl(phenyl)amino]sulfonyl}carbamoyl)piperidin-1-yl]-2-[(2-oxopyrrolidin-1-yl)methyl]nicotinate

1-{3-Cyano-5-(ethoxycarbonyl)-6-[(2-oxopyrrolidin-1-yl)methyl]pyridin-2-yl}piperidine-4-carboxylic acid (Example 35(b)) (1.0 g, 2.6 mmol) and TBTU (1.1 g, 3.3 mmol) were suspension in dry DCM (8 mL), DIPEA (1.3 mL, 7.46 mmol) was added. The reaction mixture was stirred at r.t for 1 h and N-methyl-N-phenylsulfamide (Example 57 (a)) (0.53 g, 2.8 mmol) was added. The reaction mixture was stirred at r.t over night. NaHCO₃ (aq) was added, the organic layer was separated and the aqueous layer was extracted with DCM (×2). The combined organics phases was dried (phase separator) and concentrated in vacuo. The residue was purified by HPLC (Kromasil C₈, 250×50 mmID, using an increasing gradient of 30% to 80% MeCN over 30 min. with an acidic second eluent (H2O/MeCN/FA, 95/5/0.2)) to give ethyl 5-cyano-6-[4-({[methyl(phenyl)amino]sulfonyl}carbamoyl)piperidin-1-yl]-2-[(2-oxopyrrolidin-1-yl)methyl]nicotinate as a white solid. Yield: 801 mg (55%).

¹H-NMR (400 MHz, DMSO-d₆) δ 1.27 (3H, t), 1.41-1.54 (2H, m), 1.66-1.74 (2H, m), 1.95 (2H, pentet), 2.24 (2H, t), 2.49-2.58 (1H, m), 3.09-3.19 (2H, m), 3.30 (3H, s), 3.39 (2H, t), 4.23 (2H, q), 4.36-4.43 (2H, m), 4.71 (2H, s), 7.24-7.31 (3H, m), 7.34-7.40 (2H, m), 8.34 (1H, s), 11.62 (1H, br s).

MS m/z: 569 (M+1), 567 (M−1)

GTPγS (IC₅₀ μM): 0.013

### Example 58

### Ethyl 5-cyano-6-[4-({[(4-methylcyclohexyl)methyl]sulfonyl}carbamoyl)piperidin-1-yl]-2-[(2-oxopiperidin-1-yl)methyl]nicotinate

Prepared according to Example 57(b) from 1-{3-cyano-5-(ethoxycarbonyl)-6-[(2-oxopiperidin-1-yl)methyl]pyridin-2-yl}piperidine-4-carboxylic acid (Example 56 (d)) (150 mg, 0.36 mmol) and 1-(4-methylcyclohexyl)methanesulfonamide (83 mg, 0.43 mmol) in place of N-methyl-N-phenylsulfamide to give ethyl 5-cyano-6-[4-({[(4-methylcyclohexyl)methyl]sulfonyl}carbamoyl)piperidin-1-yl]-2-[(2-oxopiperidin-1-yl)methyl]nicotinate. Yield: 13 mg (6%).

¹H-NMR (500 MHz, CDCl₃) δ 0.87-0.94 (3H, m), 1.09-1.22 (2H, m), 1.37-1.43 (4H, m), 1.50-1.65 (4H, m), 1.69-2.34 (10H, m), 2.44-2.52 (2H, m), 2.58-2.66 (1H, m), 3.23-3.47 (6H, m), 4.23-4.38 (3H, m), 4.51-4.57 (1H, m), 4.78-4.84 (1H, m), 4.98-5.03 (2H, m), 8.41 (1H, s), 9.93 (1H, s).

MS m/z: 586 (M+1), 588 (M−1).

GTPγS (IC₅₀ μM): 0.164

### Example 59

### Ethyl 5-cyano-2-[(2-oxopiperidin-1-yl)methyl]-6-[4-({[4-(trifluoromethyl)benzyl]sulfonyl}carbamoyl)piperidin-1-yl]nicotinate

Prepared according to Example 35(c) from 1-{3-cyano-5-(ethoxycarbonyl)-6-[(2-oxopiperidin-1-yl)methyl]pyridin-2-yl}piperidine-4-carboxylic acid (Example 56(d)) (150 mg, 0.36 mmol) and 1-[4-(trifluoromethyl)phenyl]methanesulfonamide (104 mg, 0.43 mmol) to give ethyl 5-cyano-2-[(2-oxopiperidin-1-yl)methyl]-6-[4-({[4-(trifluoromethyl)benzyl]sulfonyl}carbamoyl)piperidin-1-yl]nicotinate. Yield: 48 mg (21%).

¹H-NMR (500 MHz, CDCl₃) δ 1.37 (3H, t), 1.70-1.85 (8H, m), 2.19 (2H, t), 2.49-2.55 (1H, m), 3.11-3.16 (2H, m), 3.29 (2H, t), 4.31 (2H, q), 4.43-4.48 (2H, m), 4.66 (2H, s), 4.92 (2H, s), 7.47-7.64 (4H, m), 8.38 (1H, s), 10.56 (1H, br s).

GTPγS (IC₅₀ μM): 0.029

### Example 60

### Ethyl 5-cyano-6-(4-{[(cyclopentylmethyl)sulfonyl]carbamoyl}piperidin-1-yl)-2-[(2-oxopiperidin-1-yl)methyl]nicotinate

Prepared according to Example 35(c) from 1-{3-cyano-5-(ethoxycarbonyl)-6-[(2-oxopiperidin-1-yl)methyl]pyridin-2-yl}piperidine-4-carboxylic acid (Example 56(d)) (150 mg, 0.36 mmol) and 1-cyclopentylmethanesulfonamide (89 mg, 0.54 mmol) to give ethyl 5-cyano-6-(4-{[(cyclopentylmethyl)sulfonyl]carbamoyl}piperidin-1-yl)-2-[(2-oxopiperidin-1-yl)methyl]nicotinate. Yield: 45 mg (22%).

¹H-NMR (500 MHz, CDCl₃) δ 1.22-1.30 (2H, m), 1.37 (3H, t), 1.50-1.68 (4H, m), 1.76-1.97 (10H, m), 2.25-2.34 (1H, m), 2.45-2.49 (2H, m), 2.57-2.63 (1H, m), 3.19-3.25 (2H, m), 3.32-3.35 (2H, m), 3.41-3.45 (2H, m), 4.31 (2H, q), 4.47-4.52 (2H, m), 4.97 (2H, s), 8.38 (1H, s), 10.29 (1H, s).

MS m/z: 560 (M+1)

GTPγS (IC₅₀ μM): 0.111

### Example 61

### Ethyl 5-cyano-6-(4-{[(cyclohexylmethyl)sulfonyl]carbamoyl}piperidin-1-yl)-2-[(2-oxopiperidin-1-yl)methyl]nicotinate

Prepared according to Example 35(c) from 1-{3-cyano-5-(ethoxycarbonyl)-6-[(2-oxopiperidin-1-yl)methyl]pyridin-2-yl}piperidine-4-carboxylic acid (Example 56(d)) (150 mg, 0.36 mmol) and 1-cyclohexylmethanesulfonamide (96 mg, 0.54 mmol) to give ethyl 5-cyano-6-(4-{[(cyclohexylmethyl)sulfonyl]carbamoyl}piperidin-1-yl)-2-[(2-oxopiperidin-1-yl)methyl]nicotinate. Yield: 44 mg (21%).

¹H-NMR (500 MHz, CDCl₃) δ 1.06-1.33 (5H, m), 1.38 (3H, t), 1.63-2.01 (14H, m), 2.47-2.51 (2H, m), 2.58-2.65 (1H, m), 3.20-3.26 (2H, m), 3.31 (2H, d), 3.34-3.38 (2H, m), 4.33 (2H, q), 4.48-4.54 (2H, m), 4.99 (2H, s), 8.40 (1H, s), 10.33 (1H, s).

MS m/z: 574 (M+1).

GTPγS (IC₅₀ μM): 0.061

### Example 62

### Ethyl 5-cyano-6-(4-{[(4-fluorobenzyl)sulfonyl]carbamoyl}piperidin-1-yl)-2-[(2-oxopiperidin-1-yl)methyl]nicotinate

Prepared according to Example 35(c) from 1-{3-cyano-5-(ethoxycarbonyl)-6-[(2-oxopiperidin-1-yl)methyl]pyridin-2-yl}piperidine-4-carboxylic acid (Example 56(d)) (150 mg, 0.36 mmol) and 1-(4-fluorophenyl)methanesulfonamide (103 mg, 0.54 mmol) to give ethyl 5-cyano-6-(4-{[(4-fluorobenzyl)sulfonyl]carbamoyl}piperidin-1-yl)-2-[(2-oxopiperidin-1-yl)methyl]nicotinate. Yield: 53 mg (25%).

¹H-NMR (500 MHz, CDCl₃) δ 1.33 (3H, t), 1.65-1.82 (8H, m), 2.20 (2H, t), 2.45-2.52 (1H, m), 3.06-3.12 (2H, m), 3.27 (2H, t), 4.27 (2H, q), 4.42-4.46 (2H, m), 4.52 (2H, s), 4.88 (2H, s), 6.98-7.03 (2H, m), 7.25-7.28 (2H, m), 8.33 (1H, s), 10.39 (1H, br s).

MS m/z: 586 (M+1).

GTPγS (IC₅₀ μM): 0.148

### Example 63

### Ethyl 5-cyano-6-(4-{[(4-methylbenzyl)sulfonyl]carbamoyl}piperidin-1-yl)-2-[(2-oxopiperidin-1-yl)methyl]nicotinate

Prepared according to Example 35(c) from 1-{3-cyano-5-(ethoxycarbonyl)-6-[(2-oxopiperidin-1-yl)methyl]pyridin-2-yl}piperidine-4-carboxylic acid (Example 56(d)) (150 mg, 0.36 mmol) and 1-(4-methylphenyl)methanesulfonamide (101 mg, 0.54 mmol) to give ethyl 5-cyano-6-(4-{[(4-methylbenzyl)sulfonyl]carbamoyl}piperidin-1-yl)-2-[(2-oxopiperidin-1-yl)methyl]nicotinate. Yield: 40 mg (19%).

¹H-NMR (500 MHz, CDCl₃) δ 1.37 (3H, t), 1.71-1.86 (8H, m), 2.25 (2H, t), 2.34 (3H, s), 2.47-2.53 (1H, m), 3.15-3.20 (2H, m), 3.32 (2H, t), 4.32 (2H, q), 4.46-4.50 (2H, m), 4.56 (2H, s), 4.93 (2H, s), 7.15-7.21 (4H, m), 8.38 (1H, s), 9.83 (1H, s).

MS m/z: 582 (M+1)

GTPγS (IC₅₀ μM): 0.014

### Example 64

### Ethyl 6-{4-[(anilinosulfonyl)carbamoyl]piperidin-1-yl}-5-cyano-2-[(2-oxopiperidin-1-yl)methyl]nicotinate

Prepared according to Example 35(c) from 1-{3-cyano-5-(ethoxycarbonyl)-6-[(2-oxopiperidin-1-yl)methyl]pyridin-2-yl}piperidine-4-carboxylic acid (Example 56d) (400 mg, 0.97 mmol) and N-phenylsulfamide (249 mg, 1.45 mmol) to give ethyl 6-{4-[(anilinosulfonyl)carbamoyl]piperidin-1-yl}-5-cyano-2-[(2-oxopiperidin-1-yl)methyl]nicotinate. Yield: 186 mg (34%).

¹H-NMR (500 MHz, DMSO-d₆) δ 1.31 (3H, t), 1.33-1.43 (2H, m), 1.59-1.65 (2H, m), 1.70-1.79 (4H, m), 2.22 (2H, t), 2.51-2.56 (1H, m), 3.15-3.21 (2H, m), 3.29-3.36 (2H, m), 4.26 (2H, q), 4.32-4.37 (2H, m), 4.76-4.80 (2H, m), 7.07-7.32 (5H, m), 8.36 (1H, s), 10.40 (1H, s), 11.72 (1H, s).

MS m/z: 569 (M+1).

GTPγS (IC₅₀ μM): 0.14

### Example 65

### Ethyl 5-cyano-6-(4-{[(cyclohexylmethyl)sulfonyl]carbamoyl}piperidin-1-yl)-2-[(2-oxopyrrolidin-1-yl)methyl]nicotinate

Prepared according to Example 35(c) from ethyl 5-cyano-6-hydroxy-2-[(2-oxopyrrolidin-1-yl)methyl]nicotinate (Example 35(b)) (110 mg, 0.28 mmol) and 1-cyclohexylmethanesulfonamide (58 mg, 0.33 mmol) to give ethyl 5-cyano-6-(4-{[(cyclohexylmethyl)sulfonyl]carbamoyl}piperidin-1-yl)-2-[(2-oxopyrrolidin-1-yl)methyl]nicotinate. Yield: 62 mg (40%).

¹H-NMR (500 MHz, CDCl₃) δ 1.06-1.22 (3H, m), 1.24-1.35 (2H, m), 1.39 (3H, t), 1.62-1.74 (3H, m), 1.78-2.04 (7H, m), 2.08-2.15 (2H, m), 2.51 (2H, t), 2.63-2.70 (1H, m), 3.23-3.33 (4H, m), 3.48 (2H, t), 4.34 (2H, q), 4.48-4.54 (2H, m), 4.93 (2H, s), 8.40 (1H, s), 10.18 (1H, s).

MS m/z: 560 (M+1), 558 (M−1).

GTPγS (IC₅₀ μM): 0.021

### Example 66

### Ethyl 5-cyano-2-[(2-oxopyrrolidin-1-yl)methyl]-6-{4-[({4-[(trifluoromethyl)thio]benzyl}sulfonyl)carbamoyl]piperidin-1-yl}nicotinate

Prepared according to Example 35(c) from 1-{3-cyano-5-(ethoxycarbonyl)-6-[(2-oxopyrrolidin-1-yl)methyl]pyridin-2-yl}piperidine-4-carboxylic acid (Example 35(b)) (110 mg, 0.28 mmol) and 1-{4-[(trifluoromethyl)thio]phenyl}methanesulfonamide (89 mg, 0.33 mmol) to give ethyl 5-cyano-2-[(2-oxopyrrolidin-1-yl)methyl]-6-{4-[({4-[(trifluoromethyl)thio]benzyl}sulfonyl)carbamoyl]piperidin-1-yl}nicotinate. Yield: 104 mg (58%).

¹H-NMR (500 MHz, CDCl₃) δ 1.39 (3H, t), 1.72-1.88 (4H, m), 2.03-2.10 (2H, m), 2.31 (2H, t), 2.54-2.61 (1H, m), 3.13-3.20 (2H, m), 3.44 (2H, t), 4.34 (2H, q), 4.43-4.50 (2H, m), 4.66 (2H, s), 4.88 (2H, s), 7.43 (2H, d), 7.67 (2H, d), 8.40 (1H, s), 10.41 (1H, s).

MS m/z: 654 (M+1), 652 (M−1).

GTPγS (IC₅₀ μM): 0.033

### Example 67

### Ethyl 5-cyano-6-(4-{[(2,4-difluorobenzyl)sulfonyl]carbamoyl}piperidin-1-yl)-2-[(2-oxopiperidin-1-yl)methyl]nicotinate

Prepared according to Example 35(c) from 1-{3-cyano-5-(ethoxycarbonyl)-6-[(2-oxopiperidin-1-yl)methyl]pyridin-2-yl}piperidine-4-carboxylic acid (Example 56(d)) (146 mg, 0.35 mmol) and 1-(2,4-difluorophenyl)methanesulfonamide (88 mg, 0.42 mmol) to give ethyl 5-cyano-6-(4-{[(2,4-difluorobenzyl)sulfonyl]carbamoyl}piperidin-1-yl)-2-[(2-oxopiperidin-1-yl)methyl]nicotinate. Yield: 81 mg (23%).

¹H-NMR (500 MHz, CDCl₃) δ 1.39 (3H, t), 1.75-1.93 (8H, m), 2.27-2.32 (2H, m), 2.55-2.63 (1H, m), 3.16-3.35 (4H, m), 4.34 (2H, q), 4.49 (2H, m), 4.66 (2H, s), 4.94 (2H, s), 6.87-6.96 (2H, m), 7.37-7.43 (1H, m), 8.40 (1H, s), 10.45 (1H, s).

MS m/z: 604 (M+1), 602 (M−1).

GTPγS (IC₅₀ μM): 0.02

### Example 68

### Ethyl 5-cyano-6-(4-{[(4-methoxybenzyl)sulfonyl]carbamoyl}piperidin-1-yl)-2-[(2-oxopiperidin-1-yl)methyl]nicotinate

Prepared according to Example 35(c) from 1-{3-cyano-5-(ethoxycarbonyl)-6-[(2-oxopiperidin-1-yl)methyl]pyridin-2-yl}piperidine-4-carboxylic acid (Example 56(d)) (146 mg, 0.35 mmol) and 1-(4-methoxyphenyl)methanesulfonamide (85 mg, 0.42 mmol) to give ethyl 5-cyano-6-(4-{[(4-methoxybenzyl)sulfonyl]carbamoyl}piperidin-1-yl)-2-[(2-oxopiperidin-1-yl)methyl]nicotinate. Yield: 112 mg (32%).

¹H-NMR (500 MHz, CDCl₃) δ 1.38 (3H, t), 1.71-1.89 (8H, m), 2.27-2.30 (2H, m), 2.51-2.57 (1H, m), 3.14-3.20 (2H, m), 3.31-3.36 (2H, m), 3.80 (3H, s), 4.30-4.35 (2H, m), 4.48-4.54 (2H, m), 4.55 (2H, s), 4.95 (2H, s), 6.88 (2H, d), 7.24 (2H, d), 8.39 (1H, s), 10.14 (1H, s).

MS m/z: 598 (M+1), 596 (M−1).

GTPγS (IC₅₀ μM): 0.024

### Example 69

### Ethyl 5-cyano-6-(4-{[(4-isopropylbenzyl)sulfonyl]carbamoyl}piperidin-1-yl)-2-[(2-oxopiperidin-1-yl)methyl]nicotinate

Prepared according to Example 35(c) from 1-{3-cyano-5-(ethoxycarbonyl)-6-[(2-oxopiperidin-1-yl)methyl]pyridin-2-yl}piperidine-4-carboxylic acid (Example 56(d)) (146 mg, 0.35 mmol) and 1-(4-isopropylphenyl)methanesulfonamide (90 mg, 0.42 mmol) to give ethyl 5-cyano-6-(4-{[(4-isopropylbenzyl)sulfonyl]carbamoyl}piperidin-1-yl)-2-[(2-oxopiperidin-1-yl)methyl]nicotinate. Yield: 60 mg (28%).

¹H-NMR (500 MHz, CDCl₃) δ 1.24 (3H, s), 1.25 (3H, s), 1.39 (3H, t), 1.73-1.89 (8H, m), 2.25-2.31 (2H, m), 2.48-2.55 (1H, m), 2.89-2.95 (1H, m), 3.16-3.22 (2H, m), 3.32-3.36 (2H, m), 4.34 (2H, q), 4.47-4.52 (2H, m), 4.60 (2H, s), 4.96 (2H, s), 7.22-7.29 (4H, m), 8.40 (1H, s), 9.83 (1H, s).

MS m/z: 610 (M+1), 608 (M−1).

GTPγS (IC₅₀ μM): 0.082

### Example 70

### Isopropyl 6-{4-[(benzylsulfonyl)carbamoyl]piperidin-1-yl}-5-cyano-2-[(2-oxopiperidin-1-yl)methyl]nicotinate

### (a) 6-{4-[(Benzylsulfonyl)carbamoyl]piperidin-1-yl}-5-cyano-2-[(2-oxopiperidin-1-yl)methyl]nicotinic acid

Ethyl 6-{4-[(benzylsulfonyl)carbamoyl]piperidin-1-yl}-5-cyano-2-[(2-oxopiperidin-1-yl)methyl]nicotinate (253 mg, 0.45 mmol) was added to a solution of 2-propanol (2 mL) and 1 M NaOH (1.78 mL), the reaction mixture was heated to 80° C. for 5 minutes. 4 M HCl (1 mL) and water (2 mL) were added and the mixture was freeze dried to give 6-{4-[(benzylsulfonyl)carbamoyl]piperidin-1-yl}-5-cyano-2-[(2-oxopiperidin-1-yl)methyl]nicotinic acid. The crude product was used in the next step without further purification.

### (b) Isopropyl 6-{4-[(benzylsulfonyl)carbamoyl]piperidin-1-yl}-5-cyano-2-[(2-oxopiperidin-1-yl)methyl]nicotinate

N,N′-Carbonyldiimidazole (97 mg, 0.45 mmol) was added to a slurry of 6-{4-[(benzylsulfonyl)carbamoyl]piperidin-1-yl}-5-cyano-2-[(2-oxopiperidin-1-yl)methyl]nicotinic acid (241 mg, 0.45 mmol) in acetonitrile (7 mL) at rt, the reaction mixture was heated to 50° C. for 1.5 h. The mixture was cooled to rt, 2-propanol (1 mL) and DIPEA (0.24 mL, 1.34 mmol) were added. The reaction mixture was heated to 50° C. over the weekend (52 h). DCM (50 mL) was added, extracted with water (5 mL), the organic solvent was dried (phase separator) and concentrated in vacuo. The residue was purified by HPLC (Kromasil C₈, 250×50 mmID, using an increasing gradient of 20% to 100% MeCN over 30 min. with an acidic second eluent (H2O/MeCN/FA, 95/5/0.2)) to give isopropyl 6-{4-[(benzylsulfonyl)carbamoyl]piperidin-1-yl}-5-cyano-2-[(2-oxopiperidin-1-yl)methyl]nicotinate as a white solid. Yield: 179 mg (69%).

¹H-NMR (400 MHz, DMSO-d₆) δ 1.30 (3H, s), 1.31 (3H, s), 1.55-1.64 (2H, m), 1.74-1.84 (6H, m), 2.24-2.30 (2H, m), 2.56-2.63 (1H, m), 3.15-3.26 (2H, m), 3.33-3.39 (2H, m), 4.48-4.54 (2H, m), 4.68 (2H, s), 4.80 (2H, s), 5.04-5.13 (1H, m), 7.26-7.30 (2H, m), 7.37-7.40 (3H, m), 8.37 (1H, s), 11.58 (1H, s).

MS m/z: 582 (M+1), 580 (M−1).

GTPγS (IC₅₀ μM): 0.102

### Example 71

### Ethyl 5-cyano-6-[4-({[methyl(phenyl)amino]sulfonyl}carbamoyl)piperidin-1-yl]-2-[(2-oxopiperidin-1-yl)methyl]nicotinate

Ethyl 6-{4-[(anilinosulfonyl)carbamoyl]piperidin-1-yl}-5-cyano-2-[(2-oxopiperidin-1-yl)methyl]nicotinate (Example 64) (160 mg, 0.28 mmol) was added to a solution of 2-methyl tetrahydrofurane (2 mL) and DMF (4 mL), the mixture was cooled to 0° C. Sodium hydride (14 mg, 0.56 mmol) was added and the reaction mixture was stirred at 0° C. for 20 min. Iodomethane (0.02 mL, 0.3 mmol) was added and the reaction was stirred for 1 h at rt. Acidic water and DCM were added, the organic solvent was washed with water, dried and concentrated in vacuo. The residue was purified by HPLC ((Kromasil C₈, 250×50 mmID, using an increasing gradient of 50% to 100% MeCN over 30 min. with an acidic second eluent (H2O/MeCN/FA, 95/5/0.2))) to give ethyl 5-cyano-6-[4-({[methyl(phenyl)amino]sulfonyl}carbamoyl)piperidin-1-yl]-2-[(2-oxopiperidin-1-yl)methyl]nicotinate. Yield: 38 mg (23%).

¹H-NMR (500 MHz, CDCl₃) δ 1.36 (3H, t), 1.64-1.86 (8H, m), 2.30 (2H, t), 2.41-2.47 (1H, m), 3.09-3.16 (2H, m), 3.31 (2H, t), 3.45 (3H, s), 4.31 (2H, q), 4.43-4.48 (2H, m), 4.92 (2H, s), 7.25-7.36 (5H, m), 8.36 (1H, s), 9.91 (1H, br s).

MS m/z: 583 (M+1).

GTPγS (IC₅₀ μM): 0.036

### Example 72

### Ethyl 5-cyano-2-[(2-oxopyrrolidin-1-yl)methyl]-6-(4-{[(tetrahydro-2H-pyran-4-ylmethyl)sulfonyl]carbamoyl}piperidin-1-yl)nicotinate

Prepared according to Example 35(c) from 1-{3-cyano-5-(ethoxycarbonyl)-6-[(2-oxopyrrolidin-1-yl)methyl]pyridin-2-yl}piperidine-4-carboxylic acid (Example 35(b)) (150 mg, 0.38 mmol) and 1-(tetrahydro-2H-pyran-4-yl)methanesulfonamide (101 mg, 0.56 mmol) to give ethyl 5-cyano-2-[(2-oxopyrrolidin-1-yl)methyl]-6-(4-{[(tetrahydro-2H-pyran-4-ylmethyl)sulfonyl]carbamoyl}piperidin-1-yl)nicotinate. Yield: 84 mg (40%).

¹H-NMR (500 MHz, CDCl₃) δ 1.38 (3H, t), 1.42-1.50 (2H, m), 1.77-1.85 (4H, m), 1.92-1.98 (2H, m), 2.12 (2H, quintet), 2.21-2.30 (1H, m), 2.51 (2H, t), 2.61-2.67 (1H, m), 3.23-3.29 (2H, m), 3.36 (2H, d), 3.39-3.44 (2H, m), 3.47 (2H, t), 3.92-3.96 (2H, m), 4.33 (2H, q), 4.45-4.50 (2H, m), 4.92 (2H, s), 8.39 (1H, s), 10.20 (1H, br s).

MS m/z: 562 (M+1).

GTPγS (IC₅₀ μM): 0.725

### Example 73

### Isopropyl 5-cyano-2-[(2-oxopyrrolidin-1-yl)methyl]-6-(4-{[(tetrahydro-2H-pyran-4-ylmethyl)sulfonyl]carbamoyl}piperidin-1-yl)nicotinate

Prepared according to Example 35(c) from 1-{3-cyano-5-(isopropoxycarbonyl)-6-[(2-oxopyrrolidin-1-yl)methyl]pyridin-2-yl}piperidine-4-carboxylic acid (Example 45(c)) (160 mg, 0.39 mmol) and using 1-(tetrahydro-2H-pyran-4-yl)methanesulfonamide (104 mg, 0.58 mmol) to give isopropyl 5-cyano-2-[(2-oxopyrrolidin-1-yl)methyl]-6-(4-{[(tetrahydro-2H-pyran-4-ylmethyl)sulfonyl]carbamoyl}piperidin-1-yl)nicotinate. Yield: 10 mg (5%).

¹H-NMR (500 MHz, CDCl₃) δ 1.35 (6H, d), 1.42-1.51 (2H, m), 1.77-1.92 (5H, m), 1.94-2.04 (2H, m), 2.07-2.15 (2H, m), 2.47-2.53 (2H, m), 2.63-2.77 (2H, m), 3.24-3.30 (2H, m), 3.36 (2H, d), 3.39-3.54 (4H, m), 3.92-3.96 (1H, m), 4.47-4.52 (1H, m), 4.62-4.67 (1H, m), 4.92 (2H, s), 5.15-5.23 (1H, m), 8.35 (1H, s), 10.02 (1H, br s).

MS m/z: 576 (M+1).

GTPγS (IC₅₀ μM): >3.3

### Example 74

### Ethyl 5-cyano-6-(4-{[(cyclobutylmethyl)sulfonyl]carbamoyl}piperidin-1-yl)-2-[(2-oxopyrrolidin-1-yl)methyl]nicotinate

Prepared according to Example 57(b) from 1-{3-cyano-5-(ethoxycarbonyl)-6-[(2-oxopyrrolidin-1-yl)methyl]pyridin-2-yl}piperidine-4-carboxylic acid (Example 35(b)) (150 mg, 0.38 mmol) and 1-cyclobutylmethanesulfonamide (62 mg, 0.42 mmol) to give ethyl 5-cyano-6-(4-{[(cyclobutylmethyl)sulfonyl]carbamoyl}piperidin-1-yl)-2-[(2-oxopyrrolidin-1-yl)methyl]nicotinate. Yield: 92 mg (46%).

¹H-NMR (400 MHz, DMSO-d₆) δ 1.27 (3H, t), 1.48-1.60 (2H, m), 1.69-1.90 (6H, m), 1.96 (2H, pentet), 1.99-2.07 (2H, m), 2.24 (2H, t), 2.55-2.67 (2H, m), 3.12-3.22 (2H, m), 3.36-3.45 (4H, m), 4.23 (2H, q), 4.40-4.48 (2H, m), 4.71 (1H, s), 8.34 (1H, s), 11.62 (1H, br s).

MS m/z: 532 (M+1), 530 (M−1).

GTPγS (IC₅₀ μM): 0.037

### Example 75

### Ethyl 5-cyano-6-(4-{[(4-methoxybenzyl)sulfonyl]carbamoyl}piperidin-1-yl)-2-[(2-oxopyrrolidin-1-yl)methyl]nicotinate

Prepared according to Example 57(b) from 1-{3-cyano-5-(ethoxycarbonyl)-6-[(2-oxopyrrolidin-1-yl)methyl]pyridin-2-yl}piperidine-4-carboxylic acid (Example 35(b)) (150 mg, 0.38 mmol) and 1-(4-methoxyphenyl)methanesulfonamide (83 mg, 0.41 mmol) to give ethyl 5-cyano-6-(4-{[(4-methoxybenzyl)sulfonyl]carbamoyl}piperidin-1-yl)-2-[(2-oxopyrrolidin-1-yl)methyl]nicotinate. Yield: 109 mg (50%).

¹H-NMR (400 MHz, DMSO-d₆) δ 1.27 (3H, t), 1.50-1.63 (2H, m), 1.75-1.83 (2H, m), 1.96 (2H, pentet), 2.26 (2H, t), 2.53-2.61 (1H, m), 3.11-3.21 (2H, m), 3.40 (2H, t), 3.72 (3H, s), 4.23 (2H, q), 4.41-4.49 (2H, m), 4.57 (2H, s), 4.72 (2H, s), 6.92 (2H, d), 7.16 (2H, d), 8.35 (1H, s), 11.49 (1H, br s).

MS m/z: 584 (M+1), 582 (M−1).

GTPγS (IC₅₀ μM): 0.0075

### Example 76

### Ethyl 5-cyano-6-[4-({[(4-fluorophenyl)(methyl)amino]sulfonyl}carbamoyl)piperidin-1-yl]-2-[(2-oxopyrrolidin-1-yl)methyl]nicotinate

Prepared according to Example 57(b) from 1-{3-cyano-5-(ethoxycarbonyl)-6-[(2-oxopyrrolidin-1-yl)methyl]pyridin-2-yl}piperidine-4-carboxylic acid (Example 35(b)) (150 mg, 0.38 mmol) and N-(4-fluorophenyl)-N-methylsulfamide (84 mg, 0.41 mmol) to give ethyl 5-cyano-6-[4-({[(4-fluorophenyl)(methyl)amino]sulfonyl}carbamoyl)piperidin-1-yl]-2-[(2-oxopyrrolidin-1-yl)methyl]nicotinate. Yield: 109.3 mg (50%).

¹H-NMR (400 MHz, DMSO-d₆) δ 1.27 (3H, t), 1.44-1.57 (2H, m), 1.69-1.77 (2H, m), 1.95 (2H, pentet), 2.24 (2H, t), 2.51-2.60 (1H, m), 3.10-3.19 (2H, m), 3.39 (2H, t), 4.22 (2H, q), 4.39-4.46 (2H, m), 4.72 (2H, s), 7.18-7.25 (2H, m), 7.29-7.35 (2H, m), 8.35 (1H, s), 11.61 (1H, br s).

MS m/z: 587 (M+1), 585 (M−1).

GTPγS (IC₅₀ μM): 0.0073

### Example 77

### Ethyl 5-cyano-6-[4-({[(4-fluorophenyl)amino]sulfonyl}carbamoyl)piperidin-1-yl]-2-[(2-oxopyrrolidin-1-yl)methyl]nicotinate

Prepared according to Example 57(b) from 1-{3-cyano-5-(ethoxycarbonyl)-6-[(2-oxopyrrolidin-1-yl)methyl]pyridin-2-yl}piperidine-4-carboxylic acid (Example 35(b)) (150 mg, 0.38 mmol) and N-(4-fluorophenyl)sulfamide (78 mg, 0.41 mmol) to give ethyl 5-cyano-6-[4-({[(4-fluorophenyl)amino]sulfonyl}carbamoyl)piperidin-1-yl]-2-[(2-oxopyrrolidin-1-yl)methyl]nicotinate. Yield: 31.4 mg (15%).

¹H-NMR (400 MHz, DMSO-d₆) δ 1.26 (3H, t), 1.31-1.43 (2H, m), 1.56-1.64 (2H, m), 1.91 (2H, pentet), 2.20 (2H, t), 2.46-2.53 (1H, m), 3.07-3.17 (2H, m), 3.33-3.39 (2H, m), 4.22 (2H, q), 4.28-4.35 (2H, m), 4.69 (2H, s), 7.07-7.17 (4H, m), 8.32 (1H, s), 10.33 (1H, br s), 11.66 (1H, br s).

MS m/z: 573 (M+1), 571 (M−1).

GTPγS (IC₅₀ μM): 0.017

### Example 78

### Isopropyl 5-cyano-6-[4-({[methyl(phenyl)amino]sulfonyl}carbamoyl)piperidin-1-yl]-2-[(2-oxopyrrolidin-1-yl)methyl]nicotinate

Prepared according to Example 57(b) from 1-{3-cyano-5-(isopropoxycarbonyl)-6-[(2-oxopyrrolidin-1-yl)methyl]pyridin-2-yl}piperidine-4-carboxylic acid (Example 45 (c)) (150 mg, 0.38 mmol) and N-methyl-N-phenylsulfamide (Example 57 (a)) (74 mg, 0.40 mmol) to give isopropyl 5-cyano-6-[4-({[methyl(phenyl)amino]sulfonyl}carbamoyl)piperidin-1-yl]-2-[(2-oxopyrrolidin-1-yl)methyl]nicotinate. Yield: 129 mg (61%).

¹H-NMR (400 MHz, DMSO-d₆) δ 1.27 (6H, d), 1.42-1.54 (2H, m), 1.66-1.73 (2H, m), 1.95 (2H, pentet), 2.24 (2H, t), 2.49-2.58 (1H, m), 3.08-3.18 (2H, m), 3.39 (2H, t), 4.36-4.43 (2H, m), 4.71 (2H, s), 5.05 (1H, septet), 7.24-7.31 (3H, m), 7.35-7.41 (2H, m), 8.33 (1H, s), 11.61 (1H, br s).

MS m/z: 583 (M+1), 581 (M−1).

GTPγS (IC₅₀ μM): 0.021

### Example 79

### Isopropyl 5-cyano-6-[4-({[(4-fluorophenyl)(methyl)amino]sulfonyl}carbamoyl)piperidin-1-yl]-2-[(2-oxopyrrolidin-1′-yl)methyl]nicotinate

Prepared according to Example 57(b) from 1-{3-cyano-5-(isopropoxycarbonyl)-6-[(2-oxopyrrolidin-1-yl)methyl]pyridin-2-yl}piperidine-4-carboxylic acid (Example 45 (c)) (150 mg, 0.38 mmol) and N-(4-fluorophenyl)-N-methylsulfamide (81 mg, 0.40 mmol) to give isopropyl 5-cyano-6-[4-({[(4-fluorophenyl)(methyl)amino]sulfonyl}carbamoyl)piperidin-1-yl]-2-[(2-oxopyrrolidin-1-yl)methyl]nicotinate. Yield: 111 mg (51%).

¹H-NMR (400 MHz, DMSO-d₆) δ 1.27 (6H, d), 1.44-1.56 (2H, m), 1.69-1.76 (2H, m), 1.95 (2H, quintet), 2.24 (2H, t), 2.50-2.59 (1H, m), 3.09-3.19 (2H, m), 3.39 (2H, t), 4.38-4.45 (2H, m), 4.71 (2H, s), 5.06 (1H, quintet), 7.19-7.26 (2H, m), 7.29-7.25 (2H, m), 8.33 (1H, s), 11.61 (1H, br s).

MS m/z: 601 (M+1), 599 (M−1).

GTPγS (IC₅₀ μM): 0.014

### Example 80

### Isopropyl 5-cyano-6-[4-({[(4-fluorophenyl)amino]sulfonyl}carbamoyl)piperidin-1-yl]-2-[(2-oxopyrrolidin-1-yl)methyl]nicotinate

Prepared according to Example 57(b) from 1-{3-cyano-5-(isopropoxycarbonyl)-6-[(2-oxopyrrolidin-1-yl)methyl]pyridin-2-yl}piperidine-4-carboxylic acid (Example 45 (c)) (150 mg, 0.38 mmol) and N-(4-fluorophenyl)sulfamide (76 mg, 0.40 mmol) to give isopropyl 5-cyano-6-[4-({[(4-fluorophenyl)amino]sulfonyl}carbamoyl)piperidin-1-yl]-2-[(2-oxopyrrolidin-1-yl)methyl]nicotinate. Yield: 124 mg (58%).

MS m/z: 587 (M+1), 585 (M−1).

GTPγS (IC₅₀ μM): 0.038

### Example 81

### Isopropyl 5-cyano-6-(4-{[(cyclobutylmethyl)sulfonyl]carbamoyl}piperidin-1-yl)-2-[(2-oxopyrrolidin-1-yl)methyl]nicotinate

Prepared according to Example 57(b) from 1-{3-cyano-5-(isopropoxycarbonyl)-6-[(2-oxopyrrolidin-1-yl)methyl]pyridin-2-yl}piperidine-4-carboxylic acid (Example 45 (c)) (150 mg, 0.38 mmol) and 1-cyclobutylmethanesulfonamide (60 mg, 0.40 mmol) to give isopropyl 5-cyano-6-(4-{[(cyclobutylmethyl)sulfonyl]carbamoyl}piperidin-1-yl)-2-[(2-oxopyrrolidin-1-yl)methyl]nicotinate. Yield: 90.6 mg (46%).

¹H-NMR (400 MHz, DMSO-d₆) δ 1.27 (6H, d), 1.47-1.60 (2H, m), 1.69-1.80 (2H, m), 1.80-1.88 (2H, m), 1.95 (2H, quintet), 1.99-2.07 (2H, m), 2.24 (2H, t), 2.54-2.67 (2H, m), 3.13-3.22 (2H, m), 3.36-3.45 (4H, m), 4.40-4.47 (2H, m), 4.71 (2H, s), 5.05 (1H, septet), 8.32 (1H, s), 11.64 (1H, br s).

MS m/z: 546 (M+1), 544 (M−1).

GTPγS (IC₅₀ μM): 0.094

### Example 82

### Isopropyl 5-cyano-6-(4-{[(cyclohexylmethyl)sulfonyl]carbamoyl}piperidin-1-yl)-2-[(2-oxopyrrolidin-1-yl)methyl]nicotinate

Prepared according to Example 57(b) from 1-{3-cyano-5-(isopropoxycarbonyl)-6-[(2-oxopyrrolidin-1-yl)methyl]pyridin-2-yl}piperidine-4-carboxylic acid (Example 45 (c)) (150 mg, 0.38 mmol) and 1-cyclohexylmethanesulfonamide (71 mg, 0.40 mmol) to give isopropyl 5-cyano-6-(4-{[(cyclobutylmethyl)sulfonyl]carbamoyl}piperidin-1-yl)-2-[(2-oxopyrrolidin-1-yl)methyl]nicotinate. Yield: 120 mg (58%).

¹H-NMR (400 MHz, DMSO-d₆) δ 0.97-1.26 (4H, m), 1.27 (6H, d), 1.48-1.64 (5H, m), 1.71-1.79 (2H, m), 1.81-1.89 (2H, m), 1.95 (2H, quintet), 2.24 (2H, t), 2.59-2.68 (1H, m), 3.14-3.25 (4H, m), 3.39 (2H, t), 4.39-4.47 (2H, m), 4.71 (2H, s), 5.05 (1H, septet), 8.32 (1H, s), 11.69 (1H, br s).

MS m/z: 574 (M+1), 572 (M+1).

GTPγS (IC₅₀ μM): 0.028

### Example 83

### Isopropyl 5-cyano-6-(4-{[(4-methoxybenzyl)sulfonyl]carbamoyl}piperidin-1-yl)-2-[(2-oxopyrrolidin-1-yl)methyl]nicotinate

Prepared according to Example 57(b) from 1-{3-cyano-5-(isopropoxycarbonyl)-6-[(2-oxopyrrolidin-1-yl)methyl]pyridin-2-yl}piperidine-4-carboxylic acid (Example 45 (c)) (150 mg, 0.38 mmol) and 1-(4-methoxyphenyl)methanesulfonamide (80 mg, 0.40 mmol) to give isopropyl 5-cyano-6-(4-{[(4-methoxybenzyl)sulfonyl]carbamoyl}piperidin-1-yl)-2-[(2-oxopyrrolidin-1-yl)methyl]nicotinate. Yield: 100 mg (46%).

¹H-NMR (400 MHz, DMSO-d₆) δ 1.27 (6H, d), 1.50-1.62 (2H, m), 1.75-1.83 (2H, m), 1.97 (2H, quintet), 2.26 (2H, t), 2.52-2.62 (1H, m), 3.11-3.21 (2H, m), 3.40 (2H, t), 3.72 (3H, s), 4.40-4.49 (2H, m), 4.57 (2H, s), 4.72 (2H, s), 5.06 (1H, septet), 6.92 (2H, d), 7.16 (2H, d), 8.33 (1H, s), 11.49 (1H, br s).

MS m/z: 598 (M+1), 596 (M−1).

GTPγS (IC₅₀ μM): 0.016

### Example 84

### Ethyl 6-{4-[(benzylsulfonyl)carbamoyl]piperidin-1-yl}-5-bromo-2-[(2-oxopyrrolidin-1-yl)methyl]nicotinate

### (a) 5-Ethyl 1-methyl-4-(1-aminoethylidene)pent-2-enedioate

Ethyl 3-aminocrotonate (12.9 g, 100 mmol) and methyl propiolate (12.6 g, 150 mmol) were dissolved in toluene (70 mL), the reaction mixture was heated at 110° C. for 65 h. The solvent was concentrated in vacuo and the crude product was used in the next step without further purification.

¹H-NMR (500 MHz, CDCl_{3.}) δ 1.35 (3H, t), 2.25 (3H, s), 3.70 (3H, s), 4.25 (2H, q), 6.16 (1H, d), 7.64 (1H, d).

### (b) Ethyl 6-hydroxy-2-methylnicotinate

Potassium tert-butoxide (87 mg, 0.78 mmol) was added to a solution of 5-ethyl 1-methyl-4-(1-aminoethylidene)pent-2-enedioate (8.3 g, 39 mmol) in DMF (40 mL), the reaction mixture was heated at 160° C. for 16 h. The reaction was cooled to rt, water was added and the precipitate was filtered washed (IPA 2× and diethylether 2×) and dried to give ethyl 6-hydroxy-2-methylnicotinate as beige solid. Yield: 5.89 g (84%).

¹H-NMR (500 MHz, CDCl_{3.}) δ 1.36 (3H, t), 2.74 (3H, s), 4.30 (2H, q), 6.41 (1H, d), 8.03 (1H, d), 13.10 (1H, s).

### (c) Ethyl 5-bromo-6-hydroxy-2-methylnicotinate

N-Bromosuccinimide (1.1 g, 6.1 mmol) and benzoylperoxide (134 mg, 0.55 mmol) were added to a solution of ethyl 6-hydroxy-2-methylnicotinate (1 g, 5.5 mmol) in carbon tetrachloride (20 mL). The reaction mixture was refluxed for 4 h, the solution was filtered and the filtrate was concentrated in vacuo. The residue was dissolved in water and DCM, the organic phase was washed with water, dried and concentrated in vacuo. The crude product was purified by (Kromasil C₈, 250×50 mmID, using an increasing gradient of 20% to 45% MeCN. with an acidic second eluent (H₂O/MeCN/HOAc, 95/5/0.2)) to give ethyl 5-bromo-6-hydroxy-2-methylnicotinate. Yield: 1.02 g (71%).

¹H-NMR (500 MHz, CDCl_{3.}) [symbol] 1.34-1.43 (3H, m), 3.89 (3H, s), 4.28-4.38 (2H, m), 8.41 (1H, s), 12.80 (1H, bs).

MS m/z: 262 (M+1), 260 (M−1).

### (d) Ethyl 5-bromo-6-chloro-2-methylnicotinate

Ethyl 5-bromo-6-hydroxy-2-methylnicotinate (550 mg, 2.12 mmol) and phosphorus oxychloride (13.2 g, 86 mmol) was heated at 120° C. for 3.5 h. The solvent was concentrated in vacuo, water and EtOAc were added, the organic phase was separated, washed (NaHCO₃), dried and concentrated in vacuo. the residue was purified by flash chromatography, DCM:hexane 3:2 as eluent, to give ethyl 5-bromo-6-chloro-2-methylnicotinate. Yield: 500 mg (85%).

MS m/z: 278 (M+1).

### (e) Ethyl 5-bromo-2-(bromomethyl)-6-chloronicotinate

Ethyl 5-bromo-6-chloro-2-methylnicotinate (500 mg, 1.80 mmol) was added to a mixture of N-bromosuccinimide (367 mg, 2.06 mmol) and benzoylperoxide (44 mg, 0.18 mmol) in carbon tetrachloride (9 mL), the reaction mixture was refluxed for 3 h. The solution was filtered and the filtrate was washed with water and concentrated in vacuo. The residue was purified by HPLC (Kromasil C₈, 250×50 mmID, using an increasing gradient of 20% to 80% MeCN with an acidic second eluent (H2O/MeCN/HOAc, 95/5/0.2)) to give ethyl 5-bromo-2-(bromomethyl)-6-chloronicotinate. Yield 397 mg (62%).

MS m/z: 357 (M+1).

### (f) Ethyl 5-bromo-6-chloro-2-[(2-oxopyrrolidin-1-yl)methyl]nicotinate

Ethyl 5-bromo-2-(bromomethyl)-6-chloronicotinate (395 mg, 1.11 mmol) was added to a solution of 2-methoxy-1-pyrroline (329 mg, 3.32 mmol) in N-methyl-2-pyrrolidone (8 mL). The reaction was heated in the micro wave at 80° C. for 60 min, water was added and the solid material was filtered, washed and dried to give ethyl 5-bromo-6-chloro-2-[(2-oxopyrrolidin-1-yl)methyl]nicotinate. Yield: 360 mg (90%).

¹H-NMR (500 MHz, CDCl₃) δ 1.42-1.45 (3H, m), 2.10-2.20 (2H, m), 2.48-2.55 (2H, m), 3.52-3.61 (2H, m), 4.40-4.47 (2H, m), 4.91 (2H, s), 8.46 (1H, s).

### (g) 1-(tert-Butoxycarbonyl)piperidine-4-carboxylic acid

Di-tert-butyl dicarbonate (7.2 g, 33 mmol) was added in portions to a solution of isonipecotic acid (3.9 g, 30 mmol) in THF/water/NaOH (60/30/30 1 M) at rt, the mixture was stirred for 19 h. The organic solvent was concentrated in vacuo and the alkaline water phase was washed with dimethylether (2×15 mL). The solution was then acidified with 1 M KHSO₄ (70 mL) and the water phase was extracted with dimethylether (1×100+1×50 mL). The combined organic layer was washed with brine, dried (MgSO₄), filtered and concentrated in vacuo to give 1-(tert-butoxycarbonyl)piperidine-4-carboxylic acid as a white powder. Yield: 6.60 g (96%).

¹H-NMR (400 MHz, CDCl₃) δ 1.46 (9H, s), 1.60-1.71 (2H, m), 1.87-1.96 (2H, m), 2.44-2.54 (1H, m), 2.81-2.92 (2H, m), 3.95-4.09 (2H, m).

### (h) tert-Butyl 4-[(benzylsulfonyl)carbamoyl]piperidine-1-carboxylate

TEA (66 g, 0.65 mol) was added drop wise to a solution of 1-(tert-butoxycarbonyl)piperidine-4-carboxylic acid (Example 33 (c)) (50 g, 0.22 mol) and TBTU (77 g, 0.24 mol) in THF (600 mL), the reaction mixture was stirred at rt for 1.5 h. N-Phenylsulfamide (41 g, 0.24 mol) was added and the reaction was stirred over night at rt. The organic solvent was concentrated in vacuo and the residue was dissolved in EtOAc (600 mL), washed with 2 M HCl, 1M HCl, water and concentrated in vacuo. The residue was dissolved in EtOH (250 mL) and water was added (600 mL), the precipitate was filtered, washed with EtOH and dried to give tert-butyl 4-[(benzylsulfonyl)carbamoyl]piperidine-1-carboxylate. Yield: 69 g (83%).

¹H-NMR (300 MHz, DMSO-d₆) δ 1.30-1.47 (2H, m), 1.39 (9H, s), 1.61-1.71 (2H, m), 2.32-2.42 (1H, m), 2.60-2.76 (2H, m), 3.86-3.98 (2H, m), 4.68 (2H, s), 7.25-7.30 (2H, m), 7.34-7.43 (3H, m), 11.54 (1H, s).

### (i) N-(Benzylsulfonyl)piperidine-4-carboxamide

tert-Butyl 4-[(benzylsulfonyl)carbamoyl]piperidine-1-carboxylate (Example 33 (d)) (14.7 g, 38 mmol) was dissolved in formic acid (160 g, 3.5 mol), the reaction mixture was stirred at rt for 25 h. The solvent was concentrated in vacuo and water was added followed by NH₄OH until pH 6.5. The precipitate was filtered and dried to give N-(benzylsulfonyl)piperidine-4-carboxamide. Yield: 8.4 g (77%).

¹H-NMR (400 MHz, DMSO-d₆) δ 1.58-1.71 (2H, m), 1.73-1.84 (2H, m), 2.09-2.18 (1H, m), 2.77-2.87 (2H, m), 3.08-3.18 (2H, m), 4.23 (2H, s), 7.16-7.28 (5H, m).

### (j) Ethyl 6-{4-[(benzylsulfonyl)carbamoyl]piperidin-1-yl}-5-bromo-2-[(2-oxopyrrolidin-1-yl)methyl]nicotinate

Ethyl 5-bromo-6-chloro-2-[(2-oxopyrrolidin-1-yl)methyl]nicotinate (Example 84(f)) (122 mg, 0.34 mmol) was added to a solution of N-(benzylsulfonyl)piperidine-4-carboxamide (186 mg, 0.67 mmol) and DIPEA (190 g, 1.47 mmol) in ethanol (5.5 mL), the reaction mixture was heated in the microwave at 100° C. for 10 h. The solvent was concentrated in vacuo, the residue was dissolved in DCM, washed with 0.5 M HCl and brine, dried and concentrated in vacuo. The crude material was purified by HPLC (Kromasil C₈, 250×50 mmID, using an increasing gradient of 20% to 90% MeCN with an acidic second eluent (H2O/MeCN/HOAc, 95/5/0.2)) to give ethyl 6-{4-[(benzylsulfonyl)carbamoyl]piperidin-1-yl}-5-bromo-2-[(2-oxopyrrolidin-1-yl)methyl]nicotinate. Yield: 165 mg (81%).

¹H-NMR (500 MHz, CDCl₃) δ 1.38-1.43 (3H, m), 1.83-1.89 (4H, m), 2.06-2.14 (1H, m), 2.42-2.53 (2H, m), 2.90-2.98 (2H, m), 3.49 (2H, s), 3.99-4.04 (2H, m), 4.33-4.39 (2H, m), 4.67 (2H, s), 4.89 (2H, s), 7.40 (5H, s), 8.35 (1H, s), 9.37 (1H, s).

MS m/z: 609 (M+1), 607 (M−1).

GTPγS (IC₅₀ μM): 0.031

### Example 85

### Ethyl 6-{4-[(benzylsulfonyl)carbamoyl]piperidin-1-yl}-5-chloro-2-[(2-oxopyrrolidin-1-yl)methyl]nicotinate

### (a) Ethyl 5-chloro-6-hydroxy-2-methylnicotinate

Ethyl 6-hydroxy-2-methylnicotinate (Example 84(b)) (5 g, 28 mmol) was dissolved in DMF (30 mL), the mixture was heated at 100° C. and N-chlorosuccinimide (4.4 g, 33 mmol) in DMF (20 mL) was added drop wise to the solution. The reaction mixture was stirred at 80° C. for 30 min, water was added and the precipitate was filtered and washed with IPA and dried to give ethyl 5-chloro-6-hydroxy-2-methylnicotinate. Yield: 4.27 g (72%).

¹H-NMR (400 MHz, DMSO-d₆) δ 1.23 (3H, t), 2.48 (3H, s), 4.17 (2H, q), 7.95 (1H, s), 12.55 (1H, s).

MS m/z: 216 (M+1), 214 (M−1)

### (b) Ethyl 5,6-dichloro-2-methylnicotinate

Prepared according to Example 84(d) from ethyl 5-chloro-6-hydroxy-2-methylnicotinate (4.23 g, 19.6 mmol) to give ethyl 5,6-dichloro-2-methylnicotinate. Yield: 4.38 g (95%).

¹H-NMR (500 MHz, CDCl₃) δ 1.40 (3H, t), 2.79 (3H, s), 4.38 (2H, q), 8.26 (1H, s).

### (c) Ethyl 2-(bromomethyl)-5,6-dichloronicotinate

Prepared according to Example 84(d) from ethyl 5,6-dichloro-2-methylnicotinate. (4.38 g, 18.7 mmol) to give ethyl 2-(bromomethyl)-5,6-dichloronicotinate. Yield: 3.64 g (62%).

### (d) Ethyl 5,6-dichloro-2-[(2-oxopyrrolidin-1-yl)methyl]nicotinate

Prepared according to Example 84(f) from ethyl 2-(bromomethyl)-5,6-dichloronicotinate (Example 85(c)) (779 mg, 2.5 mmol) to give ethyl 5,6-dichloro-2-[(2-oxopyrrolidin-1-yl)methyl]nicotinate. Yield: 727 mg (92%).

¹H-NMR (500 MHz, CDCl_{3.}) δ 1.41 (3H, t), 2.11 (2H, m), 2.47 (2H, m), 3.52 (2H, m), 4.40 (2H, q), 4.91 (2H, s), 8.29 (1H, s).

### (e) Ethyl 6-{4-[(benzylsulfonyl)carbamoyl]piperidin-1-yl}-5-chloro-2-[(2-oxopyrrolidin-1-yl)methyl]nicotinate

Prepared according to Example 84(j) from ethyl 5,6-dichloro-2-[(2-oxopyrrolidin-1-yl)methyl]nicotinate (119 mg, 0.38 mmol) and N-(benzylsulfonyl)piperidine-4-carboxamide (Example 84(i)) (159 mg, 0.56 mmol) to give ethyl 6-{4-[(benzylsulfonyl)carbamoyl]piperidin-1-yl}-5-chloro-2-[(2-oxopyrrolidin-1-yl)methyl]nicotinate. Yield: 186 mg (88%).

¹H-NMR (500 MHz, CDCl₃) δ 1.41 (3H, t), 1.81-1.90 (4H, m), 2.07-2.15 (2H, m), 2.43-2.55 (3H, m), 2.94-3.02 (2H, m), 3.47-3.52 (2H, m), 4.04-4.10 (2H, m), 4.36 (2H, q), 4.67 (2H, s), 4.91 (2H, s), 7.36-7.42 (5H, m), 8.17 (1H, s), 9.30 (1H, s).

MS m/z: 563 (M+1), 561 (M−1).

GTPγS (IC₅₀ μM): 0.041

### Example 86

### Ethyl 6-{4-[(benzylsulfonyl)carbamoyl]piperidin-1-yl}-2-[(2-oxopyrrolidin-1-yl)methyl]nicotinate

Ethyl 6-{4-[(benzylsulfonyl)carbamoyl]piperidin-1-yl}-5-bromo-2-[(2-oxopyrrolidin-1-yl)methyl]nicotinate (Example 84(j)) (15 mg, 0.025 mmol) was dissolved in MeOH (3 mL), 10% Pd/C (17 mg) was added. The reaction mixture was hydrogenated for 2.5 h. The solvent was concentrated in vacuo, the residue was extracted with DCM and water. The organic phase was concentrated in vacuo and the residue was purified by HPLC (Kromasil C₈, 250×50 mmID, using an increasing gradient of 20% to 70% MeCN with an acidic second eluent (H2O/MeCN/HOAc, 95/5/0.2)) to give ethyl 6-{4-[(benzylsulfonyl)carbamoyl]piperidin-1-yl}-2-[(2-oxopyrrolidin-1-yl)methyl]nicotinate. Yield: 8 mg (61%).

¹H-NMR (600 MHz, CDCl₃) δ 1.36 (3H, t), 1.62-1.70 (2H, m), 1.77-1.82 (2H, m), 1.97-2.03 (2H, m), 2.24-2.28 (2H, m), 2.49-2.54 (1H, m), 2.82-2.88 (2H, m), 3.40-3.44 (2H, m), 4.19-4.24 (2H, m), 4.30 (2H, q), 4.61 (1H, s), 4.84 (2H, s), 6.42-6.46 (1H, m), 7.32-7.37 (5H, m), 8.03 (2H, d), 10.37 (1H, s).

GTPγS (IC₅₀ μM): 0.103

### Example 87

### Ethyl 5-chloro-6-(4-{[(4-methylbenzyl)sulfonyl]carbamoyl}piperidin-1-yl)-2-[(2-oxopyrrolidin-1-yl)methyl]nicotinate

### (a) Ethyl 6-[4-(tert-butoxycarbonyl)piperidin-1-yl]-5-chloro-2-[(2-oxopyrrolidin-1-yl)methyl]nicotinate

Ethyl 5,6-dichloro-2-[(2-oxopyrrolidin-1-yl)methyl]nicotinate (Example 85(d)) (3.2 g, 9.96 mmol) and tert-butyl piperidine-4-carboxylate (2.77 g, 15 mmol) were dissolved in DIPEA (4.4 mL, 24.9 mmol) and N-methyl-2-pyrrolidone (30 mL), the reaction mixture was heated at 140° C. for 1.5 h. Water and DCM were added, the organic phase was washed with 0.5 M HCl, sat. NaHCO₃ and water, dried (MgSO₄) and concentrated in vacuo. The residue was purified by flash chromatography, DCM:MeOH 100:0 to 90:10 as eluent, to give ethyl 6-[4-(tert-butoxycarbonyl)piperidin-1-yl]-5-chloro-2-[(2-oxopyrrolidin-1-yl)methyl]nicotinate. Yield: 3.1 g (67%).

¹H-NMR (500 MHz, CDCl₃) δ 1.37 (3H, t), 1.45 (9H, s), 1.80 (2H, m), 1.95 (2H, m), 2.08 (2H, m), 2.40-2.50 (3H, m), 3.00 (2H, m), 3.49 (2H, m), 4.04 (2H, m), 4.32 (2H, q), 4.87 (2H, s), 8.11 (1H, s).

### (b) 1-{3-Chloro-5-(ethoxycarbonyl)-6-[(2-oxopyrrolidin-1-yl)methyl]pyridin-2-yl}piperidine-4-carboxylic acid

Ethyl 5,6-dichloro-2-[(2-oxopyrrolidin-1-yl)methyl]nicotinate (1.37 g, 2.94 mmol) was dissolved in DCM (5 mL), TFA (5 mL, 65 mmol) was added. The reaction was stirred at rt for 5 h, the solvent was concentrated in vacuo and the crude product was used in the next step without further purification.

MS m/z: 410 (M+1), 408 (M−1).

### (c) Ethyl 5-chloro-6-(4-{[(4-methylbenzyl)sulfonyl]carbamoyl}piperidin-1-yl)-2-[(2-oxopyrrolidin-1-yl)methyl]nicotinate

Prepared according to Example 57(d) from 1-{3-chloro-5-(ethoxycarbonyl)-6-[(2-oxopyrrolidin-1-yl)methyl]pyridin-2-yl}piperidine-4-carboxylic acid (195 mg, 0.48 mmol) and 1-(4-methylphenyl)methanesulfonamide (123 mg, 0.67 mmol) to give ethyl 5-chloro-6-(4-{[(4-methylbenzyl)sulfonyl]carbamoyl}piperidin-1-yl)-2-[(2-oxopyrrolidin-1-yl)methyl]nicotinate. Yield: 107 mg (23%).

¹H-NMR (500 MHz, DMSO-d₆) δ 1.32 (3H, t), 1.59-1.70 (2H, m), 1.75-1.84 (2H, m), 2.00 (2H, quintet), 2.29 (2H, t), 2.31 (3H, s), 2.5 (1H, m), 2.94 (2H, m), 3.45 (2H, t), 4.08 (2H, m), 4.28 (2H, q), 4.63 (2H, s), 4.73 (2H, s), 7.19 (4H, quartet), 8.09 (1H, s).

MS m/z: 577 (M+1), 575 (M−1).

GTPγS (IC₅₀ μM): 0.037

### Example 88

### Ethyl 5-chloro-6-(4-{[(4-methoxybenzyl)sulfonyl]carbamoyl}piperidin-1-yl)-2-[(2-oxopyrrolidin-1-yl)methyl]nicotinate

Prepared according to Example 57(d) from 1-{3-chloro-5-(ethoxycarbonyl)-6-[(2-oxopyrrolidin-1-yl)methyl]pyridin-2-yl}piperidine-4-carboxylic acid (Example 87(b)) (195 mg, 0.48 mmol) and 1-(4-methoxyphenyl)methanesulfonamide (133 mg, 0.66 mmol) to give ethyl 5-chloro-6-(4-{[(4-methoxybenzyl)sulfonyl]carbamoyl}piperidin-1-yl)-2-[(2-oxopyrrolidin-1-yl)methyl]nicotinate. Yield: 94 mg (33%).

¹H-NMR (500 MHz, DMSO-d₆) δ 1.32 (3H, t), 1.59-1.70 (2H, m), 1.75-1.84 (2H, m), 2.00 (2H, quintet), 2.29 (2H, t), 2.5 (1H, m), 2.94 (2H, m), 3.45 (2H, t), 3.75 (3H, s), 4.08 (2H, m), 4.28 (2H, q), 4.60 (2H, s), 4.73 (2H, s), 6.95 (2H, d), 7.20 (2H, d), 8.09 (1H, s).

MS m/z: 593 (M+1), 591 (M−1).

GTPγS (IC₅₀ μM): 0.081

### Example 89

### Ethyl 5-chloro-6-(4-{[(2,4-difluorobenzyl)sulfonyl]carbamoyl}piperidin-1-yl)-2-[(2-oxopyrrolidin-1-yl)methyl]nicotinate

Prepared according to Example 57(d) from 1-{3-chloro-5-(ethoxycarbonyl)-6-[(2-oxopyrrolidin-1-yl)methyl]pyridin-2-yl}piperidine-4-carboxylic acid (Example 87b) (195 mg, 0.48 mmol) and 1-(2,4-difluorophenyl)methanesulfonamide (137 mg, 0.66 mmol) to give ethyl 5-chloro-6-(4-{[(2,4-difluorobenzyl)sulfonyl]carbamoyl}piperidin-1-yl)-2-[(2-oxopyrrolidin-1-yl)methyl]nicotinate. Yield: 97 mg (20%).

¹H-NMR (500 MHz, DMSO-d₆) δ 1.32 (3H, t), 1.58-1.69 (2H, m), 1.78-1.85 (2H, m), 2.00 (2H, quintet), 2.29 (2H, t), 2.45 (1H, m), 2.95 (2H, m), 3.45 (2H, t), 4.07 (2H, m), 4.28 (2H, q), 4.63 (2H, s), 4.72 (2H, s), 7.15 (1H, m), 7.30 (1H, m), 7.43 (1H, m), 8.08 (1H, s).

MS m/z: 599 (M+1), 597 (M−1).

GTPγS (IC₅₀ μM): 0.033

### Example 90

### Ethyl 6-{3-[(benzylsulfonyl)carbamoyl]azetidin-1-yl}-5-cyano-2-[(2-oxopyrrolidin-1-yl)methyl]nicotinate

### (a) 1-(tert-Butoxycarbonyl)azetidine-3-carboxylic acid

Di-tert-buyldicarbonate (18 g, 83 mmol) was added to a solution of 3-azetidinecarboxylic acid (7.6 g, 75 mmol) in THF (150 mL), water (75 mL) and 1M NaOH (75 mL), the reaction mixture was stirred at rt for 24 h. The organic solvent was concentrated in vacuo and the water was made acidic by addition of 4 M HCl (pH 1). The water phase was extracted with EtOAc and the combined organic phases were washed with brine, dried (MgSO₄) and concentrated in vacuo to give 1-(tert-butoxycarbonyl)azetidine-3-carboxylic acid as a white solid. Yield: 14.4 g (95%).

¹H-NMR (400 MHz, DMSO-d₆) δ 1.36 (9H, s), 3.27-3.36 (1H, m), 3.80-3.87 (2H, m), 3.93-4.02 (2H, m), 12.64 (1H, s).

### (b) tert-Butyl 3-[(benzylsulfonyl)carbamoyl]azetidine-1-carboxylate

Prepared according to Example 33(d) from 1-(tert-butoxycarbonyl)azetidine-3-carboxylic acid (20 g, 100 mmol) and N-phenylsulfamide (18 g, 105 mmol) to give tert-butyl 3-[(benzylsulfonyl)carbamoyl]azetidine-1-carboxylate. Yield: 25.4 g (72%).

¹H-NMR (400 MHz, DMSO-d₆) δ 1.39 (9H, s), 3.78-3.95 (4H, m), 4.73 (2H, s), 7.28-7.33 (2H, m), 7.37-7.42 (3H, m), 11.71 (1H, s).

### (c) N-(benzylsulfonyl)azetidine-3-carboxamide

tert-Butyl 3-[(benzylsulfonyl)carbamoyl]azetidine-1-carboxylate (122 mg, 0.34 mmol) was dissolved in DCM (3 mL), TFA (2 mL, 26 mmol) was added. The reaction mixture was stirred at rt for 30 min, the solvent was concentrated in vacuo and the crude product was used in the next step without further purifications.

### (d) Ethyl 6-{3-[(benzylsulfonyl)carbamoyl]azetidin-1-yl}-5-cyano-2-[(2-oxopyrrolidin-1-yl)methyl]nicotinate

Prepared according to Example 33(f) from ethyl 5-cyano-6-hydroxy-2-[(2-oxopyrrolidin-1-yl)methyl]nicotinate (Example 33(b)) (0.2 g, 0.69 mmol) and N-(benzylsulfonyl)azetidine-3-carboxamide (0.26 g, 1.04 mmol) to give ethyl 6-{3-[(benzylsulfonyl)carbamoyl]azetidin-1-yl}-5-cyano-2-[(2-oxopyrrolidin-1-yl)methyl]nicotinate. Yield: 135 mg (37%).

¹H-NMR (400 MHz, DMSO-d₆) [symbol] 1.30 (3H, t), 1.93-2.02 (2H, m), 2.26 (2H, t), 3.44 (2H, t), 3.50-3.59 (1H, m), 4.21-4.30 (4H, m), 4.34-4.44 (2H, m), 4.72 (4H, s), 7.30-7.40 (5H, m), 8.35 (1H, s), 11.8 (1H, s).

GTPγS (IC₅₀ μM): 0.019

### Example 91

### Ethyl 5-chloro-6-[4-({[(4-fluorophenyl)amino]sulfonyl}carbamoyl)piperidin-1-yl]-2-[(2-oxopyrrolidin-1-yl)methyl]nicotinate

Prepared according to Example 57(b) from 1-{3-chloro-5-(ethoxycarbonyl)-6-[(2-oxopyrrolidin-1-yl)methyl]pyridin-2-yl}piperidine-4-carboxylic acid (Example 87(b)) (195 mg, 0.48 mmol) and N-(4-fluorophenyl)sulfamide (127 mg, 0.67 mmol) to give ethyl 5-chloro-6-[4-({[(4-fluorophenyl)amino]sulfonyl}carbamoyl)piperidin-1-yl]-2-[(2-oxopyrrolidin-1-yl)methyl]nicotinate. Yield: 120 mg (68%).

¹H-NMR (500 MHz, DMSO-d₆) δ 1.30 (3H, t), 1.40-1.50 (2H, m), 1.55-1.65 (2H, m), 1.94 (2H, m), 2.23 (2H, t), 2.41 (1H, m), 2.89 (2H, m), 3.40 (2H, t), 3.95 (2H, m), 4.26 (2H, q), 4.69 (2H, s), 7.10-7.20 (4H, m), 8.05 (1H, s).

GTPγS (IC₅₀ μM): 0.145

### Example 92

### Ethyl 5-chloro-6-[4-({[(4-fluorophenyl)(methyl)amino]sulfonyl}carbamoyl)piperidin-1-yl]-2-[(2-oxopyrrolidin-1-yl)methyl]nicotinate

Ethyl 5-chloro-6-[4-({[(4-fluorophenyl)amino]sulfonyl}carbamoyl)piperidin-1-yl]-2-[(2-oxopyrrolidin-1-yl)methyl]nicotinate (Example 91) (120 mg, 0.21 mmol) was dissolved in DMF (4 mL), the solution was cooled to 0° C. Sodium hydride (10 mg, 0.41 mmol) was added and the reaction mixture was stirred at rt for 45 min. Iodomethane (0.014 mL, 0.23 mmol) was added at 0° C., the reaction mixture was allowed to reach rt and stirred for 1 h. EtOAc and water with some drops of acetic acid were added, the organic phase was washed with water, dried and concentrated in vacuo. The residue was purified by HPLC (Kromasil C8 10 um, 250×20 ID mm, using a gradient of MeCN with a second acidic eluent (H₂O/MeCN/FA, 95/5/0.2)) to give ethyl 5-chloro-6-[4-({[(4-fluorophenyl)(methyl)amino]sulfonyl}carbamoyl)piperidin-1-yl]-2-[(2-oxopyrrolidin-1-yl)methyl]nicotinate. Yield: 18 mg (15%).

¹H-NMR (500 MHz, CDCl₃) δ 1.36 (3H, t), 1.75-1.80 (4H, m), 2.05 (2H, m), 2.39 (3H, m), 2.85 (2H, m), 3.44 (2H, t), 3.45 (3H, s), 4.04 (2H, m), 4.32 (2H, q), 4.84 (2H, s), 7.05 (2H, m), 7.34 (2H, m), 8.11 (1H, s).

GTPγS (IC₅₀ μM): 0.077

### Example 93

### Ethyl 5-chloro-6-[4-({[methyl(phenyl)amino]sulfonyl}carbamoyl)piperidin-1-yl]-2-[(2-oxopyrrolidin-1-yl)methyl]nicotinate

Prepared according to Example 57(b) from 1-{3-chloro-5-(ethoxycarbonyl)-6-[(2-oxopyrrolidin-1-yl)methyl]pyridin-2-yl}piperidine-4-carboxylic acid (Example 87(b)) (195 mg, 0.48 mmol) and N-methyl-N-phenylsulfamide (124 mg, 0.67 mmol) to give ethyl 5-chloro-6-[4-({[methyl(phenyl)amino]sulfonyl}carbamoyl)piperidin-1-yl]-2-[(2-oxopyrrolidin-1-yl)methyl]nicotinate. Yield: 126 mg (46%).

¹H-NMR (600 MHz, CDCl_{3.}) δ 1.34 (3H, t), 1.72 (4H, m), 2.01 (2H, m), 2.31 (2H, t), 2.37 (1H, m), 2.78 (2H, m), 3.42 (2H, t), 3.45 (3H, s), 3.98 (2H, m), 4.29 (2H, q), 4.81 (2H, s), 7.20-7.35 (5H, m), 8.08 (1H, s).

GTPγS (IC₅₀ μM): 0.062

### Example 94

### Ethyl 5-chloro-6-(4-{[(cyclohexylmethyl)sulfonyl]carbamoyl}piperidin-1-yl)-2-[(2-oxopyrrolidin-1-yl)methyl]nicotinate

Prepared according to Example 57(b) from 1-{3-chloro-5-(ethoxycarbonyl)-6-[(2-oxopyrrolidin-1-yl)methyl]pyridin-2-yl}piperidine-4-carboxylic acid (Example 87(b)) (195 mg, 0.48 mmol) and 1-cyclohexylmethanesulfonamide (118 mg, 0.67 mmol) to give ethyl 5-chloro-6-(4-{[(cyclohexylmethyl)sulfonyl]carbamoyl}piperidin-1-yl)-2-[(2-oxopyrrolidin-1-yl)methyl]nicotinate. Yield: 71 mg (26%).

¹H-NMR (600 MHz, CDCl_{3.}) [symbol] 1.00-1.30 (6H, cm), 1.36 (3H, t), 1.60-2.10 (1H, m), 2.50 (3H, m), 2.90 (2H, m), 3.3 (2H, m), 3.44 (2H, t), 4.03 (2H, m), 4.32 (2H, q), 4.87 (2H, s), 8.11 (1H, s).

GTPγS (IC₅₀ μM): 0.194

### Example 95

### Propyl 5-chloro-6-[4-({[(4-fluorophenyl)(methyl)amino]sulfonyl}carbamoyl)piperidin-1-yl]-2-[(2-oxopyrrolidin-1-yl)methyl]nicotinate

Prepared according to Example 57(b) from 1-{3-chloro-5-(ethoxycarbonyl)-6-[(2-oxopyrrolidin-1-yl)methyl]pyridin-2-yl}piperidine-4-carboxylic acid (Example 87(b)) (56 mg, 0.0.131 mmol) and N-(4-fluorophenyl)-N-methylsulfamide (38 mg, 0.183 mmol) to give propyl 5-chloro-6-[4-({[(4-fluorophenyl)(methyl)amino]sulfonyl}carbamoyl)piperidin-1-yl]-2-[(2-oxopyrrolidin-1-yl)methyl]nicotinate. Yield: 50 mg (63%).

¹H-NMR (400 MHz, CD₃OD) δ 1.03 (3H, t), 1.72-1.84 (6H, m), 2.08-2.17 (2H, m), 2.39-2.49 (3H, m), 2.90-2.98 (2H, m), 3.42 (3H, s), 3.56 (2H, t), 4.11-4.18 (2H, m), 4.25 (2H, t), 4.81-4.88 (3H, m), 7.10-7.16 (2H, m), 7.37-7.43 (2H, m), 8.14 (1H, s).

GTPγS (IC₅₀ μM): 0.155

### Example 96

### Isopropyl 6-{4-[(benzylsulfonyl)carbamoyl]piperidin-1-yl}-5-chloro-2-[(2-oxopyrrolidin-1-yl)methyl]nicotinate

### (a) 6-{4-[(Benzylsulfonyl)carbamoyl]piperidin-1-yl}-5-chloro-2-[(2-oxopyrrolidin-1-yl)methyl]nicotinic acid

Ethyl 6-{4-[(benzylsulfonyl)carbamoyl]piperidin-1-yl}-5-chloro-2-[(2-oxopyrrolidin-1-yl)methyl]nicotinate (Example 85(e)) (85 mg, 0.15 mmol) was added to a solution of sodium hydroxide (110 mg, 2.75 mmol) in water (1.5 mL) and acetonitrile (1.5 mL), the reaction mixture was heated at 80° C. in a microwave oven (single node heating) for 5 min. Water and DCM were added, the water phase was made acidic, the precipitate was filtered, washed with DCM and water and dried to give 6-{4-[(benzylsulfonyl)carbamoyl]piperidin-1-yl}-5-chloro-2-[(2-oxopyrrolidin-1-yl)methyl]nicotinic acid. Yield: 80 mg (99%).

### (b) Isopropyl 6-{4-[(benzylsulfonyl)carbamoyl]piperidin-1-yl}-5-chloro-2-[(2-oxopyrrolidin-1-yl)methyl]nicotinate

6-{4-[(Benzylsulfonyl)carbamoyl]piperidin-1-yl}-5-chloro-2-[(2-oxopyrrolidin-1-yl)methyl]nicotinic acid (79 mg, 0.15 mmol) was dissolved in DCM (4 mL), TEA (60 mL, 0.59 mmol), isopropyl chloroformate (36 mg, 0.29 mmol) and DMAP (9 mg, 0.074 mmol) were added, the reaction mixture was stirred at rt for 2.5 h. DCM and sat. NH₄Cl were added, the organic phase was washed with brine, dried and concentrated in vacuo. The residue was purified by HPLC (Kromasil C8 10 um, 250×20 ID mm, using a gradient of MeCN with a second acidic eluent (H₂O/MeCN/HOAc, 95/5/0.2)) to give isopropyl 6-{4-[(benzylsulfonyl)carbamoyl]piperidin-1-yl}-5-chloro-2-[(2-oxopyrrolidin-1-yl)methyl]nicotinate. Yield: 27 mg (32%).

¹H-NMR (500 MHz, CDCl₃) δ 1.37 (6H, d), 1.80-1.87 (4H, m), 2.02-2.09 (2H, m), 2.32 (2H, t), 2.43-2.50 (1H, m), 2.84-2.92 (2H, m), 3.44 (2H, t), 4.00-4.05 (2H, m), 4.66 (2H, s), 4.86 (2H, s), 5.18-5.27 (1H, m), 7.36-7.41 (5H, m), 8.12 (1H, s), 9.80 (1H, s).

GTPγS (IC₅₀ μM): 0.178

### Example 97

### S-Ethyl 5-cyano-6-(4-{[(cyclopentylmethyl)sulfonyl]carbamoyl}piperidin-1-yl)-2-[(2-oxopyrrolidin-1-yl)methyl]pyridine-3-carbothioate

### (a) tert-Butyl 1-{3-cyano-5-[(ethylthio)carbonyl]-6-[(2-oxopyrrolidin-1-yl)methyl]pyridin-2-yl}piperidine-4-carboxylate

6-[4-(tert-Butoxycarbonyl)piperidin-1-yl]-5-cyano-2-[(2-oxopyrrolidin-1-yl)methyl]nicotinic (Example 45(a)) (137 mg, 0.32 mmol) was dissolved in THF (5 mL), N,N′-carbonyldiimidazole (73 mg, 0.45 mmol) was added. The reaction mixture was stirred at r.t over night. Ethanethiol (0.15 mL, 2.03 mmol) was added and the reaction mixture was heated at 50° C. over night. NH₄Cl (aq) was added and the mixture was extracted with DCM (×3). The combined organic phases were dried (phase separator) and concentrated in vacuo. The crude product was used in the next step without further purification.

MS m/z: 473 (M+1), 471 (M−1).

### (b) 1-{3-Cyano-5-[(ethylthio)carbonyl]-6-[(2-oxopyrrolidin-1-yl)methyl]pyridin-2-yl}piperidine-4-carboxylic acid

Prepared according to Example 35(b) from tert-butyl 1-{3-cyano-5-[(ethylthio)carbonyl]-6-[(2-oxopyrrolidin-1-yl)methyl]pyridin-2-yl}piperidine-4-carboxylate (254 mg, 0.54 mmol) give 1-{3-cyano-5-[(ethylthio)carbonyl]-6-[(2-oxopyrrolidin-1-yl)methyl]pyridin-2-yl}piperidine-4-carboxylic acid. Yield: 224 mg (100%).

MS m/z: 417 (M+1), 415 (M−1).

### (c) S-Ethyl 5-cyano-6-(4-{[(cyclopentylmethyl)sulfonyl]carbamoyl}piperidin-1-yl)-2-[(2-oxopyrrolidin-1-yl)methyl]pyridine-3-carbothioate

Prepared according to Example 35(c) from 1-{3-cyano-5-[(ethylthio)carbonyl]-6-[(2-oxopyrrolidin-1-yl)methyl]pyridin-2-yl}piperidine-4-carboxylic acid (112 mg, 0.27 mmol) and 1-cyclopentylmethanesulfonamide (48 mg, 0.29 mmol) to give S-ethyl 5-cyano-6-(4-{[(cyclopentylmethyl)sulfonyl]carbamoyl}piperidin-1-yl)-2-[(2-oxopyrrolidin-1-yl)methyl]pyridine-3-carbothioate. Yield: 6 mg (4%).

MS m/z: 562 (M+1), 560 (M−1).

GTPγS (IC₅₀ μM): 1.22

### Example 98

### S-Ethyl 5-cyano-6-(4-{[(4-methylbenzyl)sulfonyl]carbamoyl}piperidin-1-yl)-2-[(2-oxopyrrolidin-1-yl)methyl]pyridine-3-carbothioate

Prepared according to Example 35(c) from 1-{3-cyano-5-[(ethylthio)carbonyl]-6-[(2-oxopyrrolidin-1-yl)methyl]pyridin-2-yl}piperidine-4-carboxylic acid (Example 97(b)) (112 mg, 0.27 mmol) and N-phenylsulfamide (55 mg, 0.30 mmol) to give S-ethyl 5-cyano-6-(4-{[(4-methylbenzyl)sulfonyl]carbamoyl}piperidin-1-yl)-2-[(2-oxopyrrolidin-1-yl)methyl]pyridine-3-carbothioate. Yield: 5.2 mg (3%).

GTPγS (IC₅₀ μM): 4.53

### Example 99

### Isopropyl 5-cyano-2-[(2-oxopyrrolidin-1-yl)methyl]-6-[4-({[4-(trimethylsilyl)benzyl]sulfonyl}carbamoyl)piperidin-1-yl]nicotinate

Prepared according to Example 35(c) from 1-{3-cyano-5-(isopropoxycarbonyl)-6-[(2-oxopyrrolidin-1-yl)methyl]pyridin-2-yl}piperidine-4-carboxylic acid (Example 45©) (160 mg, 0.39 mmol) and 1-[4-(trimethylsilyl)phenyl]methanesulfonamide (141 mg, 0.58 mmol) to give isopropyl 5-cyano-2-[(2-oxopyrrolidin-1-yl)methyl]-6-[4-({[4-(trimethylsilyl)benzyl]sulfonyl}carbamoyl)piperidin-1-yl]nicotinate. Yield: 10 mg (4%).

¹H-NMR (500 MHz, CDCl₃) δ 0.25 (7H, m), 1.35 (6H, d), 1.83 (6H, m), 2.06 (2H, dd), 2.33 (2H, t), 2.51 (1H, t), 3.21 (2H, t), 3.44 (2H, t), 4.48 (2H, d), 4.62 (2H, s), 4.89 (2H, s), 5.19 (1H, m), 7.31 (2H, d), 7.53 (2H, d), 8.37 (1H, s).

MS m/z: 640 (M+1).

GTPγS (IC₅₀ μM): 0.497

### Example 100

### Ethyl 5-cyano-2-[(2-oxopyrrolidin-1-yl)methyl]-6-[4-({[4-(trimethylsilyl)benzyl]sulfonyl}carbamoyl)piperidin-1-yl]nicotinate

Prepared according to Example 35(c) from 1-{3-cyano-5-(ethoxycarbonyl)-6-[(2-oxopyrrolidin-1-yl)methyl]pyridin-2-yl}piperidine-4-carboxylic acid (Example 35(b)) (150 mg, 0.38 mmol) and 1-[4-(trimethylsilyl)phenyl]methanesulfonamide (137 mg, 0.56 mmol) to give ethyl 5-cyano-2-[(2-oxopyrrolidin-1-yl)methyl]-6-[4-({[4-(trimethylsilyl)benzyl]sulfonyl}carbamoyl)piperidin-1-yl]nicotinate. Yield: 103 mg (44%).

¹H-NMR (500 MHz, CDCl₃) δ 0.24 (s, 9H), 1.36 (t, 3H), 1.81 (td, 4H), 2.02 (m, 2H), 2.25 (t, 2H), 2.56 (m, 1H), 3.18 (t, 2H), 3.43 (t, 2H), 4.31 (q, 2H), 4.48 (d, 2H), 4.59 (s, 2H), 4.87 (s, 2H), 7.30 (d, 2H), 7.51 (d, 2H), 8.37 (s, 1H), 10.03 (s, 1H).

MS m/z: 626 (M+1)

GTPγS (IC₅₀ μM): 0.138

### Example 101

### Ethyl 5-cyano-2-[(2-oxopiperidin-1-yl)methyl]-6-{4-[({4-[(trifluoromethyl)thio]benzyl}sulfonyl)carbamoyl]piperidin-1-yl}nicotinate

Prepared according to Example 35(c) from 1-{3-cyano-5-(ethoxycarbonyl)-6-[(2-oxopiperidin-1-yl)methyl]pyridin-2-yl}piperidine-4-carboxylic acid (Example 56(d)) (146 mg, 0.35 mmol) and 1-{4-[(trifluoromethyl)thio]phenyl}methanesulfonamide (115 mg, 0.42 mmol) to give ethyl 5-cyano-2-[(2-oxopiperidin-1-yl)methyl]-6-{4-[({4-[(trifluoromethyl)thio]benzyl}sulfonyl)carbamoyl]piperidin-1-yl}nicotinate. Yield: 67 mg (29%).

¹H-NMR (500 MHz, CDCl₃) δ 1.39 (3H, t), 1.68-1.90 (8H, m), 2.25-2.29 (2H, m), 2.47-2.55 (1H, m), 3.10-3.17 (2H, m), 3.30-3.35 (2H, m), 4.34 (2H, q), 4.44-4.50 (2H, m), 4.67 (2H, s), 4.95 (2H, s), 7.43 (2H, d), 7.67 (2H, d), 8.41 (1H, s), 10.47 (1H, s).

GTPγS (IC₅₀ μM): 0.166

### Example 102

### Ethyl 5-cyano-2-[(2-oxopiperidin-1-yl)methyl]-6-[4-({[4-(trimethylsilyl)benzyl]sulfonyl}carbamoyl)piperidin-1-yl]nicotinate

Prepared according to Example 35(c) from 1-{3-cyano-5-(ethoxycarbonyl)-6-[(2-oxopiperidin-1-yl)methyl]pyridin-2-yl}piperidine-4-carboxylic acid (Example 56(d)) (150 mg, 0.36 mmol) and 1-[4-(trimethylsilyl)phenyl]methanesulfonamide (132 mg, 0.54 mmol) to give ethyl 5-cyano-2-[(2-oxopiperidin-1-yl)methyl]-6-[4-({[4-(trimethylsilyl)benzyl]sulfonyl}carbamoyl)piperidin-1-yl]nicotinate. Yield: 51 mg (22%).

¹H-NMR (500 MHz, CDCl₃) δ 0.20 (s, 9H), 1.31 (t, 3H), 1.76 (m, 8H), 2.17 (t, 2H), 2.48 (m, 1H), 3.12 (t, 2H), 3.27 (t, 2H), 4.26 (m, 2H), 4.44 (d, 2H), 4.54 (s, 2H), 4.89 (s, 2H), 7.25 (d, 2H), 7.46 (d, 2H), 8.36 (d, 1H), 10.06 (s, 1H).

MS m/z: 640 (M+1)

GTPγS (IC₅₀ μM): 0.934

### Example 103

### Ethyl 6-(4-{[(4-tert-butylbenzyl)sulfonyl]carbamoyl}piperidin-1-yl)-5-cyano-2-[(2-oxopiperidin-1-yl)methyl]nicotinate

Prepared according to Example 35(c) from 1-{3-cyano-5-(ethoxycarbonyl)-6-[(2-oxopiperidin-1-yl)methyl]pyridin-2-yl}piperidine-4-carboxylic acid (Example 56(d)) (150 mg, 0.36 mmol) and 1-(4-tert-butylphenyl)methanesulfonamide (123 mg, 0.54 mmol) to give ethyl 6-(4-{[(4-tert-butylbenzyl)sulfonyl]carbamoyl}piperidin-1-yl)-5-cyano-2-[(2-oxopiperidin-1-yl)methyl]nicotinate. Yield: 111 mg (31%).

¹H-NMR (500 MHz, CDCl₃) δ 1.31 (9H, s), 1.38 (3H, t), 1.80 (8H, m), 2.27 (2H, t), 2.54 (1H, m), 3.18 (2H, t), 3.33 (2H, t), 4.32 (2H, m), 4.51 (2H, d), 4.58 (2H, s), 4.95 (2H, s), 7.32 (4H, m), 8.39 (1H, s), 10.07 (1H, s).

MS m/z: 624 (M+1)

GTPγS (IC₅₀ μM): 0.376

### Example 104

### Ethyl 2-[(2-acetamidoethyl)thio]-5-chloro-6-(4-{[(4-chlorobenzyl)sulfonyl]carbamoyl}piperidin-1-yl)nicotinate

### (a) Ethyl 2,6-dichloronicotinate

2,6-Dichloronicitinic acid (3.84 g, 20 mmol) was dissolved in EtOH (16 mL), sulfuric acid (1.96 g, 20 mmol) and triethyl ortoformate (4.45 g, 30 mmol) were added. The reaction mixture was heated in a microwave oven (single node heating) at 150° C. for 15 min. The mixture was extracted with EtOAc (3×20 mL) from 10% Na₂CO₃ (20 mL). The combined organic phases were extracted with water (50 mL), dried (Na₂SO₄), filtered and concentrated in vacuo to give ethyl 2,6-dichloronicotinate. The crude material was used in the next step without further purification.

### (b) Ethyl 6-[4-(tert-butoxycarbonyl)piperidin-1-yl]-2-chloronicotinate

Ethyl 2,6-dichloronicotinate (1.25 g, 5.68 mmol) was dissolved in DMF (16 mL), 4-piperidinecarboxylic acid tert-butyl ester hydrogen chloride (1.39 g, 6.25 mmol) and DIPEA (2.9 mL, 17 mmol) were added. The reaction mixture was heated in a microwave at 150° C. in a microwave oven (single node heating) for 10 min, the solvent was concentrated in vacuo and brine (8 mL) was added and the water phase was extracted with DCM (3×), the organic phase was dried (phase separator) and concentrated in vacuo. The residue was purified by flash chromatography, heptane/Et₂O 10:1 to 4:1 as eluent, to give ethyl 6-[4-(tert-butoxycarbonyl)piperidin-1-yl]-2-chloronicotinate. Yield: 630 mg (30%).

### (c) Ethyl 6-[4-(tert-butoxycarbonyl)piperidin-1-yl]-2,5-dichloronicotinate

Ethyl 6-[4-(tert-butoxycarbonyl)piperidin-1-yl]-2-chloronicotinate (621 mg, 1.68 mmol) was dissolved in acetonitrile (6 mL), N-chlorosuccinimide (292 mg, 2.2 mmol) was added and the reaction mixture was heated in a microwave oven (single node heating) at 100° C. for 10 min. The solvent was concentrated in vacuo and the residue was purified by flash chromatography, heptane/Et₂O 6:1 to 4:1 as eluent, to give ethyl 6-[4-(tert-butoxycarbonyl)piperidin-1-yl]-2,5-dichloronicotinate as an oil. Yield: 560 mg (83%).

¹H NMR (400 MHz, CDCl₃) δ 1.38 (3H, t), 1.46 (9H, s), 1.74-1.89 (2H, m), 1.94-2.04 (2H, m), 2.43-2.52 (1H, m), 3.02-3.13 (2H, m), 4.07-4.16 (2H, m), 4.35 (2H, q), 8.07 (1H, s).

MS m/z: 403 (M+1)

### (d) Ethyl 2-[(2-acetamidoethyl)thio]-6-[4-(tert-butoxycarbonyl)piperidin-1-yl]-5-chloronicotinate

Ethyl 6-[4-(tert-butoxycarbonyl)piperidin-1-yl]-2,5-dichloronicotinate (101 mg, 0.25 mmol) was dissolved in DMSO (2 mL), N-(2-mercaptoethyl)acetamide (238 mg, 2 mmol) and DIPEA (0.24 mL, 2 mmol) were added. The reaction mixture was heated in the microwave at 120° C. for 60 min. The mixture was purified HPLC (Kromasil C8, 250 mm×50.8 ID, using a gradient of MeCN with an acidic second eluent (H2O/MeCN/HOAc, 95/5/0.1)) to give ethyl 2-[(2-acetamidoethyl)thio]-6-[4-(tert-butoxycarbonyl)piperidin-1-yl]-5-chloronicotinate. Yield: 45 mg (37%).

¹H NMR (400 MHz, CDCl₃) δ 1.38 (3H, t), 1.46 (9H, s), 1.77-1.91 (2H, m), 1.96 (3H, s), 1.96-2.03 (2H, m), 2.42-2.52 (1H, m), 3.00-3.10 (2H, m), 3.30 (2H, t), 3.57 (2H, q), 4.06-4.14 (2H, m), 4.34 (2H, q), 6.09-6.16 (1H, br t), 8.07 (1H, s).

MS m/z: 486 (M+1)

### (e) 1-{6-[(2-Acetamidoethyl)thio]-3-chloro-5-(ethoxycarbonyl)pyridin-2-yl}piperidine-4-carboxylic acid

Prepared according to Example 35(b) from appropriate intermediate ethyl 2-[(2-acetamidoethyl)thio]-6-[4-(tert-butoxycarbonyl)piperidin-1-yl]-5-chloronicotinate (45 mg, 0.093 mmol) to give 1-{6-[(2-acetamidoethyl)thio]-3-chloro-5-(ethoxycarbonyl)pyridin-2-yl}piperidine-4-carboxylic acid. Yield: 40 mg (100%).

¹H NMR (400 MHz, DMSO-d₆) δ 1.31 (3H, t), 1.61-1.74 (2H, m), 1.83 (3H, s), 1.92-2.00 (2H, m), 2.48-2.60 (1H, m), 3.04-3.14 (2H, m), 3.17 (2H, t), 3.31 (2H, q), 4.04-4.12 (2H, m), 4.26 (2H, q), 8.04 (1H, s), 8.06 (1H, br t).

MS m/z: 430 (M+1)

### (f) Ethyl 2-[(2-acetamidoethyl)thio]-5-chloro-6-(4-{[(4-chlorobenzyl)sulfonyl]carbamoyl}piperidin-1-yl)nicotinate

Prepared according to Example 35(c) from 1-{6-[(2-acetamidoethyl)thio]-3-chloro-5-(ethoxycarbonyl)pyridin-2-yl}piperidine-4-carboxylic acid (20 mg, 0.047 mmol) and 1-(4-chlorophenyl)methanesulfonamide (11 mg, 0.054 mmol) to give ethyl 2-[(2-acetamidoethyl)thio]-5-chloro-6-(4-{[(4-chlorobenzyl)sulfonyl]carbamoyl}piperidin-1-yl)nicotinate. Yield: 8 mg (28%).

¹H NMR (400 MHz, DMSO-d₆): δ 1.32 (3H, t), 1.64-1.76 (2H, m), 1.80-1.90 (2H, m), 1.82 (3H, s), 2.48-2.58 (1H, m), 2.93-3.03 (2H, m), 3.15-3.22 (2H, m), 3.31 (2H, t), 4.14-4.22 (2H, m), 4.27 (2H, q), 4.72 (2H, s), 7.32 (2H, br d), 7.51 (2H, br d), 8.05 (1H, s), 8.08 (1H, t), 11.65 (1H, s).

MS m/z: 617 (M+1)

GTPγS (IC₅₀ μM): 0.057

### Example 105

### Ethyl 2-[(2-acetamidoethyl)thio]-6-{4-[(benzylsulfonyl)carbamoyl]piperidin-1-yl}-5-chloronicotinate

Prepared according to Example 35(c) from 1-{6-[(2-acetamidoethyl)thio]-3-chloro-5-(ethoxycarbonyl)pyridin-2-yl}piperidine-4-carboxylic acid (20 mg, 0.047 mmol) and N-phenylsulfamide (9 mg, 0.054 mmol) to give ethyl 2-[(2-acetamidoethyl)thio]-6-{4-[(benzylsulfonyl)carbamoyl]piperidin-1-yl}-5-chloronicotinate. Yield: 7 mg (26%).

¹H NMR (400 MHz, DMSO-d₆) δ 1.32 (3H, t), 1.64-1.76 (2H, m), 1.80-1.87 (2H, m), 1.82 (3H, s), 2.48-2.58 (1H, m), 2.92-3.02 (2H, m), 3.15-3.22 (2H, m), 3.31 (2H, t), 4.14-4.22 (2H, m), 4.27 (2H, q), 4.72 (2H, s), 7.29-7.34 (2H, m), 7.40-7.45 (3H, m), 8.05 (1H, s), 8.08 (1H, t), 11.62 (1H, s).

MS m/z: 583 (M+1)

GTPγS (IC₅₀ μM): 0.048

### Example 106

### Ethyl 6-{4-[(benzylsulfonyl)carbamoyl]piperidin-1-yl}-5-cyano-2-(4-ethoxy-4-oxobutyl)nicotinate

### (a) Diethyl-2-[(dimethylamino)methylene]-3-oxoheptanedioate

N,N-Dimethylformamide dimethylacetal (5 g, 21.8 mmol) was added to diethyl 3-oxoheptanedioate (2.9 g, 23.9 mmol), the reaction mixture was stirred at rt over night. Toluene (5 mL) was added and the solution was concentrated in vacuo, the crude product was used without further purification in the next step.

### (b) Ethyl 5-cyano-2-(4-ethoxy-4-oxobutyl)-6-hydroxynicotinate

Diethyl-2-[(dimethylamino)methylene]-3-oxoheptanedioate (6.2 g, 21.7 mmol) dissolved in EtOH (40 mL) was added to a solution of 2-cyanoacetamid (1.83 g, 21.7 mmol) and sodium ethoxide (7.6 g, 23.4 mmol) in EtOH (50 mL). The reaction mixture was stirred at rt over night (22 h), the solvent was concentrated in vacuo and the residue was dissolved in water (40 mL) and acidified with 6 N HCl (pH 1). The precipitate was filtered and washed with water to give the product as a solid. Yield: 4.32 g (65%).

¹H NMR (400 MHz, DMSO-d₆) δ 1.15 (3H, t), 1.28 (3H, t), 1.80-1.89 (2H, m), 2.29-2.35 (2H, m), 2.47-2.51 (2H, m), 2.94-3.00 (2H, m), 4.01 (2H, q), 4.22 (2H, q), 8.43 (1H, s), 12.95 (1H, s).

### (c) Ethyl 6-chloro-5-cyano-2-(4-ethoxy-4-oxobutyl)nicotinate

Ethyl 5-cyano-2-(4-ethoxy-4-oxobutyl)-6-hydroxynicotinate (4.2 g, 12.3 mmol) was dissolved in acetonitrile (15 mL), phosphorus oxychloride (3.03 g, 19.8 mmol) was added and the reaction mixture was heated to 80° C. for 20 h. Water and methyl tert-butylether were added, the water phase was separated and extracted with methyl tert-butylether. The combined organic layer was washed with water and 5% K₂CO₃, dried and concentrated in vacuo. The residue was purified by flash chromatography, using a gradient of EtOAc in heptane as eluent to give ethyl 6-chloro-5-cyano-2-(4-ethoxy-4-oxobutyl)nicotinate as a colorless oil. Yield: 2.8 g (98%).

¹H NMR (400 MHz, CDCl₃) δ 1.25 (3H, t), 1.42 (3H, t), 2.04-2.13 (2H, m), 2.41 (2H, t), 3.24-3.29 (2H, m), 4.12 (2H, q), 4.41 (2H, q), 8.47 (1H, s).

MS m/z: 325 (M+1), 323 (M−1)

### (d) Ethyl 6-{4-[(benzylsulfonyl)carbamoyl]piperidin-1-yl}-5-cyano-2-(4-ethoxy-4-oxobutyl)nicotinate

Ethyl 6-chloro-5-cyano-2-(4-ethoxy-4-oxobutyl)nicotinate (116 mg, 0.36 mmol) and N-(benzylsulfonyl)piperidine-4-carboxamide (Example 84(i)) (111 mg, 0.39 mmol) were added to a solution of EtOH (3 mL) and TEA (108 mg, 1.1 mmol), the reaction was heated in the microwave at 120° C. for 10 min. The residue was purified by HPLC (Method A, see general Experimental Procedure) to give ethyl 6-{4-[(benzylsulfonyl)carbamoyl]piperidin-1-yl}-5-cyano-2-(4-ethoxy-4-oxobutyl)nicotinate. Yield: 91 mg (44%).

¹H NMR (600 MHz, DMSO-d₆) δ 1.14 (3H, t), 1.28 (3H, t), 1.57-1.66 (2H, m), 1.78-1.84 (2H, m), 1.88-1.95 (2H, m), 2.31-2.35 (2H, m), 2.54-2.60 (1H, m), 3.00-3.06 (2H, m), 3.09-3.17 (2H, m), 4.01 (2H, q), 4.23 (2H, q), 4.49-4.55 (2H, m), 4.67 (2H, s), 7.25-7.29 (2H, m), 7.35-7.41 (3H, m), 8.33 (1H, s), 11.58 (1H, s).

MS m/z: 571 (M+1).

GTPγS (IC₅₀ μM): 0.066

### Example 107

### Ethyl 6-{4-[(benzylsulfonyl)carbamoyl]piperidin-1-yl}-5-cyano-2-(3-ethoxy-3-oxopropyl)nicotinate

### (a) Diethyl-2-[(dimethylamino)methylene]-3-oxohexanedioate

Prepared according to Example 106(a) from 3-oxohexanedioic acid diethyl ester (4.87 g, 22.5 mmol) to give diethyl-2-[(dimethylamino)methylene]-3-oxohexanedioate. Yield: 6.0 g (98%).

### (b) Ethyl 5-cyano-2-(3-ethoxy-3-oxopropyl)-6-hydroxynicotinate

Diethyl-2-[(dimethylamino)methylene]-3-oxohexanedioate (6.0 g, 22 mmol) was dissolved in EtOH (10 mL), TEA (0.31 mL, 24 mmol) and malononitrile were added at 0° C. The reaction mixture was stirred at rt for 20 h, acetic acid (1.5 mL, 26.5 mmol) was added and the precipitate was filtered and washed with cold water to give ethyl 5-cyano-2-(3-ethoxy-3-oxopropyl)-6-hydroxynicotinate as a light yellow solid. Yield: 5.1 g (97%).

MS m/z: 293 (M+1), 291 (M−1)

### (c) Ethyl 6-chloro-5-cyano-2-(3-ethoxy-3-oxopropyl)nicotinate

Prepared according to Example 106(c) from ethyl 5-cyano-2-(3-ethoxy-3-oxopropyl)-6-hydroxynicotinate (5.2 g, 17.6 mmol) to give ethyl 6-chloro-5-cyano-2-(3-ethoxy-3-oxopropyl)nicotinate. Yield: 4.02 g (70%).

¹H NMR (400 MHz, CDCl₃) δ 1.23-1.27 (3H, m), 1.42 (3H, t), 2.83 (2H, t), 3.56 (2H, t), 4.13 (2H, q), 4.42 (2H, q), 8.49 (1H, s).

### (d) Ethyl 6-{4-[(benzylsulfonyl)carbamoyl]piperidin-1-yl}-5-cyano-2-(3-ethoxy-3-oxopropyl)nicotinate

Prepared according to Example 106(d) from ethyl 6-chloro-5-cyano-2-(3-ethoxy-3-oxopropyl)nicotinate (107 mg, 0.34 mmol) and N-(benzylsulfonyl)piperidine-4-carboxamide (Example 84(i)) (112 mg, 0.40 mmol) to give ethyl 6-{4-[(benzylsulfonyl)carbamoyl]piperidin-1-yl}-5-cyano-2-(3-ethoxy-3-oxopropyl)nicotinate. Yield: 110 mg (58%).

¹H NMR (600 MHz, DMSO-d₆) δ 1.11 (3H, t), 1.27 (3H, t), 1.52-1.62 (2H, m), 1.75-1.82 (2H, m), 2.53-2.59 (1H, m), 2.66-2.71 (2H, m), 3.06-3.13 (2H, m), 3.30-3.34 (2H, m), 3.99 (2H, q), 4.22 (2H, q), 4.44-4.50 (2H, m), 4.65 (2H, s), 7.23-7.27 (2H, m), 7.33-7.39 (3H, m), 8.31 (1H, s), 11.58 (1H, s).

MS m/z: 557 (M+1)

GTPγS (IC₅₀ μM): 0.056

### Example 108

### Ethyl 6-(4-{[(2-chloro-4-fluorobenzyl)sulfonyl]carbamoyl}piperidin-1-yl)-5-cyano-2-[(2-ethoxy-2-oxoethyl)thio]nicotinate

### (a) tert-Butyl 1-(2-cyanoethanimidoyl)piperidine-4-carboxylate

Two microwave vials was each charged with ethyl 2-cyanoethanimidoate (See McElvain, S. M.; Schroeder, J. P.; J. Am. Chem. Soc. 71, p. 40 (1949)) (841 mg, 7.7 mmol), tert-butyl piperidine-4-carboxylate (926 mg, 5 mmol), DIPEA (1.94 g, 15 mmol), EtOH (7.5 mL) and heated to 100° C. for 10 minutes in a microwave oven, single node heating. Additional ethyl 2-cyanoethanimidoate (252 mg, 4.5 mmol) and DIPEA (969 mg, 7.5 mmol) was added to each vial and the stirring was continued at r.t for 16 hours. LC-MS showed still some remaining tert-butyl piperidine-4-carboxylate and therfore ethyl 2-cyanoethanimidoate (246 mg, 2.2 mmol) was added and the mixture was again heated to 100° C. in a microwave oven for 20 minutes. The solutions from the vials was combined and used without further purification in the next step.

### (b) Ethyl 6-[4-(tert-butoxycarbonyl)piperidin-1-yl]-5-cyano-2-oxo-1,2-dihydropyridine-3-carboxylate

Diethyl (ethoxymethylene)malonate (3.24 g, 15 mmol) was added to the solution from step (a) above and the reaction mixture was stirred at r.t for 16 hours. The solvent was evaporated and NaHCO₃ (sat) (50 mL) was added and the water phase extracted with DCM (3×50 mL). The combined organic phase was washed with brine (150 mL), dried (Na₂SO₄), filtered and evaporated to give a crude product which was purified by preparative HPLC (Kromasil C₈, 10 μm, 50.8×250 mm column, flow 50 mL/minute using a gradient of 5 to 100% CH₃CN/0.1 M NH₄OOCH) to give the desired product. Yield: 1.262 g (32%).

¹H NMR (500 MHz, CDCl₃) δ 1.41 (3H, t, J=7.1 Hz), 1.46 (9H, s), 1.75-1.86 (2H, m), 1.98-2.06 (2H, m), 2.53-2.61 (1H, m), 3.29-3.37 (2H, m), 4.39 (2H, q, J=7.1 Hz), 4.53-4.61 (2H, m), 8.20 (1H, s). Not unambiguous where the NH proton is.

MS m/z: 376 (M+1)

### (c) Ethyl 6-[4-(tert-butoxycarbonyl)piperidin-1-yl]-5-cyano-2-{[(trifluoromethyl)sulfonyl]oxy}nicotinate

Ethyl 6-[4-(tert-butoxycarbonyl)piperidin-1-yl]-5-cyano-2-oxo-1,2-dihydropyridine-3-carboxylate (270 mg, 0.72 mmol) was dissolved in dry DCM (10 mL) and TEA (0.2 mL, 1.44 mmol) was added and the mixture was cooled to 0° C. under N₂. Tf₂O (0.16 mL, 0.95 mmol) was added and the reaction mixture was stirred at 0° C. for 30 min. NaHCO₃ (aq) was added and the mixture was extracted with DCM (×3). The combined organics was run through a phase separator and concentrated under reduced pressure. The crude product was used in the next step without further purification assuming quantitative yield.

MS m/z: 508 (M+1).

### (d) Ethyl 6-[4-(tert-butoxycarbonyl)piperidin-1-yl]-5-cyano-2-[(2-ethoxy-2-oxoethyl)thio]nicotinate

Ethyl 6-[4-(tert-butoxycarbonyl)piperidin-1-yl]-5-cyano-2-{[(trifluoromethyl)sulfonyl]oxy}nicotinate (846 mg, 1.67 mmol) was dissolved in THF (10 mL), ethylthioglycolate (0.2 mL, 1.82 mmol) and DIPEA (0.5 mL, 2.87 mmol) were added. The reaction mixture was heated to 140° C. for 5 min in a microwave oven. The mixture was concentrated in vacuo and the crude was used in the next step without further purification.

MS m/z: 478 (M+1).

### (e) 1-{3-Cyano-5-(ethoxycarbonyl)-6-[(2-ethoxy-2-oxoethyl)thio]pyridin-2-yl}piperidine-4-carboxylic acid

Prepared according to Example 35(b) from ethyl 6-[4-(tert-butoxycarbonyl)piperidin-1-yl]-5-cyano-2-[(2-ethoxy-2-oxoethyl)thio]nicotinate (Example 108 (c)) (427 mg, 0.89 mmol) to give 1-{3-cyano-5-(ethoxycarbonyl)-6-[(2-ethoxy-2-oxoethyl)thio]pyridin-2-yl}piperidine-4-carboxylic acid. Yield: 377 mg (100%).

MS m/z: 422 (M+1), 420 (M−1).

### (f) Ethyl 6-(4-{[(2-chloro-4-fluorobenzyl)sulfonyl]carbamoyl}piperidin-1-yl)-5-cyano-2-[(2-ethoxy-2-oxoethyl)thio]nicotinate

Prepared according to Example 57(b) from 1-{3-cyano-5-(ethoxycarbonyl)-6-[(2-ethoxy-2-oxoethyl)thio]pyridin-2-yl}piperidine-4-carboxylic acid (100 mg, 0.24 mmol) and 1-(2-chloro-4-fluorophenyl)methanesulfonamide (64 mg, 0.29 mmol) to give ethyl 6-(4-{[(2-chloro-4-fluorobenzyl)sulfonyl]carbamoyl}piperidin-1-yl)-5-cyano-2-[(2-ethoxy-2-oxoethyl)thio]nicotinate. Yield: 175 mg (74%).

¹H NMR (600 MHz, DMSO-d₆) δ 1.14 (3H, t), 1.28 (3H, t), 1.58-1.66 (2H, m), 1.84-1.88 (2H, m), 2.58-2.64 (1H, m), 3.13-3.18 (2H, m), 3.94 (2H, s), 4.05 (2H, q), 4.24 (2H, q), 4.42-4.47 (2H, m), 4.81 (2H, s), 7.29 (1H, dt), 7.49 (1H, dd), 7.52 (1H, dd), 8.28 (1H, s).

MS m/z: 627 (M+1), 625 (M−1).

GTPγS (IC₅₀ μM): 0.072

### Example 109

### Ethyl 5-cyano-6-(4-{[(2,4-dichlorobenzyl)sulfonyl]carbamoyl}piperidin-1-yl)-2-[(2-ethoxy-2-oxoethyl)thio]nicotinate

Prepared according to Example 57(b) from appropriate intermediate 1-{3-cyano-5-(ethoxycarbonyl)-6-[(2-ethoxy-2-oxoethyl)thio]pyridin-2-yl}piperidine-4-carboxylic acid (Example 108(e)) (100 mg, 0.24 mmol) and 1-(2,4-dichlorophenyl)methanesulfonamide (68 mg, 0.28 mmol) to give ethyl 5-cyano-6-(4-{[(2,4-dichlorobenzyl)sulfonyl]carbamoyl}piperidin-1-yl)-2-[(2-ethoxy-2-oxoethyl)thio]nicotinate. Yield: 105 mg (69%).

¹H NMR (600 MHz, DMSO-d₆) δ1.14 (3H, t), 1.28 (3H, t), 1.57-1.66 (2H, m), 1.83-1.89 (2H, m), 2.58-2.64 (1H, m), 3.12-3.18 (2H, m), 3.94 (2H, s), 4.05 (2H, q), 4.23 (2H, q), 4.42-4.47 (2H, m), 4.83 (2H, s), 7.45 (1H, d), 7.50 (1H, dd), 7.69 (1H, d), 8.28 (1H, s).

MS m/z: 642 (M+1).

GTPγS (IC₅₀ μM): 0.062

### Example 110

### Ethyl 5-cyano-6-(4-{[(cyclopentylmethyl)sulfonyl]carbamoyl}piperidin-1-yl)-2-[(2-ethoxy-2-oxoethyl)thio]nicotinate

Prepared according to Example 57(b) from appropriate intermediate 1-{3-cyano-5-(ethoxycarbonyl)-6-[(2-ethoxy-2-oxoethyl)thio]pyridin-2-yl}piperidine-4-carboxylic acid (Example 108(e)) (100 mg, 0.24 mmol) and 1-cyclopentylmethanesulfonamide (50 mg, 0.31 mmol) to give ethyl 5-cyano-6-(4-{[(cyclopentylmethyl)sulfonyl]carbamoyl}piperidin-1-yl)-2-[(2-ethoxy-2-oxoethyl)thio]nicotinate. Yield: 42.7 mg (32%).

¹H NMR (600 MHz, DMSO-d₆) δ 1.14 (3H, t), 1.18-1.25 (2H, m), 1.27 (3H, t), 1.43-1.50 (2H, m), 1.53-1.62 (4H, m), 1.79-1.85 (2H, m), 1.85-1.90 (2H, m), 2.08-2.16 (1H, m), 2.62-2.68 (1H, m), 3.13-3.20 (2H, m), 3.38 (2H, d), 3.92 (2H, s), 4.05 (2H, q), 4.23 (2H, q), 4.42-4.47 (2H, m), 8.28 (1H, s), 11.69 (1H, br s).

MS m/z: 567 (M+1), 565 (M−1).

GTPγS (IC₅₀ μM): 0.053

### Example 111

### Ethyl 6-{4-[(benzylsulfonyl)carbamoyl]piperidin-1-yl}-5-cyano-2-[(2-isopropoxy-2-oxoethyl)thio]nicotinate

### (a) 1-[3-Cyano-5-(ethoxycarbonyl)-6-{[(trifluoromethyl)sulfonyl]oxy}pyridin-2-yl]piperidine-4-carboxylic acid

Prepared according to Example 35(b) from appropriate intermediate ethyl 6-[4-(tert-butoxycarbonyl)piperidin-1-yl]-5-cyano-2-{[(trifluoromethyl)sulfonyl]oxy}nicotinate (Example 108(c)) (4.0 g, 7.9 mmol) to give 1-[3-cyano-5-(ethoxycarbonyl)-6-{[(trifluoromethyl)sulfonyl]oxy}pyridin-2-yl]piperidine-4-carboxylic acid. Yield: 3.6 g (100%).

MS m/z: 452 (M+1), 450 (M−1).

### (b) Ethyl 2-(1H-benzotriazol-1-yloxy)-6-{4-[(benzylsulfonyl)carbamoyl]piperidin-1-yl}-5-cyanonicotinate

1-[3-Cyano-5-(ethoxycarbonyl)-6-{[(trifluoromethyl)sulfonyl]oxy}pyridin-2-yl]piperidine-4-carboxylic acid (3.55 g, 7.86 mmol) and TBTU (3.66 g, 11.4 mmol) were dissolved in DCM (25 mL), DIPEA (5 mL, 28.7 mmol) was added and the reaction mixture was stirred at rt for 1.5 h. Phenylmethanesulfonamide (1.35 g, 7.88 mmol) was added and the reaction mixture was stirred at rt for 20 h. NaHCO₃ (aq) was added and the mixture was extracted with DCM (×3). The combined organics phases was dried (phase separator) and concentrated in vacuo. The residue was purified by HPLC (Column: Kromasil C₈ 10 μm, 50.8×300 mm, using an increasing gradient of 20% to 60% MeCN with a second eluant (MeCN/0.1M NH₄OAc (pH=7)) to give ethyl 2-(1H-benzotriazol-1-yloxy)-6-{4-[(benzylsulfonyl)carbamoyl]piperidin-1-yl}-5-cyanonicotinate as a white solid. Yield: 1.79 g (39%).

MS m/z: 590 (M+1), 588 (M−1).

### (c) Isopropyl (benzoylthio)acetate

Thiobenzoic acid (41 mg, 0.30 mmol) and isopropyl bromoacetate (59 mg, 0.30 mmol) were dissolved in EtOH (3 mL), DIPEA (0.053 mL, 0.30 mmol) was added. The reaction mixture was stirred at rt for 1 h, the reaction mixture was used in the next step without isolation.

### (d) Ethyl 6-{4-[(benzylsulfonyl)carbamoyl]piperidin-1-yl}-5-cyano-2-[(2-isopropoxy-2-oxoethyl)thio]nicotinate

Isopropyl (benzoylthio)acetate (71.5 mg, 0.3 mmol) dissolved in EtOH (3 mL) was added to ethyl 2-(1H-benzotriazol-1-yloxy)-6-{4-[(benzylsulfonyl)carbamoyl]piperidin-1-yl}-5-cyanonicotinate (Example 111(b)) (100 mg, 0.17 mmol), DIPEA (0.3 mL, 1.72 mmol) and water (2 mL). The reaction mixture was heated to 140° C. for 20 min in a microwave. NaHCO₃ (aq) was added and the mixture was extracted with DCM (×3). The combined organics phases were dried (phase separator) and concentrated in vacuo. The residue was purified by HPLC (Method A, see General Experimental Procedure) to give ethyl 6-{4-[(benzylsulfonyl)carbamoyl]piperidin-1-yl}-5-cyano-2-[(2-isopropoxy-2-oxoethyl)thio]nicotinate as a solid. Yield: 21 mg (21%).

¹H NMR (500 MHz, DMSO-d₆) δ 1.13 (6H, d), 1.28 (3H, t), 1.57-1.65 (2H, m), 1.77-1.83 (2H, m), 3.10-3.16 (2H, m), 3.93 (2H, s), 4.24 (2H, q), 4.41-4.47 (2H, m), 4.67 (2H, s), 4.86 (2H, quintet), 7.25-7.28 (2H, m), 7.35-7.39 (3H, m), 8.29 (1H, s), 11.57 (1H, br s).

MS m/z: 589 (M+1), 587 (M−1).

GTPγS (IC₅₀ μM): 0.016

### Example 112

### Ethyl 6-{4-[(benzylsulfonyl)carbamoyl]piperidin-1-yl}-5-cyano-2-(3-oxobutyl)nicotinate

### (a) Ethyl 2-(2-acetyl-5,5-dimethyl-3-oxohexyl)-6-{4-[(benzylsulfonyl)carbamoyl]piperidin-1-yl}-5-cyanonicotinate

To a solution of sodium tert-pentoxide (21.8 mg, 0.198 mmol) in EtOH (1 mL) were tert-butyl acetoacetate (31.3 mg, 0.198 mmol), ethyl 6-{4-[(benzylsulfonyl)carbamoyl]piperidin-1-yl}-2-(chloromethyl)-5-cyanonicotinate (Example 3(c)) (50 mg, 0.099 mmol) and a catalytic amount of NaI added, the reaction mixture was heated at 100° C. in microwave (single node heating) for 15 min. HCl (0.25 mL, 0.99 mmol, 4M in dioxane) and water (0.25 mL) were added, the reaction was stirred at rt over night. The solution was partition between iPrOAc (5 mL) and 1M MgCl₂ (2 mL), the organic phase was separated and concentrated in vacuo to give ethyl 2-(2-acetyl-5,5-dimethyl-3-oxohexyl)-6-{4-[(benzylsulfonyl)carbamoyl]piperidin-1-yl}-5-cyanonicotinate as orange oil. The crude material was used in the next step without further purification.

### (b) Ethyl 6-{4-[(benzylsulfonyl)carbamoyl]piperidin-1-yl}-5-cyano-2-(3-oxobutyl)nicotinate

Prepared according to Example 35(b) from ethyl 2-(2-acetyl-5,5-dimethyl-3-oxohexyl)-6-{4-[(benzylsulfonyl)carbamoyl]piperidin-1-yl}-5-cyanonicotinate (Example 112(a)) (62 mg, 0.099 mmol) to give ethyl 6-{4-[(benzylsulfonyl)carbamoyl]piperidin-1-yl}-5-cyano-2-(3-oxobutyl)nicotinate. Yield: 22 mg (42%).

¹H NMR (400 MHz, DMSO-d₆) δ 1.26 (3H, t), 1.47-1.59 (2H, m), 1.69-1.79 (2H, m), 2.09 (3H, s), 2.23-2.32 (1H, m), 2.82 (2H, t), 3.10-3.19 (2H, m), 3.22 (2H, t), 4.21 (2H, q), 4.31 (2H, s), 4.33-4.41 (2H, m), 7.16-7.28 (5H, m), 8.27 (1H, s)

MS m/z: 527.2 (M+1), 525.3 (M−1).

GTPγS (IC₅₀ μM): 0.022

### Example 113

### Ethyl 6-{4-[(benzylsulfonyl)carbamoyl]piperidin-1-yl}-5-cyano-2-[2-(1H-pyrrol-1-yl)ethoxy]nicotinate

### (a) Ethyl 6-{4-[allyl(benzylsulfonyl)carbamoyl]piperidin-1-yl}-5-cyano-2-[2-(1H-pyrrol-1-yl)ethoxy]nicotinate

1-(2-bromoethyl)pyrrole (153 mg, 0.88 mmol) was added to a solution of ethyl 6-{4-[allyl(benzylsulfonyl)carbamoyl]piperidin-1-yl}-5-cyano-2-oxo-1,2-dihydropyridine-3-carboxylate (Example 2(e)) (150 mg, 0.29 mmol) and potassium carbonate (anhydrous, 121 mg, 0.88 mmol) in DMF (3 mL), the reaction mixture was stirred at 80° C. for 1 h. DCM and water were added, the organic phase was separated, dried and concentrated in vacuo. The residue was purified by HPLC (Column: Kromasil C₈ 10 μm, 50.8×300 mm, using an increasing gradient of 20% to 90% MeCN with a second eluant (MeCN/0.1M NH₄OAc (pH=7)) to give ethyl 6-{4-[allyl(benzylsulfonyl)carbamoyl]piperidin-1-yl}-5-cyano-2-[2-(1H-pyrrol-1-yl)ethoxy]nicotinate. Yield: 127 mg (72%).

### (b) Ethyl 6-{4-[(benzylsulfonyl)carbamoyl]piperidin-1-yl}-5-cyano-2-[2-(1H-pyrrol-1-yl)ethoxy]nicotinate

Ethyl 6-{4-[allyl(benzylsulfonyl)carbamoyl]piperidin-1-yl}-5-cyano-2-[2-(1H-pyrrol-1-yl)ethoxy]nicotinate (127 mg, 0.21 mmol) was dissolved in DCM (5 mL), tetrakis(triphenylphosphine)palladium (121 mg, 0.105 mmol) and p-toluenesulfinic acid sodium salt (112 mg, 0.63 mmol) were added, the reaction mixture was stirred at rt for 15 min. Water and DCM were added, the organic phase was separated, dried (phase separator) and concentrated in vacuo. The residue was purified by HPLC (Kromasil C8, 10 μM, using a gradient of 20% to 100% MeCN with an acidic second eluent (H2O/MeCN/HOAc, 95/5/0.2) to give ethyl 6-{4-[(benzylsulfonyl)carbamoyl]piperidin-1-yl}-5-cyano-2-[2-(1H-pyrrol-1-yl)ethoxy]nicotinate. Yield: 104 mg (88%).

¹H NMR (300 MHz, CD₃OD) δ 1.36 (3H, t), 1.66-1.90 (4H, m), 2.46-2.58 (1H, m), 3.10-3.24 (3H, m), 4.27-4.34 (4H, m), 4.55-4.67 (6H, m), 5.98-6.01 (2H, m), 6.77-6.82 (2H, m), 7.37 (5H, s), 8.31 (1H, s).

GTPγS (IC₅₀ μM): 0.05

### Example 114

### Ethyl 6-{4-[(benzylsulfonyl)carbamoyl]piperidin-1-yl}-5-cyano-2-[2-(2-oxopyrrolidin-1-yl)ethoxy]nicotinate

### (a) Ethyl 6-[4-(tert-butoxycarbonyl)piperidin-1-yl]-5-cyano-2-[2-(2-oxopyrrolidin-1-yl)ethoxy]nicotinate

Diisopropyl azodicarboxylate (121 mg, 0.6 mmol) was added to a solution of ethyl 6-[4-(tert-butoxycarbonyl)piperidin-1-yl]-5-cyano-2-oxo-1,2-dihydropyridine-3-carboxylate (Example 108(b)) (150 mg, 0.4 mmol), N-(2-hydroxyethyl)-2-pyrrolidone (65 mg, 0.5 mmol) and triphenylphosphine (157 mg, 0.6 mmol) in THF (3 mL), the reaction mixture was stirred at 50° C. for 1 h and at rt over night. The solvent was concentrated in vacuo and the residue was dissolved in DCM and washed with sat. NaCO₃, dried and concentrated in vacuo. The residue was purified by HPLC (Column: Kromasil C₈ 10 μm, 50.8×300 mm, using an increasing gradient of 20% to 80% MeCN with a second eluant (MeCN/0.1M NH₄OAc (pH=7)) to give ethyl 6-[4-(tert-butoxycarbonyl)piperidin-1-yl]-5-cyano-2-[2-(2-oxopyrrolidin-1-yl)ethoxy]nicotinate. Yield: 180 mg (93%).

### (b) 1-{3-Cyano-5-(ethoxycarbonyl)-6-[2-(2-oxopyrrolidin-1-yl)ethoxy]pyridin-2-yl}piperidine-4-carboxylic acid

Prepared according to Example 35(b) from ethyl 6-[4-(tert-butoxycarbonyl)piperidin-1-yl]-5-cyano-2-[2-(2-oxopyrrolidin-1-yl)ethoxy]nicotinate (180 mg, 0.37 mmol) to give 1-{3-cyano-5-(ethoxycarbonyl)-6-[2-(2-oxopyrrolidin-1-yl)ethoxy]pyridin-2-yl}piperidine-4-carboxylic acid. Yield: 159 mg (100%).

### (c) Ethyl 6-{4-[(benzylsulfonyl)carbamoyl]piperidin-1-yl}-5-cyano-2-[2-(2-oxopyrrolidin-1-yl)ethoxy]nicotinate

Prepared according to Example 57(b) from appropriate intermediate 1-{3-cyano-5-(ethoxycarbonyl)-6-[2-(2-oxopyrrolidin-1-yl)ethoxy]pyridin-2-yl}piperidine-4-carboxylic acid (159 mg, 0.37 mmol) to give ethyl 6-{4-[(benzylsulfonyl)carbamoyl]piperidin-1-yl}-5-cyano-2-[2-(2-oxopyrrolidin-1-yl)ethoxy]nicotinate. Yield: 162 mg (75%).

¹H NMR (300 MHz, CD₃OD) δ 1.35 (3H, t), 1.71-1.92 (4H, m), 1.97-2.08 (2H, m), 2.33-2.40 (2H, m), 2.47-2.58 (1H, m), 3.14-3.26 (3H, m), 3.64-3.72 (4H, m), 4.28 (2H, q), 4.51-4.56 (2H, m), 4.61-4.71 (4H, m), 7.38 (5H, s), 8.33 (1H, s).

GTPγS (IC₅₀ μM): 0.027

### Example 115

### ({[6-{4-[(Benzylsulfonyl)carbamoyl]piperidin-1-yl}-5-cyano-3-(ethoxycarbonyl)pyridin-2-yl]methyl}thio)acetic acid

Ethyl 6-{4-[(benzylsulfonyl)carbamoyl]piperidin-1-yl}-2-(chloromethyl)-5-cyanonicotinate (Example 3(c)) (50 mg, 0.099 mmol) was dissolved in EtOH (1 mL), marcaptoacetic acid (18 mg, 0.198 mmol) was added and the reaction was stirred at 50° C. for 5 min. Sodium iodide (1.49 mg, 0.010 mmol) and DIPEA (0.07 mL, 0.4 mmol) were added and the reaction was stirred at 50° C. over night. Acetic acid (0.023 mL, 0.4 mmol) was added, the solution was partition between iPrOAc (5 mL) and 1M MgCl₂ (2 mL). The organic phase was separated, dried and concentrated in vacuo, the residue was purified by HPLC (Method A, see General Experimental Procedure) to give ({[6-{4-[(benzylsulfonyl)carbamoyl]piperidin-1-yl}-5-cyano-3-(ethoxycarbonyl)pyridin-2-yl]methyl}thio)acetic acid. Yield: 401 mg (73%).

¹H NMR (600 MHz, DMSO-d₆) δ 1.28 (3H, t), 1.58-1.67 (2H, m), 1.77-1.84 (2H, m), 3.10-3.17 (2H, m), 4.10 (2H, s), 4.23 (2H, q), 4.51-4.57 (2H, m), 4.66 (2H, s), 7.24-7.28 (2H, m), 7.34-7.40 (3H, m), 8.36 (1H, s). Note: two signals (3H) are overlapping with the DMSO-signal at δ 2.4-2.6.

MS m/z: 561 (M+1), 559 (M−1)

GTPγS (IC₅₀ μM): 0.035

### Example 116

### Ethyl 6-{4-[(benzylsulfonyl)carbamoyl]piperidin-1-yl}-5-cyano-2-({[(1-methyl-1H-imidazol-2-yl)methyl]thio}methyl)nicotinate

Prepared according to Example 115 from ethyl 6-{4-[(benzylsulfonyl)carbamoyl]piperidin-1-yl}-2-(chloromethyl)-5-cyanonicotinate (Example 3(c)) (50 mg, 0.099 mmol) and 1-methyl-2-thiomethyl-imidazole (25.4 mg, 0.198 mmol) to give ethyl 6-{4-[(benzylsulfonyl)carbamoyl]piperidin-1-yl}-5-cyano-2-({[(1-methyl-1H-imidazol-2-yl)methyl]thio}methyl)nicotinate. Yield: 27.7 mg (47%).

¹H NMR (400 MHz, CDCl₃) δ 1.24-1.36 (2H, m), 1.39 (3H, t), 1.76 (2H, d), 2.20 (3H, s), 2.28-2.40 (1H, m), 2.65-2.77 (1H, bs), 3.02 (2H, t), 3.98 (2H, s), 4.06 (2H, d), 4.35 (2H, q), 4.41 (2H, s), 5.90 (2H, s), 7.12-7.22 (3H, m), 7.29-7.33 (2H, m), 7.46 (1H, s), 7.98 (1H, s), 8.36 (1H, s).

MS m/z: 597 (M+1), 595 (M−1).

GTPγS (IC₅₀ μM): 1.05

### Example 117

### Ethyl 6-{4-[(benzylsulfonyl)carbamoyl]piperidin-1-yl}-5-cyano-2-[(1,3-thiazol-2-ylthio)methyl]nicotinate

Prepared according to Example 115 from ethyl 6-{4-[(benzylsulfonyl)carbamoyl]piperidin-1-yl}-2-(chloromethyl)-5-cyanonicotinate (Example 3(c)) (50 mg, 0.099 mmol) and 2-mercaptothiazole (23 mg, 0.198 mmol) to give ethyl 6-{4-[(benzylsulfonyl)carbamoyl]piperidin-1-yl}-5-cyano-2-[(1,3-thiazol-2-ylthio)methyl]nicotinate. Yield: 44.6 mg (73%).

¹H NMR (600 MHz, DMSO-d₆) δ 1.30 (3H, t), 1.50-1.61 (2H, m), 1.73-1.81 (2H, m), 3.07-3.15 (2H, m), 4.26 (2H, q), 4.42-4.49 (2H, m), 4.66 (2H, s), 4.86 (2H, s), 7.23-7.29 (2H, m), 7.34-7.41 (3H, m), 7.64 (1H, d), 7.71 (1H, d), 8.39 (1H, s). Note: One signal (1H) is overlapping with the solvent signal

MS m/z: 586 (M+1), 584 (M−1).

GTPγS (IC₅₀ μM): 0.035

### Example 118

### Ethyl 6-{4-[(benzylsulfonyl)carbamoyl]piperidin-1-yl}-5-cyano-2-{[(1-methyl-1H-imidazol-2-yl)thio]methyl}nicotinate

Prepared according to Example 115 from ethyl 6-{4-[(benzylsulfonyl)carbamoyl]piperidin-1-yl}-2-(chloromethyl)-5-cyanonicotinate (Example 3(c)) (50 mg, 0.099 mmol) and 2-mercapto-1-methylimidazole (22.6 mg, 0.198 mmol) to give ethyl 6-{4-[(benzylsulfonyl)carbamoyl]piperidin-1-yl}-5-cyano-2-{[(1-methyl-1H-imidazol-2-yl)thio]methyl}nicotinate. Yield: 47.6 mg (83%).

¹H NMR (600 MHz, DMSO-d₆) δ 1.28 (3H, t), 1.48-1.58 (2H, m), 1.72-1.79 (2H, m), 2.99-3.06 (2H, m), 3.41 (3H, s), 4.22 (2H, q), 4.29-4.35 (2H, m), 4.43 (2H, s), 4.67 (2H, s), 6.90 (1H, s), 7.22 (1H, s), 7.25-7.29 (2H, m), 7.34-7.40 (3H, m), 8.37 (1H, s). Note: One signal (1H) is overlapping with the solvent signal

MS m/z: 583 (M+1), 581 (M−1).

GTPγS (IC₅₀ μM): 0.052

### Example 119

### Ethyl 6-{4-[(benzylsulfonyl)carbamoyl]piperidin-1-yl}-5-cyano-2-{[(3-methoxy-3-oxopropyl)thio]methyl}nicotinate

Prepared according to Example 115 from ethyl 6-{4-[(benzylsulfonyl)carbamoyl]piperidin-1-yl}-2-(chloromethyl)-5-cyanonicotinate (Example 3(c)) (50 mg, 0.099 mmol) and methyl 3-mercaptopropionate (23.8 mg, 0.198 mmol) to give ethyl 6-{4-[(benzylsulfonyl)carbamoyl]piperidin-1-yl}-5-cyano-2-{[(3-methoxy-3-oxopropyl)thio]methyl}nicotinate. Yield: 43 mg (70%).

¹H NMR (600 MHz, DMSO-d₆) δ 1.27 (3H, t), 1.57-1.66 (2H, m), 1.78-1.84 (2H, m), 2.59 (2H, t), 2.70 (2H, t), 3.10-3.18 (2H, m), 3.55 (3H, s), 4.03 (2H, s), 4.22 (2H, q), 4.48-4.56 (2H, m), 4.66 (2H, s), 7.22-7.29 (2H, m), 7.32-7.40 (3H, m), 8.37 (1H, s), 11.58 (1H, s). Note: One signal (1H) is overlapping with the solvent signal.

MS m/z: 589 (M+1), 587 (M−1).

GTPγS (IC₅₀ μM): 0.042

### Example 120

### Ethyl 6-{4-[(benzylsulfonyl)carbamoyl]piperidin-1-yl}-5-cyano-2-[(1S)-2-ethoxy-1-methyl-2-oxoethoxy]nicotinate

### (a) Ethyl 6-[4-(tert-butoxycarbonyl)piperidin-1-yl]-5-cyano-2-[(1S)-2-ethoxy-1-methyl-2-oxoethoxy]nicotinate

Ethyl 6-[4-(tert-butoxycarbonyl)piperidin-1-yl]-5-cyano-2-{[(trifluoromethyl)sulfonyl]oxy}nicotinate (Example 108(c)) (200 mg, 0.39 mmol), tris(dibenzylideneacetone)dipalladium(0) (36 mg, 0.039 mmol) and 9,9-dimethyl-4,5-bis(diphenylphosphino)xanthene (45.6 mg, 0.079 mmol) were dissolved in dioxane (15 mL), ethyl-L-lactate (465.6 mg, 3.94 mmol) and DIPEA (0.165 mL, 0.95 mmol) were added. The reaction mixture was heated at 160° C. for 20 min in the microwave oven (single node heating), the reaction mixture was filtered and NaHCO₃ was added. The aqueous phase was extracted with DCM (×3) and the combined organic phases were dried (phase separator) and concentrated in vacuo. The crude product was used in the next step without further purification.

MS m/z: 476 (M+1)

### (b) 1-{3-Cyano-5-(ethoxycarbonyl)-6-[(1S)-2-ethoxy-1-methyl-2-oxoethoxy]pyridin-2-yl}piperidine-4-carboxylic acid

Prepared according to Example 35(b) from appropriate intermediate ethyl 6-[4-(tert-butoxycarbonyl)piperidin-1-yl]-5-cyano-2-[(1S)-2-ethoxy-1-methyl-2-oxoethoxy]nicotinate (187 mg, 0.39 mmol) to give 1-{3-cyano-5-(ethoxycarbonyl)-6-[(1S)-2-ethoxy-1-methyl-2-oxoethoxy]pyridin-2-yl}piperidine-4-carboxylic acid. Yield: 15 mg (6%).

MS m/z: 420 (M+1), 418 (M−1).

### (c) Ethyl 6-{4-[(benzylsulfonyl)carbamoyl]piperidin-1-yl}-5-cyano-2-[(1S)-2-ethoxy-1-methyl-2-oxoethoxy]nicotinate

Prepared according to Example 35(c) from 1-{3-cyano-5-(ethoxycarbonyl)-6-[(1S)-2-ethoxy-1-methyl-2-oxoethoxy]pyridin-2-yl}piperidine-4-carboxylic acid (15 mg, 0.036 mmol) and 1-phenylmethanesulfonamide (6.7 mg, 0.039 mmol) to ethyl 6-{4-[(benzylsulfonyl)carbamoyl]piperidin-1-yl}-5-cyano-2-[(1S)-2-ethoxy-1-methyl-2-oxoethoxy]nicotinate. Yield: 15.8 mg (70%).

¹H NMR (400 MHz, DMSO-d₆) δ 1.15 (3H, t), 1.33 (3H, t), 1.52 (3H, d), 1.55-1.73 (2H, m), 1.75-1.86 (2H, m), 2.53-2.62 (1H, m), 3.09-3.20 (2H, m), 4.19 (2H, q), 4.38 (2H, q), 4.47-4.54 (2H, m), 4.67 (2H, s), 5.18 (1H, q), 7.26-7.31 (2H, m), 7.34-7.42 (3H, m), 8.25 (1H, s), 11.59 (1H, s).

MS m/z: 573 (M+1), 571 (M−1).

GTPγS (IC₅₀ μM): 0.019

### Example 121

### Ethyl 6-{4-[(benzylsulfonyl)carbamoyl]piperidin-1-yl}-5-cyano-2-[(4-oxopentyl)oxy]nicotinate

### (a) Ethyl 6-[4-(tert-butoxycarbonyl)piperidin-1-yl]-5-cyano-2-[(4-oxopentyl)oxy]nicotinate

Prepared according to Example 121(a) from ethyl 6-[4-(tert-butoxycarbonyl)piperidin-1-yl]-5-cyano-2-{[(trifluoromethyl)sulfonyl]oxy}nicotinate (Example 108(c)) (200 mg, 0.39 mmol) and 3-acetyl-1-propanol (36 mg, 0.039 mmol) to give ethyl 6-[4-(tert-butoxycarbonyl)piperidin-1-yl]-5-cyano-2-[(4-oxopentyl)oxy]nicotinate. Yield: 181 mg (39%).

MS m/z: 460 (M+1)

### (b) 1-{3-Cyano-5-(ethoxycarbonyl)-6-[(4-oxopentyl)oxy]pyridin-2-yl}piperidine-4-carboxylic acid

Prepared according to Example 35(b) from appropriate intermediate ethyl 6-[4-(tert-butoxycarbonyl)piperidin-1-yl]-5-cyano-2-[(4-oxopentyl)oxy]nicotinate (181 mg, 0.39 mmol) to give 1-{3-cyano-5-(ethoxycarbonyl)-6-[(4-oxopentyl)oxy]pyridin-2-yl}piperidine-4-carboxylic acid. Yield: 41 mg (26%).

MS m/z: 404 (M+1), 402 (M−1).

### (c) Ethyl 6-{4-[(benzylsulfonyl)carbamoyl]piperidin-1-yl}-5-cyano-2-[(4-oxopentyl)oxy]nicotinate

Prepared according to Example 35(c) from 1-{3-cyano-5-(ethoxycarbonyl)-6-[(4-oxopentyl)oxy]pyridin-2-yl}piperidine-4-carboxylic acid (41 mg, 0.102 mmol) and 1-phenylmethanesulfonamide (19 mg, 0.112 mmol) to give ethyl 6-{4-[(benzylsulfonyl)carbamoyl]piperidin-1-yl}-5-cyano-2-[(4-oxopentyl)oxy]nicotinate. Yield: 33.2 mg (58%).

¹H NMR (400 MHz, DMSO-d₆) δ 1.26 (3H, t), 1.56-1.68 (2H, m), 1.79-1.86 (2H, m), 1.90 (2H, quintet), 2.09 (3H, s), 2.54-2.61 (1H, m), 2.63 (2H, t), 3.11-3.20 (2H, m), 4.20 (2H, q), 4.31 (2H, t), 4.48-4.55 (2H, m), 4.68 (2H, s), 7.26-7.31 (2H, m), 7.36-7.42 (3H, m), 8.26 (1H, s), 11.59 (1H, s).

MS m/z: 557 (M+1), 555 (M−1).

GTPγS (IC₅₀ μM): 0.017

### Example 122

### Ethyl 6-{4-[(benzylsulfonyl)carbamoyl]piperidin-1-yl}-5-cyano-2-{[(2-ethoxy-2-oxoethyl)thio]methyl}nicotinate

Thiobenzoic acid (0.023 mL, 0.198 mmol) was added to a solution of sodium tert-pentoxide (55 mg, 0.3 mmol) in EtOH (0.5 mL), the reaction mixture was heated to 50° C. Ethyl bromoacetate (0.022 mL, 0.198 mmol) in EtOH (0.5 mL) was added and the reaction mixture was heated at 50° C. for 1 h. Ethyl 6-{4-[(benzylsulfonyl)carbamoyl]piperidin-1-yl}-2-(chloromethyl)-5-cyanonicotinate (Example 3c) (50 mg, 0.099 mmol) was added and the reaction was stirred at 50° C. over night. 1M HCl was added to pH 3-4, the solution was extracted with 1M MgCl₂ and iPrOAc. The organic solvent was separated and concentrated in vacuo. The residue was purified by HPLC (Method A, see General Experimental Procedure) to give ethyl 6-{4-[(benzylsulfonyl)carbamoyl]piperidin-1-yl}-5-cyano-2-{[(2-ethoxy-2-oxoethyl)thio]methyl}nicotinate. Yield: 32.4 mg (56%).

¹H NMR (600 MHz, DMSO-d₆) δ 1.14 (3H, t), 1.27 (3H, t), 1.58-1.66 (2H, m), 1.77-1.84 (2H, m), 3.10-3.17 (2H, m), 4.03 (2H, q), 4.10 (2H, s), 4.22 (2H, q), 4.51-4.56 (2H, m), 4.66 (2H, s), 7.24-7.28 (2H, m), 7.34-7.39 (3H, m), 8.36 (1H, s). Note: Two signals (3H) are overlapping with the solvent signal.

MS m/z: 589 (M+1), 587 (M−1).

GTPγS (IC₅₀ μM): 0.077

### Example 123

### Ethyl 6-{4-[(benzylsulfonyl)carbamoyl]piperidin-1-yl}-5-cyano-2-({[2-(dimethylamino)-2-oxoethyl]thio}methyl)nicotinate

Prepared according to Example 122 from ethyl 6-{4-[(benzylsulfonyl)carbamoyl]piperidin-1-yl}-2-(chloromethyl)-5-cyanonicotinate (Example 3(c)) (50 mg, 0.099 mmol) and 2-chloro-N,N-dimethylacetamide (0.02 mL, 0.198 mmol) to give ethyl 6-{4-[(benzylsulfonyl)carbamoyl]piperidin-1-yl}-5-cyano-2-({[2-(dimethylamino)-2-oxoethyl]thio}methyl)nicotinate. Yield: 13.8 mg (24%).

¹H NMR (600 MHz, DMSO-d₆) δ 1.28 (3H, t), 1.57-1.66 (2H, m), 1.78-1.84 (2H, m), 2.77 (3H, s), 2.92 (3H, s), 3.10-3.18 (2H, m), 3.45 (2H, s), 4.09 (2H, s), 4.22 (2H, q), 4.51-4.58 (2H, m), 4.66 (2H, s), 7.24-7.28 (2H, m), 7.34-7.40 (3H, m), 8.35 (1H, s). Note: One signal (1H) is overlapping with the solvent signal.

MS m/z: 588.1 (M+1), 586.4 (M−1)

GTPγS (IC₅₀ μM): 0.057

### Example 124

### Ethyl 2-[(2-azetidin-1-yl-2-oxoethyl)thio]-6-{4-[(benzylsulfonyl)carbamoyl]piperidin-1-yl}-5-cyanonicotinate

### (a) {[6-{4-[(Benzylsulfonyl)carbamoyl]piperidin-1-yl}-5-cyano-3-(ethoxycarbonyl)pyridin-2-yl]thio}acetic acid

Ethyl 2-(1H-benzotriazol-1-yloxy)-6-{4-[(benzylsulfonyl)carbamoyl]piperidin-1-yl}-5-cyanonicotinate (Example 111(b)) (270 mg, 0.46 mmol) was dissolved in THF (10 mL), mercaptoacetic acid (0.44 mL, 6.3 mmol) and DIPEA (0.6 mL, 3.45 mmol) were added. The reaction mixture was heated to 120° C. for 15 min in a microwave oven. Water (0.9 mL, 50 mmol) was added and the reaction mixture was heated to 130° C. for 10 min in a microwave oven. NH₄Cl (aq) was added, the mixture was made acidic by addition of conc. HCl until pH=4 and the mixture was extracted with DCM (×3). The combined organics was dried (phase separator) and concentrated in vacuo. The crude was purified by HPLC ((Column: Kromasil C₈ 10 μm, 50.8×300 mm, using an increasing gradient of 20% to 60% MeCN with a second eluant (MeCN/0.1M NH₄OAc (pH=7))) to give {[6-{4-[(benzylsulfonyl)carbamoyl]piperidin-1-yl}-5-cyano-3-(ethoxycarbonyl)pyridin-2-yl]thio}acetic acid as a white solid. Yield: 196 mg (78%).

¹H NMR (500 MHz, DMSO-d₆): 1.30 (3H, t), 1.54-1.63 (2H, m), 1.74-1.80 (2H, m), 2.20-2.27 (1H, m), 3.18-3.26 (2H, m), 3.67 (2H, s), 4.24 (2H, s), 4.25 (2H, q), 4.41-4.47 (2H, m), 7.18-7.27 (5H, m), 8.22 (1H, s).

MS m/z: 547 (M+1), 545 (M−1).

### (b) Ethyl 2-[(2-azetidin-1-yl-2-oxoethyl)thio]-6-{4-[(benzylsulfonyl)carbamoyl]piperidin-1-yl}-5-cyanonicotinate

Prepared according to Example 35(c) from {[6-{4-[(benzylsulfonyl)carbamoyl]piperidin-1-yl}-5-cyano-3-(ethoxycarbonyl)pyridin-2-yl]thio}acetic acid (86 mg, 0.157 mmol) and azetidine (42.3 mg, 0.74 mmol) to give ethyl 2-[(2-azetidin-1-yl-2-oxoethyl)thio]-6-{4-[(benzylsulfonyl)carbamoyl]piperidin-1-yl}-5-cyanonicotinate. Yield: 32 mg (35%).

¹H NMR (500 MHz, DMSO-d₆): 1.31 (3H, t), 1.62-1.72 (2H, m), 1.84-1.90 (2H, m), 2.22 (2H, p), 2.55-2.63 (1H, m), 3.17-3.25 (2H, m), 3.78 (2H, s), 3.87 (2H, t), 4.20 (2H, t), 4.27 (2H, q), 4.49-4.55 (2H, m), 4.67 (2H, s), 7.28-7.32 (2H, m), 7.35-7.42 (3H, m), 8.29 (1H, s), 11.61 (1H, br s).

MS m/z: 586 (M+1), 584 (M−1).

GTPγS (IC₅₀ μM): 0.013

### Example 125

### Ethyl 6-{4-[(benzylsulfonyl)carbamoyl]piperidin-1-yl}-5-cyano-2-[2-(dimethylamino)-2-oxoethoxy]nicotinate

### (a) Ethyl 6-{4-[allyl(benzylsulfonyl)carbamoyl]piperidin-1-yl}-5-cyano-2-[2-(dimethylamino)-2-oxoethoxy]nicotinate

Prepared in a similar way as Example 113(a) from ethyl 6-{4-[allyl(benzylsulfonyl)carbamoyl]piperidin-1-yl}-5-cyano-2-oxo-1,2-dihydropyridine-3-carboxylate (Example 2(e)) (81 mg, 0.13 mmol) and 2-iodo-N,N-dimethylacetamide (143 mg, 0.67 mmol) to ethyl 6-{4-[allyl(benzylsulfonyl)carbamoyl]piperidin-1-yl}-5-cyano-2-[2-(dimethylamino)-2-oxoethoxy]nicotinate. Yield: 83 mg (100%).

MS m/z: 598 (M+1)

### (b) Ethyl 6-{4-[(benzylsulfonyl)carbamoyl]piperidin-1-yl}-5-cyano-2-[2-(dimethylamino)-2-oxoethoxy]nicotinate

Prepared according to Example 113(b) from ethyl 6-{4-[allyl(benzylsulfonyl)carbamoyl]piperidin-1-yl}-5-cyano-2-[2-(dimethylamino)-2-oxoethoxy]nicotinate (83 mg, 0.139 mmol) to give ethyl 6-{4-[(benzylsulfonyl)carbamoyl]piperidin-1-yl}-5-cyano-2-[2-(dimethylamino)-2-oxoethoxy]nicotinate as a white solid. Yield: 16.3 mg (21%).

¹H NMR (400 MHz, DMSO-d₆) δ 1.27 (3H, t), 1.50-1.61 (2H, m), 1.73-1.81 (2H, m), 2.81 (3H, s), 3.00 (3H, s), 3.11-3.20 (2H, m), 4.21 (2H, q), 4.33-4.41 (2H, m), 4.51-4.60 (2H, m), 5.05 (2H, s), 7.23-7.38 (5H, m), 8.28 (1H, s). Note: One signal (1H) is overlapping with the solvent signal.

MS m/z: 558 (M+1), 556 (M−1).

GTPγS (IC₅₀ μM): 0.065

### Example 126

### Ethyl 6-{4-[(benzylsulfonyl)carbamoyl]piperidin-1-yl}-5-cyano-2-[(2-ethoxy-2-oxoethyl)thio]nicotinate

Prepared according to Example 111(d) from ethyl 2-(1H-benzotriazol-1-yloxy)-6-{4-[(benzylsulfonyl)carbamoyl]piperidin-1-yl}-5-cyanonicotinate (Example 111(b)) (375 mg, 0.64 mmol) and ethyl thioglycolate (110 mg, 0.9 mmol) to give ethyl 6-{4-[(benzylsulfonyl)carbamoyl]piperidin-1-yl}-5-cyano-2-[(2-ethoxy-2-oxoethyl)thio]nicotinate as a white solid. Yield: 278 mg (76%).

¹H NMR (500 MHz, DMSO-d₆) δ 1.18 (3H, t), 1.32 (3H, t), 1.60-1.69 (2H, m), 1.81-1.87 (2H, m), 2.57-2.65 (1H, m), 3.13-3.21 (2H, m), 3.98 (2H, s), 4.09 (2H, q), 4.27 (2H, q), 4.44-4.50 (2H, m), 4.70 (2H, s), 7.28-7.32 (2H, m), 7.38-7.43 (3H, m), 8.32 (1H, s), 11.60 (1H, br s).

MS m/z: 575 (M+1), 573 (M−1).

GTPγS (IC₅₀ μM): 0.011

### Example 127

### Ethyl 6-{4-[(benzylsulfonyl)carbamoyl]piperidin-1-yl}-5-fluoro-2-[(2-oxopyrrolidin-1-yl)methyl]nicotinate

### (a) 2,6-Dichloro-5-fluoronicotinoyl chloride

A suspension of 2,6-dichloro-5-fluoronicotinic acid (4.3 g, 20.5 mmol) in toluene (20 mL) and thionyl chloride (20 mL, 276 mmol) was refluxed under an N₂-atmosphere for 3 hours. The mixture was cooled and the solvent was concentrated in vacuo and the residue was co-evaporated twice with toluene to give 2,6-dichloro-5-fluoronicotinoyl chloride as a yellow oil which was used in the next step without further purification assuming quantitative yield of the product.

### (b) Ethyl 2,6-dichloro-5-fluoronicotinate

Cold ethanol (40 mL) was added to 2,6-dichloro-5-fluoronicotinoyl chloride (4.7 g, 20.5 mmol)) at 0° C., the mixture was stirred for 15 minutes at 0° C. followed by 1 hour at reflux under an N₂-atmosphere. The EtOH was concentrated in vacuo and the residue was dissolved in EtOAc (130 mL) and the organic phase was washed with KHCO₃ (15 mL), water (15 mL), brine (15 mL) and dried (MgSO₄) and concentrated in vacuo to give ethyl 2,6-dichloro-5-fluoronicotinate as oil. The crude product was used in the next step without further purification. Yield: 4.64 g (95%).

¹H NMR (400 MHz, CDCl₃) δ 1.42 (3H, t), 4.44 (2H, q), 8.00 (1H, d).

### (c) Ethyl 6-[4-(tert-butoxycarbonyl)piperidin-1-yl]-2-chloro-5-fluoronicotinate

Ethyl 2,6-dichloro-5-fluoronicotinate (2.1 g, 8.8 mmol) and tert-butyl piperidine-4-carboxylate (2.062 g, 11.1 mmol) were added to DIPEA (3.1 mL, 17.6 mmol) and EtOH (10 mL), the reaction mixture was heated to 100° C. for 30 minutes. EtOAc (150 mL) was added and the organic phase was washed with 1 M KHSO₄ (2×10 mL), NaHCO₃ (aq, sat) 10 mL, Brine (10 mL), dried (Na₂SO₄), filtered and concentrated in vacuo. The residue was purified by flash chromatography, petroleum ether/EtOAc 9:1 to 7: as eluent, to give ethyl 6-[4-(tert-butoxycarbonyl)piperidin-1-yl]-2-chloro-5-fluoronicotinate as oil. Yield: 2.56 g (75%)

¹H NMR (400 MHz, CDCl₃) δ 1.38 (3H, t), 1.45 (9H, s), 1.71-1.82 (2H, m), 1.94-2.01 (2H, m), 2.44-2.54 (1H, m), 3.09-3.19 (2H, m), 4.28-4.37 (4H, m), 7.77 (1H, d).

### (d) Ethyl 6-[4-(tert-butoxycarbonyl)piperidin-1-yl]-5-fluoro-2-[(2-oxopyrrolidin-1-yl)methyl]nicotinate

Ethyl 6-[4-(tert-butoxycarbonyl)piperidin-1-yl]-2-chloro-5-fluoronicotinate (193 mg, 0.5 mmol), 1-[(trimethylstannyl)methyl]pyrrolidin-2-one (144 mg, 0.55 mmol), tris(dibenzylideneacetone)dipalladium(0) (9 mg, 0.01 mmol), tri-t-butylphosphonium tetrafluoroborate (12 mg, 0.04 mmol), cesium fluoride (167 mg, 1.1 mmol) and dioxane (2.5 mL) were added to a microwave vial and the reaction mixture was heated at 100° C. for 30 minutes in the micro wave oven (single node heating) followed by 100 degrees in a conventional oil bath over night (18 hours). The heterogeneous mixture was filtered and the solids washed with DCM (30 mL). The organic phase was washed with water (5 mL) and dried by passing through a phase separator. The residue was used without further purification in the next step.

### (e) 1-{5-(Ethoxycarbonyl)-3-fluoro-6-[(2-oxopyrrolidin-1-yl)methyl]pyridin-2-yl}piperidine-4-carboxylic acid

Prepared according to Example 35(b) from the crude product above i.e. ethyl 6-[4-(tert-butoxycarbonyl)piperidin-1-yl]-5-fluoro-2-[(2-oxopyrrolidin-1-yl)methyl]nicotinate (225 mg, crude) followed by purification by HPLC (Kromasil C₈, using an increasing gradient of MeCN with an acidic second eluent (H₂O/MeCN/FA, 95/5/0.2) to give 1-{5-(ethoxycarbonyl)-3-fluoro-6-[(2-oxopyrrolidin-1-yl)methyl]pyridin-2-yl}piperidine-4-carboxylic acid. Yield: 40 mg (20%) over two steps.

¹H NMR (400 MHz, CDCl₃) δ 1.37 (3H, t), 1.73-1.84 (2H, m), 1.95-2.02 (2H, m), 2.03-2.13 (2H, m), 2.52 (2H, t), 2.56-2.64 (1H, m), 3.10-3.19 (2H, m), 3.47 (2H, t), 4.22-4.34 (4H, m), 4.88 (2H, s), 7.77 (1H, d).

### (f) Ethyl 6-{4-[(benzylsulfonyl)carbamoyl]piperidin-1-yl}-5-fluoro-2-[(2-oxopyrrolidin-1-yl)methyl]nicotinate

Prepared according to Example 57(b) from 1-{5-(ethoxycarbonyl)-3-fluoro-6-[(2-oxopyrrolidin-1-yl)methyl]pyridin-2-yl}piperidine-4-carboxylic acid (40 mg, 0.102 mmol) and 1-phenylmethanesulfonamide (24 mg, 0.14 mmol) to give ethyl 6-{4-[(benzylsulfonyl)carbamoyl]piperidin-1-yl}-5-fluoro-2-[(2-oxopyrrolidin-1-yl)methyl]nicotinate as a white solid. Yield: 38 mg (68%).

¹H NMR (400 MHz, DMSO-d₆) δ 1.30 (3H, t), 1.51-1.63 (2H, m), 1.72-1.80 (2H, m), 1.93-2.04 (2H, m), 2.28 (2H, t), 2.52-2.60 (1H, m), 2.98-3.08 (2H, m), 3.43 (2H, t), 4.21-4.29 (4H, m), 4.68 (4H, d), 7.25-7.30 (2H, m), 7.36-7.42 (3H, m), 7.82 (1H, d), 11.56 (1H, s).

MS m/z: 547 (M+1), 545 (M−1).

GTPγS (IC₅₀ μM): 0.079

### Example 128

### Ethyl 6-{4-[(benzylsulfonyl)(methyl)carbamoyl]piperidin-1-yl}-5-cyano-2-[(2-oxopiperidin-1-yl)methyl]nicotinate

Prepared as Example 92 from appropriate intermediate ethyl 6-{4-[(benzylsulfonyl)carbamoyl]piperidin-1-yl}-5-cyano-2-[(2-oxopiperidin-1-yl)methyl]nicotinate (Example 56) (151 mg, 0.27 mmol) to give ethyl 6-{4-[(benzylsulfonyl)(methyl)carbamoyl]piperidin-1-yl}-5-cyano-2-[(2-oxopiperidin-1-yl)methyl]nicotinate as a white solid. Yield: 26.6 mg (17%).

¹H NMR (400 MHz, CDCl₃) δ 1.30-1.42 (3H, m), 1.47-1.71 (4H, m), 1.76-1.94 (4H, m), 2.31-2.51 (2H, m), 2.63-2.75 (1H, m), 2.92-3.07 (2H, m), 3.10 (3H, s), 3.24-3.48 (2H, m), 4.29-4.37 (2H, m), 4.44-4.65 (4H, m), 4.83-5.13 (2H, m), 7.30-7.49 (5H, m), 8.36 (1H, s).

MS m/z: 582 (M+1), 580 (M−1).

GTPγS (IC₅₀ μM): 1.14

### Example 129

### Methyl 6-{4-[(benzylsulfonyl)carbamoyl]piperidin-1-yl}-5-chloro-2-[(2-oxopiperidin-1-yl)methyl]nicotinate

### (a) Methyl 6-hydroxy-2-methylnicotinate

Methyl 3-aminocrotonate (12.6 g, 110 mmol) and methyl propiolate (9.8 g, 100 mmol) were added to MeOH (100 mL), the reaction mixture was stirred at 70° C. over night. Added seed material and cooled to 5° C., the precipitation was filtered and washed with IPA to give methyl 6-hydroxy-2-methylnicotinate as a colorless solid.

¹H NMR (400 MHz, DMSO-d₆) δ 2.52 (3H, s), 3.74 (3H, s), 6.20 (1H, d), 7.81 (1H, d), 12.05 (1H, bs).

MS m/z: 182 (M−1)

### (b) Methyl 5-chloro-6-hydroxy-2-methylnicotinate

Prepared according to Example 85(a) from appropriate intermediate methyl 6-hydroxy-2-methylnicotinate (28 g, 168 mmol) to give methyl 5-chloro-6-hydroxy-2-methylnicotinate. Yield: 17.3 g (51%).

¹H NMR (400 MHz, DMSO-d₆) δ 2.53 (3H, s), 3.76 (3H, s), 8.01 (1H, s), 12.65-12.59 (1H, m).

### (c) Methyl 5,6-dichloro-2-methylnicotinate

Prepared according to Example 84(d) from appropriate intermediate methyl 5-chloro-6-hydroxy-2-methylnicotinate (17.3 g, 86 mmol) to give methyl 5,6-dichloro-2-methylnicotinate. Yield: 16.5 g (88%).

¹H NMR (400 MHz, DMSO-d₆) δ 2.68 (3H, s), 3.87 (3H, s), 8.40 (1H, s).

### (d) Methyl 2-(bromomethyl)-5,6-dichloronicotinate

Prepared according to Example 84(e) from appropriate intermediate methyl 5,6-dichloro-2-methylnicotinate (21 g, 95 mmol) to give methyl 2-(bromomethyl)-5,6-dichloronicotinate. Yield: 12.4 g (42%).

¹H NMR (400 MHz, DMSO-d₆): 3.91 (3H, s), 4.93 (2H, s), 8.51 (1H, s).

MS m/z: 299 (M+1)

### (f) Methyl 5,6-dichloro-2-[(2-oxopiperidin-1-yl)methyl]nicotinate

Prepared according to Example 84(f) from appropriate methyl 2-(bromomethyl)-5,6-dichloronicotinate (2.04 g, 6.8 mmol) and 6-methoxy-2,3,4,5-tetrahydropyridine (2.31 g, 20.4 mmol) to give methyl 5,6-dichloro-2-[(2-oxopiperidin-1-yl)methyl]nicotinate. Yield: 1.4 g (63%).

¹H NMR (400 MHz, DMSO-d₆) δ 1.77 (4H, m), 2.25 (2H, m), 3.34 (2H, m), 3.88 (3H, s), 4.83 (2H, m), 8.43 (1H, s).

MS m/z: 317 (M+1)

### (g) Methyl 6-[4-(tert-butoxycarbonyl)piperidin-1-yl]-5-chloro-2-[(2-oxopiperidin-1-yl)methyl]nicotinate

Prepared in a similarly way as Example 106(d) from methyl 5,6-dichloro-2-[(2-oxopiperidin-1-yl)methyl]nicotinate (1.4 g, 4.24 mmol) and tert-butyl piperidine-4-carboxylate (1.18 g, 6.36 mmol) to give methyl 6-[4-(tert-butoxycarbonyl)piperidin-1-yl]-5-chloro-2-[(2-oxopiperidin-1-yl)methyl]nicotinate as oil.

¹H NMR (400 MHz, CDCl₃) δ 1.46 (9H, s), 1.75-1.89 (6H, m), 1.92-2.00 (2H, m), 2.40-2.50 (3H, m), 2.96-3.06 (2H, m), 3.35-3.42 (2H, m), 3.86 (3H, s), 4.03-4.10 (2H, m), 4.94 (2H, s), 8.10 (1H, s).

MS m/z: 466 (M+1)

### (h) 1-{3-chloro-5-(methoxycarbonyl)-6-[(2-oxopiperidin-1-yl)methyl]pyridin-2-yl}piperidine-4-carboxylic acid

Prepared according to Example 35(b) from appropriate intermediate methyl 6-[4-(tert-butoxycarbonyl)piperidin-1-yl]-5-chloro-2-[(2-oxopiperidin-1-yl)methyl]nicotinate (216 mg, 0.47 mmol) to give 1-{3-chloro-5-(methoxycarbonyl)-6-[(2-oxopiperidin-1-yl)methyl]pyridin-2-yl}piperidine-4-carboxylic acid.

### (i) Methyl 6-{4-[(benzylsulfonyl)carbamoyl]piperidin-1-yl}-5-chloro-2-[(2-oxopiperidin-1-yl)methyl]nicotinate

Prepared as Example 35(c) from appropriate intermediate 1-{3-chloro-5-(methoxycarbonyl)-6-[(2-oxopiperidin-1-yl)methyl]pyridin-2-yl}piperidine-4-carboxylic acid (48 mg, 0.12 mmol) to give methyl 6-{4-[(benzylsulfonyl)carbamoyl]piperidin-1-yl}-5-chloro-2-[(2-oxopiperidin-1-yl)methyl]nicotinate. Yield: 10 mg (15%).

¹H NMR (400 MHz, DMSO-d₆) δ 1.56-1.67 (2H, m), 1.71-1.81 (6H, m), 2.22-2.27 (2H, m), 2.88-2.98 (2H, m), 3.34-3.39 (2H, m), 3.80 (3H, s), 4.02-4.12 (2H, m), 4.5-4.65 (2H, m), 4.76 (2H, s), 7.23-7.39 (5H, m), 8.06 (1H, s), 11.54 (1H, bs). One signal (1H) is overlapping with the solvent signal.

MS m/z: 563 (M+1), 561 (M−1)

GTPγS (IC₅₀ μM): 1.58

### Example 130

### Propyl 6-{4-[(benzylsulfonyl)carbamoyl]piperidin-1-yl}-5-fluoro-2-[(2-oxopyrrolidin-1-yl)methyl]nicotinate

This compound could be prepared in essentially the same way as described in Example 127(a) to (f) replacing EtOH for nPrOH in step (b).

¹H NMR (CDCl₃, 400 MHz) δ 1.04 (3H, t, J=7.4 Hz), 1.67-1.83 (6H, m), 1.95-2.05 (2H, m), 2.25 (2H, t, J=8.0 Hz), 2.46-2.56 (1H, m), 2.87-2.98 (2H, m), 3.40 (2H, t, J=7.0 Hz), 4.19-4.27 (4H, m), 4.61 (2H, s), 4.81 (2H, s), 7.31-7.38 (5H, m), 7.76 (1H, d, J=14.2 Hz), 10.30 (1H, br s).

GTPγS (IC₅₀ μM): 0.122

1. A compound of formula I or a pharmaceutically acceptable salt thereof: 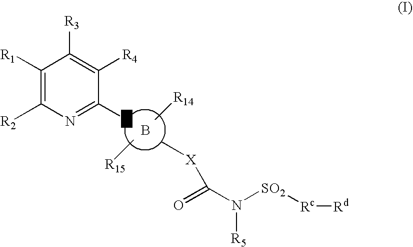
wherein
R₁ represents R₆OC(O) or R₁₆SC(O);
R₂ represents substituted (C₁-C₁₂)alkyl optionally interrupted by sulphur, substituted (C₁-C₁₂)alkoxy or substituted (C₁-C₁₂)alkylthio, wherein any one of these groups is substituted by one or more of azido, carboxy, cyano, (C₁-C₁₂)alkylcarbonyloxy, hydroxy(C₁-C₁₂)alkylcarbonyloxy, arylcarbonyloxy, heterocyclylcarbonyloxy, (C₁-C₁₂)alkyloxycarbonyl, (C₁-C₁₂)alkyl(C(S)), (C₁-C₁₂)alkyl(S(CO)), (C₁-C₁₂)alkylthio, hydroxy(C₁-C₁₂)alkylthio, (C₁-C₁₂)alkylsulfinyl, (C₁-C₁₂)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₁₂)alkylthio, aryl(C₁-C₁₂)alkylsulfinyl, aryl(C₁-C₁₂)alkylsulfonyl, heterocyclyl(C₁-C₁₂)alkyl thio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, (C₁-C₁₂)alkylcarbonyl, arylcarbonyl, heterocyclylcarbonyl, aryl(C₁-C₁₂)alkylcarbonyl, heterocyclyl(C₁-C₁₂)alkylcarbonyl or of a group of formula NR^{a(2)}R^{b(2)} or —(CO)NR^{a(2)}R^{b(2)}, in which R^{a(2)} and R^{b(2)} each and independently represent H, (C₁-C₁₂)alkyl, (C₁-C₁₂)alkylcarbonyl or R^{a(2)} and R^{b(2)} together with the nitrogen atom represent piperidine, pyrrolidine, azetidine or aziridine or any one of the groups 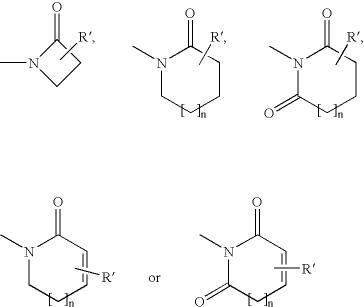
wherein n is an integer chosen from 0, 1 and 2, and R′ is H, CN, OH, a halogen atom, or one of the groups (C₁-C₈)alkyl, aryl, (C₁-C₈)alkoxy, (C₁-C₈)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen atoms; or
R₂ represents substituted (C₁-C₁₂)alkoxy or substituted (C₁-C₁₂)alkylthio, wherein any one of these groups is substituted by one or more of any one of OH, aryl, (C₃-C₆)cycloalkyl or heterocyclyl; or
R₂ represents (C₁-C₁₂)alkylthio, substituted by one or more halogen atom(s); or
R₂ represents (C₁-C₁₂)alkylcarbonyloxy, aryl carbonyloxy, heterocyclylcarbonyloxy of which any one optionally is substituted by one or more of any one of the following groups or atoms: azido, cyano, halogen atom(s), OH, (C₁-C₁₂)alkoxy, (C₁-C₁₂)alkylthio, (C₁-C₁₂)alkylsulfinyl, (C₁-C₁₂)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₁₂)alkylthio, aryl(C₁-C₁₂)alkylsulfinyl, aryl(C₁-C₁₂)alkylsulfonyl, heterocyclyl(C₁-C₁₂)alkyl thio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, (C₁-C₁₂)alkylcarbonyl, arylcarbonyl, heterocyclylcarbonyl, aryl(C₁-C₁₂)alkylcarbonyl and heterocyclyl(C₁-C₁₂)alkylcarbonyl; or
R₂ represents unsubstituted (C₁-C₁₂)alkyl with the proviso that at the same time R₅ represents carboxy(C₁-C₁₂)alkyl; or
R₂ represents a group of formula ((R^{a(2)})N(R^{b(2)}))(CO)—, in which R^{a(2)} and R^{b(2)} each and independently represent H, (C₁-C₁₂)alkyl, aryl, aryl(C₁-C₁₂)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine;
R₃ represents H, CN, NO₂, halogen, (C₁-C₁₂)alkyl optionally interrupted by oxygen and/or optionally substituted by OH, aryl, cycloalkyl, heterocyclyl or one or more halogen atoms; or
R₃ represents (C₁-C₁₂)alkoxy optionally substituted by one or more halogen atoms; or
R₃ represents (C₃-C₆)cycloalkyl, hydroxy(C₁-C₁₂)alkyl, (C₁-C₁₂)alkylC(O), (C₁-C₁₂)alkylthioC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(3)}R^{b(3)} in which R^{a(3)} and R^{b(3)} independently represent H, (C₁-C₁₂)alkyl, (C₁-C₁₂)alkylC(O) or R^{a(3)} and R^{b(3)} together with the nitrogen atom represent piperidine, pyrrolidine, azetidine or aziridine;
R₄ represents H, CN, NO₂, halogen, (C₁-C₁₂)alkyl optionally interrupted by oxygen and/or optionally substituted by OH, COOH, (C₁-C₆)alkoxycarbonyl, aryl, cycloalkyl, heterocyclyl or one or more halogen atoms; or
R₄ represents (C₃-C₆)cycloalkyl, hydroxy(C₁-C₁₂)alkyl, (C₁-C₁₂)alkylC(O), (C₁-C₁₂)alkylcycloalkyl, (C₁-C₁₂)alkoxy wherein the alkoxy group may optionally be substituted by one or more halogen atoms, OH and/or COOH and/or (C₁-C₆)alkoxycarbonyl; or
R₄ represents (C₁-C₁₂)alkylthioC(O), (C₁-C₁₂)alkylC(S), (C₁-C₁₂)alkoxyC(O), (C₃-C₆)cycloalkoxy, aryl, aryl(C₁-C₁₂)alkoxy, aryl(C₁-C₁₂)alkyl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkoxy, (C₃-C₆)cycloalkyl(C₁-C₁₂)alkylsulfinyl, (C₃-C₆)cycloalkyl(C₁-C₁₂)alkylsulfonyl or a group of formula NR^{a(4)}R^{b(4)} in which R^{a(4)} and R^{b(4)} independently represent H, (C₁-C₁₂)alkyl, (C₁-C₁₂)alkylC(O) or R^{a(4)} and R^{b(4)} together with the nitrogen atom represent piperidine, pyrrolidine, azetidine or aziridine;
R₅ represents H or (C₁-C₁₂)alkyl or carboxy(C₁-C₆)alkyl; with the proviso that when R₂ is unsubstituted (C₁-C₁₂)alkyl, R₅ represents carboxy(C₁-C₁₂)alkyl;
R₆ represents (C₁-C₁₂)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen atoms; or
R₆ represents (C₂-C₁₂)alkenyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen atoms; or
R₆ represents (C₂-C₁₂)alkynyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen atoms; or
R₆ represents (C₃-C₆)cycloalkyl, hydroxy(C₂-C₁₂)alkyl, aryl or heterocyclyl;
R₁₄ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{e}; wherein R^{e} represents aryl, cycloalkyl, heterocyclyl or (C₁-C₁₂)alkyl optionally substituted by one or more of halogen atoms, OH, aryl, cycloalkyl and heterocyclyl; further R₁₄ represents aryl, aryl(C₁-C₁₂)alkoxy, aryl(C₁-C₁₂)alkyl, (C₃-C₆)cycloalkyl(C₁-C₁₂)alkoxy, heterocyclyl, a halogen atom, (C₃-C₆)cycloalkyl, hydroxy(C₁-C₁₂)alkyl, (C₁-C₁₂)alkoxy, (C₃-C₆)cycloalkoxy, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkoxy, (C₃-C₆)cycloalkyl(C₁-C₁₂)alkylsulfinyl or (C₃-C₆)cycloalkyl(C₁-C₁₂)alkylsulfonyl, a group of formula NR^{a(14)}R^{b(14)} in which R^{a(14)} and R^{b(14)} independently represent H, (C₁-C₁₂)alkyl, (C₁-C₁₂)alkylC(O), (C₁-C₁₂)alkoxyC(O) or R^{a(14)} and R^{b(14)} together with the nitrogen atom represent piperidine, pyrrolidine, azetidine or aziridine;
R₁₅ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{e}; wherein R^{e} represents aryl, cycloalkyl, heterocyclyl or (C₁-C₁₂)alkyl optionally substituted by one or more of halogen atoms, OH, aryl, cycloalkyl and heterocyclyl; or
R₁₅ represents aryl, aryl(C₁-C₁₂)alkoxy, aryl(C₁-C₁₂)alkyl, (C₃-C₆)cycloalkyl(C₁-C₁₂)alkoxy, heterocyclyl, a halogen atom, (C₃-C₆)cycloalkyl, hydroxy(C₁-C₁₂)alkyl, (C₁-C₁₂)alkoxy, (C₃-C₆)cycloalkoxy, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkoxy, (C₃-C₆)cycloalkyl(C₁-C₁₂)alkylsulfinyl, (C₃-C₆)cycloalkyl(C₁-C₁₂)alkylsulfonyl or a group of formula NR^{a(15)}R^{b(15)} in which R^{a(15)} and R^{b(15)} independently represent H, (C₁-C₁₂)alkyl, (C₁-C₁₂)alkylC(O)), (C₁-C₁₂)alkoxyC(O) or R^{a(15)} and R^{b(15)} together with the nitrogen atom represent piperidine, pyrrolidine, azetidine or aziridine;
R₁₆ represents (C₁-C₁₂)alkyl optionally interrupted by oxygen (with the proviso that any such oxygen must be at least 2 carbon atoms away from the thioester-sulfur connecting the R₁₆ group) and/or optionally substituted by OH, aryl, cycloalkyl, heterocyclyl or one or more halogen atoms; or
R₁₆ represents (C₃-C₆)cycloalkyl, hydroxy(C₂-C₁₂)alkyl, (C₁-C₁₂)alkoxy, (C₃-C₆)cycloalkoxy, aryl or heterocyclyl;
R^{c} is a single bond or represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno, hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; or
R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above;
R₁₉ represents H or (C₁-C₄)alkyl;
R^{d} represents (C₁-C₁₂)alkyl, (C₃-C₈)cycloalkyl, aryl or heterocyclyl, and any one of these groups optionally substituted with one or more halogen atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, halogen substituted (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, tri(C₁-C₄)alkylsilyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine;
X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; or
X may represent a group (—CH₂-)n wherein n=2-6, which optionally is unsaturated and/or substituted by one or more substituent chosen among halogen, hydroxyl or (C₁-C₆)alkyl; and
B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions, and wherein the substituents R₁₄ and R₁₅ are connected to the B ring/ring system in such a way that no quarternary ammonium compounds are formed (by these connections).

2. A compound according to claim 1 wherein:
R₂ represents substituted (C₁-C₆)alkyl optionally interrupted by sulphur, substituted (C₁-C₆)alkoxy or substituted (C₁-C₆)alkylthio, wherein any one of these groups is substituted by one or more of azido, carboxy, cyano, (C₁-C₆)alkylcarbonyloxy, hydroxy(C₁-C₆)alkylcarbonyloxy, arylcarbonyloxy, heterocyclylcarbonyloxy, (C₁-C₆)alkyloxycarbonyl, (C₁-C₆)alkyl(C(S)), (C₁-C₆)alkyl(S(CO)), (C₁-C₆)alkylthio, hydroxy(C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl, heterocyclyl(C₁-C₆)alkylcarbonyl or of a group of formula NR^{a(2)}R^{b(2)} or —(CO)NR^{a(2)}R^{b(2)}, in which R^{a(2)} and R^{b(2)} each and independently represent H, (C₁-C₆)alkyl, (C₁-C₆)alkylcarbonyl or R^{a(2)} and R^{b(2)} together with the nitrogen atom represent piperidine, pyrrolidine, azetidine or aziridine or any one of the groups 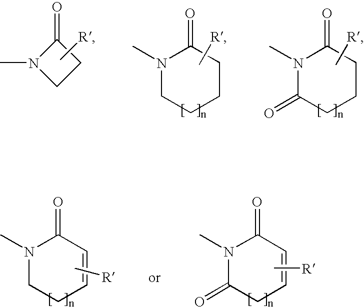
wherein n is an integer chosen from 0, 1 and 2, and R′ is H, CN, OH, a halogen atom, or one of the groups (C₁-C₈)alkyl, aryl, (C₁-C₈)alkoxy, (C₁-C₈)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen atoms; or
R₂ represents substituted (C₁-C₆)alkoxy or substituted (C₁-C₆)alkylthio, wherein any one of these groups is substituted by one or more of any one of OH, aryl, (C₃-C₆)cycloalkyl or heterocyclyl; or
R₂ represents (C₁-C₆)alkylthio, substituted by one or more halogen atom(s); or
R₂ represents (C₁-C₆)alkylcarbonyloxy, aryl carbonyloxy, heterocyclylcarbonyloxy of which any one optionally is substituted by one or more of any one of the following groups or atoms; atoms: azido, cyano, halogen atom(s), OH, (C₁-C₆)alkoxy, (C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl and heterocyclyl(C₁-C₆)alkylcarbonyl; or
R₂ represents unsubstituted (C₁-C₆)alkyl with the proviso that at the same time R₅ represents carboxy(C₁-C₆)alkyl; or
R₂ represents a group of formula ((R^{a(2)})N(R^{b(2)}))(CO)—, in which R^{a(2)} and R^{b(2)} each and independently represent H, (C₁-C₆)alkyl, aryl, aryl(C₁-C₆)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine;
R₃ represents H, CN, NO₂, halogen, (C₁-C₆)alkyl optionally interrupted by oxygen and/or optionally substituted by OH, aryl, cycloalkyl, heterocyclyl or one or more halogen atoms; or
R₃ represents (C₁-C₆)alkoxy optionally substituted by one or more halogen atoms; or
R₃ represents (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;
R₄ represents H, CN, NO₂, halogen, (C₁-C₆)alkyl optionally interrupted by oxygen and/or optionally substituted by OH, COOH, (C₁-C₆)alkoxycarbonyl, aryl, cycloalkyl, heterocyclyl or one or more halogen atoms; or
R₄ represents (C₃-C₆)cycloalkyl, hydroxy(C₁-C₆)alkyl, (C₁-C₆)alkylC(O), (C₁-C₆)alkylcycloalkyl, (C₁-C₆)alkoxy wherein the alkoxy group may optionally be substituted by one or more halogen atoms, OH and/or COOH and/or (C₁-C₆)alkoxycarbonyl; or
R₄ represents (C₁-C₆)alkylthioC(O), (C₁-C₆)alkylC(S), (C₁-C₆)alkoxyC(O), (C₃-C₆)cycloalkoxy, aryl, aryl(C₁-C₆)alkoxy, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkoxy, (C₃-C₆)cycloalkyl(C₁-C₆)alkylsulfinyl, (C₃-C₆)cycloalkyl(C₁-C₆)alkylsulfonyl or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;
R₅ represents H or (C₁-C₆)alkyl or carboxy(C₁-C₆)alkyl; with the proviso that when R₂ is unsubstituted (C₁-C₆)alkyl, R₅ represents carboxy(C₁-C₆)alkyl;
R₆ represents (C₁-C₆)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen atoms; or
R₆ represents (C₂-C₆)alkenyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen atoms; or
R₆ represents (C₂-C₆)alkynyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen atoms; or
R₆ represents (C₃-C₆)cycloalkyl, hydroxy(C₂-C₆)alkyl, aryl or heterocyclyl;
R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atoms, OH, aryl, cycloalkyl and heterocyclyl; or
R₁₄ represents aryl, aryl(C₁-C₆)alkoxy, aryl(C₁-C₆)alkyl, (C₃-C₆)cycloalkyl(C₁-C₆)alkoxy, heterocyclyl, a halogen atom, (C₃-C₆)cycloalkyl, hydroxy(C₁-C₆)alkyl, (C₁-C₆)alkoxy, (C₃-C₆)cycloalkoxy, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkoxy, (C₃-C₆)cycloalkyl(C₁-C₆)alkylsulfinyl or (C₃-C₆)cycloalkyl(C₁-C₆)alkylsulfonyl, a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;
R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atoms, OH, aryl, cycloalkyl and heterocyclyl; or
R₁₅ represents aryl, aryl(C₁-C₆)alkoxy, aryl(C₁-C₆)alkyl, (C₃-C₆)cycloalkyl(C₁-C₆)alkoxy, heterocyclyl, a halogen atom, (C₃-C₆)cycloalkyl, hydroxy(C₁-C₆)alkyl, (C₁-C₆)alkoxy, (C₃-C₆)cycloalkoxy, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkoxy, (C₃-C₆)cycloalkyl(C₁-C₆)alkylsulfinyl, (C₃-C₆)cycloalkyl(C₁-C₆)alkylsulfonyl or a group of formula NR^{a(15)}R^{b(15)} in which R^{a(15)} and R^{b(15)} independently represent H, (C₁-C₆)alkyl, (C₁-C₆)alkylC(O), (C₁-C₆)alkoxyC(O) or R^{a(15)} and R^{b(15)} together with the nitrogen atom represent piperidine, pyrrolidine, azetidine or aziridine;
R₁₆ represents (C₁-C₆)alkyl optionally interrupted by oxygen and/or optionally substituted by OH, aryl, cycloalkyl, heterocyclyl or one or more halogen atoms; or
R₁₆ represents (C₃-C₆)cycloalkyl, hydroxy(C₂-C₆)alkyl, (C₁-C₆)alkoxy, (C₃-C₆)cycloalkoxy, aryl or heterocyclyl; and
R^{d} represents (C₁-C₆)alkyl, (C₃-C₈)cycloalkyl, aryl or heterocyclyl, and any one of these groups optionally substituted with one or more halogen atoms and/or one or more of the following groups, groups: OH, CN, NO₂, (C₁-C₆)alkyl, (C₁-C₆)alkoxyC(O), (C₁-C₆)alkoxy, halogen substituted (C₁-C₆)alkyl, (C₃-C₆)cycloalkyl, aryl, heterocyclyl, (C₁-C₆)alkylsulfinyl, (C₁-C₆)alkylsulfonyl, (C₁-C₆)alkylthio, halogen substituted (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, tri(C₁-C₄)alkylsilyl or a group of formula NR^{a(Rd)}R^{b(Rd)} in which R^{a(Rd)} and R^{b(Rd)} independently represent H, (C₁-C₆)alkyl, (C₁-C₆)alkylC(O) or R^{a(Rd)} and R^{b(Rd)} together with the nitrogen atom represent piperidine, pyrrolidine, azetidine or aziridine.

3. A compound according to claim 2 wherein:
R₂ represents substituted (C₁-C₆)alkyl optionally interrupted by sulphur, substituted (C₁-C₆)alkoxy or substituted (C₁-C₆)alkylthio, wherein any one of these groups is substituted by one or more of azido, carboxy, cyano, (C₁-C₆)alkylcarbonyloxy, hydroxy(C₁-C₆)alkylcarbonyloxy, arylcarbonyloxy, heterocyclylcarbonyloxy, (C₁-C₆)alkyloxycarbonyl, (C₁-C₆)alkyl(C(S)), (C₁-C₆)alkyl(S(CO)), (C₁-C₆)alkylthio, hydroxy(C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl, heterocyclyl(C₁-C₆)alkylcarbonyl or of a group of formula NR^{a(2)}R^{b(2)} or —(CO)NR^{a(2)}R^{b(2)}, in which R^{a(2)} and R^{b(2)} each and independently represent H, (C₁-C₆)alkyl, (C₁-C₆)alkylcarbonyl or R^{a(2)} and R^{b(2)} together with the nitrogen atom represent piperidine, pyrrolidine, azetidine or aziridine or any one of the groups 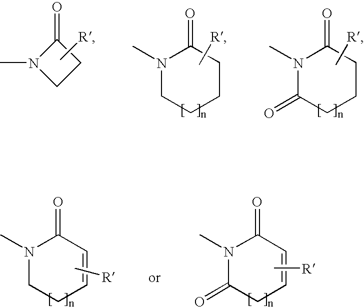
wherein n is an integer chosen from 0, 1 and 2, and R′ is H, CN, OH, a halogen atom, or one of the groups (C₁-C₆)alkyl, aryl, (C₁-C₆)alkoxy, (C₁-C₆)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen atoms; or
R₂ represents substituted (C₁-C₆)alkoxy or substituted (C₁-C₆)alkylthio, wherein any one of these groups is substituted by one or more of any one of OH, aryl, (C₃-C₆)cycloalkyl or heterocyclyl; or
R₂ represents (C₁-C₆)alkylthio, substituted by one or more halogen atom(s); or
R₂ represents (C₁-C₆)alkylcarbonyloxy, aryl carbonyloxy, heterocyclylcarbonyloxy of which any one optionally is substituted by one or more of any one of the following groups or atoms: azido, cyano, halogen atom(s), OH, (C₁-C₆)alkoxy, (C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl and heterocyclyl(C₁-C₆)alkylcarbonyl; or
R₂ represents unsubstituted (C₁-C₆)alkyl with the proviso that at the same time R₅ represents carboxy(C₁-C₆)alkyl; or
R₂ represents a group of formula ((R^{a(2)})N(R^{b(2)}))(CO)—, in which R^{a(2)} and R^{b(2)} each and independently represent H, (C₁-C₆)alkyl, aryl, aryl(C₁-C₆)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine;
R₃ represents H, CN, NO₂, halogen, (C₁-C₆)alkyl optionally interrupted by oxygen and/or optionally substituted by OH, aryl, cycloalkyl, heterocyclyl or one or more halogen atoms; or
R₃ represents (C₁-C₆)alkoxy optionally substituted by one or more halogen atoms; or
R₃ represents (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;
R₄ represents H, CN, NO₂, halogen, (C₁-C₆)alkyl optionally interrupted by oxygen and/or optionally substituted by OH, COOH, aryl, cycloalkyl, heterocyclyl or one or more halogen atoms; or
R₄ represents (C₃-C₆)cycloalkyl, hydroxy(C₁-C₆)alkyl, (C₁-C₆)alkylC(O), (C₁-C₆)alkoxy wherein the alkoxy group may optionally be substituted by one or more halogen atoms, OH and/or COOH and/or methoxycarbonyl; or
R₄ represents (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O) or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;
R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atoms, OH, aryl, cycloalkyl and heterocyclyl; or
R₁₄ represents aryl, aryl(C₁-C₆)alkoxy, aryl(C₁-C₆)alkyl, (C₃-C₆)cycloalkyl(C₁-C₆)alkoxy, heterocyclyl, a halogen atom, (C₃-C₆)cycloalkyl, hydroxy(C₁-C₆)alkyl, (C₁-C₆)alkoxy, (C₃-C₆)cycloalkoxy, or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;
R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atoms, OH, aryl, cycloalkyl and heterocyclyl; or
R₁₅ represents aryl, aryl(C₁-C₆)alkoxy, aryl(C₁-C₆)alkyl, (C₃-C₆)cycloalkyl(C₁-C₆)alkoxy, heterocyclyl, a halogen atom, (C₃-C₆)cycloalkyl, hydroxy(C₁-C₆)alkyl, (C₁-C₆)alkoxy, (C₃-C₆)cycloalkoxy, or a group of formula NR^{a(15)}R^{b(15)} in which R^{a(15)} and R^{b(15)} independently represent H, (C₁-C₆)alkyl, (C₁-C₆)alkylC(O), (C₁-C₆)alkoxyC(O) or R^{a(15)} and R^{b(15)} together with the nitrogen atom represent piperidine, pyrrolidine, azetidine or aziridine; and
R₁₆ represents (C₁-C₆)alkyl optionally interrupted by oxygen and/or optionally substituted by OH, aryl, cycloalkyl, heterocyclyl or one or more halogen atoms; or
R₁₆ represents (C₃-C₆)cycloalkyl, hydroxy(C₂-C₆)alkyl, (C₁-C₆)alkoxy, (C₃-C₆)cycloalkoxy, aryl or heterocyclyl.

4. A compound according to claim 3 wherein; wherein:
R₂ represents substituted (C₁-C₆)alkyl optionally interrupted by sulphur, substituted (C₁-C₆)alkoxy or substituted (C₁-C₆)alkylthio, wherein any one of these groups is substituted by one or more of azido, carboxy, cyano, (C₁-C₆)alkylcarbonyloxy, hydroxy(C₁-C₆)alkylcarbonyloxy, arylcarbonyloxy, heterocyclylcarbonyloxy, (C₁-C₆)alkyloxycarbonyl, (C₁-C₆)alkyl(C(S)), (C₁-C₆)alkyl(S(CO)), (C₁-C₆)alkylthio, hydroxy(C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl, heterocyclyl(C₁-C₆)alkylcarbonyl or of a group of formula NR^{a(2)}R^{b(2)} or —(CO)NR^{a(2)}R^{b(2)}, in which R^{a(2)} and R^{b(2)} each and independently represent H, (C₁-C₆)alkyl, (C₁-C₆)alkylcarbonyl or R^{a(2)} and R^{b(2)} together with the nitrogen atom represent piperidine, pyrrolidine, azetidine or aziridine or any one of the groups 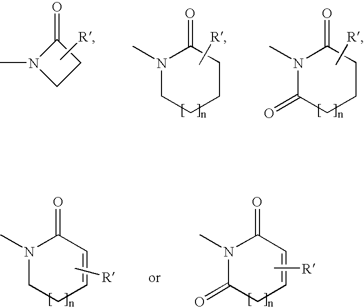
wherein n is an integer chosen from 0, 1 and 2, and R′ is H, CN, OH, a halogen atom, or one of the groups (C₁-C₄)alkyl, aryl, (C₁-C₄)alkoxy, (C₁-C₄)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen atoms; or
R₂ represents substituted (C₁-C₆)alkoxy or substituted (C₁-C₆)alkylthio, wherein any one of these groups is substituted by one or more of any one of OH, aryl, (C₃-C₆)cycloalkyl or heterocyclyl; or
R₂ represents (C₁-C₆)alkylthio, substituted by one or more halogen atom(s); or
R₂ represents (C₁-C₆)alkylcarbonyloxy, aryl carbonyloxy, heterocyclylcarbonyloxy of which any one optionally is substituted by one or more of any one of the following groups or atoms: azido, cyano, halogen atom(s), OH, (C₁-C₆)alkoxy, (C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl and heterocyclyl(C₁-C₆)alkylcarbonyl; or
R₂ represents unsubstituted (C₁-C₆)alkyl with the proviso that at the same time R₅ represents carboxy(C₁-C₆)alkyl;
R₃ represents H or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;
R₄ represents CN, halogen; or
R₄ represents (C₁-C₆)alkylC(O), (C₁-C₆)alkoxy wherein the alkoxy group may optionally be substituted by one or more halogen atoms, OH and/or COOH and/or methoxycarbonyl;
R₅ represents H or carboxy(C₁-C₆)alkyl; with the proviso that when R₂ is unsubstituted (C₁-C₆)alkyl, R₅ represents carboxy(C₁-C₆)alkyl;
R₆ represents (C₁-C₆)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen atoms; or
R₆ represents (C₃-C₆)cycloalkyl or hydroxy(C₂-C₆)alkyl;
R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atoms, OH, aryl, cycloalkyl and heterocyclyl; or
R₁₄ represents or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine; and
R₁₅ represents H.

5. A compound according to claim 1 wherein:
R₁ is selected from methoxycarbonyl, ethoxycarbonyl, n-propyloxycarbonyl, isopropyloxycarbonyl and ethylthiocarbonyl;
R₂ is selected from methyl, acetoxy, pyrrolidin-1-yl-methyl, (2-oxopyrrolidin-1-yl)methyl, 2-(2-oxopyrrolidin-1-yl)ethoxy, (2-oxopiperidin-1-yl)methyl, (dimethylamino)methyl, acetoxymethyl, 3-oxobutyl, 2-ethoxy-2-oxoethoxy, 3-ethoxy-3-oxopropyl, 4-ethoxy-4-oxobutyl, azidomethyl, (methylsulfonyl)methyl, (2-ethoxy-2-oxoethyl)thio, ((2-ethoxy-2-oxoethyl)thio)methyl, (2-isopropoxy-2oxoethyl)thio, (2,5-dioxopyrrolidin-1-yl)methyl, (glycoloyloxy)methyl, 2-cyanoethoxy, 2-hydroxyethoxy, ethylthiomethyl, (2-hydroxyethyl)thio, (2-hydroxyethyl)thiomethyl, 2-acetamidoethoxy, (2-acetamidoethyl)thio, (2-acetamidoethyl)thiomethyl, 2-amino-2-oxoethoxy, oxetan-2-yl-methoxy, carboxymethyloxy, 2-(methylamino)-2-oxoethoxy and (carboxy)methylthio, ((carboxy)methylthio)methyl, ethylthiomethyl, 2-(1H-pyrrol-1-yl)ethoxy, (((1-methyl-1H-imidazol-2-yl)methyl)thio)methyl, (1,3-thiazol-2-ylthio)methyl, ((3-methoxy-3-oxopropyl)thio)methyl, (1S)-2-ethoxy-1-methyl-2-oxoethoxy, 4-oxopentyl, ((2-(dimethylamino)-2-oxoethyl)thio)methyl, (2-azetidin-1-yl-2-oxoethyl)thio, 2-(dimethylamino)-2-oxoethoxy with the proviso that when R₂ is methyl R₅ is carboxymethyl;
R₃ is H;
R₄ is selected from hydrogen, fluoro, chloro, bromo and cyano;
R₅ is H, methyl or carboxymethyl;
R₆ is selected from methyl, ethyl, isopropyl and n-propyl;
R₁₄ is H;
R₁₅ is H;
R₁₆ is ethyl;
R^{c} is selected from methylene (—CH₂—), imino (—NH—) and methylimino (—N(CH₃)—);
R₁₉ is H or methyl;
R^{d} is selected from cyclobutyl, cyclopentyl, cyclohexyl, 4-methylcyclohexyl, tetrahydro-2H-pyran-4-yl, phenyl, 4-methylphenyl, 4-isopropylphenyl, 4-tert-butylphenyl, 4-methoxyphenyl, 4-fluorophenyl, 4-chlorophenyl, 2,4-difluorophenyl, 2,4-dichlorophenyl, 2-chloro-4-fluoro-phenyl, 4-chloro-2-fluoro-phenyl, 4-trifluoromethylphenyl, 4-((trifluoromethyl)thio)-phenyl and 4-(trimethylsilyl)-phenyl;
X represents a single bond; and
B is selected from 4-piperidin-1-ylene and 3-azetidin-1-ylene, and the substituents R₁₄ and R₁₅ are connected to the B ring/ring system, in such a way that no quarternary ammonium compounds are formed (by these connections).

6. A compound according to claim 1 which is of the formula (Ia):

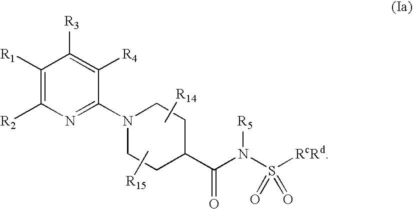

7. A compound according to claim 1 which is of the formula (Ib):

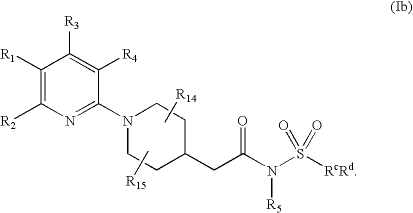

8. A compound according to claim 1 which is of the formula (Ic):

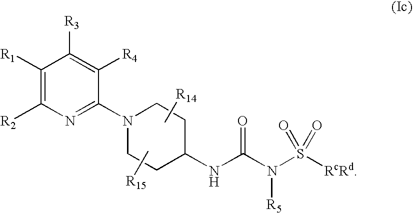

9. A compound according to claim 1 which is of the formula (Id):

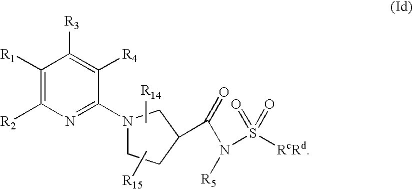

10. A compound according to claim 1 which is of the formula (Ie):

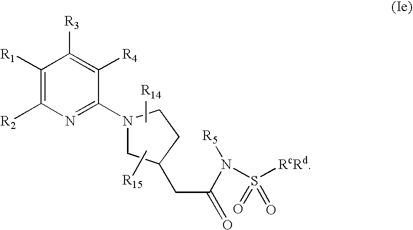

11. A compound according to claim 1 which is of the formula (If):

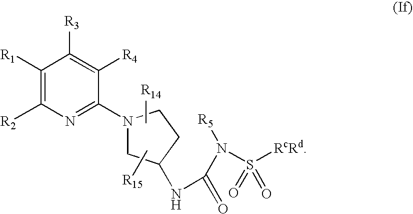

12. A compound according to claim 1 which is of the formula (Ig):

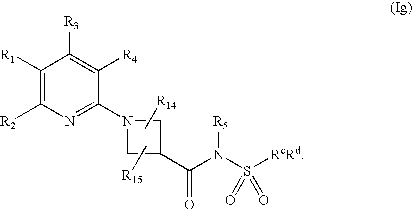

13. A compound according to claim 1 which is of the formula (Ih):

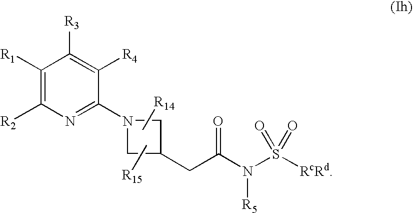

14. A compound according to claim 1 which is of the formula (Ii):

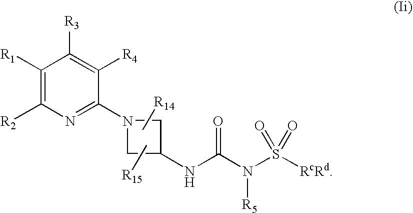

15. A compound according to claim 1 wherein R₁ represents R₆OC(O).

16. A compound according to claim 1 wherein R₁ represents R₁₆SC(O).

17. A compound according to claim 15 which is of the formula (Iaa):

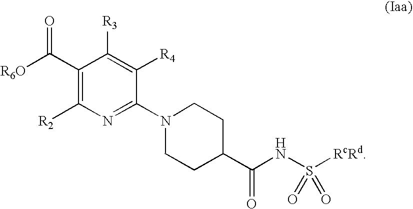

18. A compound according to claim 15 which is of the formula (Iab):

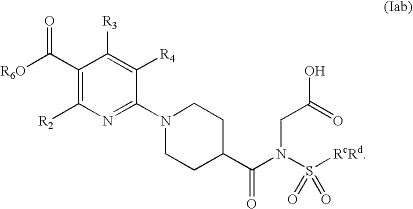

19. A compound according to claim 15 which is of the formula (Iac):

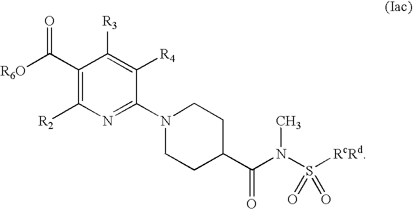

20. A compound according to claim 16 which is of the formula (Iad):

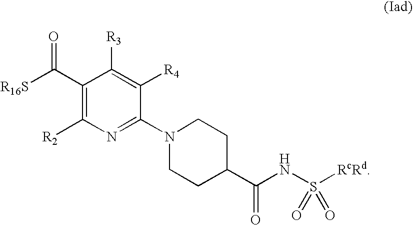

21. A compound according to claim 15 which is of the formula (Igg):

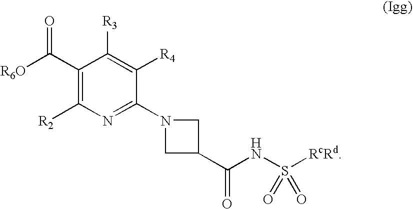

22. A compound selected from:
N-(benzylsulfonyl)-N-({1-[3-cyano-5-(ethoxycarbonyl)-6-methylpyridin-2-yl]piperidin-4-yl}carbonyl)glycine
ethyl 2-acetoxy-6-{4-[(benzylsulfonyl)carbamoyl]piperidin-1-yl}-5-cyanonicotinate
ethyl 6-{4-[(benzylsulfonyl)carbamoyl]piperidin-1-yl}-5-cyano-2-(pyrrolidin-1-ylmethyl)nicotinate
ethyl 6-{4-[(benzylsulfonyl)carbamoyl]piperidin-1-yl}-5-cyano-2-[(dimethylamino)methyl]nicotinate
ethyl 2-(acetoxymethyl)-6-{4-[(benzylsulfonyl)carbamoyl]piperidin-1-yl}-5-cyanonicotinate
ethyl 6-{3-[(benzylsulfonyl)carbamoyl]azetidin-1-yl}-5-cyano-2-[(dimethylamino)methyl]nicotinate
ethyl 2-(acetoxymethyl)-6-{3-[(benzylsulfonyl)carbamoyl]azetidin-1-yl}-5-cyanonicotinate
ethyl 6-{4-[(benzylsulfonyl)carbamoyl]piperidin-1-yl}-5-cyano-2-(2-ethoxy-2-oxoethoxy)nicotinate
ethyl 2-(azidomethyl)-6-{3-[(benzylsulfonyl)carbamoyl]azetidin-1-yl}-5-cyanonicotinate
ethyl 6-{3-[(benzylsulfonyl)carbamoyl]azetidin-1-yl}-5-cyano-2-[(methylsulfonyl)methyl]nicotinate
ethyl 2-(azidomethyl)-6-{4-[(benzylsulfonyl)carbamoyl]piperidin-1-yl}-5-cyanonicotinate
ethyl 6-{4-[(benzylsulfonyl)carbamoyl]piperidin-1-yl}-5-cyano-2-[(methylsulfonyl)methyl]nicotinate
ethyl 6-{3-[(benzylsulfonyl)carbamoyl]azetidin-1-yl}-5-cyano-2-[(2,5-dioxopyrrolidin-1-yl)methyl]nicotinate
ethyl 6-{3-[(benzylsulfonyl)carbamoyl]azetidin-1-yl}-5-cyano-2-[(glycoloyloxy)methyl]nicotinate
ethyl 6-{4-[(benzylsulfonyl)carbamoyl]piperidin-1-yl}-5-cyano-2-[(2,5-dioxopyrrolidin-1-yl)methyl]nicotinate
ethyl 6-{4-[(benzylsulfonyl)carbamoyl]piperidin-1-yl}-5-cyano-2-[(glycoloyloxy)methyl]nicotinate
ethyl 6-{4-[(benzylsulfonyl)carbamoyl]piperidin-1-yl}-5-cyano-2-(2-cyanoethoxy)nicotinate
ethyl 6-{4-[(benzylsulfonyl)carbamoyl]piperidin-1-yl}-5-cyano-2-(2-hydroxyethoxy)nicotinate
ethyl 6-{4-[(benzylsulfonyl)carbamoyl]piperidin-1-yl}-5-cyano-2-[(2-hydroxyethyl)thio]nicotinate
ethyl 2-(2-acetamidoethoxy)-6-{4-[(benzylsulfonyl)carbamoyl]piperidin-1-yl}-5-cyanonicotinate
ethyl 2-[(2-acetamidoethyl)thio]-6-{4-[(benzylsulfonyl)carbamoyl]piperidin-1-yl}-5-cyanonicotinate
ethyl 2-(2-amino-2-oxoethoxy)-6-{4-[(benzylsulfonyl)carbamoyl]piperidin-1-yl}-5-cyanonicotinate
ethyl 6-{4-[(benzylsulfonyl)carbamoyl]piperidin-1-yl}-5-cyano-2-(oxetan-2-ylmethoxy)nicotinate
{[6-{4-[(benzylsulfonyl)carbamoyl]piperidin-1-yl}-5-cyano-3-(ethoxycarbonyl)pyridin-2-yl]oxy}acetic acid
Ethyl 6-{4-[(benzylsulfonyl)carbamoyl]piperidin-1-yl}-5-cyano-2-[2-(methylamino)-2-oxoethoxy]nicotinate
{[6-{4-[(benzylsulfonyl)carbamoyl]piperidin-1-yl}-5-cyano-3-(ethoxycarbonyl)pyridin-2-yl]thio}acetic acid
Ethyl 6-{4-[(benzylsulfonyl)carbamoyl]piperidin-1-yl}-5-cyano-2-[(ethylthio)methyl]nicotinate
Ethyl 6-{4-[(benzylsulfonyl)carbamoyl]piperidin-1-yl}-5-cyano-2-{[(2-hydroxyethyl)thio]methyl}nicotinate
Ethyl 2-{[(2-acetamidoethyl)thio]methyl}-6-{4-[(benzylsulfonyl)carbamoyl]piperidin-1-yl}-5-cyanonicotinate
Ethyl 6-{3-[(benzylsulfonyl)carbamoyl]azetidin-1-yl}-5-cyano-2-[(ethylthio)methyl]nicotinate
Ethyl 6-{3-[(benzylsulfonyl)carbamoyl]azetidin-1-yl}-5-cyano-2-{[(2-hydroxyethyl)thio]methyl}nicotinate
Ethyl 2-{[(2-acetamidoethyl)thio]methyl}-6-{3-[(benzylsulfonyl)carbamoyl]azetidin-1-yl}-5-cyanonicotinate
ethyl 6-{4-[(benzylsulfonyl)carbamoyl]piperidin-1-yl}-5-cyano-2-[(2-oxopyrrolidin-1-yl)methyl]nicotinate
isopropyl 6-{4-[(benzylsulfonyl)carbamoyl]piperidin-1-yl}-5-cyano-2-[(2-oxopyrrolidin-1-yl)methyl]nicotinate
ethyl 5-cyano-2-[(2-oxopyrrolidin-1-yl)methyl]-6-[4-({[4-(trifluoromethyl)benzyl]sulfonyl}carbamoyl)piperidin-1-yl]nicotinate
ethyl 5-cyano-6-(4-{[(cyclopentylmethyl)sulfonyl]carbamoyl}piperidin-1-yl)-2-[(2-oxopyrrolidin-1-yl)methyl]nicotinate
ethyl 5-cyano-6-[4-({[(4-methylcyclohexyl)methyl]sulfonyl}carbamoyl)piperidin-1-yl]-2-[(2-oxopyrrolidin-1-yl)methyl]nicotinate
ethyl 5-cyano-6-(4-{[(2,4-difluorobenzyl)sulfonyl]carbamoyl}piperidin-1-yl)-2-[(2-oxopyrrolidin-1-yl)methyl]nicotinate
ethyl 6-(4-{[(4-chlorobenzyl)sulfonyl]carbamoyl}piperidin-1-yl)-5-cyano-2-[(2-oxopyrrolidin-1-yl)methyl]nicotinate
ethyl 5-cyano-6-(4-{[(4-fluorobenzyl)sulfonyl]carbamoyl}piperidin-1-yl)-2-[(2-oxopyrrolidin-1-yl)methyl]nicotinate
ethyl 5-cyano-6-(4-{[(2,4-dichlorobenzyl)sulfonyl]carbamoyl}piperidin-1-yl)-2-[(2-oxopyrrolidin-1-yl)methyl]nicotinate
ethyl 5-cyano-6-(4-{[(4-methylbenzyl)sulfonyl]carbamoyl}piperidin-1-yl)-2-[(2-oxopyrrolidin-1-yl)methyl]nicotinate
ethyl 6-(4-{[(2-chloro-4-fluorobenzyl)sulfonyl]carbamoyl}piperidin-1-yl)-5-cyano-2-[(2-oxopyrrolidin-1-yl)methyl]nicotinate
ethyl 6-(4-{[(4-chloro-2-fluorobenzyl)sulfonyl]carbamoyl}piperidin-1-yl)-5-cyano-2-[(2-oxopyrrolidin-1-yl)methyl]nicotinate
isopropyl 5-cyano-6-(4-{[(4-methylbenzyl)sulfonyl]carbamoyl}piperidin-1-yl)-2-[(2-oxopyrrolidin-1-yl)methyl]nicotinate
isopropyl 5-cyano-6-(4-{[(2,4-difluorobenzyl)sulfonyl]carbamoyl}piperidin-1-yl)-2-[(2-oxopyrrolidin-1-yl)methyl]nicotinate
isopropyl 5-cyano-6-(4-{[(cyclopentylmethyl)sulfonyl]carbamoyl}piperidin-1-yl)-2-[(2-oxopyrrolidin-1-yl)methyl]nicotinate
isopropyl 6-(4-{[(4-chlorobenzyl)sulfonyl]carbamoyl}piperidin-1-yl)-5-cyano-2-[(2-oxopyrrolidin-1-yl)methyl]nicotinate
isopropyl 6-(4-{[(2-chloro-4-fluorobenzyl)sulfonyl]carbamoyl}piperidin-1-yl)-5-cyano-2-[(2-oxopyrrolidin-1-yl)methyl]nicotinate
isopropyl 5-cyano-2-[(2-oxopyrrolidin-1-yl)methyl]-6-[4-({[4-(trifluoromethyl)benzyl]sulfonyl}carbamoyl)piperidin-1-yl]nicotinate
isopropyl 5-cyano-6-(4-{[(4-fluorobenzyl)sulfonyl]carbamoyl}piperidin-1-yl)-2-[(2-oxopyrrolidin-1-yl)methyl]nicotinate
isopropyl 6-(4-{[(4-chloro-2-fluorobenzyl)sulfonyl]carbamoyl}piperidin-1-yl)-5-cyano-2-[(2-oxopyrrolidin-1-yl)methyl]nicotinate
isopropyl 5-cyano-6-(4-{[(2,4-dichlorobenzyl)sulfonyl]carbamoyl}piperidin-1-yl)-2-[(2-oxopyrrolidin-1-yl)methyl]nicotinate
isopropyl 5-cyano-6-[4-({[(4-methylcyclohexyl)methyl]sulfonyl}carbamoyl)piperidin-1-yl]-2-[(2-oxopyrrolidin-1-yl)methyl]nicotinate
ethyl 6-{4-[(anilinosulfonyl)carbamoyl]piperidin-1-yl}-5-cyano-2-[(2-oxopyrrolidin-1-yl)methyl]nicotinate
ethyl 6-{4-[(benzylsulfonyl)carbamoyl]piperidin-1-yl}-5-cyano-2-[(2-oxopiperidin-1-yl)methyl]nicotinate
ethyl 5-cyano-6-[4-({[methyl(phenyl)amino]sulfonyl}carbamoyl)piperidin-1-yl]-2-[(2-oxopyrrolidin-1-yl)methyl]nicotinate
ethyl 5-cyano-6-[4-({[(4-methylcyclohexyl)methyl]sulfonyl}carbamoyl)piperidin-1-yl]-2-[(2-oxopiperidin-1-yl)methyl]nicotinate
ethyl 5-cyano-2-[(2-oxopiperidin-1-yl)methyl]-6-[4-({[4-(trifluoromethyl)benzyl]sulfonyl}carbamoyl)piperidin-1-yl]nicotinate
ethyl 5-cyano-6-(4-{[(cyclopentylmethyl)sulfonyl]carbamoyl}piperidin-1-yl)-2-[(2-oxopiperidin-1-yl)methyl]nicotinate
ethyl 5-cyano-6-(4-{[(cyclohexylmethyl)sulfonyl]carbamoyl}piperidin-1-yl)-2-[(2-oxopiperidin-1-yl)methyl]nicotinate
ethyl 5-cyano-6-(4-{[(4-fluorobenzyl)sulfonyl]carbamoyl}piperidin-1-yl)-2-[(2-oxopiperidin-1-yl)methyl]nicotinate
ethyl 5-cyano-6-(4-{[(4-methylbenzyl)sulfonyl]carbamoyl}piperidin-1-yl)-2-[(2-oxopiperidin-1-yl)methyl]nicotinate
ethyl 6-{4-[(anilinosulfonyl)carbamoyl]piperidin-1-yl}-5-cyano-2-[(2-oxopiperidin-1-yl)methyl]nicotinate
ethyl 5-cyano-6-(4-{[(cyclohexylmethyl)sulfonyl]carbamoyl}piperidin-1-yl)-2-[(2-oxopyrrolidin-1-yl)methyl]nicotinate
ethyl 5-cyano-2-[(2-oxopyrrolidin-1-yl)methyl]-6-{4-[({4-[(trifluoromethyl)thio]benzyl}sulfonyl)carbamoyl]piperidin-1-yl}nicotinate
ethyl 5-cyano-6-(4-{[(2,4-difluorobenzyl)sulfonyl]carbamoyl}piperidin-1-yl)-2-[(2-oxopiperidin-1-yl)methyl]nicotinate
ethyl 5-cyano-6-(4-{[(4-methoxybenzyl)sulfonyl]carbamoyl}piperidin-1-yl)-2-[(2-oxopiperidin-1-yl)methyl]nicotinate
ethyl 5-cyano-6-(4-{[(4-isopropylbenzyl)sulfonyl]carbamoyl}piperidin-1-yl)-2-[(2-oxopiperidin-1-yl)methyl]nicotinate
isopropyl 6-{4-[(benzylsulfonyl)carbamoyl]piperidin-1-yl}-5-cyano-2-[(2-oxopiperidin-1-yl)methyl]nicotinate
ethyl 5-cyano-6-[4-({[methyl(phenyl)amino]sulfonyl}carbamoyl)piperidin-1-yl]-2-[(2-oxopiperidin-1-yl)methyl]nicotinate
ethyl 5-cyano-2-[(2-oxopyrrolidin-1-yl)methyl]-6-(4-{[(tetrahydro-2H-pyran-4-ylmethyl)sulfonyl]carbamoyl}piperidin-1-yl)nicotinate
isopropyl 5-cyano-2-[(2-oxopyrrolidin-1-yl)methyl]-6-(4-{[(tetrahydro-2H-pyran-4-ylmethyl)sulfonyl]carbamoyl}piperidin-1-yl)nicotinate
ethyl 5-cyano-6-(4-{[(cyclobutylmethyl)sulfonyl]carbamoyl}piperidin-1-yl)-2-[(2-oxopyrrolidin-1-yl)methyl]nicotinate
ethyl 5-cyano-6-(4-{[(4-methoxybenzyl)sulfonyl]carbamoyl}piperidin-1-yl)-2-[(2-oxopyrrolidin-1-yl)methyl]nicotinate
ethyl 5-cyano-6-[4-({[(4-fluorophenyl)(methyl)amino]sulfonyl}carbamoyl)piperidin-1-yl]-2-[(2-oxopyrrolidin-1-yl)methyl]nicotinate
ethyl 5-cyano-6-[4-({[(4-fluorophenyl)amino]sulfonyl}carbamoyl)piperidin-1-yl]-2-[(2-oxopyrrolidin-1-yl)methyl]nicotinate
isopropyl 5-cyano-6-[4-({[methyl(phenyl)amino]sulfonyl}carbamoyl)piperidin-1-yl]-2-[(2-oxopyrrolidin-1-yl)methyl]nicotinate
isopropyl 5-cyano-6-[4-({[(4-fluorophenyl)(methyl)amino]sulfonyl}carbamoyl) piperidin-1-yl]-2-[(2-oxopyrrolidin-1-yl)methyl]nicotinate
isopropyl 5-cyano-6-[4-({[(4-fluorophenyl)amino]sulfonyl}carbamoyl)piperidin-1-yl]-2-[(2-oxopyrrolidin-1-yl)methyl]nicotinate
isopropyl 5-cyano-6-(4-{[(cyclobutylmethyl)sulfonyl]carbamoyl}piperidin-1-yl)-2-[(2-oxopyrrolidin-1-yl)methyl]nicotinate
isopropyl 5-cyano-6-(4-{[(cyclohexylmethyl)sulfonyl]carbamoyl}piperidin-1-yl)-2-[(2-oxopyrrolidin-1-yl)methyl]nicotinate
isopropyl 5-cyano-6-(4-{[(4-methoxybenzyl)sulfonyl]carbamoyl}piperidin-1-yl)-2-[(2-oxopyrrolidin-1-yl)methyl]nicotinate
ethyl 6-{4-[(benzylsulfonyl)carbamoyl]piperidin-1-yl}-5-bromo-2-[(2-oxopyrrolidin-1-yl)methyl]nicotinate
ethyl 6-{4-[(benzylsulfonyl)carbamoyl]piperidin-1-yl}-5-chloro-2-[(2-oxopyrrolidin-1-yl)methyl]nicotinate
ethyl 6-{4-[(benzylsulfonyl)carbamoyl]piperidin-1-yl}-2-[(2-oxopyrrolidin-1-yl)methyl]nicotinate
ethyl 5-chloro-6-(4-{[(4-methylbenzyl)sulfonyl]carbamoyl}piperidin-1-yl)-2-[(2-oxopyrrolidin-1-yl)methyl]nicotinate
ethyl 5-chloro-6-(4-{[(4-methoxybenzyl)sulfonyl]carbamoyl}piperidin-1-yl)-2-[(2-oxopyrrolidin-1-yl)methyl]nicotinate
ethyl 5-chloro-6-(4-{[(2,4-difluorobenzyl)sulfonyl]carbamoyl}piperidin-1-yl)-2-[(2-oxopyrrolidin-1-yl)methyl]nicotinate
ethyl 6-{3-[(benzylsulfonyl)carbamoyl]azetidin-1-yl}-5-cyano-2-[(2-oxopyrrolidin-1-yl)methyl]nicotinate
ethyl 5-chloro-6-[4-({[(4-fluorophenyl)amino]sulfonyl}carbamoyl)piperidin-1-yl]-2-[(2-oxopyrrolidin-1-yl)methyl]nicotinate
ethyl 5-chloro-6-[4-({[(4-fluorophenyl)(methyl)amino]sulfonyl}carbamoyl)piperidin-1-yl]-2-[(2-oxopyrrolidin-1-yl)methyl]nicotinate
ethyl 5-chloro-6-[4-({[methyl(phenyl)amino]sulfonyl}carbamoyl)piperidin-1-yl]-2-[(2-oxopyrrolidin-1-yl)methyl]nicotinate
ethyl 5-chloro-6-(4-{[(cyclohexylmethyl)sulfonyl]carbamoyl}piperidin-1-yl)-2-[(2-oxopyrrolidin-1-yl)methyl]nicotinate
propyl 5-chloro-6-[4-({[(4-fluorophenyl)(methyl)amino]sulfonyl}carbamoyl)piperidin-1-yl]-2-[(2-oxopyrrolidin-1-yl)methyl]nicotinate
isopropyl 6-{4-[(benzylsulfonyl)carbamoyl]piperidin-1-yl}-5-chloro-2-[(2-oxopyrrolidin-1-yl)methyl]nicotinate
S-ethyl 5-cyano-6-(4-{[(cyclopentylmethyl)sulfonyl]carbamoyl}piperidin-1-yl)-2-[(2-oxopyrrolidin-1-yl)methyl]pyridine-3-carbothioate
S-ethyl 5-cyano-6-(4-{[(4-methylbenzyl)sulfonyl]carbamoyl}piperidin-1-yl)-2-[(2-oxopyrrolidin-1-yl)methyl]pyridine-3-carbothioate
isopropyl 5-cyano-2-[(2-oxopyrrolidin-1-yl)methyl]-6-[4-({[4-(trimethylsilyl)benzyl]sulfonyl}carbamoyl)piperidin-1-yl]nicotinate
ethyl 5-cyano-2-[(2-oxopyrrolidin-1-yl)methyl]-6-[4-({[4-(trimethylsilyl)benzyl]sulfonyl}carbamoyl)piperidin-1-yl]nicotinate
ethyl 5-cyano-2-[(2-oxopiperidin-1-yl)methyl]-6-{4-[({4-[(trifluoromethyl)thio]benzyl}sulfonyl)carbamoyl]piperidin-1-yl}nicotinate
ethyl 5-cyano-2-[(2-oxopiperidin-1-yl)methyl]-6-[4-({[4-(trimethylsilyl)benzyl]sulfonyl}carbamoyl)piperidin-1-yl]nicotinate
ethyl 6-(4-{[(4-tert-butylbenzyl)sulfonyl]carbamoyl}piperidin-1-yl)-5-cyano-2-[(2-oxopiperidin-1-yl)methyl]nicotinate
ethyl 2-[(2-acetamidoethyl)thio]-5-chloro-6-(4-{[(4-chlorobenzyl)sulfonyl]carbamoyl}piperidin-1-yl)nicotinate
ethyl 2-[(2-acetamidoethyl)thio]-6-{4-[(benzylsulfonyl)carbamoyl]piperidin-1-yl}-5-chloronicotinate
ethyl 6-{4-[(benzylsulfonyl)carbamoyl]piperidin-1-yl}-5-cyano-2-(4-ethoxy-4-oxobutyl)nicotinate
ethyl 6-{4-[(benzylsulfonyl)carbamoyl]piperidin-1-yl}-5-cyano-2-(3-ethoxy-3-oxopropyl)nicotinate
ethyl 6-(4-{[(2-chloro-4-fluorobenzyl)sulfonyl]carbamoyl}piperidin-1-yl)-5-cyano-2-[(2-ethoxy-2-oxoethyl)thio]nicotinate
ethyl 5-cyano-6-(4-{[(2,4-dichlorobenzyl)sulfonyl]carbamoyl}piperidin-1-yl)-2-[(2-ethoxy-2-oxoethyl)thio]nicotinate
ethyl 5-cyano-6-(4-{[(cyclopentylmethyl)sulfonyl]carbamoyl}piperidin-1-yl)-2-[(2-ethoxy-2-oxoethyl)thio]nicotinate
ethyl 6-{4-[(benzylsulfonyl)carbamoyl]piperidin-1-yl}-5-cyano-2-[(2-isopropoxy-2-oxoethyl)thio]nicotinate
ethyl 6-{4-[(benzylsulfonyl)carbamoyl]piperidin-1-yl}-5-cyano-2-(3-oxobutyl)nicotinate
ethyl 6-{4-[(benzylsulfonyl)carbamoyl]piperidin-1-yl}-5-cyano-2-[2-(1H-pyrrol-1-yl)ethoxy]nicotinate
ethyl 6-{4-[(benzylsulfonyl)carbamoyl]piperidin-1-yl}-5-cyano-2-[2-(2-oxopyrrolidin-1-yl)ethoxy]nicotinate ({[6-{4-[(benzylsulfonyl)carbamoyl]piperidin-1-yl}-5-cyano-3-(ethoxycarbonyl)pyridin-2-yl]methyl}thio)acetic acid
ethyl 6-{4-[(benzylsulfonyl)carbamoyl]piperidin-1-yl}-5-cyano-2-({[(1-methyl-1H-imidazol-2-yl)methyl]thio}methyl)nicotinate
ethyl 6-{4-[(benzylsulfonyl)carbamoyl]piperidin-1-yl}-5-cyano-2-[(1,3-thiazol-2-ylthio)methyl]nicotinate
ethyl 6-{4-[(benzylsulfonyl)carbamoyl]piperidin-1-yl}-5-cyano-2-{[(1-methyl-1H-imidazol-2-yl)thio]methyl}nicotinate
ethyl 6-{4-[(benzylsulfonyl)carbamoyl]piperidin-1-yl}-5-cyano-2-{[(3-methoxy-3-oxopropyl)thio]methyl}nicotinate
ethyl 6-{4-[(benzylsulfonyl)carbamoyl]piperidin-1-yl}-5-cyano-2-[(1S)-2-ethoxy-1-methyl-2-oxoethoxy]nicotinate
ethyl 6-{4-[(benzylsulfonyl)carbamoyl]piperidin-1-yl}-5-cyano-2-[(4-oxopentyl)oxy]nicotinate
ethyl 6-{4-[(benzylsulfonyl)carbamoyl]piperidin-1-yl}-5-cyano-2-{[(2-ethoxy-2-oxoethyl)thio]methyl}nicotinate
ethyl 6-{4-[(benzylsulfonyl)carbamoyl]piperidin-1-yl}-5-cyano-2-({[2-(dimethylamino)-2-oxoethyl]thio}methyl)nicotinate
ethyl 2-[(2-azetidin-1-yl-2-oxoethyl)thio]-6-{4-[(benzylsulfonyl)carbamoyl]piperidin-1-yl}-5-cyanonicotinate
ethyl 6-{4-[(benzylsulfonyl)carbamoyl]piperidin-1-yl}-5-cyano-2-[2-(dimethylamino)-2-oxoethoxy]nicotinate
ethyl 6-{4-[(benzylsulfonyl)carbamoyl]piperidin-1-yl}-5-cyano-2-[(2-ethoxy-2-oxoethyl)thio]nicotinate
ethyl 6-{4-[(benzylsulfonyl)carbamoyl]piperidin-1-yl}-5-fluoro-2-[(2-oxopyrrolidin-1-yl)methyl]nicotinate
ethyl 6-{4-[(benzylsulfonyl)(methyl)carbamoyl]piperidin-1-yl}-5-cyano-2-[(2-oxopiperidin-1-yl)methyl]nicotinate
methyl 6-{4-[(benzylsulfonyl)carbamoyl]piperidin-1-yl}-5-chloro-2-[(2-oxopiperidin-1-yl)methyl]nicotinate, and
propyl 6-{4-[(benzylsulfonyl)carbamoyl]piperidin-1-yl}-5-fluoro-2-[(2-oxopyrrolidin-1-yl)methyl]nicotinate;
or a pharmaceutically acceptable salt thereof.

23. A pharmaceutical composition comprising a compound according to claim 1 and a pharmaceutically acceptable adjuvant, diluent, and/or carrier.

24-26. (canceled)

27. A method of treatment of a platelet aggregation disorder comprising administering to a patient suffering from such a disorder a therapeutically effective amount of a compound according to claim 1.
